(19) United States
(12) Patent Application Publication
(10) Pub. No.: US 2025/0154528 A1
(43) Pub. Date: May 15, 2025

(54) ADENO-ASSOCIATED VIRAL VECTORS AND USES THEREOF

(71) Applicant: The Broad Institute, Inc., Cambridge, MA (US)

(72) Inventors: Ken Y. CHAN, Cambridge, MA (US); Benjamin E. DEVERMAN, Cambridge, MA (US); FatmaElzahraa Abdelmouty EID, Cambridge, MA (US)

(73) Assignee: The Broad Institute, Inc., Cambridge, MA (US)

(21) Appl. No.: 18/834,583

(22) PCT Filed: Jan. 31, 2023
(86) PCT No.: PCT/IB2023/050844

§ 371 Date: Jul. 30, 2024

Related U.S. Application Data
(60) Provisional application No. 63/305,508, filed on Feb. 1, 2022.
(60) Provisional application No. 63/342,001, filed on May 13, 2022.
(60) Provisional application No. 63/343,010, filed on May 17, 2022.
(60) Provisional application No. 63/476,705, filed on Dec. 22, 2022.

(51) Int. Cl. C12N 15/86; C07K 14/005; C12N 15/10

(52) U.S. Cl. CPC C12N 15/86; C07K 14/005; C12N 15/1034; C12N 2750/14122; C12N 2750/14143; C12N 2750/14145

(57) ABSTRACT

The invention provides adeno-associated viral vectors and methods of using such vectors for cell transduction.

### CROSS REFERENCE TO RELATED APPLICATIONS

The present application claims priority to U.S. Provisional Applications No. 63/476,705, filed Dec. 22, 2022, 63/343,010, filed May 17, 2022, 63/342,001, filed May 13, 2022, and 63/305,508, filed Feb. 1, 2022, the entire contents of which are hereby incorporated by reference in their entirety.

### STATEMENT OF RIGHTS TO INVENTIONS MADE UNDER FEDERALLY SPONSORED RESEARCH

This invention was made with government support under Grants No. UG3MH120096, UG3MH120096, U42 OD027094, and P51-OD0101107 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Dec. 21, 2022, is named 167741_049102_PRO3_SL.xml and is 174,116,947 bytes in size.

### BACKGROUND OF THE INVENTION

Engineering novel functions into proteins while retaining desired traits is a key challenge for developers of viral vectors, antibodies, and inhibitors of medical and industrial value. For instance, to be harnessed as a viable gene therapy vector, an adeno-associated virus (AAV) capsid must simultaneously exhibit high production yield and efficiently target the cell type(s) relevant to a specific disease across preclinical models to patients. A common approach for developing AAV capsids with novel tropisms is to funnel a random library of peptide-modified capsids through multiple rounds of selection to identify a few top-performing candidates. This approach has produced modified capsids that more efficiently transduce cells throughout the central nervous system (CNS), photoreceptors, brain endothelial cells, and skeletal muscle. These rare capsids can then be diversified to screen even more enhanced tropisms, high production yield, or cross-species functionality. However, variants optimized for one trait can be difficult to optimize for other traits, and the protein sequence space is too vast to effectively sample by chance for rare variants that are enhanced across multiple traits. As a result, AAV engineering teams often devote many years and significant resources to developing capsids that ultimately fail to be optimized across multiple traits essential for preclinical and clinical translation.

Therefore, there is a need for improved adeno-associated viral vectors for multiple traits; for example, capsids that work across species to target organs of interest.

### SUMMARY OF THE INVENTION

As described below, the present invention features adeno-associated viral vectors and methods of using such vectors.

In one aspect, the disclosure features an adeno-associated virus (AAV) capsid polypeptide containing a peptide inserted within the capsid polypeptide. The peptide contains an amino acid sequence selected from one or more of RPNRDTS (SEQ ID NO: 144800); MDGQRRI (SEQ ID NO: 132518); ETNRAGR (SEQ ID NO: 116028); TGRVDSR (SEQ ID NO: 149619); NMTRARD (SEQ ID NO: 136472); GEKPKFT (SEQ ID NO: 164722); MEPRQRT (SEQ ID NO: 132640); and variants thereof containing a substitution or deletion of one or two amino acids.

In another aspect, the disclosure features an adeno-associated virus (AAV) capsid polypeptide containing a peptide inserted within the capsid polypeptide. The peptide contains a motif selected from those listed in Tables 2-27.

In another aspect, the disclosure features a viral particle containing the AAV capsid polypeptide of any of the aspects provided herein, or embodiments thereof.

In another aspect, the disclosure features a polynucleotide encoding the capsid polypeptide of any of the aspects provided herein, or embodiments thereof.

In another aspect, the disclosure features a library of adeno-associated virus (AAV) capsid polypeptides or polynucleotides encoding the same, where the library contains two or more capsid polypeptides of any of the aspects provided herein, or embodiments thereof.

In another aspect, the disclosure features a library of adeno-associated virus (AAV) capsid polypeptides or polynucleotides encoding the same, where the library contains two or more capsid polypeptides each containing a peptide with a sequence selected from one or more of SEQ ID NOs: 1-199427 and 200028-201544.

In another aspect, the disclosure features a composition containing an adeno-associated virus (AAV) capsid any one of the aspects provided herein, or embodiments thereof.

In another aspect, the disclosure features a method for screening a library of adeno-associated virus (AAV) capsid polypeptides for a trait of interest. The method involves A) administering to an organism or contacting a population of cells with AAV particles containing the library of any of the aspects provided herein, or embodiments thereof. The method also involves B) identifying in the library those particles demonstrating the trait of interest in the organism and/or in/on the cells.

In another aspect, the disclosure features a viral particle identified by the method of any of the aspects provided herein, or embodiments thereof.

In another aspect, the disclosure features a kit suitable for use in the method of any of the aspects provided herein, or embodiments thereof, where the kit contains adeno-associated virus (AAV) particles containing the capsid polypeptides of any of the aspects provided herein, or embodiments thereof, or polynucleotides encoding the same.

In any of the aspects provided herein, or embodiments thereof, the capsid is an AAV1, AAV2, AAV3, AAV3b, AAV4, AAV5, AAV6, AAV7, AAV8, AAV9, AAV9 K449R, rh.10, rh.8, or LK03 capsid polypeptide.

In any of the aspects provided herein, or embodiments thereof, the peptide is inserted in Loop VIII of the capsid polypeptide. In any of the aspects provided herein, or embodiments thereof, the peptide is inserted between amino acids 565 and 605 of an AAV9 K449R amino acid sequence, or at an equivalent insertion position in another AAV polypeptide. In any of the aspects provided herein, or embodiments thereof, the peptide is inserted between amino acids 575 and 595 and 600 of an AAV9 K449R amino acid sequence, or at an equivalent insertion position in another AAV polypeptide. In any of the aspects provided herein, or embodiments thereof, the peptide is inserted between amino acids 588 and 589 of an AAV9 K449R amino acid sequence, or at an equivalent insertion position in another AAV polypeptide.

In any of the aspects provided herein, or embodiments thereof, a viral particle containing an AAV capsid containing the peptide has increased transduction efficiency for a cell of interest relative to an AAV capsid lacking the peptide. In any of the aspects provided herein, or embodiments thereof, the cell of interest is a liver cell, brain cell, brain endothelial cell, kidney cell, spinal cord cell, spleen cell, nerve cell, or a cell of the spinal cord, heart, or lungs.

In any of the aspects provided herein, or embodiments thereof, transduction efficiency is increased by at least about 10%, 25%, 50%, 100%, 200% or more relative to an AAV capsid lacking the peptide.

In any of the aspects provided herein, or embodiments thereof, a viral particle containing an AAV capsid containing the peptide has increased production fitness relative to an AAV capsid lacking the peptide. In any of the aspects provided herein, or embodiments thereof, production fitness is increased by at least about 10%, 25%, 50%, 100%, 200% or more relative to an AAV capsid lacking the peptide.

In any of the aspects provided herein, or embodiments thereof, a viral particle containing an AAV capsid containing the peptide has increased biodistribution in an organ of interest relative to an AAV capsid lacking the peptide. In any of the aspects provided herein, or embodiments thereof, the organ of interest is selected from one or more of brain, heart, lung, kidney, spleen, and liver.

In any of the aspects provided herein, or embodiments thereof, the AAV capsid polypeptide is an AAV9 K449R capsid polypeptide and shares at least 85% sequence identity to an amino acid sequence selected from one or more of:

AAV-BI151
(SEQ ID NO: 199456)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQRPNRDTSAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI152
(SEQ ID NO: 199457)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQMDGQRRIAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI153
(SEQ ID NO: 199458)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQETNRAGRAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI154
(SEQ ID NO: 199459)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQTGRVDSRAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI155
(SEQ ID NO: 199460)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQNMTRARDAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI156
(SEQ ID NO: 199461)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQGEKPKFTAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
and
AAV-BI157
(SEQ ID NO: 199462)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQMEPRQRTAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*.

In any of the aspects provided herein, or embodiments thereof, the capsid polypeptides are each capable of encapsidating a polynucleotide sequence to form viral particles.

In any of the aspects provided herein, or embodiments thereof, the peptide sequences are selected from one or more of SEQ ID NOs: 1-157927 and 200028-201544.

In any of the aspects provided herein, or embodiments thereof, where the capsid is an AAV1, AAV2, AAV3, AAV3b, AAV4, AAV5, AAV6, AAV7, AAV8, AAV9, AAV9 K449R, rh.10, rh.8, or LK03 capsid polypeptide. In any of the aspects provided herein, or embodiments thereof, the capsid polypeptide is derived from an AAV9 K449R capsid polypeptide.

In any of the aspects provided herein, or embodiments thereof, the library contains an amino acid sequence motif selected from one or more of:
[RQMT] [RNQP] [RQPS] [ANQGST] **G; [QTV] [ANQGKP] [RNGSTY] * [ARGT] * [GP];
[RKSV] [FPS] ** [RT] [QGS] [GS]; [GST] * [QT] [NQ]R*G; [TV] ** [NS]R [QGM]G;
[RNQT] [ANPS] [RNS] ** [NQGS]G; T [NGS] **R [AQS]G; NR** [NG] [QG]A;
R**QGGG (SEQ ID NO: 199501); [QF] [KS] [RN] ** [NP] [AP]; QR**S [TV]A (SEQ ID NO: 199502); [QKM] *G[RSV] [KT] *G;
[QMTV] [RN] * [ANV] [RQS] * [GS]; [NQ]R [NGP] [NQS] **A;
[QT] * [RT] [RT] * [NQG] [AG]; NTR**SA (SEQ ID NO: 199503);
QRP* [AS] * [AS]; RQ**TNA (SEQ ID NO: 199504); QRP** [MV] [AS];
[RN] *N [RS] * [QG]G; [MTV]R [PS] * [QT] *G; [RY]S* [QK] *Q [GS]; MRG**MG (SEQ ID NO: 199505); [GV] * [NT] *R [QS]G; [ST] [RQ] [RS]T**A; R*S*STP (SEQ ID NO: 199506); QR**TNG (SEQ ID NO: 199507); Q*RQT*P (SEQ ID NO: 199508); [NQ]RQ* [GS] *A; TR**NNA (SEQ ID NO: 199509);
[RT]S* [RQ] [QS] *A; GQ*RV*G (SEQ ID NO: 199510); T*TSR*G (SEQ ID NO: 199511); TRG**TG (SEQ ID NO: 199512); NR* [GT] * [TV]G; T*RT*SA (SEQ ID NO: 199513); MG*R*GA (SEQ ID NO: 199514); [NQ]R* [NQ]S*A;
RQ*PT*A (SEQ ID NO: 199515); T*T*RSG (SEQ ID NO: 199516); T*RGS*P (SEQ ID NO: 199517); TR**TMG (SEQ ID NO: 199518); R*TS*SP (SEQ ID NO: 199519); N**QRSA (SEQ ID NO: 199520); [QT] [RK] *S*[TY]A;
QR*PA*G (SEQ ID NO: 199521); RS*S*GG (SEQ ID NO: 199522); RTS*S*P (SEQ ID NO: 199523); TRQ*T*G (SEQ ID NO: 199524); QR*S*TG (SEQ ID NO: 199525); R*NS*SP (SEQ ID NO: 199526); MR*G*QS (SEQ ID NO: 199527); N**SRQG (SEQ ID NO: 199528); NR*ST*A (SEQ ID NO: 199529);
RSQ*G*G (SEQ ID NO: 199530); T*RTN*A (SEQ ID NO: 199531); T*S*RMG (SEQ ID NO: 199532); TR**TQA (SEQ ID NO: 199533); and TRT**SG (SEQ ID NO: 199534); YSGK**G (SEQ ID NO: 199535); or polynucleotides encoding the same, where “*” represents any amino acid and square brackets denote a list of amino acids that may occur at a position.

In any of the aspects provided herein, or embodiments thereof, the library contains an amino acid sequence motif selected from one or more of:
[RQGKST] [ARNQGFPST] ** [ARNQGKMTV] [ANQGMSV]G;
[\hat{ }ADCEHILKPW] [ARNQGKPS] [ARNQGKST] [ARNQKPSTV] ** [NDGPS];
[NQKMFSTY] [RNGKT] [\hat{ }CEHILKFSWY] ** [RNQKMPSTV] [ANDEPS];
[\hat{ }ARDCEHILW] [RGLKPSY] * [NQGKPSTV] * [ANQGFST]G;
[\hat{ }ARDCEHILPW] [ARQGKPT] [RQGKPST] ** [ANGMST]G;
[\hat{ }ADCQEHILW] [NQGLKMPST] * [\hat{ }NCEHILMFWY] [ARNQGMTV] * [GS];
[RGMY] [ANQGPS] [NQKPST] ** [ANGKMST]G;
[NQGKFSTV] [RNQGKS] ** [ARNGKT] [NQGKFSYV] [ANDGS];
[\hat{ }ARDCEHILFW] * [ARNQGKMPT] [RQGKPSTV] * [AQGST]G;
[\hat{ }ARDCEHILPW] * [RNGKPST] * [ARNGKSV] [NQGKMST]G;
[RNKT] [RNQPS] * [RQGPS] [RQGS] * [APS];
[RNMPY] [RNQKPS] * [ANQTV] * [ANQGKTV] [DGPS];
[NQGKMFTV] [ARQGKMPTV] [RNQGKPTY] * [ARQKMST] *G;
[RNQMFST] [RNQGKT] * [ARNQPST] * [AQGSTY] [APS];
[QGKMSTV] * [ARNQGKMPT] [RNQGKT] [ARNSTV] *G;
[RMT] * [NQKT] * [ARQGT] [ANQS] [GP];
[RKMSV] **[ANQGKPS] [NGMSTV] [ANGKSTV] [DGS];
[NQKMPTYV] * [ARGKPTV] [NGST] [RNQKST] * [GPS];
[RNQKMSY] [RQKST] [ADQKPST] * [AKSTV] * [ADPS]; [RK] ** [NG] [AS] [GT]A;
[RQ] * [RNPST] [AQGST] [AQGSTV] * [GPS]; R [QPS] *S* [QGV]G;
[RKV] [NGKP] *N [ANGT] *G; [NQGLKFT] [NGKS] [QKMS] ** [ARQKSV] [ANDEG];
[RQMS] * [RNQKST] [NPST] * [ARNQGMS] [DGP]; [RKT] ** [PST] [QT] [ANG]G;
[NQKMT] [ARDKPST] [RNGKP] *G*G;
[RNQKMFT] [RDGKPST] [RNQGKPT] * [NQGST] * [AS];
[RNQMST] [RNDQKST] [RGKPST] [AQGKPST] ** [APSV];
[QKT] * [KS] * [AQGS] [NQS] [AS]; [KFV] * [ANK] * [ST] [KS] [AD];
[RQKMTYV] [RNDKPS] [RQGPS] [NDQGPST] ** [GP];
[NQMT]R* [NGPTV] [AQGS] * [GS]; [QMSY] [RKP] **S [AQGKV] [ADGS];
R [NS] ** [ANQ] [AQT]A; R [QS] **T [NQ] [AS]; [RQ] * [KPT] [GST] * [ANQGT]G;
[RN] [RQP] * [GPS]T*A; [RNMPT] * [RNGKST] [ANDQKPST] [RNGST] *A;
K [QPS] * [NMS] * [QV] [GS]; K* [NQG] * [NT] [AST]G; RN*Q*SG (SEQ ID NO: 199536); K* [ANGST] [NST] * [AMS] [GS]; KT*S*GA (SEQ ID NO: 199537);
[RKMT] [RQKPST]S* [ARSTV] *G; [KM] *N*GNA (SEQ ID NO: 199538);
N**S [GT] [GM]A; K* [GPS] [GT] [AT] *A; [QG]K* [GTV] [AMS] * [AN];
[RQT] * [RNQPT] [RNST] * [QGS]A; [NQ] [RK] ** [GS]TA; [TV] **NRQG (SEQ ID NO: 199539); R [AGPS] [NQPT] * [NGST] *G; R [MST]N** [QT] [GP];
[RNQ] * [PT] [GT] [RS] *A; RS**NTG (SEQ ID NO: 199540);
[RK] *[PS]G*[NQGT]A; Q*KSA*A (SEQ ID NO: 199541); KN*G*TA (SEQ ID NO: 199542); K*S*[AS]GA (SEQ ID NO: 199543); [NG]K*[AS] [GT] *A;
[GV]NS* [RK] *G; KT*S*SA (SEQ ID NO: 199544); [RM] [KP] *[AS]S*G;
VK**STG (SEQ ID NO: 199545); [QKT] [RQGMSTV] [AGKP] [NGST] **A;
PR*AT*G (SEQ ID NO: 199546); TRS*T*M (SEQ ID NO: 199547); T**RQQA (SEQ ID NO: 199548); KN*S*SG (SEQ ID NO: 199549); RNS*[AG] *G (SEQ ID NO: 199550); R*S* [AS]T [PS]; [RG] [KS]S* [GT] * [AG]; QR*NS*A (SEQ ID NO: 199551); R*SN*TG (SEQ ID NO: 199552); R* [NS] * [GT]GG;
RAP**NS (SEQ ID NO: 199553); R*NNS*G (SEQ ID NO: 199554);
[NK] * [GKP] [GST] * [GS]A; QK*GT*G (SEQ ID NO: 199555); T**NRGG (SEQ ID NO: 199556); VK*AS*A (SEQ ID NO: 199557); K*P*TGG (SEQ ID NO: 199558); K**QNQG (SEQ ID NO: 199559); R*S*TAP (SEQ ID NO: 199560);
K*S[NG] *[GS] [GS]; R*SN*NA (SEQ ID NO: 199561); RMP**GA (SEQ ID NO: 199562); [RW] [ANP]N[AKS] **A; KT**SGG (SEQ ID NO: 199563); R*P*TGA (SEQ ID NO: 199564); N**QRSA (SEQ ID NO: 199520); K*[NQ]ST*A (SEQ ID NO: 199565); MKN[TV] **A (SEQ ID NO: 199566); KGNN**G (SEQ ID NO: 199567); TKP**AA (SEQ ID NO: 199568); TR*GT*G (SEQ ID NO: 199569); KN*G*SA (SEQ ID NO: 199570); K*ASS*A (SEQ ID NO: 199571);
KLNS**G (SEQ ID NO: 199572); N**SRQG (SEQ ID NO: 199528); and
QNR*A*P (SEQ ID NO: 199573); R*P*AGA (SEQ ID NO: 199574); RT**STP (SEQ ID NO: 199575); SRTT*NG (SEQ ID NO: 199576); T*RT*NA (SEQ ID NO: 199577); TK*NS*G (SEQ ID NO: 199578); TRP**AA (SEQ ID NO: 199579); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position.

In any of the aspects provided herein, or embodiments thereof, a viral particle having a capsid containing the motif has increased binding to a cell of interest relative to a control viral particle.

In any of the aspects provided herein, or embodiments thereof, the library contains an amino acid sequence motif selected from one or more of: QSRT**P (SEQ ID NO: 199580); K[HP] [NT] *P* [NS]; RN*P*TS (SEQ ID NO: 199581); K**GPKD (SEQ ID NO: 199582); NRGQ**A (SEQ ID NO: 199583); A**NEKR (SEQ ID NO: 199584); TG**RSG (SEQ ID NO: 199585); TAN*R*G (SEQ ID NO: 199586); T*TNR*G (SEQ ID NO: 199587); QSR**NP (SEQ ID NO: 199588); T*T*RSG (SEQ ID NO: 199516); K**NPAN (SEQ ID NO: 199589); KM**PKD (SEQ ID NO: 199590); MSRN**A (SEQ ID NO: 199591); NDA**KK (SEQ ID NO: 199592); QR*GP*M (SEQ ID NO: 199593); RS*P*NA (SEQ ID NO: 199594); T*S*RMG (SEQ ID NO: 199532); T*TSR*G (SEQ ID NO: 199511); VAR*H*G (SEQ ID NO: 199595); or polynucleotides encoding the same, where “*” represents any amino acid and square brackets denote a list of amino acids that may occur at a position.

In any of the aspects provided herein, or embodiments thereof, a viral particle having a capsid containing the motif has increased binding to a liver cell relative to a control viral particle.

In any of the aspects provided herein, or embodiments thereof, the library contains an amino acid sequence motif selected from one or more of:
[GKMFSTYV] [NGKST] ** [ARNGKSV] [AQMFST]G;
[RNQKTY] * [NQGKPST] [ANGPST] * [NQGTV]G; [QLMFPSTYV]K* [GPSTV] * [QGST]G;
[RNQGKMPTY] [ARNQGPST] [ARNQGKPT] ** [AGKMSTV]G;
[RQGMTV] * [RQGKPST] [QGPST] * [ARQMPS] [DGPS];
[RNQGKMPST] * [AQGKMPSTV] [RNQGST] [ARNQGKST] *G;
[NGKTV] **[RNQGPSTV] [ARQGKMST] [ANQGKFST] [AGPS];
[RNGKMFSTY] [ANQLKMPT] * [ARDQGSTV] [ARQGKTV] * [GS];
[RQGKMFTV] [ARNQGPST] [RGKPST] * [\hat{ }DCEILFPWY] *G;
[RNQGIKMST] [RDLKMFST] [ANDQHMPST] ** [ARNGKMSV] [ANDEKPS];
[RNQGIKFV] * [ARNQPST] * [ARGKMTV] [ARNQGT] [DEGS];
[\hat{ }ARNCEHILP] [ANQGKMPST] [RNQGHKMST] [ARNQGKPTV] ** [ARGS];
[RNQGIMFT] [RKPSTV] [AQGHKPST] * [ARNIKST] * [EGPS];
[NQKMFSTV] [RKS] **[AGST] [ARNDKSTV] [ADEK];
[RNQKMSTY] [RNGKST] * [ARNQMPSTV] * [\hat{ }CEHILKFPWV] [ADPST];
K* [GMP] [QST] * [AQM] [GS]; [RQKTY] * [ARQKPST] [QGST] [ANGST] * [ADPS];
[NQFTY] * [KST] * [ARGS] [AQGKMTV]G; [RT] [RPT] ** [ANST] [NQT] [APS];
[RNKMPY] [ARNGLKS] * [ANQKST] * [AQGSV]G; [RNQSY] [KPST] ** [RNGMS] [QG]G;
[RKT] [PS] *[RPST] [QGS] *A; [RNQEKMS] [RNQT] [RGIPST] [NQGKPT] **[AEGFP];
[NQT]K* [AGPST] [GMS] * [AS]; [RNK] [RDLS] [RNPT]S** [GP];
[RK] * [NP] [GV] * [GT]A; [RQGMFSY] [KS] [NQGMPS] ** [NQGS] [GS];
[MFPV]K [ANPS]S** [GS]; [MF]K [NT] ** [QPT]A;
[GFT] * [GKT] * [RNQK] [KSTV] [GS]; [QGM] [RKS] [NGPS] * [AST] *A;
[RT] * [KP] * [QT] [QG]A; [GM] [HP]K** [AP]A; [TV] * [NK] *S [NS] [AS];
[QGMT] [RK] * [GPTV] [AS] *G; [RV] [KP] *NT*G; M*SKS*A (SEQ ID NO: 199596); A**NEKR (SEQ ID NO: 199584); PR*AT*G (SEQ ID NO: 199546);
NK [AGP] [AQV] ** [DGS]; [MS] **K [ST] [GT]G; [RQK] [ARF] **T [AGS]G;
[QF]K** [AN] [QS]S; [NMT]K[ANP] *G*G; R**KEEK (SEQ ID NO: 199597);
[RGV] [KP] *[AS] [ST] *[AS]; NK[NS] *G*A (SEQ ID NO: 200021); QK*GT*G (SEQ ID NO: 199555); TK*NS*G (SEQ ID NO: 199578); TGK**[AT]A (SEQ ID NO: 199598); R**AGVG (SEQ ID NO: 199599); [NT]K*[TV] *KD; MKS**TG (SEQ ID NO: 199600); G*KSV*G (SEQ ID NO: 199601);
[KM] * [NS] * [GS] [NG]A; TNK**QG (SEQ ID NO: 199602);
K [DKPS] [RNDQ] * [GK] *[AD]; NAR*T*G (SEQ ID NO: 199603); MR*NQ*G (SEQ ID NO: 199604); [FT] *K[AT] *QA; KT**GGA (SEQ ID NO: 199605);
V*NKV*G (SEQ ID NO: 199606); FKG**SA (SEQ ID NO: 199607); FNK**QG (SEQ ID NO: 199608); GPK*T*A (SEQ ID NO: 199609); KQS*S*P (SEQ ID NO: 199610); [NS] *KG*[ST]A; GP*G*KG (SEQ ID NO: 199611); RDKS**A (SEQ ID NO: 199612); R*S*STP (SEQ ID NO: 199506); KT**AGG (SEQ ID NO: 199613); FK**TQG (SEQ ID NO: 199614); FGK**TG (SEQ ID NO: 199615); NKTG**A (SEQ ID NO: 199616); TK**TYG (SEQ ID NO: 199617);
TKPG**G (SEQ ID NO: 199618); KP*T*GG (SEQ ID NO: 199619); TGK**SA (SEQ ID NO: 199620); KPN*S*A (SEQ ID NO: 199621); TGKS**A (SEQ ID NO: 199622); T*RT*SA (SEQ ID NO: 199513); V*KS*TG (SEQ ID NO: 199623); F*K*TSA (SEQ ID NO: 199624); FGK**SG (SEQ ID NO: 199625);
K*GG*AG (SEQ ID NO: 199626); KPS*N*A (SEQ ID NO: 199627); QR*NS*A (SEQ ID NO: 199551); R**QGGG (SEQ ID NO: 199501); RP*N*GG (SEQ ID NO: 199628); TK**TQG (SEQ ID NO: 199629); TKSS**A (SEQ ID NO: 199630); and V*KSQ*G (SEQ ID NO: 199631); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position.

In any of the aspects provided herein, or embodiments thereof, a viral particle having a capsid containing the motif has increased transduction efficiency for a liver cell relative to a control viral particle.

In any of the aspects provided herein, or embodiments thereof, the library contains an amino acid sequence motif selected from one or more of:
[ARDTV] **[NKP] [REG] [QEGK] [RGK]; NAR**GG (SEQ ID NO: 199632);
N[AR] [RG]Q** [AG]; VR**SSA (SEQ ID NO: 199633); TG**RSG (SEQ ID NO: 199585); R*KDS*A (SEQ ID NO: 199634); T*T*RSG (SEQ ID NO: 199516);
T*TNR*G (SEQ ID NO: 199587); T*TSR*G (SEQ ID NO: 199511); G**SIRS (SEQ ID NO: 199635); GQSS**R (SEQ ID NO: 199636); M*KP*RD (SEQ ID NO: 199637); NDA**KK (SEQ ID NO: 199592); NK*DR*G (SEQ ID NO: 199638); QRP*A*A (SEQ ID NO: 199639); SP**RGG (SEQ ID NO: 199640);
V*N*SSA (SEQ ID NO: 199641); or polynucleotides encoding the same, where “*” represents any amino acid and square brackets denote a list of amino acids that may occur at a position.

In any of the aspects provided herein, or embodiments thereof, a viral particle having a capsid containing the motif has increased binding to a liver cell relative to a control viral particle.

In any of the aspects provided herein, or embodiments thereof, the library contains an amino acid sequence motif selected from one or more of
[NDGILST]K* [RNGHKPSTV] * [ANDGKFSY] [ADQST];
[RQGKMFPS] [RNGHPST] [RQGKPST] ** [ARQGMPSTV] [AEGS];
[MFTYV]K** [ANST] [AQSTY]G;
[NQGKMFSYV] [RQGLKS] * [ANDQKPS] [ARNQGMSTV] *G;
[RDQEK] [ARGLMST] * [ANGKPT] * [RNKMS] [AELM];
[\hat{ }ARDCEHFPWV]K [ANDQMPST] ** [RQGKMT] [NDEG];
[\hat{ }ACGHILPW] [RNDQGLKS] [\hat{ }DCELKFWYV] [\hat{ }RNCEILMFWY] ** [RNDEGKFPS];
[NKMSTW] [ARKSTV] [NQGPS] [AQGKSV] **A;
[RNQGKTYV] * [ANQGMPST] [ANQGKST] * [AQGKMS] [AEGS];
[NQGIKFSTY]K [NDQHPS] * [ARNGKT] * [ADEPS];
[NMFTYV] [KPS] * [AQGKPTV] * [AQGKS] [DEGS];
[\hat{ }ADCEHILPW] [ARNQGKFPT] ** [ARNQKMSTY] [ARNQGMS] [ADEGPS];
[NQIKFY] [ADQLK] [ANGHM] **[ARQK] [NDGK];
[ARNDQGSTV] ** [RNQGKP] [RQEGKMT] [NEGKFS] [ARGK];
[NQGMT]K* [AQGPSTV] [AGMST] * [ANGS]; [DQKS] [QKMST] ** [AGST]K [DEK];
[NQWV]K [GST] **SG; K** [ANQS] [ANQT] [ADGK] [ARDEG];
K**[NQSTY]S[AQGKT] [EGPS]; [RNQGT] *[AGST] [QGKT] [RGKT] *G;
[RNQGMST] [RNQGKPSV] [ANGPST] * [ARQGIKST] * [AGS];
[RNQGKTV] [RNQGKPS] * [ANQGSTV] [NGKMST] * [AGPS];
[QGKMT] [GFP]K* [NQEGTY] * [AQGY]; [NT] [AD] *K [RS] * [LP];
[NQMFTV] *K* [AQGMS] [AQGKTV] [EG];
[RGIKF] * [ARNQGT] * [AKMT] [ARGKS] [DEG];
[QMFT] [RNQGMPS] [RKP] [ANGST] **G; [NGMFT] [NGKS]K[DQT] **[GSW];
[GKFTY] * [AKPT] * [RNQST] [AGKST] [GS]; [QFS]K [GT] ** [ST]A;
T[RNS] [ARGK] ** [RQGT] [EG]; T*N*SKG (SEQ ID NO: 199642);
[GSTYV] *K [QT] [NGS] * [GS]; [KMFPSY] [LK] *S* [ANQIST]G; K [KS] [DST] *S*G;
[RNQMPTV] * [GKT]S [ARQKS] * [AGS]; G*K*TAA (SEQ ID NO: 199643);
K* [ANS] [GS] [GT] * [ADP]; [RQGKP] * [GKP] [NDPT] [AQMS] *A;
[MFT] *K[APT] *[RQ] [ADS]; M*SKS*A (SEQ ID NO: 199596);
TK [NMP] ** [ANQ]A; [NQGS]K**G [QGF]G; [NQF] [QS]K* [AS] *G;
GK [GT] [QT] **G; [RGT] [DGP]K [NQS] **A; TGK** [AST]A (SEQ ID NO: 199644); K [DS] [RN] *G* [AG]; [GMPTV] *K [ASTV] * [QST]G;
[ST]K* [AQ] * [QS]A; K*A [TV] * [RK]D; [MF]KN** [QP]A; Q [RQY] [KP] [ST] **A;
[NT] *KS* [ST] [AP]; TG**MKG (SEQ ID NO: 199645);
[QT] [RG] *S* [KT] [AP]; [FV] *K*TSA (SEQ ID NO: 199646);
QK[GT] [NS] **A; T**KS [GS]G (SEQ ID NO: 200022); [NP]K*S*GG (SEQ ID NO: 199647); NK**G[ST]A (SEQ ID NO: 199648); [QG]KN**SA (SEQ ID NO: 199649); TKS**SN (SEQ ID NO: 199650); MKNT**A (SEQ ID NO: 199651); Q*K[GS] *[NV]G; Q**KSNA (SEQ ID NO: 199652); NN**SKG (SEQ ID NO: 199653); M*N*GNA (SEQ ID NO: 199654); KTQ*S*S (SEQ ID NO: 199655); T**SGTP (SEQ ID NO: 199656); RS*S*GG (SEQ ID NO: 199522);
MG*R*GA (SEQ ID NO: 199514); [QT] *K[NS] [ST] *G; Q*KP*QG (SEQ ID NO: 199657); KA**GDR (SEQ ID NO: 199658); V*N*SSA (SEQ ID NO: 199641);
K*TG*RE (SEQ ID NO: 199659); M*K*TSG (SEQ ID NO: 199660); T*K*SSA (SEQ ID NO: 199661); YQK*S*S (SEQ ID NO: 199662); MK*T*TA (SEQ ID NO: 199663); KPN*S*A (SEQ ID NO: 199621); TK**GMG (SEQ ID NO: 199664); MPK*S*S (SEQ ID NO: 199665); DL*KP*K (SEQ ID NO: 199666);
KGNT**A (SEQ ID NO: 199667); MK*G*TG (SEQ ID NO: 199668); N**STMA (SEQ ID NO: 199669); NK**SDK (SEQ ID NO: 199670); T*K*SNS (SEQ ID NO: 199671); T*KG*VG (SEQ ID NO: 199672); TKQ**SA (SEQ ID NO: 199673); and V*NKV*G (SEQ ID NO: 199606); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position.

In any of the aspects provided herein, or embodiments thereof, a viral particle having a capsid containing the motif has increased binding to a liver cell relative to a control viral particle.

In any of the aspects provided herein, or embodiments thereof, the library contains an amino acid sequence motif selected from one or more of: KQ**AKD (SEQ ID NO: 199674); NR**GGA (SEQ ID NO: 199675); KKD**RD (SEQ ID NO: 199676); QRNS**A (SEQ ID NO: 199677); NRGQ**A (SEQ ID NO: 199583); KKD**KD (SEQ ID NO: 199678); R*KDS*A (SEQ ID NO: 199634); RN**SGA (SEQ ID NO: 199679); RQ*PT*A (SEQ ID NO: 199515); or polynucleotides encoding the same, where “*” represents any amino acid and square brackets denote a list of amino acids that may occur at a position.

In any of the aspects provided herein, or embodiments thereof, a viral particle having a capsid containing the motif has increased binding to a brain endothelial cell relative to a control viral particle.

In any of the aspects provided herein, or embodiments thereof, the library contains an amino acid sequence motif selected from one or more of:
[QKMPT] [NKMPT] [ARGKS] [AQGT] ** [AG]; [TV]KPS**G (SEQ ID NO: 199680);
[QT] [KS] *G [NKT] *G; [KMT] [RT] [GS] ** [NMT]G;
[RQMT] [RQGK] [QPST] * [RGTV] *G; [GM]K [PS] ** [NT]G; KPT**MA (SEQ ID NO: 199681); [QGKT] * [GPT] [NQGT] [RKT] *G; [KT] [KS]SS** [AG];
[NGS] [AK] * [GKS] [NST] * [AP]; [GK] *T* [RT] [QS]G;
[QKT] [GKF] ** [RGT] [GSY]G; [QKS] [RGKT] * [NT] [ANGT] *G; T**NRGG (SEQ ID NO: 199556); [NK] [RY] *T*[TV]G; Q*PTS*A (SEQ ID NO: 199682);
[FS] [NG]K*[GS] *G; QR**STA (SEQ ID NO: 199683); [QMS]K*G*[NT]G;
K*ST*NG (SEQ ID NO: 199684); QRSS**G (SEQ ID NO: 199685);
[KV] [NK] * [GV] *S [AG]; N [RT] [AR] * [NT] *A; KN*GQ*G (SEQ ID NO: 199686); K*S*QSA (SEQ ID NO: 199687); [RN] [ST] [KS] *S* [GP]; QKN*A*A (SEQ ID NO: 199688); RP**MAG (SEQ ID NO: 199689); N**STMA (SEQ ID NO: 199669); N*K*GGG (SEQ ID NO: 199690); KTT**GG (SEQ ID NO: 199691); YKQ**GG (SEQ ID NO: 199692); N*K*SNP (SEQ ID NO: 199693);
NKN*G*A (SEQ ID NO: 199694); S**KTGG (SEQ ID NO: 199695); GN*VK*G (SEQ ID NO: 199696); M*SKS*A (SEQ ID NO: 199596); MK**SAG (SEQ ID NO: 199697); N*K*SQG (SEQ ID NO: 199698); NRPS**P (SEQ ID NO: 199699); QKT**GG (SEQ ID NO: 199700); RS*Q*QS (SEQ ID NO: 199701);
and T*RT*GG (SEQ ID NO: 199702); or polynucleotides encoding the same, where “*” represents any amino acid and square brackets denote a list of amino acids that may occur at a position.

In any of the aspects provided herein, or embodiments thereof, a viral particle having a capsid containing the motif has increased binding to a brain endothelial cell relative to a control viral particle.

In any of the aspects provided herein, or embodiments thereof, the library contains an amino acid sequence motif selected from one or more of: QSRT**P (SEQ ID NO: 199580); MSRN**A (SEQ ID NO: 199591); KKD**RD (SEQ ID NO: 199676); NRGQ**A (SEQ ID NO: 199583); KKD**KD (SEQ ID NO: 199678); KKD*K*D (SEQ ID NO: 199703); NR**GGA (SEQ ID NO: 199675); and R*KDS*A (SEQ ID NO: 199634); or polynucleotides encoding the same, where “*” represents any amino acid and square brackets denote a list of amino acids that may occur at a position.

In any of the aspects provided herein, or embodiments thereof, a viral particle having a capsid containing the motif has increased binding to a brain endothelial cell relative to a control viral particle.

In any of the aspects provided herein, or embodiments thereof, the library an amino acid sequence motif selected from one or more of: NR**GGA (SEQ ID NO: 199675); [STY] [KP] * [QS] [GSV] *G; [GM] ** [GT] [GK] [NF]G; QRPN**A (SEQ ID NO: 199704); [QS]K[GT] *S*G; Q*K*AQG (SEQ ID NO: 199705); Q* [GK] [NS] [KS] *G; [QY] [RK]P* [AT] * [AP]; [QM]K*G*TG (SEQ ID NO: 199706); TK*N*QG (SEQ ID NO: 199707); K*ST*SG (SEQ ID NO: 199708); KN*G*SA (SEQ ID NO: 199570); KN*GQ*G (SEQ ID NO: 199686); MK**SQG (SEQ ID NO: 199709); TGT*R*G (SEQ ID NO: 199710); GK*ST*A (SEQ ID NO: 199711); MK*GS*G (SEQ ID NO: 199712); MSK**AG (SEQ ID NO: 199713); K*PTT*G (SEQ ID NO: 199714); KP*T*GG (SEQ ID NO: 199619); MP**SGS (SEQ ID NO: 199715); Q*K*SNG (SEQ ID NO: 199716); QKY*T*G (SEQ ID NO: 199717); R*PS*QG (SEQ ID NO: 199718); T**PTAG (SEQ ID NO: 199719); and TGKS**A (SEQ ID NO: 199622); or polynucleotides encoding the same, where “*” represents any amino acid and square brackets denote a list of amino acids that may occur at a position.

In any of the aspects provided herein, or embodiments thereof, a viral particle having a capsid containing the motif has increased transduction efficiency for a brain endothelial cell relative to a control viral particle.

In any of the aspects provided herein, or embodiments thereof, the library contains an amino acid sequence motif selected from one or more of: N**SRQG (SEQ ID NO: 199528); SP**RGG (SEQ ID NO: 199640); NQ**RSA (SEQ ID NO: 199720); QKY*T*G (SEQ ID NO: 199717); S*QQR*G (SEQ ID NO: 199721); MRG**MG (SEQ ID NO: 199505); NR**GGA (SEQ ID NO: 199675); NRGQ**A (SEQ ID NO: 199583); T**NRGG (SEQ ID NO: 199556); T*S*RMG (SEQ ID NO: 199532); T*TNR*G (SEQ ID NO: 199587); and TAN*R*G (SEQ ID NO: 199586); or polynucleotides encoding the same, where “*” represents any amino acid and square brackets denote a list of amino acids that may occur at a position.

In any of the aspects provided herein, or embodiments thereof, a viral particle having a capsid containing the motif has increased binding to a brain endothelial cell relative to a control viral particle.

In any of the aspects provided herein, or embodiments thereof, the library contains an amino acid sequence motif selected from one or more of:
[QKMFT] [RNQP] [RQKPS] [ARDG] **G;
[RQGMSTY] [ARNKP] [RNGKPST] ** [RNQGKMST] [AEG];
[QGF] [GPT] [RK] * [NMS] *G; [RK] [NQ] [PT] ** [AT]G;
[RMT] [RQGT] [NPST] * [RQV] *G; [KMST] * [NQGT] [RQGS] [RQT] *G;
[RNY] [RKP]Q** [QGT] [AG]; [NQKT] [RKP] ** [NGS] [NQGTV]A;
[RKMST] [NGKPS] * [ARQS] * [ANQGTY] [AS]; [RKMP] [NKS] * [ANQS] * [AGSV]G;
[RGMFT] [NK] * [AGPV] [NGKTV] *G; [RKMTY] * [RKPST] [AGST] [GST] * [GPS];
SK*GN*A (SEQ ID NO: 199722); KP [AP] **G [AG]; [NST] [RK] *T* [NGV]G;
[RNT] * [GKT] * [ARGK] [QMST]G; [GK] [NP] [KS] * [NS] * [AS]; QK*GT*G (SEQ ID NO: 199555); [RQK] ** [ANT] [RT] [GT]G; [RK] [NF] ** [GTV]SG;
[GK] [KP] * [SV]S* [NP]; TG*NK*A (SEQ ID NO: 199723);
[NT] [RT] [PS] [KS] ** [AP]; QKY*T*G (SEQ ID NO: 199717); RS**TQS (SEQ ID NO: 199724); [RN] * [NT] [RS] *GG; KTQ**AS (SEQ ID NO: 199725);
RS**GGG (SEQ ID NO: 199726); KPP*T*G (SEQ ID NO: 199727);
[KY] * [QP]T* [GT]G; S*TT*NG (SEQ ID NO: 199728); KSPT**A (SEQ ID NO: 199729); SRTT**G (SEQ ID NO: 199730); [GY]K[QT] [QT] **G; Q**KSNA (SEQ ID NO: 199652); K*NST*A (SEQ ID NO: 199731); TRS*T*G (SEQ ID NO: 199732); K*S*SGA (SEQ ID NO: 199733); N**SRQG (SEQ ID NO: 199528); VRP*T*G (SEQ ID NO: 199734); Y*T*SKG (SEQ ID NO: 199735);
TR*VS*G (SEQ ID NO: 199736); TG**RSG (SEQ ID NO: 199585); K*P*SGG (SEQ ID NO: 199737); KTS**GG (SEQ ID NO: 199738); N**GQKG (SEQ ID NO: 199739); N*GNR*A (SEQ ID NO: 199740); QR*NS*A (SEQ ID NO: 199551); QRP*S*A (SEQ ID NO: 199741); R*QN*TG (SEQ ID NO: 199742);
RP*T*AP (SEQ ID NO: 199743); SRTT*NG (SEQ ID NO: 199576); and
TKSS**A (SEQ ID NO: 199630); or polynucleotides encoding the same, where “*” represents any amino acid and square brackets denote a list of amino acids that may occur at a position.

In any of the aspects provided herein, or embodiments thereof, a viral particle having a capsid containing the motif has increased transduction efficiency for a brain endothelial cell relative to a control viral particle.

In any of the aspects provided herein, or embodiments thereof, the library contains an amino acid sequence motif selected from one or more of:
[\hat{ }ADCEGHILWY] * [\hat{ }DCEGHILFWY] [ARNGKMPST] [ARNQGISTV] *G;
[\hat{ }ADCEHILMWY] [\hat{ }DCEHILFPWY] [\hat{ }RCEHILMFW] * [\hat{ }NDCEHLFPWY] *G;
[\hat{ }ADCEHILMW] [\hat{ }CEGHIFWYV] [\hat{ }CEHILFPW] [\hat{ }CEHILKMFWY] **G;
[RNDQKMPV] [ARNQMPST] ** [ARNQGKST] [ARNDQGKTV] [ARDIKPS];
[\hat{ }ARDCEHILW] [ARQGKPST] ** [ARNQKMSTV] [ANQGKMTYV]G;
[\hat{ }ACEHILMFSW] [\hat{ }CEHIKFWYV] * [\hat{ }DCQEHLFWY] [\hat{ }DCEHLFTWY] * [AEGLKP];
[RQKMSTV] ** [ARNQKPSTV] [ARNQGMSTV] [ARNQGSTV]G;
[\hat{ }RDCEHLPW] [\hat{ }CEHILMPSWV] [\hat{ }ANCEILMFWV] * [\hat{ }DCQILMFWV] * [NDQEGMPSY];
[RNQGKMS] [RQKST] [\hat{ }RDCEHILFWY] * [ARNMSTV] * [APS];
[\hat{ }ADCEHLFPW] * [ARNQGPST] [RQGKSTY] * [ANGKMSTV]G;
[\hat{ }ADCEHIK] [\hat{ }CQEHILFWY] [ARNQGKMPS] ** [ANQGKMSV]G;
[\hat{ }ADCEHPTWV] [\hat{ }ARCEIFWYV] [\hat{ }CILFTWY] ** [ARQGKPSTV] [ANDQEGKMS];
[RNQGSTV] **[RNGKSTV] [\hat{ }ADCHLMFPWY] [\hat{ }CHILKPWYV] [AGKPSV];
[RNGKFTYV] * [ARNQGPST] * [ARNQKPSTV] [ADQGKMSTV]G;
[ANQMFSTV] *K* [AQGST] [ANQGMST]G;
[\hat{ }ADCEHILW] * [\hat{ }ACEHILFWYV] [ANQGKPSTV] * [\hat{ }DCEHILKFWY] [ANDGPSY];
[\hat{ }ADCEHITW] [\hat{ }DCEGHIMFWY] * [ANQGMPSTV] * [\hat{ }RDCEHLPTWY]G;
[RNQILKFSV] [QGKPST]T** [ARQGKMST] [ADEGS];
[\hat{ }ACHILKW] [\hat{ }CEHILMFPWY] [\hat{ }NDCELFWYV] [\hat{ }RNCEILMFWY] ** [\hat{ }CQHILKMPTY];
[RNQGKMSY] [\hat{ }RDCGHILMWY] ** [ARGKMSTV] [\hat{ }NDCEHLPWYV] [DGPS];
[\hat{ }ARCEHILWY] [ARQGKST] * [\hat{ }CQEHILMFWY] [ARNGPST] * [NDGPS];
TK [ANQMST] ** [QKS] [NDS];
[NQGKMST] * [ARNQGKPS] [ARNQGPTV] [RNQHKMT] * [NGPS];
[NGFST]K**[ANGSTY] [ANGMPT]A; [RQMFS] [RKPT] **[NQGT] [NQS] [NGS];
[\hat{ }ADCEHPTW] [RNQLKMPST] * [\hat{ }CEILKFWY] * [\hat{ }CEHILPSWY] [ANDQEKPS];
[QT] * [QT]R*QA; [RNGT] * [RNGKST] [ARNQGT] [RNQGKMT] * [AG];
[DQEMPST] [ARGK] * [ARNQGKS] * [ADQGKSY] [ADLMPT];
[NGIKFTV] * [ARNGKTY] * [NQKMSTV] [ARNIKSV] [ADEPS];
[RNQKMFST] [RDQKS] [RNQPST] * [AGS] * [AS];
[RQGKS] [QKST] [RNQST] ** [NT] [GPS];
K**[ANGSTY] [QGMST] [ANGKMT] [ADEGP];
[RKM] [RNQGPS] *[ANQST] [NQST] * [GS]; [RQGIKT] [PS] [RKPS] *[ANGMT] *G;
[RQKM] *[NPS] *[AQGT] [ANQGST] [APS];
[ADK] ** [NGPST] [AEGMST] [ANQGKT] [ARS]; [QKMPTV] * [NKPS] [QGKPT]S*A;
[RNQGST] * [RNQGKSV] * [RNQGK] [AGKTV]G; [NQGKT] [RNK] * [ANGST] [ANGMT] *A;
[RNQGKMY] [RNQGS] * [NQGKS] * [AQGST] [AG]; [NK] **G[QT] [KS]G;
[RKMTY] [RP] [RNGKPS] * [ANQST] * [GPST];
[QKMFPYV] [RNGKPST] [RNGPST] [ARDQGKST] ** [GPS];
[RNQGKWV] [ANQKPSTV] [ARNQGKST] [AQGKST] ** [AP]; TK* [GSTV] * [QGV]G;
T[RGS] [RGKT] ** [RNST] [EGP]; [RGV]P[RNK] * [QST] *A;
[RKMV] * [ANPST]S [GKST] * [DGPS];
[NQMSTV] [RNQGHK] ** [ARGIKMST] [ARQKSV] [ADE]; [NQK] * [NKT] *S [NV]G;
[RNQFT] [RQK] [QGKST]N** [APS]; [NT] [GK] [NKMS] ** [ANQT]A;
[RQK] * [ANQGPST] [NGS] [ANGKPT] * [AGP]; [TYV] * [RK] [QGT]S* [GPS];
[NK] [RNP] [PT] [NS] ** [GP]; K* [NG] [AY] *KE; [NQ]R [NQGT] ** [NQT]A;
MKN* [GS] *G (SEQ ID NO: 199744); [QGKM] * [GS] * [KS] [NG] [AG];
[RNGMTYV] [RGKS] * [AQKPS] [NGS] *A; [QMTYV] [GK] * [NGKS] *TG;
[QKM] [RKMS] [NDP] [GSTV] **A; [NQSY]K* [QS] [ANQSV] *G; [QMT]RP** [AMS]A;
K*T [GI] * [RK] [DE]; [RQP] * [KPT] [DQGS] [AS] *A; TKP** [AQ]A (SEQ ID NO: 199745); T [RK] ** [NGL] [NMS] [AG]; K**Q [AS] [GK] [DS]; RQ* [NP]T*A (SEQ ID NO: 199746); [KS] [GY] *T* [TV]G; K* [QT] [GT] [GS] * [AS]; RT*T*SA (SEQ ID NO: 199747); K*A [NV] * [RS] [DG]; [NK] **Q [AR]S [AS];
[QG] [RP] [KP]N**A; [RT] [GS] ** [RN] [ST]G; MRPN**G (SEQ ID NO: 199748); G*KSV*G (SEQ ID NO: 199601); NK*T*SA (SEQ ID NO: 199749);
[NF] * [NK] * [NG]T [AS]; [GK] [KP] [NQT] * [GS] *A; MRT**SP (SEQ ID NO: 199750); [NM]K*QT*[GS]; GN**KNG (SEQ ID NO: 199751);
[NT] [GT] [RK] *N*A; K*ASS*A (SEQ ID NO: 199571); RT*GT*G (SEQ ID NO: 199752); [RQS] [AR] [PT] ** [NV] [GS]; RPT*S* [GS](SEQ ID NO: 199753);
K**GKSA (SEQ ID NO: 199754); [GV]KP**NA (SEQ ID NO: 199755);
TK*T* [KS] [AD]; TGK*G*A (SEQ ID NO: 199756); KP*GT*G (SEQ ID NO: 199757); RP*QQ*A (SEQ ID NO: 199758); KPNN**P (SEQ ID NO: 199759);
TGK**SA (SEQ ID NO: 199620); G**QKSG (SEQ ID NO: 199760); TG**KTA (SEQ ID NO: 199761); TN**RQG (SEQ ID NO: 199762); TG*K*SG (SEQ ID NO: 199763); I*AR*KE (SEQ ID NO: 199764); N*K*NNG (SEQ ID NO: 199765); R*S*STP (SEQ ID NO: 199506); KGNN**G (SEQ ID NO: 199567);
TK*N*QG (SEQ ID NO: 199707); G**QKGG (SEQ ID NO: 199766); K*AT*KD (SEQ ID NO: 199767); K*NQS*G (SEQ ID NO: 199768); KPS*N*A (SEQ ID NO: 199627); R*S*NVA (SEQ ID NO: 199769); RP*GT*A (SEQ ID NO: 199770); SRTT*NG (SEQ ID NO: 199576); TKQ**SA (SEQ ID NO: 199673);
and TS**RTP (SEQ ID NO: 199771); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position.

In any of the aspects provided herein, or embodiments thereof, a viral particle having a capsid containing the motif has increased biodistribution to liver relative to a control viral particle.

In any of the aspects provided herein, or embodiments thereof, the library contains an amino acid sequence motif selected from one or more of:
RQSA**T (SEQ ID NO: 199772); RAHS**A (SEQ ID NO: 199773); DG*K*KL (SEQ ID NO: 199774); NR*A*DK (SEQ ID NO: 199775); G*N*ANR (SEQ ID NO: 199776); RQ**NST (SEQ ID NO: 199777); [RKT] [STV] [RQE] **R[ADE];
T*GG*RN (SEQ ID NO: 199778); Q*G*VRG (SEQ ID NO: 199779); MDK**QR (SEQ ID NO: 199780); NGG**GR (SEQ ID NO: 199781); V*RQ*AG (SEQ ID NO: 199782); L*REG*R (SEQ ID NO: 199783); QAG**RG (SEQ ID NO: 199784); QR*VV*A (SEQ ID NO: 199785); RE**ARG (SEQ ID NO: 199786);
RQMA**A (SEQ ID NO: 199787); S**REIR (SEQ ID NO: 199788); TK*R*DT (SEQ ID NO: 199789); V*R*SAG (SEQ ID NO: 199790); V*RS*GG (SEQ ID NO: 199791); and VQR*S*G (SEQ ID NO: 199792); or polynucleotides encoding the same, where “*” represents any amino acid and square brackets denote a list of amino acids that may occur at a position.

In any of the aspects provided herein, or embodiments thereof, a viral particle having a capsid containing the motif has increased transduction efficiency for a liver cell relative to a control viral particle.

In any of the aspects provided herein, or embodiments thereof, the library contains an amino acid sequence motif selected from one or more of:
[RNGPS] * [GKSTY] [RNGKST] * [ANGMSTV]A;
[RNGKST] [NQGKP] * [NQGKPS] [NQKMSTV] *A; K** [AS] [AS] [AK] [EPS];
[RNQGKPSTV] [ARNGKPSTV] [ANDQGPSTY] [NQGKPST] ** [AMPSV];
[RQGKPST] [RQLKPT] [NDQST] * [ANQGSV] * [AGSV];
[RGKMST] [NKPST] ** [AQGKSTV] [ANDQG] [RDGPS];
[RKMPST] [ARGKPST] [ANQPSTYV] ** [ARNGKMST] [ADMPS];
[NDGPT]K* [NQGKPT] * [NQG]A; [KP] [RNGPT] * [QGST] [ANQT] * [GPS];
[QGPS] [QK] ** [ANST] [GMST]A; [NGPT] [NQGK] [NKV] **[ANGST]A;
[NPST] * [NK] [AQKT] * [QS] [PS]; [RNGK] * [NQST] [ANQGKST] [ARNGPT] *A;
[RNQPST] [QGK] *S* [ANQGKS] [GP]; K [NPT] * [ANGS] * [QTV]A;
[RQPT] [NDGKT] [KS] [AGST] **A; [RNQK] [PST] [NQKT] ** [NQT] [APS];
[KP] [RQP] *T [GT] *A; [RKP] [QS] [NGKPS] * [GT] *A; K* [NS] [AQGT] * [QGS]A;
[RN] [ANSV] [NGK] ** [QMV]G; [NK] [KST] ** [NGP] [AQGT]A;
[NGK] * [QKT] * [NQGP] [QGMTV] [AGS]; [QPST]K* [NGPS] [AMST] * [AG];
[KT] [RT] *SG* [PV]; KQ [GT] [QT] **A; [NKMT] * [RNQPS] [NGK] [ARGT] * [GMPV];
K*N* [NG] [NGV]A; R [PS] * [PS]G*A; [GST] [QGS]K*N*A;
[KS] * [NT] * [GS] [IS] [AP]; NKPA**S (SEQ ID NO: 199793);
[GKPS] [RKST] * [AQS] * [NS] [AGS]; [NK] ** [NPST] [GPT] [ANM]A;
[NQP] *K [PST] [ST] *A; [PT] *KG [AG] *A; SK [MT] **Q [AP]; NTR**SA (SEQ ID NO: 199503); PKS**SG (SEQ ID NO: 199794); TK*PS*S (SEQ ID NO: 199795); [NG] *K[ST] [QT] *[GP]; FGKQ**S (SEQ ID NO: 199796);
[NQ] [RKPSTV] [ARNKT] * [NGT] * [AGM]; [RK] * [NT] [NS] * [NS]P; K*N [QT]S*A (SEQ ID NO: 199797); [KMS] ** [GS] [AN] [NQT] [AG]; [NQ]K* [AT] [AG] *A;
KQ**AKD (SEQ ID NO: 199674); K* [PT]S* [AQG]A; [NMS] *K [PS] * [NG]G;
NAKS**G (SEQ ID NO: 199798); KQ**TQA (SEQ ID NO: 199799);
[NK] [ST] [NK] * [AT] *P; K*TQ*GA (SEQ ID NO: 199800); T**QSGF (SEQ ID NO: 199801); KTQ*T*G (SEQ ID NO: 199802); QK**GAP (SEQ ID NO: 199803); SK**NAA (SEQ ID NO: 199804); K*AT*KD (SEQ ID NO: 199767);
R*N*ANP (SEQ ID NO: 199805); GKQ*S*P (SEQ ID NO: 199806); PQKS**A (SEQ ID NO: 199807); N[GK] *[KT] *SA; SQ**TNP (SEQ ID NO: 199808);
PR*Q*SP (SEQ ID NO: 199809); NG*R*TP (SEQ ID NO: 199810); TKM**QA (SEQ ID NO: 199811); NK*TN*S (SEQ ID NO: 199812); N*K*NNG (SEQ ID NO: 199765); PK*GN*A (SEQ ID NO: 199813); FQKA**G (SEQ ID NO: 199814); G*KT*QA (SEQ ID NO: 199815); K**TSQS (SEQ ID NO: 199816);
K*ASS*A (SEQ ID NO: 199571); MNQ*R*P (SEQ ID NO: 199817); RQ*A*TS (SEQ ID NO: 199818); SK**NQP (SEQ ID NO: 199819); and TKN**QS (SEQ ID NO: 199820); or polynucleotides encoding the same, where “*” represents any amino acid and square brackets denote a list of amino acids that may occur at a position.

In any of the aspects provided herein, or embodiments thereof, a viral particle having a capsid containing the motif has increased biodistribution to heart relative to a control viral particle.

In any of the aspects provided herein, or embodiments thereof, the library contains an amino acid sequence motif selected from one or more of:
[GFTW] [AKM] [NQKPT] [QGKP] ** [AG]; [MT] [RGP] [RQP] [KS] **G;
[QMT] [RGP] [RQGP] * [QKMT] *G; [QG] * [AGP] [GT]K*G; [RF] [KP] ** [AQ] [NQ]G;
KT[AS] ** [GK] [AEP]; G**TKMG (SEQ ID NO: 199821);
[GK] * [PT] [QG] * [QS]G; [GT] [NG] ** [RK]SG; [QT] * [RG]S [KT] *G;
[QK] * [GP] * [KST] [GSV]G; [RM]SS [KT] ** [AS]; [RM] [NQG]S* [GKV] *G;
[MT] *STK*G (SEQ ID NO: 200023); K[GT]S [QT] **G; K*P [NS] *GA (SEQ ID
NO: 199822); K[MST] [PS] [GT] **A; K[DT] [AR]S** [AG]; [IY]KP** [KS] [ND];
R[NQ]S [AS] **G; M*SKS*A (SEQ ID NO: 199596); K*S [AN] * [AGS] [AS];
[QGF] [NKS] * [GPV] [NGK] *G; [RK] [DST] [RS] * [GS] * [AGP];
[KT] [GY] * [KT] *TG; R*SN*TG (SEQ ID NO: 199552); AKY*K*E (SEQ ID NO: 199823); [KT] [QG] [AT] **[ST]G; MR*NQ*G (SEQ ID NO: 199604); K*PT*TG (SEQ ID NO: 199824); NRPS**P (SEQ ID NO: 199699); KRPD**G (SEQ ID NO: 199825); R*SSV*G (SEQ ID NO: 199826); KSSS**G (SEQ ID NO: 199827); TK*S*YA (SEQ ID NO: 199828); FK*S*QG (SEQ ID NO: 199829);
K**NTTG (SEQ ID NO: 199830); FK*P*QG (SEQ ID NO: 199831); FKM**QG (SEQ ID NO: 199832); GK*VS*N (SEQ ID NO: 199833); K*SSV*G (SEQ ID NO: 199834); K*ST*NG (SEQ ID NO: 199684); KF**TSG (SEQ ID NO: 199835); KGSS**P (SEQ ID NO: 199836); N*K*GMG (SEQ ID NO: 199837);
and YKP*T*P (SEQ ID NO: 199838); or polynucleotides encoding the same, where “*” represents any amino acid and square brackets denote a list of amino acids that may occur at a position.

In any of the aspects provided herein, or embodiments thereof, a viral particle having a capsid containing the motif has increased biodistribution to spleen relative to a control viral particle.

In any of the aspects provided herein, or embodiments thereof, the library contains an amino acid sequence motif selected from one or more of:
[NEGKMFTWY] [ARDKPST] [ARNQGST] [ANQKPST] **[AGFS];
[ARQLKM] [ANGHIT] * [RQGHP] * [AGKST] [ARDKS];
[GKFTY] [GKT] **[ARGV] [AQFS]G;
[NGKT] **[NGKMPT] [LKPST] [NGHKMFT] [RDGSY];
[RNQKMT] [RNGIL]P [NDHKST] ** [ANGKPV];
[DEGKMFT] [GKMFP] [ARHMF] ** [NQGKST] [DGKP];
[RQGFYV] * [RGLKPS] [RNGS] * [ANDQPT] [RGKPY];
[RQKM] [RGT] *[NGP] [AQGPV] *[GMY];
[RQGIKMTYV] [RNDQGHPST] [\hat{ }DCEILMFWYV] * [\hat{ }DCEHLKFWY] * [ANGKFPS];
[RGKMSTV] *[ARNHMST] [NQKST] [ARGKTV] *[ARG]; K*PG*QG (SEQ ID NO: 199839); [QGFT] [ANK] * [APTV] [ARNGKP] * [GV]; [TY]KP*T* [PY];
[QGTY] [RK]P**[AGMS] [ANG]; [GLMTY]K*[HPS] *[NFSY] [AQG];
[NKT] [RGY] * [KT] * [TV]G; [RM] [HP] [KP] ** [HP] [AM];
[RKMTY] * [GPT] * [ARNST] [QGKS]G; Q* [RP] [QG] [KT] * [GP]; S*NAH*R (SEQ ID NO: 199840); R**VRDV (SEQ ID NO: 199841); KM**PKD (SEQ ID NO: 199590); RP**[QG] [NG]G; SP**RGG (SEQ ID NO: 199640);
[MST] [HKY] [GP] [AQ] ** [KY]; [QFV] [QP] [RNG]H** [KV];
[RY]P* [KS]S* [AGS]; FK* [PS] *QG (SEQ ID NO: 199842); MGS*K*G (SEQ ID NO: 199843); [NK] * [RS] * [TV] [TY] [GM]; G*TQ*SG (SEQ ID NO: 199844);
TR*VS*G (SEQ ID NO: 199736); YKPP**G (SEQ ID NO: 199845); QR*S*TG (SEQ ID NO: 199525); SK**YGA (SEQ ID NO: 199846); TK*VS*N (SEQ ID NO: 199847); QK*PS*A (SEQ ID NO: 199848); GK*ST*A (SEQ ID NO: 199711); QKS*S*G (SEQ ID NO: 199849); K*TI*KD (SEQ ID NO: 199850);
GK*PS*A (SEQ ID NO: 199851); AKY*K*E (SEQ ID NO: 199823); FK*T*MS (SEQ ID NO: 199852); GK*VS*N (SEQ ID NO: 199833); K*MQ*QG (SEQ ID NO: 199853); Q*KPH*N (SEQ ID NO: 199854); QKT**GG (SEQ ID NO: 199700); QPRG**G (SEQ ID NO: 199855); R**SVAG (SEQ ID NO: 199856);
R*Q*APF (SEQ ID NO: 199857); and SK**NQP (SEQ ID NO: 199819); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position.

In any of the aspects provided herein, or embodiments thereof, a viral particle having a capsid containing the motif has increased biodistribution to kidney relative to a control viral particle.

In any of the aspects provided herein, or embodiments thereof, the library contains an amino acid sequence motif selected from one or more of: YMNN**K (SEQ ID NO: 199858); I*RS*TG (SEQ ID NO: 199859); NGG**GR (SEQ ID NO: 199781); and RQMA**A (SEQ ID NO: 199787); or polynucleotides encoding the same, where “*” represents any amino acid and square brackets denote a list of amino acids that may occur at a position.

In any of the aspects provided herein, or embodiments thereof, a viral particle having a capsid containing the motif is capable of transducing kidney cells in vivo.

In any of the aspects provided herein, or embodiments thereof, the library contains an amino acid sequence motif selected from one or more of:
GK** [ST] [NMT] [AP]; [NK] * [NS] [KT] [NS] *A; [NGP] [QKS] [KT] * [NGS] * [AG];
K*N [AN] * [NQG] [AP]; [NQKPS] [NQKT] [AQGKST] [AQGST] ** [AGS];
[KP] [KPST] ** [ANK] [ANDG] [ADPS]; [NKV] [NG] * [GK] * [KST] [AG];
[GP] [RNK] [NKPT] ** [GS] [AM]; K*TS* [AS] [AV];
[NGPST] [GK] * [NQGKS] [NKT] *A; PR*Q*SP (SEQ ID NO: 199809);
[NP] [RKS] [NK] * [GT] * [APV]; [NG] [QK] [KT]N**A;
[RPST] [KT] [NMS] ** [AQT] [PS]; [NT] *KS* [ST] [AP];
[KM] [NPT] **G [QG] [ARS]; K*N* [GS] [NI]A;
[RNPS] [ARNK] [ANG] [QGKS] ** [AGP]; K [LKPT] [ND] * [AGS] *A;
KT [NQ] ** [AQGMS] [AS]; K [NQT] * [GPS] [AQGV] * [APV]; [PS] *K [AQ] *QS;
[NP] *KS [ST] *A; KQ [GT] * [TV] *A; K*TS*QA (SEQ ID NO: 199860);
[KT] * [NKP] [NG] [AG] * [AMP]; P*KG*GV (SEQ ID NO: 199861);
[NP] [RG] * [QS] [QK] *P; KP [QS] [NT] ** [AM]; K**PGMA (SEQ ID NO: 199862); N*K*NNG (SEQ ID NO: 199765); RPNN**P (SEQ ID NO: 199863);
K*T*QQA (SEQ ID NO: 199864); [PS]K[PT] **[QG] [AS]; PK*PS*A (SEQ ID NO: 199865); KTV**KD (SEQ ID NO: 199866); KA*TN*A (SEQ ID NO: 199867); KN*A*QA (SEQ ID NO: 199868); K*QTG*A (SEQ ID NO: 199869);
KTNG**P (SEQ ID NO: 199870); K*T*NTS (SEQ ID NO: 199871); KTN*A*P (SEQ ID NO: 199872); KST**TA (SEQ ID NO: 199873); DK*K*GA (SEQ ID NO: 199874); KPNN**P (SEQ ID NO: 199759); KTQ*S*S (SEQ ID NO: 199655); N*QKT*G (SEQ ID NO: 199875); K**TGNA (SEQ ID NO: 199876);
K*N*NVA (SEQ ID NO: 199877); KP*TN*S (SEQ ID NO: 199878); KPA**GA (SEQ ID NO: 199879); P*KG*SA (SEQ ID NO: 199880); QK**GAP (SEQ ID NO: 199803); SK*S*QP (SEQ ID NO: 199881); or polynucleotides encoding the same, where “*” represents any amino acid and square brackets denote a list of amino acids that may occur at a position.

In any of the aspects provided herein, or embodiments thereof, a viral particle having a capsid containing the motif has increased biodistribution to serum relative to a control viral particle.

In any of the aspects provided herein, or embodiments thereof, the library contains an amino acid sequence motif selected from one or more of:
[RQKP] [RQPT] ** [ANGT] [ANGS] [AS];
[RNQKP] [RGST] [RNQKPV] **[ARNGKMST] [RDGMPS]; K*[NQT] *[QGS] [NQIM]A;
[EGKMPST] [RGKPST] [ANQPSTYV] ** [ANGST] [AKPS];
[RNQGKPW] [ARNQGPST] [ANDQGKS] [NQGKT] ** [AKMPS];
[NGT] * [KS] [NGKS] [ANGMT] *A;
[RNGHKMP] * [NQGPSTYV] [ARNQGK] * [ANQGKS] [ARDG];
[NKT] *[NQPS] [NGK] [ARGT] *[GMP]; [NGPS] *[NQK] [AQKT] *[NQST] [GPS];
[NQGMPSV] [KS] [ANKSTY] [ANQGT] ** [AP];
[RGKMPSV] [QKPST] [NDQKST] * [ANQGSV] *A; [KMPT] [RPT] [NK] * [GS] * [GPSV];
[QGPSTV]K* [NQGPSV] [ANMST] * [AGPS]; [QK] * [NG] [NS] * [NGV] [GP];
[RGTV] ** [NQS] [QGTY] [NMT] [AKP]; [NPS] *K [GS] * [ST]A;
K** [QG]A [QS] [AS]; [NQPT] [KV] [NQKT] * [AGS] * [GMS];
[RNGKT] [QGPST] * [NQGKS] [ANQGKTV] * [AGV]; [QGT] [GT]K** [AT]A;
[NDKS] * [RKT] * [NGMS] [NPST] [KPS]; [NK] [AGT] * [GKS] [AKS] *P;
[GK] * [QHK] * [NP] [LTV] [AQ]; [QKP] * [ANK] [QST]S*A;
[NV] [RS] [AKP] * [NT] *A; [NST]K [MPT] ** [QG] [AP];
[NQKST] [RNK] [ANDQST]S**A; T**SSNR (SEQ ID NO: 199882);
[KP] [ARQ] *T [NGT] *A; [NQG] [QK] * [KST] [ANGS] * [AM]; [SV] [RK]TS** [GP];
K**GTNA (SEQ ID NO: 199883); NK [NQ] * [AG] *A; K* [PT]S* [QGS] [AV];
[KTW] [NKT] [QGS] **Q[ANKS]; [RNKP] [RNGT] *[QGKPS] *[GST] [APS]; PKS**SG (SEQ ID NO: 199794); RDKS**A (SEQ ID NO: 199612);
[RY] * [ST] [KS] * [ST] [AP]; [GF] *K [QT]T* [PS]; [RN] * [AP] [GK]S* [AG];
RNGS**G (SEQ ID NO: 199884); KT[NQG] * [AS] * [GPS]; K*NSS* [PT](SEQ ID NO: 199885); Q*GSK*G (SEQ ID NO: 199886); KVS*T*A (SEQ ID NO: 199887); NSK*T*P (SEQ ID NO: 199888); [NGP]K*[NGPST] *[ANQG]A;
K [NT] * [AG] *QA; [GKP] [QKT] ** [ANST] [NGMT] [AP]; QK**GAP (SEQ ID NO: 199803); FQK*A*G (SEQ ID NO: 199889); K*QTG*A (SEQ ID NO: 199869);
[KS] [QK] ** [QT]Q [AP]; KQTQ**A (SEQ ID NO: 199890);
[GP] *K[GT] * [QG] [AV]; [SV] * [RK] *SAG; MN**GQR (SEQ ID NO: 199891);
[NS]K*A*S[AG]; K**TGNA (SEQ ID NO: 199876); N*K*GQG (SEQ ID NO: 199892); TK*V*QG (SEQ ID NO: 199893); QK*TS*P (SEQ ID NO: 199894);
QK*S*[QS] [PS]; NKV**SA (SEQ ID NO: 199895); K*SGT*A (SEQ ID NO: 199896); PK*S*AG (SEQ ID NO: 199897); G*Q*TQG (SEQ ID NO: 199898);
K**TSQS (SEQ ID NO: 199816); K*TST*A (SEQ ID NO: 199899); KNGS**G (SEQ ID NO: 199900); KQG*T*A (SEQ ID NO: 199901); NVK**QG (SEQ ID NO: 199902); PR*QQ*P (SEQ ID NO: 199903); SN**KSA (SEQ ID NO: 199904); and T*KS*TP (SEQ ID NO: 199905); or polynucleotides encoding the same, where “*” represents any amino acid and square brackets denote a list of amino acids that may occur at a position.

In any of the aspects provided herein, or embodiments thereof, a viral particle having a capsid containing the motif has increased biodistribution to brain relative to a control viral particle.

In any of the aspects provided herein, or embodiments thereof, the library contains an amino acid sequence motif selected from one or more of:
[NPT]K[PY] [GP] **A; KT**PQA (SEQ ID NO: 199906); KT**AKE (SEQ ID NO: 199907); [KPTY] [RQKT] [APSY] **[AGKS] [ANE];
[RQK] [QPST] [KS] [MST] **G; K* [AGS] [NS] [AST] * [AP];
[RK] [GST] [AS] [GST] ** [APS]; Q*GSK*G (SEQ ID NO: 199886);
[RKMT] [RQGKS] [PS] * [QKSTV] *G; [RKY] [QKT] [PST] * [STV] * [AP];
[NK] [RMS]P [GST] ** [AP]; [MT]PK* [AT] *G; K*TTG*G (SEQ ID NO: 199908);
[TV]KP [GS] **G; PK**NGA (SEQ ID NO: 199909); PK*PS*A (SEQ ID NO: 199865); TPR*G*G (SEQ ID NO: 199910); KTQ**QA (SEQ ID NO: 199911);
QPK*G*G (SEQ ID NO: 199912); RNS**NG (SEQ ID NO: 199913); K**NTTG (SEQ ID NO: 199830); K*ST*[NS]G (SEQ ID NO: 199914); YKPP**G (SEQ ID NO: 199845); K*SSV*G (SEQ ID NO: 199834);
[RNQ] * [GKS] * [GK] [MTV]G; KQ*SV*A (SEQ ID NO: 199915); PR*Q*SP (SEQ ID NO: 199809); R*SN* [NT] [AG]; K*PS*QA (SEQ ID NO: 199916);
FK*TA*G (SEQ ID NO: 199917); GK*PS*A (SEQ ID NO: 199851); K*GS*MS (SEQ ID NO: 199918); K*SN*GS (SEQ ID NO: 199919); FK**AQG (SEQ ID NO: 199920); GQ*RV*G (SEQ ID NO: 199510); K*ASG*A (SEQ ID NO: 199921); K*SN*AA (SEQ ID NO: 199922); KY*T*TG (SEQ ID NO: 199923);
P*R*NGA (SEQ ID NO: 199924); QPR*M*G (SEQ ID NO: 199925); RPQ**TG (SEQ ID NO: 199926); T**VNRG (SEQ ID NO: 199927); and TPRS**G (SEQ ID NO: 199928); or polynucleotides encoding the same, where “*” represents any amino acid and square brackets denote a list of amino acids that may occur at a position.

In any of the aspects provided herein, or embodiments thereof, where a viral particle having a capsid containing the motif has increased biodistribution to lung relative to a control viral particle.

In any of the aspects provided herein, or embodiments thereof, the library contains an amino acid sequence motif selected from one or more of:
[RNGKS] [KPTV] [NQGS] * [ANQST] *A; K** [NST] [QS] [AQK] [DS];
[NGMPSTW] [RNQGK] [QGKMSTV] **[AQKST] [ARNDGKP];
[RGKP] [QKPT] **[NQST] [NQGMT]A; [NMPS] *[NQKS] [AQGHK] *[QGS] [RIPSV];
[RNLPS] [RGHKS] * [AQGKPT] * [ANGS] [ARGP];
[RQGMS] [NDQK] **[NGKS] [NQGS] [ARQKPT];
[RNQGKPSW] [ARNGKPT] [ANQGST] [ANQGKST] **[AGKMPS];
K[NQT] * [AGS] *Q[AS]; [RKT] [KT] [NQV] ** [ARQKT] [DPS];
[RNGKMPS] [RQST] [NGHKT] * [NQGTV] * [AKYV]; [GT] ** [QS] [SY] [NM] [RK];
[KFSY] * [ANHKP] [NQS] [AGST] * [RMST]; [NKP] [ARGK] [GHPS] ** [AG] [RMST];
[GP]K* [NS] * [AQ] [AG]; [RKP] ** [QT] [QGS] [NG]A;
[QGKP] * [NKS] [KPST] [AS] *A; [GK] * [NQHT] * [NGPV] [QLMTV] [AQS];
[GK] * [NKPS] [ANGTV] * [AQT] [AM]; K*TS* [AQ]A (SEQ ID NO: 199929);
N* [ST] [GK] [RN] *A; [GK]Q [QGT] [QGT] ** [AM]; K*NN*NP (SEQ ID NO: 199930); K[PT] [QS] ** [AQS]A; [QKP] [NST] [KT] ** [GT]A;
[KV] [QT] [QK] *S* [AS]; [RNQGKS] [AQGKPT] * [NGKST] [ANGKTV] * [AMSV];
KN*G*SA (SEQ ID NO: 199570); T**GPGR (SEQ ID NO: 199931); K*TG*KD (SEQ ID NO: 199932); K**G [AT] [NQ]A; [QT] [NQK]K [DT] **A;
[NK]K [ND] *G*A; N*QKT*G (SEQ ID NO: 199875); K [GT]N** [GT]A; GKN**SA (SEQ ID NO: 199933); K [ST] ** [AK] [ND] [DP]; PK* [NGP] [ANS] *A; TK*V*QG (SEQ ID NO: 199893); K*SGT*A (SEQ ID NO: 199896); N*K*[NS]N[GP];
[NG]KT*G* [AM]; [MP] [GK]T*S* [RG]; [NP] *KS* [GS]A; KP** [AG] [AG]S;
SK*S*QP (SEQ ID NO: 199881); S*T*GSP (SEQ ID NO: 199934); HQKP**L (SEQ ID NO: 199935); N*N*GTA (SEQ ID NO: 199936); SRTP**A (SEQ ID NO: 199937); PR*QQ*P (SEQ ID NO: 199903); P*KG*SA (SEQ ID NO: 199880); VK*NN*P (SEQ ID NO: 199938); K*N*SIA (SEQ ID NO: 199939);
KPT*P*S (SEQ ID NO: 199940); T*KS*TP (SEQ ID NO: 199905); N*KST*A (SEQ ID NO: 199941); R*TS*SP (SEQ ID NO: 199519); K*N*GNA (SEQ ID NO: 199942); KTN**GS (SEQ ID NO: 199943); N**GRTA (SEQ ID NO: 199944); NKPP**A (SEQ ID NO: 199945); SK**TNS (SEQ ID NO: 199946);
SK*V*TS (SEQ ID NO: 199947); and TK*QN*A (SEQ ID NO: 199948);
TKQ*S*S (SEQ ID NO: 199949); or polynucleotides encoding the same, where “*” represents any amino acid and square brackets denote a list of amino acids that may occur at a position.

In any of the aspects provided herein, or embodiments thereof, a viral particle having a capsid containing the motif has increased biodistribution to spinal cord relative to a control viral particle.

In any of the aspects provided herein, or embodiments thereof, the library contains an amino acid sequence motif selected from one or more of: GAG**MR (SEQ ID NO: 199950); LQSN**R (SEQ ID NO: 199951); NN*TT*R (SEQ ID NO: 199952); NQ*Q*TK (SEQ ID NO: 199953); and RVG**DK (SEQ ID NO: 199954); Y*AG*SR (SEQ ID NO: 199955); or polynucleotides encoding the same, where “*” represents any amino acid and square brackets denote a list of amino acids that may occur at a position.

In any of the aspects provided herein, or embodiments thereof, a viral particle having a capsid containing the motif has increased biodistribution to kidney relative to a control viral particle.

In any of the aspects provided herein, or embodiments thereof, the library contains an amino acid sequence motif selected from one or more of:
WNG**QK (SEQ ID NO: 199956); [DY] [KT] [FS] ** [QK] [GK];
[QSWY] [NIKM] [NGP] [NQH] ** [KY]; IKH*R*E (SEQ ID NO: 199957);
[IT] [LK] * [DH] * [KV] [RN]; [KF] * [KS] * [PT] [NY] [LM]; YKD**PR (SEQ ID NO: 199958); HN*N*GK (SEQ ID NO: 199959); KFK*E*Y (SEQ ID NO: 199960); NGQ**AR (SEQ ID NO: 199961); QI*H*AK (SEQ ID NO: 199962);
R**NGIQ (SEQ ID NO: 199963); REKP**M (SEQ ID NO: 199964); Y*H*MKG (SEQ ID NO: 199965); or polynucleotides encoding the same, where “*” represents any amino acid and square brackets denote a list of amino acids that may occur at a position.

In any of the aspects provided herein, or embodiments thereof, a viral particle having a capsid containing the motif has increased liver transduction efficiencies relative to a control viral particle.

In any of the aspects provided herein, or embodiments thereof, the library contains an amino acid sequence motif selected from one or more of:
[\hat{ }ADCEHILKW] [ARNQGKST] [ARNQGKPTV] [ARNQKPSTV] **[NGPS];
[RNQKSV] * [ANQGPST] * [ARNQKT] [ANQGKT] [EGPS];
[\hat{ }ANDCEHILFW] * [ANQGKPST] [ARDQGKPST] [ARNQGKSV] * [ADGS];
[NQGFST] * [NGKT] * [ARNQKST] [ANQGKSTV] [GP];
[NQGKMSTY] [RQGKPST] * [ARDQGKSTV] [\hat{ }ADCEHILFWY] *G;
[RNGKMPT] **[AQGKST] [ARNQGSV] [AQGKMST] [EGPS];
[NQGKFST] [ARQGKPST] [ADQGKPST] * [ARNQIKST] *G;
[RNQGHKPTV] * [ARNQGMPST] [ARNQKST] * [ANQGKMS] [DEGPSV];
[\hat{ }ARDCEHILKW]K* [ANQGST] * [ANQIKMSTV] [DGPS];
[RNGKTV] [ANQGSV] ** [ARGKSTV] [AQGKMS]G;
[\hat{ }ADCQEHFWY] [ARNQGKMPS] [\hat{ }CEHILKFWYV] ** [ANQGKMSV] [NDGPS];
[RNQGMPSTV] [ARDQGKPST] [NQGKST] [ANGKPST] ** [APS];
[RNGKMST] [RGKST] [RNQMT] ** [ARNQMST] [ADPS];
[RNQGMPSTV] * [AQGKST] [AQGKPSTV] * [NQGKSTV] [GPSV];
[NQST] * [QKT] * [GKT] [NQMS] [GP]; [RNQKSTY] [RKPT] [ANQGST] ** [ANQGT]G;
[RNQMTYV] [NQKP] [RNQGS]G**G; [RGKMT] ** [NT] [RNGKS] [ANGT]G;
[RNGT] * [ANGKT] [ARNKMST] [RNQGKS] *G;
[RQGKMPST] [NQKPST] ** [ARNKST] [\hat{ }RCQEHILFPW] [DGPS];
[RKS] [NQGPST] [ANQGKT] [ANQGT] **[APS];
[RNMPSTY] [ARNQGKSV] * [ANQKST] * [ANGKSTV] [GPS];
[NQGKPSTV] [ARQGKST] *[AQGKPST] [ANKSTV] *[APS];
[RNQGKTY] [RNQGLPST] [ARNQKPST] * [ANQSTV] * [APS];
[NQGFST] [ANGSV] [RK] ** [NQSV] [GP]; K [NQGLKST] [NDQGMPT] [NQST] **G;
[NQGKMFPST] [RQGKPS] [RDQKPST] *G* [AM];
[RQGKMST] [QKP] [NDQGS] * [ANKST] * [ADEPS]; K* [QT] * [QG] [QMT]A;
[NKMST] [ANGKP] [NGKP] * [ARNMPST] *G; [RNQPST] [RNK] ** [ARNGS] [ANGSTV]A;
[QGS]K** [GT] [AMT] [APS]; [NQGMST] [NK] ** [AQGSV] [QKMFS] [RDGPS];
[RDKST] [NQKP] * [AGKS] * [AGSTV]A;
[NQGKSTV] [RNQGK] * [NQGKS] [ANQKST] * [GPS]; [QK] [NKSV] [NDGKT] [ST] **A;
[RKMT] ** [GPS] [AT] [ANGS]G; [RKM] * [NPST] [AGT]T* [GV];
[RKV] ** [NGT] [AGST] [NQG]A; [QKST] [QKT] * [NG]G* [DG];
[RQGKT] * [RNQGPST] [RQGST] * [QGMS]A; [KV] *N* [NS] [AISV]A;
[NQKPT] [RKP] * [AGST]G* [AP]; [RNQKS] [ANMPSV] [RNQK] *G*G;
[RNST] * [QKMST] [RGS] [ARST] * [AP]; [NKS] [KST] [NK] * [NG] * [AS];
[RNGT] * [RQGKST] [NKST] [RNMT] * [AG]; [RKT] [RQT] ** [GT] [NQG]A;
[RNIK] [RNQV] * [AGS] * [NQGT] [GS]; [RGKP] [NKT] [NQTV] **G[AS];
[RNQST] [RQK]S [ANQKS] **G; R [NST] * [ANQG] [ANTV] *G; [QK] [QT] **N [GS]A;
[QPT] [RGK] *N [AKS] *A; [QGT] [QGT]K** [AT]A; RP* [NG] [NT] *G;
[FT]K**NQ[NG]; [NGS] * [GKS] [RGKS] * [AGST]A;
[ANGS] [RGK] * [NKPT] * [ANGS]A; [MT]K* [QM] *QA; [KP] [NP] [KS] ** [AG]A;
[NM] * [NK] [NK] * [QT]G; [RK] **NT [GS]G; RN* [AN] [AN] *A;
N** [NQS] [RGK] [QGS]A; [KT] * [NGT] * [GSV] [ANST] [GS];
K* [NQM] [NG] [ANG] * [GP]; [NQ] * [AGK]G [QT] *G; QKN**SA (SEQ ID NO: 199966); Q* [NK]G* [AN]G; K[NT] * [AG] *QA; F*KT*QA (SEQ ID NO: 199967);
[NST]K[GPT] *G*G; RP*[ST]G*A (SEQ ID NO: 200024); PTK**SG (SEQ ID NO: 199968); R**QQNA (SEQ ID NO: 199969); QPK**GG (SEQ ID NO: 199970); KN*ST*A (SEQ ID NO: 199971); RQ*PT*A (SEQ ID NO: 199515);
K [NQG] *N* [AT]G; R[AN] [QS] * [AT] *G; [NQ] [RN] [GKP] [NQS] **A;
KP [GP]G**G (SEQ ID NO: 199972); KPS [NGS] ** [AM]; RS*PG*A (SEQ ID NO: 199973); K* [NQT]ST*A (SEQ ID NO: 199974); [RK] * [NS] [AG]T*A;
KPSQ**P (SEQ ID NO: 199975); [NKM] *N*[GS] [NT]A; RSS*G*A (SEQ ID NO: 199976); RN*PG*G (SEQ ID NO: 199977); K[NQ] *[GS] *SG;
[NT] *K* [QPS] [NGS]A; N*RNT*P (SEQ ID NO: 199978); K*[NG]A*[QG]A;
K*GNQ*A (SEQ ID NO: 199979); S**STQS (SEQ ID NO: 199980);
NK**[GT]GG (SEQ ID NO: 199981); RN**NQA (SEQ ID NO: 199982);
NR**GQA (SEQ ID NO: 199983); K* [NT] [NS] *AA; [RK] [NT] *N*QA; NR*N*QG (SEQ ID NO: 199984); N**STMA (SEQ ID NO: 199669); N*KA*AG (SEQ ID NO: 199985); NKQ*A*A (SEQ ID NO: 199986); MKQ*G*G (SEQ ID NO: 199987); RGN*T*G (SEQ ID NO: 199988); K*NSS*P (SEQ ID NO: 199989);
K*NNP*A (SEQ ID NO: 199990); MKN*G*G (SEQ ID NO: 199991); RN*NG*G (SEQ ID NO: 199992); G*K*NVA (SEQ ID NO: 199993); K*GG*AG (SEQ ID NO: 199626); N**GTKG (SEQ ID NO: 199994); NN**NKG (SEQ ID NO: 199995); RDK**GG (SEQ ID NO: 199996); RQ*N*QG (SEQ ID NO: 199997);
SRTT*NG (SEQ ID NO: 199576); and TSKG**G (SEQ ID NO: 199998); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position.

In any of the aspects provided herein, or embodiments thereof, a capsid containing the motif has reduced spleen biodistribution relative to a control viral particle.

In any of the aspects provided herein, or embodiments thereof, the capsid polypeptides are capable of forming viral particles with two or more of the following traits: 1) binding to liver cell; 2) transducing liver cell; and 3) biodistributing to the liver of an organism. The library contains an amino acid sequence motif selected from one or more of:
[NQGIKT] [DGKFPS] [NEHKS] * [RDEHY] * [RQEKY];
[RDS] [DQE] *[RPT] [RNK] *[RKV];
[RDKMSY] ** [RNKSV] [ARDEP] [ARNDEIKS] [ARNKMYV]; Q [RE] ** [RK] [DI] [IK];
[NMT] [RDK] *[ARGTV] *[RDSY] [ANKT]; [GLFTY] *[HKY] *[NMPY] [NKS] [AQGL];
[ANGST] * [ADEG] [RNGH] * [RK] [RNESV];
[REKFY] [ARLTYV] [DGHKP] [RDHIKS] **[ARNEIFT];
[ANQKT] [ARKPS] [RGKM] ** [RSV] [DEGY]; [RMS] * [EK] * [RV] [DPT] [DKT];
[QY] *G*V [RK]G; [IPT] [DEGP]R [AQHV] ** [ANK]; IRA**EK (SEQ ID NO: 199999); [DK] *R [NE] * [KT] [AQ]; [QI] [RD] *K [MP] * [RE]; D*KPR*Q (SEQ ID NO: 200000); [ST] [QY] [EK] * [RS] * [NK]; [EY] [ET] *K*RN; ILH**KN (SEQ ID NO: 200001); V*RSD*K (SEQ ID NO: 200002);
[ADE] [ADG] * [GK] *K [LMY]; [KV] [QLT]R* [DS] * [GI]; TD*KR*L (SEQ ID NO: 200003); [RELF] [EK] ** [AQ] [RD] [DGKS]; EK**TRQ (SEQ ID NO: 200004);
[MV] *R* [SV] [AD] [GK]; IL*H*KN (SEQ ID NO: 200005); V*KG*YN (SEQ ID NO: 200006); G**HKQL (SEQ ID NO: 200007); Y*SH*KG (SEQ ID NO: 200008); D[NK] [RH] [KV] ** [RV]; I*RS*TG (SEQ ID NO: 199859);
[IK] [GP]R* [DT] * [AG]; QGR*L*A (SEQ ID NO: 200009); YTS**KG (SEQ ID NO: 200010); G**ETRK (SEQ ID NO: 200011); V*RQ*AG (SEQ ID NO: 199782); DL*K*RA (SEQ ID NO: 200012); D*RPK*V (SEQ ID NO: 200013);
DR**VKQ (SEQ ID NO: 200014); G**TEKK (SEQ ID NO: 200015); I*R*MRE (SEQ ID NO: 200016); NDMR**K (SEQ ID NO: 200017); NE*KR*V (SEQ ID NO: 200018); QAR*E*R (SEQ ID NO: 200019); V*RS*GG (SEQ ID NO: 199791); YTE**KK (SEQ ID NO: 200020); or polynucleotides encoding the same, where “*” represents any amino acid and square brackets denote a list of amino acids that may occur at a position.

In any of the aspects provided herein, or embodiments thereof, each peptide has a net charge of +1.

In any of the aspects provided herein, or embodiments thereof, the capsid polypeptides are capable of forming viral particles with all of the following traits: 1) binding to liver cells; 2) transducing liver cells; and 3) biodistributing to the liver of an organism, and where each of the peptides has a net charge of +1.

In any of the aspects provided herein, or embodiments thereof, the composition further contains a carrier, excipient, or diluent.

In any of the aspects provided herein, or embodiments thereof, the organism is a mammal. In any of the aspects provided herein, or embodiments thereof, the mammal is a mouse or a macaque.

In any of the aspects provided herein, or embodiments thereof, the trait of interest is selected from one or traits selected from one or more of the following: binding to liver cells; biodistributing to the liver; production fitness; immune cell binding; immune cell transduction; brain endothelial cell binding; brain endothelial cell transduction; liver cell transduction; heart biodistribution; spleen biodistribution; kidney biodistribution; kidney transduction; serum biodistribution; brain biodistribution; lung biodistribution; spinal cord biodistribution; and spinal cord transduction.

In any of the aspects provided herein, or embodiments thereof, the cells contain hepatocytes. In any of the aspects provided herein, or embodiments thereof, the cells contain immune cells, brain cells, pulmonary cells, or liver cells.

In any of the aspects provided herein, or embodiments thereof, the trait of interest is increased biodistribution and/or transduction in the liver, kidney, spleen, brain, spinal cord, serum, heart, and/or lungs.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “AAV1 polypeptide” is meant an AAV1 protein with at least about 85% amino acid sequence identity to the amino acid sequence provided below, or a fragment thereof capable of multimerization to form a capsid.

>AAV1_AAD27757.1
(SEQ ID NO: 199428)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEEVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDEDKFFPMSGVMIFGKESAGA
SNTALDNVMITDEEEIKATNPVATERFGTVAVNFQSSSTDPATGDVHAMG
ALPGMVWQDRDVYLQGPIWAKIPHTDGHFHPSPLMGGFGLKNPPPQILIK
NTPVPANPPAEFSATKFASFITQYSTGQVSVEIEWELQKENSKRWNPEVQ
YTSNYAKSANVDFTVDNNGLYTEPRPIGTRYLTRPL*

By “AAV1 polynucleotide” is meant a nucleic acid molecule encoding an AAV1 polypeptide. An exemplary AAV1 nucleotide sequence is provided below.

>AAV1_AAD27757.1
(SEQ ID NO: 199429)
ATGGCTGCCGATGGTTATCTTCCAGATTGGCTCGAGGACAACCTCTCTGA
GGGCATTCGCGAGTGGTGGGACTTGAAACCTGGAGCCCCGAAGCCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAACGTCCGGTAGAGCAGTCGCCA
CAAGAGCCAGACTCCTCCTCGGGCATCGGCAAGACAGGCCAGCAGCCCGC
TAAAAAGAGACTCAATTTTGGTCAGACTGGCGACTCAGAGTCAGTCCCCG
ATCCACAACCTCTCGGAGAACCTCCAGCAACCCCCGCTGCTGTGGGACCT
ACTACAATGGCTTCAGGCGGTGGCGCACCAATGGCAGACAATAACGAAGG
CGCCGACGGAGTGGGTAATGCCTCAGGAAATTGGCATTGCGATTCCACAT
GGCTGGGCGACAGAGTCATCACCACCAGCACCCGCACCTGGGCCTTGCCC
ACCTACAATAACCACCTCTACAAGCAAATCTCCAGTGCTTCAACGGGGGC
CAGCAACGACAACCACTACTTCGGCTACAGCACCCCCTGGGGGTATTTTG
ATTTCAACAGATTCCACTGCCACTTTTCACCACGTGACTGGCAGCGACTC
ATCAACAACAATTGGGGATTCCGGCCCAAGAGACTCAACTTCAAACTCTT
CAACATCCAAGTCAAGGAGGTCACGACGAATGATGGCGTCACAACCATCG
CTAATAACCTTACCAGCACGGTTCAAGTCTTCTCGGACTCGGAGTACCAG
CTTCCGTACGTCCTCGGCTCTGCGCACCAGGGCTGCCTCCCTCCGTTCCC
GGCGGACGTGTTCATGATTCCGCAATACGGCTACCTGACGCTCAACAATG
GCAGCCAAGCCGTGGGACGTTCATCCTTTTACTGCCTGGAATATTTCCCT
TCTCAGATGCTGAGAACGGGCAACAACTTTACCTTCAGCTACACCTTTGA
GGAAGTGCCTTTCCACAGCAGCTACGCGCACAGCCAGAGCCTGGACCGGC
TGATGAATCCTCTCATCGACCAATACCTGTATTACCTGAACAGAACTCAA
AATCAGTCCGGAAGTGCCCAAAACAAGGACTTGCTGTTTAGCCGTGGGTC
TCCAGCTGGCATGTCTGTTCAGCCCAAAAACTGGCTACCTGGACCCTGTT
ATCGGCAGCAGCGCGTTTCTAAAACAAAAACAGACAACAACAACAGCAAT
TTTACCTGGACTGGTGCTTCAAAATATAACCTCAATGGGCGTGAATCCAT
CATCAACCCTGGCACTGCTATGGCCTCACACAAAGACGACGAAGACAAGT
TCTTTCCCATGAGCGGTGTCATGATTTTTGGAAAAGAGAGCGCCGGAGCT
TCAAACACTGCATTGGACAATGTCATGATTACAGACGAAGAGGAAATTAA
AGCCACTAACCCTGTGGCCACCGAAAGATTTGGGACCGTGGCAGTCAATT
TCCAGAGCAGCAGCACAGACCCTGCGACCGGAGATGTGCATGCTATGGGA
GCATTACCTGGCATGGTGTGGCAAGATAGAGACGTGTACCTGCAGGGTCC
CATTTGGGCCAAAATTCCTCACACAGATGGACACTTTCACCCGTCTCCTC
TTATGGGCGGCTTTGGACTCAAGAACCCGCCTCCTCAGATCCTCATCAAA
AACACGCCTGTTCCTGCGAATCCTCCGGCGGAGTTTTCAGCTACAAAGTT
TGCTTCATTCATCACCCAATACTCCACAGGACAAGTGAGTGTGGAAATTG
AATGGGAGCTGCAGAAAGAAAACAGCAAGCGCTGGAATCCCGAAGTGCAG
TACACATCCAATTATGCAAAATCTGCCAACGTTGATTTTACTGTGGACAA
CAATGGACTTTATACTGAGCCTCGCCCCATTGGCACCCGTTACCTTACCC
GTCCCCTGTAA

By “AAV2 polypeptide” is meant an AAV2 protein with at least about 85% amino acid sequence identity to the amino acid sequence provided below, or a fragment thereof capable of multimerization to form a capsid.

>AAV2_AAC03780.1
(SEQ ID NO: 199430)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL*

By “AAV2 polynucleotide” is meant a nucleic acid molecule encoding an AAV2 polypeptide. An exemplary AAV2 nucleotide sequence is provided below.

>AAV2_AAC03780.1
(SEQ ID NO: 199431)
ATGGCTGCCGATGGTTATCTTCCAGATTGGCTCGAGGACACTCTCTCTGA
AGGAATAAGACAGTGGTGGAAGCTCAAACCTGGCCCACCACCACCAAAGC
CCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGA
GGCAGACGCCGCGGCCCTCGAGCACGACAAAGCCTACGACCGGCAGCTCG
ACAGCGGAGACAACCCGTACCTCAAGTACAACCACGCCGACGCGGAGTTT
CAGGAGCGCCTTAAAGAAGATACGTCTTTTGGGGGCAACCTCGGACGAGC
AGTCTTCCAGGCGAAAAAGAGGGTTCTTGAACCTCTGGGCCTGGTTGAGG
AACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGC
AAGAAAAAGATTGAATTTTGGTCAGACTGGAGACGCAGACTCAGTACCTG
ACCCCCAGCCTCTCGGACAGCCACCAGCAGCCCCCTCTGGTCTGGGAACT
AATACGATGGCTACAGGCAGTGGCGCACCAATGGCAGACAATAACGAGGG
CGCCGACGGAGTGGGTAATTCCTCGGGAAATTGGCATTGCGATTCCACAT
GGATGGGCGACAGAGTCATCACCACCAGCACCCGAACCTGGGCCCTGCCC
ACCTACAACAACCACCTCTACAAACAAATTTCCAGCCAATCAGGAGCCTC
GAACGACAATCACTACTTTGGCTACAGCACCCCTTGGGGGTATTTTGACT
TCAACAGATTCCACTGCCACTTTTCACCACGTGACTGGCAAAGACTCATC
AACAACAACTGGGGATTCCGACCCAAGAGACTCAACTTCAAGCTCTTTAA
CATTCAAGTCAAAGAGGTCACGCAGAATGACGGTACGACGACGATTGCCA
ATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGC
AGACGTCTTCATGGTGCCACAGTATGGATACCTCACCCTGAACAACGGGA
GTCAGGCAGTAGGACGCTCTTCATTTTACTGCCTGGAGTACTTTCCTTCT
CAGATGCTGCGTACCGGAAACAACTTTACCTTCAGCTACACTTTTGAGGA
CGTTCCTTTCCACAGCAGCTACGCTCACAGCCAGAGTCTGGACCGTCTCA
TGAATCCTCTCATCGACCAGTACCTGTATTACTTGAGCAGAACAAACACT
CCAAGTGGAACCACCACGCAGTCAAGGCTTCAGTTTTCTCAGGCCGGAGC
GAGTGACATTCGGGACCAGTCTAGGAACTGGCTTCCTGGACCCTGTTACC
GCCAGCAGCGAGTATCAAAGACATCTGCGGATAACAACAACAGTGAATAC
TCGTGGACTGGAGCTACCAAGTACCACCTCAATGGCAGAGACTCTCTGGT
GAATCCGGGCCCGGCCATGGCAAGCCACAAGGACGATGAAGAAAAGTTTT
TTCCTCAGAGCGGGGTTCTCATCTTTGGGAAGCAAGGCTCAGAGAAAACA
AATGTGGACATTGAAAAGGTCATGATTACAGACGAAGAGGAAATCAGGAC
AACCAATCCCGTGGCTACGGAGCAGTATGGTTCTGTATCTACCAACCTCC
AGAGAGGCAACAGACAAGCAGCTACCGCAGATGTCAACACACAAGGCGTT
CTTCCAGGCATGGTCTGGCAGGACAGAGATGTGTACCTTCAGGGGCCCAT
CTGGGCAAAGATTCCACACACGGACGGACATTTTCACCCCTCTCCCCTCA
TGGGTGGATTCGGACTTAAACACCCTCCTCCACAGATTCTCATCAAGAAC
ACCCCGGTACCTGCGAATCCTTCGACCACCTTCAGTGCGGCAAAGTTTGC
TTCCTTCATCACACAGTACTCCACGGGACAGGTCAGCGTGGAGATCGAGT
GGGAGCTGCAGAAGGAAAACAGCAAACGCTGGAATCCCGAAATTCAGTAC
ACTTCCAACTACAACAAGTCTGTTAATGTGGACTTTACTGTGGACACTAA
TGGCGTGTATTCAGAGCCTCGCCCCATTGGCACCAGATACCTGACTCGTA
ATCTGTAA

By “AAV3 polypeptide” is meant an AAV3 protein with at least about 85% amino acid sequence identity to the amino acid sequence provided below, or a fragment thereof capable of multimerization to form a capsid.

>AAV3
(SEQ ID NO: 199432)
MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEF
QERLQEDTSFGGNLGRAVFQAKKRILEPLGLVEEAAKTAPGKKGAVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVRGVTQNDGTTTIANNLTSTVQVFTDSEYQL
PYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTGTVNHQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDTNGVYSEPRPIGTRYLTRNL*

By “AAV3 polynucleotide” is meant a nucleic acid molecule encoding an AAV3 polypeptide. An exemplary AAV3 nucleotide sequence is provided below.

>AAV3
(SEQ ID NO: 199433)
ATGGCTGCTGACGGTTATCTTCCAGATTGGCTCGAGGACAACCTTTCTGA
AGGCATTCGTGAGTGGTGGGCTCTGAAACCTGGAGTCCCTCAACCCAAAG
CGAACCAACAACACCAGGACAACCGTCGGGGTCTTGTGCTTCCGGGTTAC
AAATACCTCGGACCCGGTAACGGACTCGACAAAGGAGAGCCGGTCAACGA
GGCGGACGCGGCAGCCCTCGAACACGACAAAGCTTACGACCAGCAGCTCA
AGGCCGGTGACAACCCGTACCTCAAGTACAACCACGCCGACGCCGAGTTT
CAGGAGCGTCTTCAAGAAGATACGTCTTTTGGGGGCAACCTTGGCAGAGC
AGTCTTCCAGGCCAAAAAGAGGATCCTTGAGCCTCTTGGTCTGGTTGAGG
AAGCAGCTAAAACGGCTCCTGGAAAGAAGGGGGCTGTAGATCAGTCTCCT
CAGGAACCGGACTCATCATCTGGTGTTGGCAAATCGGGCAAACAGCCTGC
CAGAAAAAGACTAAATTTCGGTCAGACTGGAGACTCAGAGTCAGTCCCAG
ACCCTCAACCTCTCGGAGAACCACCAGCAGCCCCCACAAGTTTGGGATCT
AATACAATGGCTTCAGGCGGTGGCGCACCAATGGCAGACAATAACGAGGG
TGCCGATGGAGTGGGTAATTCCTCAGGAAATTGGCATTGCGATTCCCAAT
GGCTGGGCGACAGAGTCATCACCACCAGCACCAGAACCTGGGCCCTGCCC
ACTTACAACAACCATCTCTACAAGCAAATCTCCAGCCAATCAGGAGCTTC
AAACGACAACCACTACTTTGGCTACAGCACCCCTTGGGGGTATTTTGACT
TTAACAGATTCCACTGCCACTTCTCACCACGTGACTGGCAGCGACTCATT
AACAACAACTGGGGATTCCGGCCCAAGAAACTCAGCTTCAAGCTCTTCAA
CATCCAAGTTAGAGGGGTCACGCAGAACGATGGCACGACGACTATTGCCA
ATAACCTTACCAGCACGGTTCAAGTGTTTACGGACTCGGAGTATCAGCTC
CCGTACGTGCTCGGGTCGGCGCACCAAGGCTGTCTCCCGCCGTTTCCAGC
GGACGTCTTCATGGTCCCTCAGTATGGATACCTCACCCTGAACAACGGAA
GTCAAGCGGTGGGACGCTCATCCTTTTACTGCCTGGAGTACTTCCCTTCG
CAGATGCTAAGGACTGGAAATAACTTCCAATTCAGCTATACCTTCGAGGA
TGTACCTTTTCACAGCAGCTACGCTCACAGCCAGAGTTTGGATCGCTTGA
TGAATCCTCTTATTGATCAGTATCTGTACTACCTGAACAGAACGCAAGGA
ACAACCTCTGGAACAACCAACCAATCACGGCTGCTTTTTAGCCAGGCTGG
GCCTCAGTCTATGTCTTTGCAGGCCAGAAATTGGCTACCTGGGCCCTGCT
ACCGGCAACAGAGACTTTCAAAGACTGCTAACGACAACAACAACAGTAAC
TTTCCTTGGACAGCGGCCAGCAAATATCATCTCAATGGCCGCGACTCGCT
GGTGAATCCAGGACCAGCTATGGCCAGTCACAAGGACGATGAAGAAAAAT
TTTTCCCTATGCACGGCAATCTAATATTTGGCAAAGAAGGGACAACGGCA
AGTAACGCAGAATTAGATAATGTAATGATTACGGATGAAGAAGAGATTCG
TACCACCAATCCTGTGGCAACAGAGCAGTATGGAACTGTGGCAAATAACT
TGCAGAGCTCAAATACAGCTCCCACGACTGGAACTGTCAATCATCAGGGG
GCCTTACCTGGCATGGTGTGGCAAGATCGTGACGTGTACCTTCAAGGACC
TATCTGGGCAAAGATTCCTCACACGGATGGACACTTTCATCCTTCTCCTC
TGATGGGAGGCTTTGGACTGAAACATCCGCCTCCTCAAATCATGATCAAA
AATACTCCGGTACCGGCAAATCCTCCGACGACTTTCAGCCCGGCCAAGTT
TGCTTCATTTATCACTCAGTACTCCACTGGACAGGTCAGCGTGGAAATTG
AGTGGGAGCTACAGAAAGAAAACAGCAAACGTTGGAATCCAGAGATTCAG
TACACTTCCAACTACAACAAGTCTGTTAATGTGGACTTTACTGTAGACAC
TAATGGTGTTTATAGTGAACCTCGCCCTATTGGAACCCGGTATCTCACAC
GAAACTTGTGA

By “AAV3B polypeptide” is meant an AAV3B protein with at least about 85% amino acid sequence identity to the amino acid sequence provided below, or a fragment thereof capable of multimerization to form a capsid.

>AAV3B_AAB95452.1
(SEQ ID NO: 199434)
MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEF
QERLQEDTSFGGNLGRAVFQAKKRILEPLGLVEEAAKTAPGKKRPVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDTNGVYSEPRPIGTRYLTRNL*

By “AAV3B polynucleotide” is meant a nucleic acid molecule encoding an AAV3B polypeptide. An exemplary AAV3B nucleotide sequence is provided below.

>AAV3B_AAB95452.1
(SEQ ID NO: 199435)
ATGGCTGCTGACGGTTATCTTCCAGATTGGCTCGAGGACAACCTTTCTGA
AGGCATTCGTGAGTGGTGGGCTCTGAAACCTGGAGTCCCTCAACCCAAAG
CGAACCAACAACACCAGGACAACCGTCGGGGTCTTGTGCTTCCGGGTTAC
AAATACCTCGGACCCGGTAACGGACTCGACAAAGGAGAGCCGGTCAACGA
GGCGGACGCGGCAGCCCTCGAACACGACAAAGCTTACGACCAGCAGCTCA
AGGCCGGTGACAACCCGTACCTCAAGTACAACCACGCCGACGCCGAGTTT
CAGGAGCGTCTTCAAGAAGATACGTCTTTTGGGGGCAACCTTGGCAGAGC
AGTCTTCCAGGCCAAAAAGAGGATCCTTGAGCCTCTTGGTCTGGTTGAGG
AAGCAGCTAAAACGGCTCCTGGAAAGAAGAGGCCTGTAGATCAGTCTCCT
CAGGAACCGGACTCATCATCTGGTGTTGGCAAATCGGGCAAACAGCCTGC
CAGAAAAAGACTAAATTTCGGTCAGACTGGCGACTCAGAGTCAGTCCCAG
ACCCTCAACCTCTCGGAGAACCACCAGCAGCCCCCACAAGTTTGGGATCT
AATACAATGGCTTCAGGCGGTGGCGCACCAATGGCAGACAATAACGAGGG
TGCCGATGGAGTGGGTAATTCCTCAGGAAATTGGCATTGCGATTCCCAAT
GGCTGGGCGACAGAGTCATCACCACCAGCACCAGAACCTGGGCCCTGCCC
ACTTACAACAACCATCTCTACAAGCAAATCTCCAGCCAATCAGGAGCTTC
AAACGACAACCACTACTTTGGCTACAGCACCCCTTGGGGGTATTTTGACT
TTAACAGATTCCACTGCCACTTCTCACCACGTGACTGGCAGCGACTCATT
AACAACAACTGGGGATTCCGGCCCAAGAAACTCAGCTTCAAGCTCTTCAA
CATCCAAGTTAAAGAGGTCACGCAGAACGATGGCACGACGACTATTGCCA
ATAACCTTACCAGCACGGTTCAAGTGTTTACGGACTCGGAGTATCAGCTC
CCGTACGTGCTCGGGTCGGCGCACCAAGGCTGTCTCCCGCCGTTTCCAGC
GGACGTCTTCATGGTCCCTCAGTATGGATACCTCACCCTGAACAACGGAA
GTCAAGCGGTGGGACGCTCATCCTTTTACTGCCTGGAGTACTTCCCTTCG
CAGATGCTAAGGACTGGAAATAACTTCCAATTCAGCTATACCTTCGAGGA
TGTACCTTTTCACAGCAGCTACGCTCACAGCCAGAGTTTGGATCGCTTGA
TGAATCCTCTTATTGATCAGTATCTGTACTACCTGAACAGAACGCAAGGA
ACAACCTCTGGAACAACCAACCAATCACGGCTGCTTTTTAGCCAGGCTGG
GCCTCAGTCTATGTCTTTGCAGGCCAGAAATTGGCTACCTGGGCCCTGCT
ACCGGCAACAGAGACTTTCAAAGACTGCTAACGACAACAACAACAGTAAC
TTTCCTTGGACAGCGGCCAGCAAATATCATCTCAATGGCCGCGACTCGCT
GGTGAATCCAGGACCAGCTATGGCCAGTCACAAGGACGATGAAGAAAAAT
TTTTCCCTATGCACGGCAATCTAATATTTGGCAAAGAAGGGACAACGGCA
AGTAACGCAGAATTAGATAATGTAATGATTACGGATGAAGAAGAGATTCG
TACCACCAATCCTGTGGCAACAGAGCAGTATGGAACTGTGGCAAATAACT
TGCAGAGCTCAAATACAGCTCCCACGACTAGAACTGTCAATGATCAGGGG
GCCTTACCTGGCATGGTGTGGCAAGATCGTGACGTGTACCTTCAAGGACC
TATCTGGGCAAAGATTCCTCACACGGATGGACACTTTCATCCTTCTCCTC
TGATGGGAGGCTTTGGACTGAAACATCCGCCTCCTCAAATCATGATCAAA
AATACTCCGGTACCGGCAAATCCTCCGACGACTTTCAGCCCGGCCAAGTT
TGCTTCATTTATCACTCAGTACTCCACTGGACAGGTCAGCGTGGAAATTG
AGTGGGAGCTACAGAAAGAAAACAGCAAACGTTGGAATCCAGAGATTCAG
TACACTTCCAACTACAACAAGTCTGTTAATGTGGACTTTACTGTAGACAC
TAATGGTGTTTATAGTGAACCTCGCCCTATTGGAACCCGGTATCTCACAC
GAAACTTGTAA

By “AAV4 polypeptide” is meant an AAV4 protein with at least about 85% amino acid sequence identity to the amino acid sequence provided below, or a fragment thereof capable of multimerization to form a capsid.

>AAV4_AAC58045
(SEQ ID NO: 199436)
MTDGYLPDWLEDNLSEGVREWWALQPGAPKPKANQQHQDNARGLVLPGYK
YLGPGNGLDKGEPVNAADAAALEHDKAYDQQLKAGDNPYLKYNHADAEFQ
QRLQGDTSFGGNLGRAVFQAKKRVLEPLGLVEQAGETAPGKKRPLIESPQ
QPDSSTGIGKKGKQPAKKKLVFEDETGAGDGPPEGSTSGAMSDDSEMRAA
AGGAAVEGGQGADGVGNASGDWHCDSTWSEGHVTTTSTRTWVLPTYNNHL
YKRLGESLQSNTYNGFSTPWGYFDFNRFHCHFSPRDWQRLINNNWGMRPK
AMRVKIFNIQVKEVTTSNGETTVANNLTSTVQIFADSSYELPYVMDAGQE
GSLPPFPNDVFMVPQYGYCGLVTGNTSQQQTDRNAFYCLEYFPSQMLRTG
NNFEITYSFEKVPFHSMYAHSQSLDRLMNPLIDQYLWGLQSTTTGTTLNA
GTATTNFTKLRPTNFSNFKKNWLPGPSIKQQGFSKTANQNYKIPATGSDS
LIKYETHSTLDGRWSALTPGPPMATAGPADSKFSNSQLIFAGPKQNGNTA
TVPGTLIFTSEEELAATNATDTDMWGNLPGGDQSNSNLPTVDRLTALGAV
PGMVWQNRDIYYQGPIWAKIPHTDGHFHPSPLIGGFGLKHPPPQIFIKNT
PVPANPATTFSSTPVNSFITQYSTGQVSVQIDWEIQKERSKRWNPEVQFT
SNYGQQNSLLWAPDAAGKYTEPRAIGTRYLTHHL

By “AAV4 polynucleotide” is meant a nucleic acid molecule encoding an AAV4 polypeptide. An exemplary AAV4 nucleotide sequence is provided below.

>AAV4_U89790.1
(SEQ ID NO: 199437)
ATGACTGACGGTTACCTTCCAGATTGGCTAGAGGACAACCTCTCTGAAGG
CGTTCGAGAGTGGTGGGCGCTGCAACCTGGAGCCCCTAAACCCAAGGCAA
ATCAACAACATCAGGACAACGCTCGGGGTCTTGTGCTTCCGGGTTACAAA
TACCTCGGACCCGGCAACGGACTCGACAAGGGGGAACCCGTCAACGCAGC
GGACGCGGCAGCCCTCGAGCACGACAAGGCCTACGACCAGCAGCTCAAGG
CCGGTGACAACCCCTACCTCAAGTACAACCACGCCGACGCGGAGTTCCAG
CAGCGGCTTCAGGGCGACACATCGTTTGGGGGCAACCTCGGCAGAGCAGT
CTTCCAGGCCAAAAAGAGGGTTCTTGAACCTCTTGGTCTGGTTGAGCAAG
CGGGTGAGACGGCTCCTGGAAAGAAGAGACCGTTGATTGAATCCCCCCAG
CAGCCCGACTCCTCCACGGGTATCGGCAAAAAAGGCAAGCAGCCGGCTAA
AAAGAAGCTCGTTTTCGAAGACGAAACTGGAGCAGGCGACGGACCCCCTG
AGGGATCAACTTCCGGAGCCATGTCTGATGACAGTGAGATGCGTGCAGCA
GCTGGCGGAGCTGCAGTCGAGGGCGGACAAGGTGCCGATGGAGTGGGTAA
TGCCTCGGGTGATTGGCATTGCGATTCCACCTGGTCTGAGGGCCACGTCA
CGACCACCAGCACCAGAACCTGGGTCTTGCCCACCTACAACAACCACCTC
TACAAGCGACTCGGAGAGAGCCTGCAGTCCAACACCTACAACGGATTCTC
CACCCCCTGGGGATACTTTGACTTCAACCGCTTCCACTGCCACTTCTCAC
CACGTGACTGGCAGCGACTCATCAACAACAACTGGGGCATGCGACCCAAA
GCCATGCGGGTCAAAATCTTCAACATCCAGGTCAAGGAGGTCACGACGTC
GAACGGCGAGACAACGGTGGCTAATAACCTTACCAGCACGGTTCAGATCT
TTGCGGACTCGTCGTACGAACTGCCGTACGTGATGGATGCGGGTCAAGAG
GGCAGCCTGCCTCCTTTTCCCAACGACGTCTTTATGGTGCCCCAGTACGG
CTACTGTGGACTGGTGACCGGCAACACTTCGCAGCAACAGACTGACAGAA
ATGCCTTCTACTGCCTGGAGTACTTTCCTTCGCAGATGCTGCGGACTGGC
AACAACTTTGAAATTACGTACAGTTTTGAGAAGGTGCCTTTCCACTCGAT
GTACGCGCACAGCCAGAGCCTGGACCGGCTGATGAACCCTCTCATCGACC
AGTACCTGTGGGGACTGCAATCGACCACCACCGGAACCACCCTGAATGCC
GGGACTGCCACCACCAACTTTACCAAGCTGCGGCCTACCAACTTTTCCAA
CTTTAAAAAGAACTGGCTGCCCGGGCCTTCAATCAAGCAGCAGGGCTTCT
CAAAGACTGCCAATCAAAACTACAAGATCCCTGCCACCGGGTCAGACAGT
CTCATCAAATACGAGACGCACAGCACTCTGGACGGAAGATGGAGTGCCCT
GACCCCCGGACCTCCAATGGCCACGGCTGGACCTGCGGACAGCAAGTTCA
GCAACAGCCAGCTCATCTTTGCGGGGCCTAAACAGAACGGCAACACGGCC
ACCGTACCCGGGACTCTGATCTTCACCTCTGAGGAGGAGCTGGCAGCCAC
CAACGCCACCGATACGGACATGTGGGGCAACCTACCTGGCGGTGACCAGA
GCAACAGCAACCTGCCGACCGTGGACAGACTGACAGCCTTGGGAGCCGTG
CCTGGAATGGTCTGGCAAAACAGAGACATTTACTACCAGGGTCCCATTTG
GGCCAAGATTCCTCATACCGATGGACACTTTCACCCCTCACCGCTGATTG
GTGGGTTTGGGCTGAAACACCCGCCTCCTCAAATTTTTATCAAGAACACC
CCGGTACCTGCGAATCCTGCAACGACCTTCAGCTCTACTCCGGTAAACTC
CTTCATTACTCAGTACAGCACTGGCCAGGTGTCGGTGCAGATTGACTGGG
AGATCCAGAAGGAGCGGTCCAAACGCTGGAACCCCGAGGTCCAGTTTACC
TCCAACTACGGACAGCAAAACTCTCTGTTGTGGGCTCCCGATGCGGCTGG
GAAATACACTGAGCCTAGGGCTATCGGTACCCGCTACCTCACCCACCACC
TG

By “AAV5 polypeptide” is meant an AAV5 protein with at least about 85% amino acid sequence identity to the amino acid sequence provided below, or a fragment thereof capable of multimerization to form a capsid.

>AAV5_AAD13756.1
(SEQ ID NO: 199438)
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTYNLQEIVPGSVWMERD
VYLQGPIWAKIPETGAHFHPSPAMGGFGLKHPPPMMLIKNTPVPGNITSF
SDVPVSSFITQYSTGQVTVEMEWELKKENSKRWNPEIQYTNNYNDPQFVD
FAPDSTGEYRTTRPIGTRYLTRPL

By “AAV5 polynucleotide” is meant a nucleic acid molecule encoding an AAV5 polypeptide. An exemplary AAV5 nucleotide sequence is provided below.

>AAV5_AF085716.1
(SEQ ID NO: 199439)
ATGTCTTTTGTTGATCACCCTCCAGATTGGTTGGAAGAAGTTGGTGAAGG
TCTTCGCGAGTTTTTGGGCCTTGAAGCGGGCCCACCGAAACCAAAACCCA
ATCAGCAGCATCAAGATCAAGCCCGTGGTCTTGTGCTGCCTGGTTATAAC
TATCTCGGACCCGGAAACGGTCTCGATCGAGGAGAGCCTGTCAACAGGGC
AGACGAGGTCGCGCGAGAGCACGACATCTCGTACAACGAGCAGCTTGAGG
CGGGAGACAACCCCTACCTCAAGTACAACCACGCGGACGCCGAGTTTCAG
GAGAAGCTCGCCGACGACACATCCTTCGGGGGAAACCTCGGAAAGGCAGT
CTTTCAGGCCAAGAAAAGGGTTCTCGAACCTTTTGGCCTGGTTGAAGAGG
GTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGA
CGCCGAAGCTGGACCCAGCGGATCCCAGCAGCTGCAAATCCCAGCCCAAC
CAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGA
TTGGCATTGCGATTCCACGTGGATGGGGGACAGAGTCGTCACCAAGTCCA
CCCGAACCTGGGTGCTGCCCAGCTACAACAACCACCAGTACCGAGAGATC
AAAAGCGGCTCCGTCGACGGAAGCAACGCCAACGCCTACTTTGGATACAG
CACCCCCTGGGGGTACTTTGACTTTAACCGCTTCCACAGCCACTGGAGCC
CCCGAGACTGGCAAAGACTCATCAACAACTACTGGGGCTTCAGACCCCGG
TCCCTCAGAGTCAAAATCTTCAACATTCAAGTCAAAGAGGTCACGGTGCA
GGACTCCACCACCACCATCGCCAACAACCTCACCTCCACCGTCCAAGTGT
TTACGGACGACGACTACCAGCTGCCCTACGTCGTCGGCAACGGGACCGAG
GGATGCCTGCCGGCCTTCCCTCCGCAGGTCTTTACGCTGCCGCAGTACGG
TTACGCGACGCTGAACCGCGACAACACAGAAAATCCCACCGAGAGGAGCA
GCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTT
CGCTCCCAGTCAGAACCTGTTCAAGCTGGCCAACCCGCTGGTGGACCAGT
ACTTGTACCGCTTCGTGAGCACAAATAACACTGGCGGAGTCCAGTTCAAC
AAGAACCTGGCCGGGAGATACGCCAACACCTACAAAAACTGGTTCCCGGG
GCCCATGGGCCGAACCCAGGGCTGGAACCTGGGCTCCGGGGTCAACCGCG
CCAGTGTCAGCGCCTTCGCCACGACCAATAGGATGGAGCTCGAGGGCGCG
AGTTACCAGGTGCCCCCGCAGCCGAACGGCATGACCAACAACCTCCAGGG
CAGCAACACCTATGCCCTGGAGAACACTATGATCTTCAACAGCCAGCCGG
CGAACCCGGGCACCACCGCCACGTACCTCGAGGGCAACATGCTCATCACC
AGCGAGAGCGAGACGCAGCCGGTGAACCGCGTGGCGTACAACGTCGGCGG
GCAGATGGCCACCAACAACCAGAGCTCCACCACTGCCCCCGCGACCGGCA
CGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCA
CTTTCACCCCTCTCCGGCCATGGGCGGATTCGGACTCAAACACCCACCGC
CCATGATGCTCATCAAGAACACGCCTGTGCCCGGAAATATCACCAGCTTC
TCGGACGTGCCCGTCAGCAGCTTCATCACCCAGTACAGCACCGGGCAGGT
CACCGTGGAGATGGAGTGGGAGCTCAAGAAGGAAAACTCCAAGAGGTGGA
ACCCAGAGATCCAGTACACAAACAACTACAACGACCCCCAGTTTGTGGAC
TTTGCCCCGGACAGCACCGGGGAATACAGAACCACCAGACCTATCGGAAC
CCGATACCTTACCCGACCCCTTTAA

By “AAV6 polypeptide” is meant an AAV6 protein with at least about 85% amino acid sequence identity to the amino acid sequence provided below, or a fragment thereof capable of multimerization to form a capsid.

>AAV6_AAB95450.1
(SEQ ID NO: 199440)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPFGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEDVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDKDKFFPMSGVMIFGKESAGA
SNTALDNVMITDEEEIKATNPVATERFGTVAVNLQSSSTDPATGDVHVMG
ALPGMVWQDRDVYLQGPIWAKIPHTDGHFHPSPLMGGFGLKHPPPQILIK
NTPVPANPPAEFSATKFASFITQYSTGQVSVEIEWELQKENSKRWNPEVQ
YTSNYAKSANVDFTVDNNGLYTEPRPIGTRYLTRPL*

By “AAV6 polynucleotide” is meant a nucleic acid molecule encoding an AAV6 polypeptide. An exemplary AAV6 nucleotide sequence is provided below.

>AAV6_AAB95450.1
(SEQ ID NO: 199441)
ATGGCTGCCGATGGTTATCTTCCAGATTGGCTCGAGGACAACCTCTCTGA
GGGCATTCGCGAGTGGTGGGACTTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGATGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGAGGGTTCTCGAACCTTTTGGTCTGGTTGAGG
AAGGTGCTAAGACGGCTCCTGGAAAGAAACGTCCGGTAGAGCAGTCGCCA
CAAGAGCCAGACTCCTCCTCGGGCATTGGCAAGACAGGCCAGCAGCCCGC
TAAAAAGAGACTCAATTTTGGTCAGACTGGCGACTCAGAGTCAGTCCCCG
ACCCACAACCTCTCGGAGAACCTCCAGCAACCCCCGCTGCTGTGGGACCT
ACTACAATGGCTTCAGGCGGTGGCGCACCAATGGCAGACAATAACGAAGG
CGCCGACGGAGTGGGTAATGCCTCAGGAAATTGGCATTGCGATTCCACAT
GGCTGGGCGACAGAGTCATCACCACCAGCACCCGAACATGGGCCTTGCCC
ACCTATAACAACCACCTCTACAAGCAAATCTCCAGTGCTTCAACGGGGGC
CAGCAACGACAACCACTACTTCGGCTACAGCACCCCCTGGGGGTATTTTG
ATTTCAACAGATTCCACTGCCATTTCTCACCACGTGACTGGCAGCGACTC
ATCAACAACAATTGGGGATTCCGGCCCAAGAGACTCAACTTCAAGCTCTT
CAACATCCAAGTCAAGGAGGTCACGACGAATGATGGCGTCACGACCATCG
CTAATAACCTTACCAGCACGGTTCAAGTCTTCTCGGACTCGGAGTACCAG
TTGCCGTACGTCCTCGGCTCTGCGCACCAGGGCTGCCTCCCTCCGTTCCC
GGCGGACGTGTTCATGATTCCGCAGTACGGCTACCTAACGCTCAACAATG
GCAGCCAGGCAGTGGGACGGTCATCCTTTTACTGCCTGGAATATTTCCCA
TCGCAGATGCTGAGAACGGGCAATAACTTTACCTTCAGCTACACCTTCGA
GGACGTGCCTTTCCACAGCAGCTACGCGCACAGCCAGAGCCTGGACCGGC
TGATGAATCCTCTCATCGACCAGTACCTGTATTACCTGAACAGAACTCAG
AATCAGTCCGGAAGTGCCCAAAACAAGGACTTGCTGTTTAGCCGGGGGTC
TCCAGCTGGCATGTCTGTTCAGCCCAAAAACTGGCTACCTGGACCCTGTT
ACCGGCAGCAGCGCGTTTCTAAAACAAAAACAGACAACAACAACAGCAAC
TTTACCTGGACTGGTGCTTCAAAATATAACCTTAATGGGCGTGAATCTAT
AATCAACCCTGGCACTGCTATGGCCTCACACAAAGACGACAAAGACAAGT
TCTTTCCCATGAGCGGTGTCATGATTTTTGGAAAGGAGAGCGCCGGAGCT
TCAAACACTGCATTGGACAATGTCATGATCACAGACGAAGAGGAAATCAA
AGCCACTAACCCCGTGGCCACCGAAAGATTTGGGACTGTGGCAGTCAATC
TCCAGAGCAGCAGCACAGACCCTGCGACCGGAGATGTGCATGTTATGGGA
GCCTTACCTGGAATGGTGTGGCAAGACAGAGACGTATACCTGCAGGGTCC
TATTTGGGCCAAAATTCCTCACACGGATGGACACTTTCACCCGTCTCCTC
TCATGGGCGGCTTTGGACTTAAGCACCCGCCTCCTCAGATCCTCATCAAA
AACACGCCTGTTCCTGCGAATCCTCCGGCAGAGTTTTCGGCTACAAAGTT
TGCTTCATTCATCACCCAGTATTCCACAGGACAAGTGAGCGTGGAGATTG
AATGGGAGCTGCAGAAAGAAAACAGCAAACGCTGGAATCCCGAAGTGCAG
TATACATCTAACTATGCAAAATCTGCCAACGTTGATTTCACTGTGGACAA
CAATGGACTTTATACTGAGCCTCGCCCCATTGGCACCCGTTACCTCACCC
GTCCCCTGTAA

By “AAV7 polypeptide” is meant an AAV7 protein with at least about 85% amino acid sequence identity to the amino acid sequence provided below, or a fragment thereof capable of multimerization to form a capsid.

>AAV7_AAN03855.1
(SEQ ID NO: 199442)
MAADGYLPDWLEDNLSEGIREWWDLKPGAPKPKANQQKQDNGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPAKKRPVEPSP
QRSPDSSTGIGKKGQQPARKRLNFGQTGDSESVPDPQPLGEPPAAPSSVG
SGTVAAGGGAPMADNNEGADGVGNASGNWHCDSTWLGDRVITTSTRTWAL
PTYNNHLYKQISSETAGSTNDNTYFGYSTPWGYFDFNRFHCHFSPRDWQR
LINNNWGFRPKKLRFKLFNIQVKEVTTNDGVTTIANNLTSTIQVFSDSEY
QLPYVLGSAHQGCLPPFPADVFMIPQYGYLTLNNGSQSVGRSSFYCLEYF
PSQMLRTGNNFEFSYSFEDVPFHSSYAHSQSLDRLMNPLIDQYLYYLART
QSNPGGTAGNRELQFYQGGPSTMAEQAKNWLPGPCFRQQRVSKTLDQNNN
SNFAWTGATKYHLNGRNSLVNPGVAMATHKDDEDRFFPSSGVLIFGKTGA
TNKTTLENVLMTNEEEIRPTNPVATEEYGIVSSNLQAANTAAQTQVVNNQ
GALPGMVWQNRDVYLQGPIWAKIPHTDGNFHPSPLMGGFGLKHPPPQILI
KNTPVPANPPEVFTPAKFASFITQYSTGQVSVEIEWELQKENSKRWNPEI
QYTSNFEKQTGVDFAVDSQGVYSEPRPIGTRYLTRNL*

By “AAV7 polynucleotide” is meant a nucleic acid molecule encoding an AAV7 polypeptide. An exemplary AAV7 nucleotide sequence is provided below.

>AAV7_AAN03855.1
(SEQ ID NO: 199443)
ATGGCTGCCGATGGTTATCTTCCAGATTGGCTCGAGGACAACCTCTCTGA
GGGCATTCGCGAGTGGTGGGACCTGAAACCTGGAGCCCCGAAACCCAAAG
CCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCATTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGCAAAGAAGAGACCGGTAGAGCCGTCACCT
CAGCGTTCCCCCGACTCCTCCACGGGCATCGGCAAGAAAGGCCAGCAGCC
CGCCAGAAAGAGACTCAATTTCGGTCAGACTGGCGACTCAGAGTCAGTCC
CCGACCCTCAACCTCTCGGAGAACCTCCAGCAGCGCCCTCTAGTGTGGGA
TCTGGTACAGTGGCTGCAGGCGGTGGCGCACCAATGGCAGACAATAACGA
AGGTGCCGACGGAGTGGGTAATGCCTCAGGAAATTGGCATTGCGATTCCA
CATGGCTGGGCGACAGAGTCATTACCACCAGCACCCGAACCTGGGCCCTG
CCCACCTACAACAACCACCTCTACAAGCAAATCTCCAGTGAAACTGCAGG
TAGTACCAACGACAACACCTACTTCGGCTACAGCACCCCCTGGGGGTATT
TTGACTTTAACAGATTCCACTGCCACTTCTCACCACGTGACTGGCAGCGA
CTCATCAACAACAACTGGGGATTCCGGCCCAAGAAGCTGCGGTTCAAGCT
CTTCAACATCCAGGTCAAGGAGGTCACGACGAATGACGGCGTTACGACCA
TCGCTAATAACCTTACCAGCACGATTCAGGTATTCTCGGACTCGGAATAC
CAGCTGCCGTACGTCCTCGGCTCTGCGCACCAGGGCTGCCTGCCTCCGTT
CCCGGCGGACGTCTTCATGATTCCTCAGTACGGCTACCTGACTCTCAACA
ATGGCAGTCAGTCTGTGGGACGTTCCTCCTTCTACTGCCTGGAGTACTTC
CCCTCTCAGATGCTGAGAACGGGCAACAACTTTGAGTTCAGCTACAGCTT
CGAGGACGTGCCTTTCCACAGCAGCTACGCACACAGCCAGAGCCTGGACC
GGCTGATGAATCCCCTCATCGACCAGTACTTGTACTACCTGGCCAGAACA
CAGAGTAACCCAGGAGGCACAGCTGGCAATCGGGAACTGCAGTTTTACCA
GGGCGGGCCTTCAACTATGGCCGAACAAGCCAAGAATTGGTTACCTGGAC
CTTGCTTCCGGCAACAAAGAGTCTCCAAAACGCTGGATCAAAACAACAAC
AGCAACTTTGCTTGGACTGGTGCCACCAAATATCACCTGAACGGCAGAAA
CTCGTTGGTTAATCCCGGCGTCGCCATGGCAACTCACAAGGACGACGAGG
ACCGCTTTTTCCCATCCAGCGGAGTCCTGATTTTTGGAAAAACTGGAGCA
ACTAACAAAACTACATTGGAAAATGTGTTAATGACAAATGAAGAAGAAAT
TCGTCCTACTAATCCTGTAGCCACGGAAGAATACGGGATAGTCAGCAGCA
ACTTACAAGCGGCTAATACTGCAGCCCAGACACAAGTIGTCAACAACCAG
GGAGCCTTACCTGGCATGGTCTGGCAGAACCGGGACGTGTACCTGCAGGG
TCCCATCTGGGCCAAGATTCCTCACACGGATGGCAACTTTCACCCGTCTC
CTTTGATGGGCGGCTTTGGACTTAAACATCCGCCTCCTCAGATCCTGATC
AAGAACACTCCCGTTCCCGCTAATCCTCCGGAGGTGTTTACTCCTGCCAA
GTTTGCTTCGTTCATCACACAGTACAGCACCGGACAAGTCAGCGTGGAAA
TCGAGTGGGAGCTGCAGAAGGAAAACAGCAAGCGCTGGAACCCGGAGATT
CAGTACACCTCCAACTTTGAAAAGCAGACTGGTGTGGACTTTGCCGTTGA
CAGCCAGGGTGTTTACTCTGAGCCTCGCCCTATTGGCACTCGTTACCTCA
CCCGTAATCTGTAA

By “AAV8 polypeptide” is meant an AAV8 protein with at least about 85% amino acid sequence identity to the amino acid sequence provided below, or a fragment thereof capable of multimerization to form a capsid.

>AAV8_AAN03857.1
(SEQ ID NO: 199444)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESVPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQQQNTAPQIGTVNS
QGALPGMVWQNRDVYLQGPIWAKIPHTDGNFHPSPLMGGFGLKHPPPQIL
IKNTPVPADPPTTFNQSKLNSFITQYSTGQVSVEIEWELQKENSKRWNPE
IQYTSNYYKSTSVDFAVNTEGVYSEPRPIGTRYLTRNL*

By “AAV8 polynucleotide” is meant a nucleic acid molecule encoding an AAV8 polypeptide. An exemplary AAV8 nucleotide sequence is provided below.

>AAV8_AAN03857.1
(SEQ ID NO: 199445)
ATGGCTGCCGATGGTTATCTTCCAGATTGGCTCGAGGACAACCTCTCTGA
GGGCATTCGCGAGTGGTGGGCGCTGAAACCTGGAGCCCCGAAGCCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTGC
AGGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCCATCACCC
CAGCGTTCTCCAGACTCCTCTACGGGCATCGGCAAGAAAGGCCAACAGCC
CGCCAGAAAAAGACTCAATTTTGGTCAGACTGGCGACTCAGAGTCAGTTC
CAGACCCTCAACCTCTCGGAGAACCTCCAGCAGCGCCCTCTGGTGTGGGA
CCTAATACAATGGCTGCAGGCGGTGGCGCACCAATGGCAGACAATAACGA
AGGCGCCGACGGAGTGGGTAGTTCCTCGGGAAATTGGCATTGCGATTCCA
CATGGCTGGGCGACAGAGTCATCACCACCAGCACCCGAACCTGGGCCCTG
CCCACCTACAACAACCACCTCTACAAGCAAATCTCCAACGGGACATCGGG
AGGAGCCACCAACGACAACACCTACTTCGGCTACAGCACCCCCTGGGGGT
ATTTTGACTTTAACAGATTCCACTGCCACTTTTCACCACGTGACTGGCAG
CGACTCATCAACAACAACTGGGGATTCCGGCCCAAGAGACTCAGCTTCAA
GCTCTTCAACATCCAGGTCAAGGAGGTCACGCAGAATGAAGGCACCAAGA
CCATCGCCAATAACCTCACCAGCACCATCCAGGTGTTTACGGACTCGGAG
TACCAGCTGCCGTACGTTCTCGGCTCTGCCCACCAGGGCTGCCTGCCTCC
GTTCCCGGCGGACGTGTTCATGATTCCCCAGTACGGCTACCTAACACTCA
ACAACGGTAGTCAGGCCGTGGGACGCTCCTCCTTCTACTGCCTGGAATAC
TTTCCTTCGCAGATGCTGAGAACCGGCAACAACTTCCAGTTTACTTACAC
CTTCGAGGACGTGCCTTTCCACAGCAGCTACGCCCACAGCCAGAGCTTGG
ACCGGCTGATGAATCCTCTGATTGACCAGTACCTGTACTACTTGTCTCGG
ACTCAAACAACAGGAGGCACGGCAAATACGCAGACTCTGGGCTTCAGCCA
AGGTGGGCCTAATACAATGGCCAATCAGGCAAAGAACTGGCTGCCAGGAC
CCTGTTACCGCCAACAACGCGTCTCAACGACAACCGGGCAAAACAACAAT
AGCAACTTTGCCTGGACTGCTGGGACCAAATACCATCTGAATGGAAGAAA
TTCATTGGCTAATCCTGGCATCGCTATGGCAACACACAAAGACGACGAGG
AGCGTTTTTTTCCCAGTAACGGGATCCTGATTTTTGGCAAACAAAATGCT
GCCAGAGACAATGCGGATTACAGCGATGTCATGCTCACCAGCGAGGAAGA
AATCAAAACCACTAACCCTGTGGCTACAGAGGAATACGGTATCGTGGCAG
ATAACTTGCAGCAGCAAAACACGGCTCCTCAAATTGGAACTGTCAACAGC
CAGGGGGCCTTACCCGGTATGGTCTGGCAGAACCGGGACGTGTACCTGCA
GGGTCCCATCTGGGCCAAGATTCCTCACACGGACGGCAACTTCCACCCGT
CTCCGCTGATGGGCGGCTTTGGCCTGAAACATCCTCCGCCTCAGATCCTG
ATCAAGAACACGCCTGTACCTGCGGATCCTCCGACCACCTTCAACCAGTC
AAAGCTGAACTCTTTCATCACGCAATACAGCACCGGACAGGTCAGCGTGG
AAATTGAATGGGAGCTGCAGAAGGAAAACAGCAAGCGCTGGAACCCCGAG
ATCCAGTACACCTCCAACTACTACAAATCTACAAGTGTGGACTTTGCTGT
TAATACAGAAGGCGTGTACTCTGAACCCCGCCCCATTGGCACCCGTTACC
TCACCCGTAATCTGTAA

By “AAV9 K549R polypeptide” is meant an AAV9 K549R protein with at least about 85% amino acid sequence identity to the amino acid sequence provided below, or a fragment thereof capable of multimerization to form a capsid.

>AAV9 K549R
(SEQ ID NO: 199446)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL*

By “AAV9 K549R polynucleotide” is meant a nucleic acid molecule encoding an AAV9 K549R polypeptide. An exemplary AAV9 K549R nucleotide sequence is provided below.

>AAV9 K449R
(SEQ ID NO: 199447)
ATGGCTGCCGATGGTTATCTTCCAGATTGGCTCGAGGACAACCTTAGTGA
AGGAATTCGCGAGTGGTGGGCTTTGAAACCTGGAGCCCCTCAACCCAAGG
CAAATCAACAACATCAAGACAACGCTCGAGGTCTTGTGCTTCCGGGTTAC
AAATACCTTGGACCCGGCAACGGACTCGACAAGGGGGAGCCGGTCAACGC
AGCAGACGCGGCGGCCCTCGAGCACGACAAGGCCTACGACCAGCAGCTCA
AGGCCGGAGACAACCCGTACCTCAAGTACAACCACGCCGACGCCGAGTTC
CAGGAGCGGCTCAAAGAAGATACGTCTTTTGGGGGCAACCTCGGGCGAGC
AGTCTTCCAGGCCAAAAAGAGGCTTCTTGAACCTCTTGGTCTGGTTGAGG
AAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGC
TAAAAAGAGACTCAATTTCGGTCAGACTGGCGACACAGAGTCAGTCCCAG
ACCCTCAACCAATCGGAGAACCTCCCGCAGCCCCCTCAGGTGTGGGATCT
CTTACAATGGCTTCAGGTGGTGGCGCACCAGTGGCAGACAATAACGAAGG
TGCCGATGGAGTGGGTAGTTCCTCGGGAAATTGGCATTGCGATTCCCAAT
GGCTGGGGGACAGAGTCATCACCACCAGCACCCGAACCTGGGCCCTGCCC
ACCTACAACAATCACCTCTACAAGCAAATCTCCAACAGCACATCTGGAGG
ATCTTCAAATGACAACGCCTACTTCGGCTACAGCACCCCCTGGGGGTATT
TTGACTTCAACAGATTCCACTGCCACTTCTCACCACGTGACTGGCAGCGA
CTCATCAACAACAACTGGGGATTCCGGCCTAAGCGACTCAACTTCAAGCT
CTTCAACATTCAGGTCAAAGAGGTTACGGACAACAATGGAGTCAAGACCA
TCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTT
CCCAGCGGACGTTTTCATGATTCCTCAGTACGGGTATCTGACGCTTAATG
ATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTT
TGAGAACGTACCTTTCCATAGCAGCTACGCTCACAGCCAAAGCCTGGACC
GACTAATGAATCCACTCATCGACCAATACTTGTACTATCTCTCTAGAACT
ATTAACGGTTCTGGACAGAATCAACAAACGCTAAAATTCAGTGTGGCCGG
ACCCAGCAACATGGCTGTCCAGGGAAGAAACTACATACCTGGACCCAGCT
ACCGACAACAACGTGTCTCAACCACTGTGACTCAAAACAACAACAGCGAA
TTTGCTTGGCCTGGAGCTTCTTCTTGGGCTCTCAATGGACGTAATAGCTT
GATGAATCCTGGACCTGCTATGGCCAGCCACAAAGAAGGAGAGGACCGTT
TCTTTCCTTTGTCTGGATCTTTAATTTTTGGCAAACAAGGTACCGGCAGA
GACAACGTGGATGCGGACAAAGTCATGATAACCAACGAAGAAGAAATTAA
AACTACTAACCCGGTAGCAACGGAGTCCTATGGACAAGTGGCCACAAACC
ACCAGAGTGCACAAGCGCAGGCTCAAACCGGTTGGGTTCAAAACCAAGGA
ATACTTCCGGGTATGGTTTGGCAGGACAGAGATGTGTACCTGCAAGGACC
CATTTGGGCCAAAATTCCTCACACGGACGGCAACTTTCACCCTTCTCCGC
TGATGGGAGGGTTTGGAATGAAGCACCCGCCTCCTCAGATCCTCATCAAA
AACACACCTGTACCTGCGGATCCTCCAACGGCCTTCAACAAGGACAAGCT
GAACTCTTTCATCACCCAGTATTCTACTGGCCAAGTCAGCGTGGAGATCG
AGTGGGAGCTGCAGAAGGAAAACAGCAAGCGCTGGAACCCGGAGATCCAG
TACACTTCCAACTATTACAAGTCTAATAATGTTGAATTTGCTGTTAATAC
TGAAGGTGTATATAGTGAACCCCGCCCCATTGGCACCAGATACCTGACTC
GTAATCTGTAA

By “AAV9 polypeptide” is meant an AAV9 protein with at least about 85% amino acid sequence identity to the amino acid sequence provided below, or a fragment thereof capable of multimerization to form a capsid.

>AAV9
(SEQ ID NO: 199448)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL*

By “AAV9 polynucleotide” is meant a nucleic acid molecule encoding an AAV9 polypeptide. An exemplary AAV9 nucleotide sequence is provided below.

>AAV9
(SEQ ID NO: 199449)
ATGGCTGCCGATGGTTATCTTCCAGATTGGCTCGAGGACAACCTTAGTGA
AGGAATTCGCGAGTGGTGGGCTTTGAAACCTGGAGCCCCTCAACCCAAGG
CAAATCAACAACATCAAGACAACGCTCGAGGTCTTGTGCTTCCGGGTTAC
AAATACCTTGGACCCGGCAACGGACTCGACAAGGGGGAGCCGGTCAACGC
AGCAGACGCGGCGGCCCTCGAGCACGACAAGGCCTACGACCAGCAGCTCA
AGGCCGGAGACAACCCGTACCTCAAGTACAACCACGCCGACGCCGAGTTC
CAGGAGCGGCTCAAAGAAGATACGTCTTTTGGGGGCAACCTCGGGCGAGC
AGTCTTCCAGGCCAAAAAGAGGCTTCTTGAACCTCTTGGTCTGGTTGAGG
AAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGC
TAAAAAGAGACTCAATTTCGGTCAGACTGGCGACACAGAGTCAGTCCCAG
ACCCTCAACCAATCGGAGAACCTCCCGCAGCCCCCTCAGGTGTGGGATCT
CTTACAATGGCTTCAGGTGGTGGCGCACCAGTGGCAGACAATAACGAAGG
TGCCGATGGAGTGGGTAGTTCCTCGGGAAATTGGCATTGCGATTCCCAAT
GGCTGGGGGACAGAGTCATCACCACCAGCACCCGAACCTGGGCCCTGCCC
ACCTACAACAATCACCTCTACAAGCAAATCTCCAACAGCACATCTGGAGG
ATCTTCAAATGACAACGCCTACTTCGGCTACAGCACCCCCTGGGGGTATT
TTGACTTCAACAGATTCCACTGCCACTTCTCACCACGTGACTGGCAGCGA
CTCATCAACAACAACTGGGGATTCCGGCCTAAGCGACTCAACTTCAAGCT
CTTCAACATTCAGGTCAAAGAGGTTACGGACAACAATGGAGTCAAGACCA
TCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTT
CCCAGCGGACGTTTTCATGATTCCTCAGTACGGGTATCTGACGCTTAATG
ATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTT
TGAGAACGTACCTTTCCATAGCAGCTACGCTCACAGCCAAAGCCTGGACC
GACTAATGAATCCACTCATCGACCAATACTTGTACTATCTCTCAAAGACT
ATTAACGGTTCTGGACAGAATCAACAAACGCTAAAATTCAGTGTGGCCGG
ACCCAGCAACATGGCTGTCCAGGGAAGAAACTACATACCTGGACCCAGCT
ACCGACAACAACGTGTCTCAACCACTGTGACTCAAAACAACAACAGCGAA
TTTGCTTGGCCTGGAGCTTCTTCTTGGGCTCTCAATGGACGTAATAGCTT
GATGAATCCTGGACCTGCTATGGCCAGCCACAAAGAAGGAGAGGACCGTT
TCTTTCCTTTGTCTGGATCTTTAATTTTTGGCAAACAAGGAACTGGAAGA
GACAACGTGGATGCGGACAAAGTCATGATAACCAACGAAGAAGAAATTAA
AACTACTAACCCGGTAGCAACGGAGTCCTATGGACAAGTGGCCACAAACC
ACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACC
CATTTGGGCCAAAATTCCTCACACGGACGGCAACTTTCACCCTTCTCCGC
TGATGGGAGGGTTTGGAATGAAGCACCCGCCTCCTCAGATCCTCATCAAA
AACACACCTGTACCTGCGGATCCTCCAACGGCCTTCAACAAGGACAAGCT
GAACTCTTTCATCACCCAGTATTCTACTGGCCAAGTCAGCGTGGAGATCG
AGTGGGAGCTGCAGAAGGAAAACAGCAAGCGCTGGAACCCGGAGATCCAG
TACACTTCCAACTATTACAAGTCTAATAATGTTGAATTTGCTGTTAATAC
TGAAGGTGTATATAGTGAACCCCGCCCCATTGGCACCAGATACCTGACTC
GTAATCTGTAA

By “AAVrh.10 polypeptide” is meant an AAVrh.10 protein with at least about 85% amino acid sequence identity to the amino acid sequence provided below, or a fragment thereof capable of multimerization to form a capsid.

>AAVrh10_AAO88201.1
(SEQ ID NO: 199450)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYQFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFSQAKLASFITQYSTGQVSVEIEWELQKENSKRWNPE
IQYTSNYYKSTNVDFAVNTDGTYSEPRPIGTRYLTRNL*

By “AAVrh.10 polynucleotide” is meant a nucleic acid molecule encoding an AAVrh.10 polypeptide. An exemplary AAVrh.10 nucleotide sequence is provided below.

>AAVRH10_AAO88201.1
(SEQ ID NO: 199451)
ATGGCTGCCGATGGTTATCTTCCAGATTGGCTCGAGGACAACCTCTCTGA
GGGCATTCGCGAGTGGTGGGACTTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCCATCACCC
CAGCGTTCTCCAGACTCCTCTACGGGCATCGGCAAGAAAGGCCAGCAGCC
CGCGAAAAAGAGACTCAACTTTGGGCAGACTGGCGACTCAGAGTCAGTGC
CCGACCCTCAACCAATCGGAGAACCCCCCGCAGGCCCCTCTGGTCTGGGA
TCTGGTACAATGGCTGCAGGCGGTGGCGCTCCAATGGCAGACAATAACGA
AGGCGCCGACGGAGTGGGTAGTTCCTCAGGAAATTGGCATTGCGATTCCA
CATGGCTGGGCGACAGAGTCATCACCACCAGCACCCGAACCTGGGCCCTC
CCCACCTACAACAACCACCTCTACAAGCAAATCTCCAACGGGACTTCGGG
AGGAAGCACCAACGACAACACCTACTTCGGCTACAGCACCCCCTGGGGGT
ATTTTGACTTTAACAGATTCCACTGCCACTTCTCACCACGTGACTGGCAG
CGACTCATCAACAACAACTGGGGATTCCGGCCCAAGAGACTCAACTTCAA
GCTCTTCAACATCCAGGTCAAGGAGGTCACGCAGAATGAAGGCACCAAGA
CCATCGCCAATAACCTTACCAGCACGATTCAGGTCTTTACGGACTCGGAA
TACCAGCTCCCGTACGTCCTCGGCTCTGCGCACCAGGGCTGCCTGCCTCC
GTTCCCGGCGGACGTCTTCATGATTCCTCAGTACGGGTACCTGACTCTGA
ACAATGGCAGTCAGGCCGTGGGCCGTTCCTCCTTCTACTGCCTGGAGTAC
TTTCCTTCTCAAATGCTGAGAACGGGCAACAACTTTGAGTTCAGCTACCA
GTTTGAGGACGTGCCTTTTCACAGCAGCTACGCGCACAGCCAAAGCCTGG
ACCGGCTGATGAACCCCCTCATCGACCAGTACCTGTACTACCTGTCTCGG
ACTCAGTCCACGGGAGGTACCGCAGGAACTCAGCAGTTGCTATTTTCTCA
GGCCGGGCCTAATAACATGTCGGCTCAGGCCAAAAACTGGCTACCCGGGC
CCTGCTACCGGCAGCAACGCGTCTCCACGACACTGTCGCAAAATAACAAC
AGCAACTTTGCCTGGACCGGTGCCACCAAGTATCATCTGAATGGCAGAGA
CTCTCTGGTAAATCCCGGTGTCGCTATGGCAACCCACAAGGACGACGAAG
AGCGATTTTTTCCGTCCAGCGGAGTCTTAATGTTTGGGAAACAGGGAGCT
GGAAAAGACAACGTGGACTATAGCAGCGTTATGCTAACCAGTGAGGAAGA
AATTAAAACCACCAACCCAGTGGCCACAGAACAGTACGGCGTGGTGGCCG
ATAACCTGCAACAGCAAAACGCCGCTCCTATTGTAGGGGCCGTCAACAGT
CAAGGAGCCTTACCTGGCATGGTCTGGCAGAACCGGGACGTGTACCTGCA
GGGTCCTATCTGGGCCAAGATTCCTCACACGGACGGAAACTTTCATCCCT
CGCCGCTGATGGGAGGCTTTGGACTGAAACACCCGCCTCCTCAGATCCTG
ATTAAGAATACACCTGTTCCCGCGGATCCTCCAACTACCTTCAGTCAAGC
TAAGCTGGCGTCGTTCATCACGCAGTACAGCACCGGACAGGTCAGCGTGG
AAATTGAATGGGAGCTGCAGAAAGAAAACAGCAAACGCTGGAACCCAGAG
ATTCAATACACTTCCAACTACTACAAATCTACAAATGTGGACTTTGCTGT
TAACACAGATGGCACTTATTCTGAGCCTCGCCCCATCGGCACCCGTTACC
TCACCCGTAATCTGTAA

By “AAVrh.8 polypeptide” is meant an AAVrh.8 protein with at least about 85% amino acid sequence identity to the amino acid sequence provided below, or a fragment thereof capable of multimerization to form a capsid.

>AAVrh8 AAO88183.1
(SEQ ID NO: 199452)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKTGQQPAKKRLNFGQTGDSESVPDPQPLGEPPAAPSGLGP
NTMASGGGAPMADNNEGADGVGNSSGNWHCDSTWLGDRVITTSTRTWALP
TYNNHLYKQISNGTSGGSTNDNTYFGYSTPWGYFDFNRFHCHFSPRDWQR
LINNNWGFRPKRLNFKLFNIQVKEVTTNEGTKTIANNLTSTVQVFTDSEY
QLPYVLGSAHQGCLPPFPADVFMVPQYGYLTLNNGSQALGRSSFYCLEYF
PSQMLRTGNNFQFSYTFEDVPFHSSYAHSQSLDRLMNPLIDQYLYYLVRT
QTTGTGGTQTLAFSQAGPSSMANQARNWVPGPCYRQQRVSTTTNQNNNSN
FAWTGAAKFKLNGRDSLMNPGVAMASHKDDDDRFFPSSGVLIFGKQGAGN
DGVDYSQVLITDEEEIKATNPVATEEYGAVAINNQAANTQAQTGLVHNQG
VIPGMVWQNRDVYLQGPIWAKIPHTDGNFHPSPLMGGFGLKHPPPQILIK
NTPVPADPPLTFNQAKLNSFITQYSTGQVSVEIEWELQKENSKRWNPEIQ
YTSNYYKSTNVDFAVNTEGVYSEPRPIGTRYLTRNL*

By “AAVrh.8 polynucleotide” is meant a nucleic acid molecule encoding an AAVrh.8 polypeptide. An exemplary AAVrh.8 nucleotide sequence is provided below.

>AAVRH8_AAO88183.1
(SEQ ID NO: 199453)
ATGGCTGCCGATGGTTATCTTCCAGATTGGCTCGAGGACAACCTCTCTGA
GGGCATTCGCGAGTGGTGGGACTTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAAGCCTACGACCAGCAGCTCA
AAGCGGGTGACAATCCGTACCTGCGGTATAATCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCAGTCGCCA
CAAGAGCCAGACTCCTCCTCGGGCATCGGCAAGACAGGCCAGCAGCCCGC
TAAAAAGAGACTCAATTTTGGTCAGACTGGCGACTCAGAGTCAGTCCCCG
ACCCACAACCTCTCGGAGAACCTCCAGCAGCCCCCTCAGGTCTGGGACCT
AATACAATGGCTTCAGGCGGTGGCGCTCCAATGGCAGACAATAACGAAGG
CGCCGACGGAGTGGGTAATTCCTCGGGAAATTGGCATTGCGATTCCACAT
GGCTGGGGGACAGAGTCATCACCACCAGCACCCGAACCTGGGCCCTGCCC
ACCTACAACAACCACCTCTACAAGCAAATCTCCAACGGCACCTCGGGAGG
AAGCACCAACGACAACACCTATTTTGGCTACAGCACCCCCTGGGGGTATT
TTGACTTCAACAGATTCCACTGTCACTTTTCACCACGTGACTGGCAACGA
CTCATCAACAACAATTGGGGATTCCGGCCCAAAAGACTCAACTTCAAGCT
GTTCAACATCCAGGTCAAGGAAGTCACGACGAACGAAGGCACCAAGACCA
TCGCCAATAATCTCACCAGCACCGTGCAGGTCTTTACGGACTCGGAGTAC
CAGTTACCGTACGTGCTAGGATCCGCTCACCAGGGATGTCTGCCTCCGTT
CCCGGCGGACGTCTTCATGGTTCCTCAGTACGGCTATTTAACTTTAAACA
ATGGAAGCCAAGCCCTGGGACGTTCCTCCTTCTACTGTCTGGAGTATTTC
CCATCGCAGATGCTGAGAACCGGCAACAACTTTCAGTTCAGCTACACCTT
CGAGGACGTGCCTTTCCACAGCAGCTACGCGCACAGCCAGAGCCTGGACA
GGCTGATGAATCCCCTCATCGACCAGTACCTGTACTACCTGGTCAGAACG
CAAACGACTGGAACTGGAGGGACGCAGACTCTGGCATTCAGCCAAGCGGG
TCCTAGCTCAATGGCCAACCAGGCTAGAAATTGGGTGCCCGGACCTTGCT
ACCGGCAGCAGCGCGTCTCCACGACAACCAACCAGAACAACAACAGCAAC
TTTGCCTGGACGGGAGCTGCCAAGTTTAAGCTGAACGGCCGAGACTCTCT
AATGAATCCGGGCGTGGCAATGGCTTCCCACAAGGATGACGACGACCGCT
TCTTCCCTTCGAGCGGGGTCCTGATTTTTGGCAAGCAAGGAGCCGGGAAC
GATGGAGTGGATTACAGCCAAGTGCTGATTACAGATGAGGAAGAAATCAA
GGCTACCAACCCCGTGGCCACAGAAGAATATGGAGCAGTGGCCATCAACA
ACCAGGCCGCCAATACGCAGGCGCAGACCGGACTCGTGCACAACCAGGGG
GTGATTCCCGGCATGGTGTGGCAGAATAGAGACGTGTACCTGCAGGGTCC
CATCTGGGCCAAAATTCCTCACACGGACGGCAACTTTCACCCGTCTCCCC
TGATGGGCGGCTTTGGACTGAAGCACCCGCCTCCTCAAATTCTCATCAAG
AACACACCGGTTCCAGCGGACCCGCCGCTTACCTTCAACCAGGCCAAGCT
GAACTCTTTCATCACGCAGTACAGCACCGGACAGGTCAGCGTGGAAATCG
AGTGGGAGCTGCAGAAAGAAAACAGCAAACGCTGGAATCCAGAGATTCAA
TACACTTCCAACTACTACAAATCTACAAATGTGGACTTTGCTGTCAACAC
GGAGGGGGTTTATAGCGAGCCTCGCCCCATTGGCACCCGTTACCTCACCC
GCAACCTGTAA

By “LK03 polypeptide” is meant an LK03 protein with at least about 85% amino acid sequence identity to the amino acid sequence provided below, or a fragment thereof capable of multimerization to form a capsid.

>LK03
(SEQ ID NO: 199454)
MAADGYLPDWLEDNLSEGIREWWALQPGAPKPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDINGVYSEPRPIGTRYLTRPL*

By “LK03 polynucleotide” is meant a nucleic acid molecule encoding an LK03 polypeptide. An exemplary LK03 nucleotide sequence is provided below.

>LK03
(SEQ ID NO: 199455)
ATGGCTGCTGACGGTTATCTTCCAGATTGGCTCGAGGACAACCTTTCTGA
AGGCATTCGAGAGTGGTGGGCGCTGCAACCTGGAGCCCCTAAACCCAAGG
CAAATCAACAACATCAGGACAACGCTCGGGGTCTTGTGCTTCCGGGTTAC
AAATACCTCGGACCCGGCAACGGACTCGACAAGGGGGAACCCGTCAACGC
AGCGGACGCGGCAGCCCTCGAGCACGACAAGGCCTACGACCAGCAGCTCA
AGGCCGGTGACAACCCCTACCTCAAGTACAACCACGCCGACGCCGAGTTC
CAGGAGCGGCTCAAAGAAGATACGTCTTTTGGGGGCAACCTCGGGCGAGC
AGTCTTCCAGGCCAAAAAGAGGCTTCTTGAACCTCTTGGTCTGGTTGAGG
AAGCGGCTAAGACGGCTCCTGGAAAGAAGAGGCCTGTAGATCAGTCTCCT
CAGGAACCGGACTCATCATCTGGTGTTGGCAAATCGGGCAAACAGCCTGC
CAGAAAAAGACTAAATTTCGGTCAGACTGGCGACTCAGAGTCAGTCCCAG
ACCCTCAACCTCTCGGAGAACCACCAGCAGCCCCCACAAGTTTGGGATCT
AATACAATGGCTTCAGGCGGTGGCGCACCAATGGCAGACAATAACGAGGG
TGCCGATGGAGTGGGTAATTCCTCAGGAAATTGGCATTGCGATTCCCAAT
GGCTGGGCGACAGAGTCATCACCACCAGCACCAGAACCTGGGCCCTGCCC
ACTTACAACAACCATCTCTACAAGCAAATCTCCAGCCAATCAGGAGCTTC
AAACGACAACCACTACTTTGGCTACAGCACCCCTTGGGGGTATTTTGACT
TTAACAGATTCCACTGCCACTTCTCACCACGTGACTGGCAGCGACTCATT
AACAACAACTGGGGATTCCGGCCCAAGAAACTCAGCTTCAAGCTCTTCAA
CATCCAAGTTAAAGAGGTCACGCAGAACGATGGCACGACGACTATTGCCA
ATAACCTTACCAGCACGGTTCAAGTGTTTACGGACTCGGAGTATCAGCTC
CCGTACGTGCTCGGGTCGGCGCACCAAGGCTGTCTCCCGCCGTTTCCAGC
GGACGTCTTCATGGTCCCTCAGTATGGATACCTCACCCTGAACAACGGAA
GTCAAGCGGTGGGACGCTCATCCTTTTACTGCCTGGAGTACTTCCCTTCG
CAGATGCTAAGGACTGGAAATAACTTCCAATTCAGCTATACCTTCGAGGA
TGTACCTTTTCACAGCAGCTACGCTCACAGCCAGAGTTTGGATCGCTTGA
TGAATCCTCTTATTGATCAGTATCTGTACTACCTGAACAGAACGCAAGGA
ACAACCTCTGGAACAACCAACCAATCACGGCTGCTTTTTAGCCAGGCTGG
GCCTCAGTCTATGTCTTTGCAGGCCAGAAATTGGCTACCTGGGCCCTGCT
ACCGGCAACAGAGACTTTCAAAGACTGCTAACGACAACAACAACAGTAAC
TTTCCTTGGACAGCGGCCAGCAAATATCATCTCAATGGCCGCGACTCGCT
GGTGAATCCAGGACCAGCTATGGCCAGTCACAAGGACGATGAAGAAAAAT
TTTTCCCTATGCACGGCAATCTAATATTTGGCAAAGAAGGGACAACGGCA
AGTAACGCAGAATTAGATAATGTAATGATTACGGATGAAGAAGAGATTCG
TACCACCAATCCTGTGGCAACAGAGCAGTATGGAACTGTGGCAAATAACT
TGCAGAGCTCAAATACAGCTCCCACGACTAGAACTGTCAATGATCAGGGG
GCCTTACCTGGCATGGTGTGGCAAGATCGTGACGTGTACCTTCAAGGACC
TATCTGGGCAAAGATTCCTCACACGGATGGACACTTTCATCCTTCTCCTC
TGATGGGAGGCTTTGGACTGAAACATCCGCCTCCTCAAATCATGATCAAA
AATACTCCGGTACCGGCAAATCCTCCGACGACTTTCAGCCCGGCCAAGTT
TGCTTCATTTATCACTCAGTACTCCACTGGACAGGTCAGCGTGGAAATTG
AGTGGGAGCTACAGAAAGAAAACAGCAAACGTTGGAATCCAGAGATTCAG
TACACTTCCAACTACAACAAGTCTGTTAATGTGGACTTTACTGTAGACAC
TAATGGTGTTTATAGTGAACCTCGCCCCATTGGCACCCGTTACCTTACCC
GTCCCCTGTAA.

By “administering” is meant giving, supplying, dispensing a composition, agent, therapeutic product, and the like to a subject, or applying or bringing the composition and the like into contact with the subject. Administering or administration may be accomplished by any of a number of routes, such as, for example, without limitation, parenteral or systemic, intravenous (IV), (injection), subcutaneous, intrathecal, intracranial, intramuscular, dermal, intradermal, inhalation, rectal, intravaginal, topical, oral, subcutaneous, intramuscular, or intraocular. In embodiments, administration is systemic, such as by inoculation, injection, or intravenous injection.

By “agent” is meant any viral particle comprising a therapeutic molecule (e.g., antibody, nucleic acid molecule, or polypeptide, or fragments thereof). A non-limiting example of an agent is an AAV of the present disclosure.

By “alteration” is meant a change in the expression levels or activity of a gene or polypeptide as detected by standard art known methods such as those described herein. The alteration can be an increase or a decrease. As used herein, an alteration includes a 10% change in expression levels, preferably a 25% change, more preferably a 40% change, and most preferably a 50% or greater change in expression levels.”

By “analog” is meant a molecule that is not identical but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence, or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a polypeptide or nucleic acid molecule. This portion contains at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “gene” is meant a region of a polynucleotide that is transcribed as a single unit. Typically, a gene is transcribed to produce a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increase” is meant to alter positively by at least 5% relative to a reference. An increase may be by 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a developmental state, condition, disease, or disorder.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “manufacturability,” “production fitness,” “production,” or “produces” with reference to a capsid polypeptide is meant how well a capsid polynucleotide is expressed in a cell and the amount of viral particles produced from the expressed capsid polypeptides that are capable of delivering a payload to a cell. In embodiments, the production efficiency of a capsid polypeptide may be measured as the number of functional viral particles produced using a particular amount of a polynucleotide encoding the capsid polypeptide. In some cases, an AAV capsid with good production is an AAV capsid that yields greater or comparable levels of functional AAV viral particles relative to a reference AAV viral capsid. Production fitness of a capsid polypeptide can be assessed using methods provided herein.

The term “recombinant” as used herein in the context of proteins or nucleic acids refers to proteins or nucleic acids that do not occur in nature or in a naturally occurring protein or nucleic acid sequence, but are the product of human engineering, often or typically utilizing molecular biological or molecular genetic tools and techniques practiced by the skilled practitioner in the art. For example, in some embodiments, a recombinant protein or nucleic acid molecule comprises an amino acid or nucleotide sequence that comprises at least one, at least two, at least three, at least four, at least five, at least six, at least seven, or at least eight mutations as compared to any naturally occurring sequence.

By “reduce” is meant to alter negatively by at least 5% relative to a reference. A reduction may be by 5%, 10%, 25%, 30%, 50%, 75%, or even by 100%.

By “reference” is meant a standard or control condition. In embodiments, a reference is a cell or animal that does not express a particular recombinase (e.g., Cre or FLP). In some embodiments, the reference is a cell or animal that has not been contacted with or administered a viral particle. In some cases, a reference is a capsid polypeptide that does not comprise a peptide insert of the present disclosure.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that can be transcribed into an mRNA molecule or that encodes a polypeptide of the invention or a fragment thereof. In embodiments, the mRNA contains a sequence corresponding to a barcode and/or invertible spacer of the present disclosure. In embodiments, nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M., and S. L. Berger (1987) Methods Enzymol. 152:399; Kimmel, A. R. (1987) Methods Enzymol. 152:507). In some instances, the nucleic acid molecule encodes a polypeptide that is not endogenous to a target cell or animal. In some cases, the nucleic acid molecule encodes a capsid polypeptide of the present disclosure or a fragment thereof.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 g/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence or nucleic acid sequence. In embodiments, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an organism. In embodiments, the organism is a mammal. Non-limiting examples of a subject include a human or non-human mammal, such as a non-human primate (e.g., a marmoset), or a non-human mammal, such as a bovine, equine, canine, ovine, or feline mammal, or a sheep, goat, llama, camel, or a rodent (rat, mouse), ferret, gerbil, hamster, or zebrafish.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Transduction” refers to a process by which a polynucleotide is introduced or transferred into a cell. In embodiments, a cell is transduced by a virus or viral vector. In embodiments, the transduced polynucleotide (e.g., RNA, DNA) is expressed in the transduced cell.

As used herein, the term “vehicle” refers to a solvent, diluent, or carrier component of a pharmaceutical composition.

By “viral genome” is meant a polynucleotide molecule suitable for encapsidation by a viral capsid. A non-limiting example of a viral genome is a polynucleotide (e.g., single-stranded DNA) containing and/or flanked by two adeno-associated virus inverted terminal repeats (ITR's). In some cases, a viral genome contains a rep open reading frame and/or a cap open reading frame. In embodiments, the viral capsid is an adeno-associated virus capsid or a lentivirus capsid. In various instances, the viral genome is of sufficient size for encapsidation by a viral capsid (e.g., less than 4.7 kilobases long).

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

In any of the above aspects, or embodiments thereof, the cells form part of an organoid or virtual organ. In any of the above aspects, or embodiments thereof, the cells contain two or more different cell types.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1E provide illustrations showing an overview of a systematic multi-trait protein optimization paradigm. FIG. 1A provides an illustration of an insertion-modified AAV virus library that uniformly samples the 7-mer sequence space (1.28 billion possible variants) and is designed and used to produce AAV particles. Variant production fitness is measured via NGS of nuclease-resistant Cap-containing genomes (VRPM) relative to the number of genomes in the DNA library (DRPM). FIG. 1B provides an illustration of a fitness predictor and graph showing that the production fitness data is used to train a sequence-to-production-fitness ML model that is then used to design the Fit4Function library, which uniformly and exclusively samples the high production fitness sequence space. A sequence-to-production-fitness ML model was built and used to create the Fit4Function library, which uniformly and exclusively samples the high production fitness sequence space. FIG. 1C provides an illustration showing that the Fit4Function library can be screened in vivo or in vivo for functions of interest, and the data are used to derive ML models that predict these functions from random 7-mer sequences. FIG. 1D is an illustration showing that the production fitness and functional models are used in combination to populate MultiFunction libraries consisting of variants predicted to perform well across the desired traits (see checkered areas that represent the overlap between the functional sequence spaces of interest). FIG. 1E is an illustration showing that the MultiFunction libraries were screened for all functions of interest, The top performing variants were then individually validated.

FIG. 2 provides a series of heatmaps showing that production fitness replication quality improved upon hierarchical aggregation of replicates. The heatmaps show replication quality between replicates, where replication quality was defined as the Pearson correlation of log 2 reads per million (RPM) between replicates. Going from left-to-right in FIG. 2, data was collapsed by technical replicates, then biological replicates, then by researchers, with replication quality increasing as replicates were collapsed.

FIGS. 3A-3G provide scatter plots, histograms, heatmaps, and a plot showing mapping and learning the 7-mer production fitness landscape. FIG. 3A provides a scatter plot showing a correlation between the production fitness score of codon replicate pairs. Each pair was aggregated across 12 replications. The vertical and horizontal distributions correspond to ‘missing’ cases, where only one codon replicate of a pair was detected. FIG. 3B provides a histogram showing the production fitness distribution of the training library representing the variants detected in at least one of the 24 replicates (92.4% of total variants). The distributions representing low versus high production fitness are depicted. FIG. 3C provides a heatmap showing the AA distribution by position for the variants in the 70K most abundant sequences in an NNK library versus the high fit distribution of the training library (27K). FIG. 3I) provides a scatter plot showing production fitness replication quality of the control set (10K) shared between the training and assessment libraries. FIGS. 3E and 3F provide scatter plots showing measured versus predicted fitness score when the model is trained on a subset of the training library and tested on another subset of the same library (FIG. 3E) versus when tested on the independent assessment library, not including the overlapping 10K set (FIG. 3F). FIG. 3G provides a plot showing performance of the fitness prediction model across different training set sizes.

FIGS. 4A-4C provide histograms and a stacked bar graph showing codon usage of 7-mer insertions minimally affected capsid fitness. FIG. 4A provides a histogram showing the distribution of the difference in fitness scores measured between codon replicate pairs and between technical replicate pairs are similar (Kullback-Leibler divergence=0.006±0.007). FIG. 4B provides a histogram showing the variants with a single codon replicate detected (missing matching codon) had fitness scores on the low end of the fitness bimodal distribution. FIG. 4C provides a bar graph showing codon usage distribution in the training library followed the expected uniform distribution for each amino acid.

FIG. 5 provides a bar chart and histogram distinguishing high- and low-production fitness distributions. The production fitness of detected stop-codon containing variants in the training library, presumably arising due to cross-packaging, versus the production fitness landscape of the detected library non-control variants (codon replicates not aggregated). 40.1% of the stop codon-containing sequences were undetected in the virus library.

FIGS. 6A-6H provide a schematic, a histogram, a heat map, bar graphs, and scatter plots showing Fit4Function libraries evenly sampled the high fit production space and enabled more accurate functional screening and prediction. FIG. 6A provides a schematic showing the composition of the Fit4Function library. FIG. 6B provides a histogram showing a calibrated distribution of the measured fitness scores for the Fit4Function library versus the training library. FIG. 6C provides a heatmap showing the AA distribution by position for the variants in the Fit4Function library, high fit distribution of the training library, and 240K most abundant sequences in an NNK library. FIG. 6D provides a bar graph showing a distribution of Hamming distances between pairs of variants in NNK vs the Fit4Function library. FIG. 6E provides a bar graph showing a quantitative comparison of pairwise Pearson correlations among biological triplicates for functional screens using the Fit4Function library (240K) versus an NNK library (top 240K variants). hCMEC/d3=human brain endothelial cell line, mBMVEC=C57 primary brain microvascular endothelial cells, hBMVEC=human primary brain microvascular endothelial cells. FIG. 6F provides scatter plots showing measured versus predicted log 2 enrichment scores for models trained on Fit4Function versus NNK library data. FIG. 6G provides a bar graph showing replication quality between pairs of animals for the biodistribution in eight organs. FIG. 6H provides scatter plots showing prediction performance of models trained on in vivo biodistribution of Fit4Function library across 8 organs.

FIG. 7 provides a heatmap showing Fit4Function variant biodistribution correlation between organs.

FIGS. 8A and 8B provide plots showing replicability of five assays for hepatocyte MultiFunction training from Fit4Function screens. Pairwise correlations between biological triplicates for (FIG. 8A) production fitness and (FIG. 8B) in vitro assays of HepG2 binding or transduction and THLE binding or transduction.

FIGS. 9A-9D provide scatter plots, histograms, a bar graph, and a heatmap relating to MultiFunction library generation from functional screens of the Fit4Function Library. FIG. 9A provides a series of scatter plots showing Pearson correlation of measured versus predicted enrichment for production fitness and functional assays relevant to hepatocyte cross-species targeting. FIG. 9B provides histograms showing the distribution of enrichment across variants sampled from the Uniform (3K), Fit4Function (10K), Positive Control (Fit4Function variants satisfying the six conditions), and MultiFunction libraries. Histograms are density-normalized, including non-detected variants (ND). FIG. 9C provides a bar graph showing hit rate for variants satisfying the six conditions in each listed variant set. Positive control variants were selected to all meet the six conditions and are not plotted. FIG. 9D provides a heatmap showing the AA distribution by position for the variants in the MultiFunction library.

FIGS. 10A-10C provide plots showing replicability of MultiFunction library across in vitro and in vivo assays. FIG. 10A provides plots of production fitness. FIG. 10B provides plots of human in vivo cell binding and transduction. FIG. 10C provides plots of in vivo liver biodistribution in C57BL/6J mice.

FIGS. 11A-11F provide a schematic, web plots, histograms, and a bar graph showing individual validation of MultiFunction capsids with enhanced cross-species hepatocyte transduction. FIG. 11A provides a schematic and a collection of web plots showing on-target and off-target measurements for the seven selected capsids (BI151-157) and AAV9 in the MultiFunction library pool, shown as normalized log 2 enrichments of the selected capsid (2 codon replicates) as compared to AAV9 (4 codon replicates). Measured enrichment was linearly normalized according to the maximum and minimum enrichment values for each assay across all capsids. Individual codon replicates are plotted as points, and the average normalized enrichments across replicates are plotted as polygon vertices. FIG. 11B provides provides histograms showing C57BL/6J liver transduction by AAV9 or MultiFunction capsids. Mice were injected with 1×10¹⁰ vg of the indicated capsid packaging AAV-CAG-GFP-2A-Luc-WPRE-pA and assessed for GFP expression three weeks later (n=5 mice for each AAV treatment condition, n=3 mice for the no AAV control, mean±s.d., all BI capsids were not significantly different from AAV9 in unpaired, one-sided t-tests with Bonferroni correction). The distributions of median GFP pixel intensity per DAPI+ nuclei, combined across n=5 animals for each AAV treatment condition, and n=3 animals for the no AAV control are shown. The vertical lines within each distribution represent the mean of each animal. FIG. 11C provides a bar graph showing HepG2 and THLE transduction assessed 24 hours post transduction with 3000 vg/cell using a luciferase assay. Luciferase relative light units were normalized to AAV9. N=4 per group, mean±s.d., ***p<0.001, unpaired, one-sided t-tests corrected for multiple-hypotheses (Bonferroni). For each pair of bars in FIG. 11C, the bar on the left corresponds to THLE and the bar on the right corresponds to HEPG2. FIG. 11D provides a histogram showing on-target and off-target measurements for the seven selected capsids (BI151-157) and AAV9 in the MultiFunction library pool, shown as normalized log 2 enrichments of the selected capsid (two 7-mer replicates) as compared to AAV9 (four 7-mer replicates). Measured enrichment was linearly normalized according to the maximum and minimum enrichment values for each assay across all capsids. Individual 7-mer replicates are plotted as points, and the average normalized enrichments across replicates are plotted as polygon vertices. FIG. 11E provides a bar graph and illustration that HepG2 and THLE transduction were assessed 24 hours post-transduction at 3000 vg/cell using a luciferase assay (n=4 transduction replicates per group, mean±s.d., ****p<1e-4, unpaired, one-sided t-tests on log-transformed values, and Bonferroni corrected for multiple-hypotheses). Luciferase relative light units were normalized to AAV9. FIG. 11F provides a plot showing macaque liver transduction efficiency for the seven individually characterized liver MultiFunction variants (n=2 rhesus macaques). In the virus library, each variant was represented by two 7-mer replicates while AAV9 was represented by three replicates.

FIGS. 12A-12C provide bar graphs showing individual assessment of liver MultiFunction capsids for production and cell transduction. FIG. 12A provides a bar graph of production yields for the selected capsids when individually manufactured. FIG. 12B provides a bar graph presenting data from an experiment where AAV9 or the indicated AAV capsid was used to transduce C57BL/6J mice at 1×10¹⁰ vg/mouse. Three weeks after AAV administration, liver transduction was measured by RT-qPCR of AAV transcripts from extracted tissue. ΔΔCt was obtained by normalizing against the reference gene (GAPDH), and then against the control (AAV9). N=5/group; mean±s.d., unpaired one-sided t-tests corrected for multiple-hypotheses (Bonferroni). FIG. 12C provides a bar graph showing normalized luciferase activity in human liver cell line (THLE, HepG2) and HEK293 transduction 24 hours after exposure to 5000 vg/cell of the capsid packaging AAV-CAG-GFP-2A-Lue-WPRE-pA. N=4 per group, mean±s.d., *p<0.05, **p<0.01, ***p<0.001, unpaired one-sided t-tests corrected for multiple-hypotheses (Bonferroni). For each set of three bars in FIG. 12C, the left bar corresponds to THLE, the middle bar corresponds to HEPG2, and the right bar corresponds to HEK293.

FIG. 13 provides a set of histograms showing production fitness distributions of AAV9 capsid variants modified with 7mer insertions between amino acid 588 and 589. Production fitness was measured by the enrichment (fold change) in virus production for a variant relative to its starting plasmid reported (the packaged virus DNA RPM/plasmid DNA RPM). The vertical line and text indicate the number of capsid variants that were positively enriched. Experiments 1 and 2 show distributions of a library of capsids that uniformly sampled the 7mer amino acid (AA) sequence space. Experiments 3 and 4 show the production fitness distributions of capsids that sample the high fitness sequence space. Enrichment was averaged across technical and biological replicates for each experiment and reported as log 2(enrichment).

FIG. 14 provides a collection of histograms showing in vivo binding and transduction distributions of AAV9 capsid variants modified with 7mer insertions between amino acid 588 and 589. A Fit4Function library comprising 240K unique high production fit capsids was screened on the indicated human and mouse primary cells and established cell lines. The vertical line and text indicate the number of capsid variants that were positively enriched for each assay and for production fitness. Enrichment was measured and shown as in FIG. 13.

FIG. 15 provides a set of histograms showing AAV9 capsid loop VIII 7-mer variant in vivo biodistribution and transduction. A Fit4Function library comprising 240K unique high production fit capsids was administered intravenously to C57BL/6J mice. Two hours later, DNA was isolated from serum or indicated organs and AAV capsid sequences were recovered through PCR amplification and NGS sequencing. The plots show the distribution of enrichment for the specific assay. The vertical line and text indicate the number of capsid variants that were both positively enriched for each assay and for production fitness (not shown). Enrichment was measured and shown as in FIG. 13.

FIG. 16 provides a set of bar graphs showing charge distribution by position within the 7-mer and in total for the 30K MultiFunction liver capsid variants. The plots show the frequency of positively charged amino acid (AA) (+1; R or K), negatively charged AA (−1; D and E), and neutral (0, includes H). Nearly all of the liver MultiFunction capsids had a 7-mer with an net charge of +1 (bottom left).

FIG. 17 provides a schematic showing an overview of an embodiment of a systematic multi-parameter protein optimization paradigm. FIG. 17 discloses SEQ ID NOS 200025-200027 and 200025-200027, respectively in order of appearance.

FIG. 18 provides images showing in vivo mouse liver transduction by each MultiFunction capsid. Representative GFP images of liver slices for the no AAV control, AAV9, and BI variants. Images were chosen from the median replicate of the median animal per condition. All images were taken at the same exposure and rescaled to the same intensity range. Scale bar in all images=100 μm.

FIG. 19 provides a histogram showing macaque detargeted AAV variants with production fitness above WT.

FIG. 20 provides a series of plots showing that Fit4Function enabled top candidate selection from a single round of screening. A Fit4Function library containing 90K unique 7-mers was injected intravenously into a single cynomolgus macaque and tissues were collected 4 hours later for biodistribution analysis. The plots show the correlations between two biological 7-mer replicates for the most enriched sequences in each organ. Variants with a log 2 enrichment at least two-fold greater than that of AAV9 within each organ are shown. The top two AAV9 replicates were selected in each organ separately to set a more stringent threshold for top hit identification.

### DETAILED DESCRIPTION OF THE INVENTION

As described below, the present invention features adeno-associated viral vectors and methods of using such vectors.

The invention of the disclosure is based, at least in part, upon the design of new adeno-associated virus (AAV) capsids and libraries comprising the same. Systematically identifying protein variants with multiple enhanced traits remains a major protein engineering challenge. Focusing on adeno-associated virus (AAV) capsids, a machine learning-compatible Fit4Function library was designed that evenly samples the sequence space of high production fit variants. With this library, generalizable MIL models were trained to predict gene therapy-relevant traits including production yield, in vivo biodistribution, and binding and transduction of human cells. The models were used to design a library that efficiently explored capsids predicted to possess multiple traits important for hepatocyte gene delivery. Upon validation, 90% of the library variants met all predetermined criteria. Individually tested capsids exhibited efficient cross-species hepatocyte transduction. In embodiments, the Fit4Function approach is applicable to the multi-trait enhancement of other proteins amenable to quantitative, high-throughput engineering.

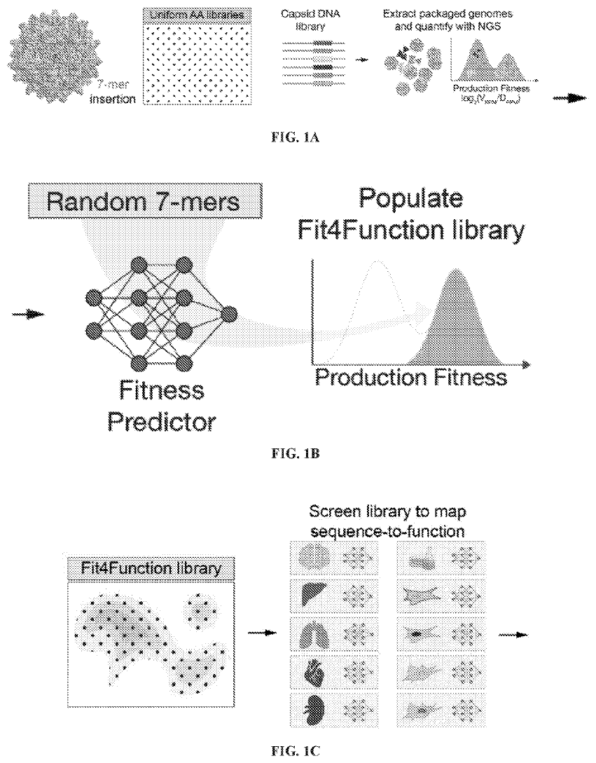

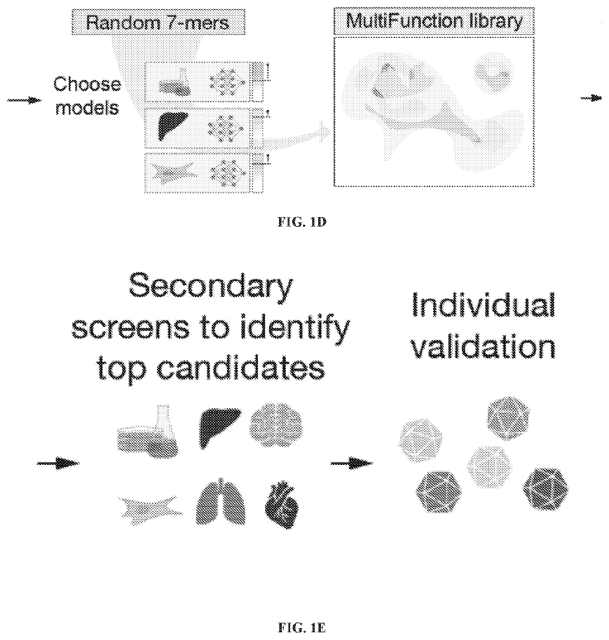

As described above, the invention of the disclosure is based, at least in part, upon the development of a generalizable machine learning-guided approach to systematically and simultaneously map 7-mer-modified AAV9 capsid sequences to multiple functions. To generate high-quality data that would enable the training of accurate ML models, a low bias, high diversity library composed only of capsid variants with high production fitness was created (FIG. 1). This “Fit4Function” library was subjected to in vitro and in vivo screens for traits relevant to gene therapy, which, as anticipated, resulted in highly reproducible data that could be used to train robust machine learning models. The models trained using the Fit4Function data were of sufficient accuracy that they could be leveraged in combination to search the much larger, untested, theoretical high production fitness sequence space in silico for rare multi-trait variants. It was first demonstrated that six of these models relating to liver-targeting, when combined, resulted in a high 88.5% validation rate. Then, despite being trained only on mouse in vivo and human in vitro data, this combination of models was translated to the macaque, and all individually validated variants performed well across human cells, mice and macaque compared to AAV9. Notably, the combination of in vivo and in vitro functional predictors boosted the precision of cross-species prediction compared to the use of any individual model. In other words, value was observed in training models on human cell in vitro data to predict variants that exhibit the traits of interest in mice and macaque in vivo. The Fit4Function approach allowed for the systemic and ready identification of combination of traits important in predicting a given function of interest. Appropriate screening models can be identified and used to enrich for multi-trait capsids prior to more costly studies in NHPs or clinical trials. This strategy can inform intelligent searches for AAV capsids that are functional across species and likely to translate from preclinical models to investigational human gene therapies.

In various embodiments, the capsids and/or capsid libraries of the present disclosure possess one or more of the following traits: enhanced on-target delivery; reduced delivery to common accumulation sites; resistance to pre-existing antibodies (e.g., pre-existing circulating antibodies in a subject); and/or improved or maintained manufacturability. In embodiments, the capsids and/or capsid libraries of the present disclosure are suitable for infecting human cells. In some instances, the capsids and/or capsid libraries of the present disclosure are resistant to a polyclonal response. In some embodiments, capsids and/or capsid libraries of the present invention are suitable for infecting one or more species (e.g., a mouse and a primate, such as a human). In some cases, the present disclosure provides capsids or libraries containing the same that have increased immune evasion.

### Capsid Libraries and Screening

In various aspects, the disclosure features capsid libraries containing polypeptides or polynucleotides encoding the same. In aspects, the disclosure features methods for screening the capsid libraries. In aspects, the present disclosure features viral particles containing capsid polypeptides. In various cases, the capsid libraries are prepared by inserting peptides of a predetermined length into a parent/reference adeno-associated virus capsid polypeptide (e.g., an AAV9 K449R polypeptide). In embodiments, the peptides are 2-mers, 3-mers, 4-mers, 5-mers, 6-mers, 7-mers, 8-mers, 9-mers, 10-mers, 11-mers, 12-mers, 13-mers, 14-mers, 15-mers, or longer n-mers. The peptides can be inserted at any of various locations in the capsid polypeptide, such as within a loop of the capsid polypeptide (e.g., Loop VIII of the polypeptide); for example, the peptide may be inserted after or before amino acid position 577, 586, 587, 588, 589, or 590 of the polypeptide. In some cases, the capsid polypeptide is an AAV1, AAV2, AAV3, AAv3b, AAV4, AAV5, AAV6, AAV7, AAV8, AAV9, AAV9 K449R, rh.10, rh.8, or LK03 polypeptide. Non-limiting examples of representative insertion sites are provided in Table 1 below. A capsid library can contain about, or at least about 2, 5, 10, 50, 100, 500, 1e3, 5e3, 1e4, 5e4, 1e5, 2e5, 3e5, 4e5, 5e5, 6e5, 7e5, 8e5, 9e6, 1e6, 5e6, or 1e7 unique insertions.

TABLE 1
Variable region VIII insertion sites in alternative AAV capsids.
Equivalent insertion indicates the position within the indicated
capsid that best aligns with the insertion site after AA 588
of AAV9 K449R. Insertions may alternatively be placed after
the indicated adjacent amino acids within Loop VIII.

| | Equivalent insertion | |
|---|---|---|
| | (where the insertion is | |
| | between the listed | |
| | amino acid and the | |
| | following n + 1 amino | |
| | acid (e.g., between AA | Loop VIII amino |
| Capsid | 588 and 589)) | acids |
| AAV1 | 588 | [586-592] |
| AAV2 | 586 | [584-590] |
| AAV3 | 587 | [585-591] |
| AAV3b | 587 | [585-591] |
| AAV4 | 586 | [584-590] |
| AAV5 | 577 | [575-581] |
| AAV6 | 588 | [586-592] |
| AAV7 | 589 | [587-593] |
| AAV8 | 590 | [588-594] |
| AAV9 | 588 | [586-592] |
| AAV9 | 588 | [586-592] |
| K449R | | |
| rh. 10 | 590 | [588-594] |
| rh. 8 | 588 | [586-592] |
| LK03 | 588 | [586-592] |

In embodiments, a capsid library of the present disclosure contains two or more capsids individually containing unique 7-mers selected from SEQ ID NOs: 1-199427. In embodiments, the library contains 2, 5, 10, 50, 100, 500, 1e3, 5e3, 1e4, 5e4, 1e5, or 199,427 unique 7-mers selected from SEQ ID NOs: 1-199,427. In embodiments, all of the sequences in the capsid library are capable of forming viral particles sharing 1, 2, 3, 4, 5, 6 or more common traits selected from one or more of those described herein, such as binding a cell of interest (e.g., liver cell, hepatocyte, HepG2, THLE, T cell; HEK293 cell, brain endothelial cell; C57 brain endothelial cell; hCME CD3; kidney cell; spinal cord cell); transducing a cell of interest (e.g., liver cell, hepatocyte, HepG2, THLE, T cell; HEK293 cell, brain endothelial cell; C57 brain endothelial cell; hCME CD3; kidney cell; spinal cord cell); biodistributing to the liver of an organism (e.g., human, rodent); production fitness; heart biodistribution; spleen biodistribution; kidney biodistribution; serum biodistribution; brain biodistribution; lung biodistribution; spinal cord biodistribution; and spinal cord transduction. In embodiments, the common trait(s) is increased relative to a reference viral particle. In some cases, the reference viral particle is selected from a viral particle containing a capsid polypeptide selected from one or more of the following and not including any peptide insert: AAV1, AAV2, AAV3, AAv3b, AAV4, AAV5, AAV6, AAV7, AAV8, AAV9, AAV9 K449R, rh.10, rh.8, or LK03. Further non-limiting examples of common traits include binding to or transfecting one or more of the following cell types: HEK293 T cells, primary mouse brain microvascular endothelial cells, primary human BMVEC cells, and/or human brain endothelial cell line hCMEC/D3 cells. In some cases, the common trait(s) include increased biodistribution relative to a reference viral particle in one or more of the following organs: liver, kidney, spleen, brain, spinal cord, serum, heart, and/or lungs. In embodiments, a capsid library is enriched for capsids capable of forming viral particles with the common trait(s) relative to reference capsid library (e.g., a randomly selected library of capsid sequences and/or a library of capsid sequences containing a random collection of 7-mer peptides inserted at a particular amino acid location).

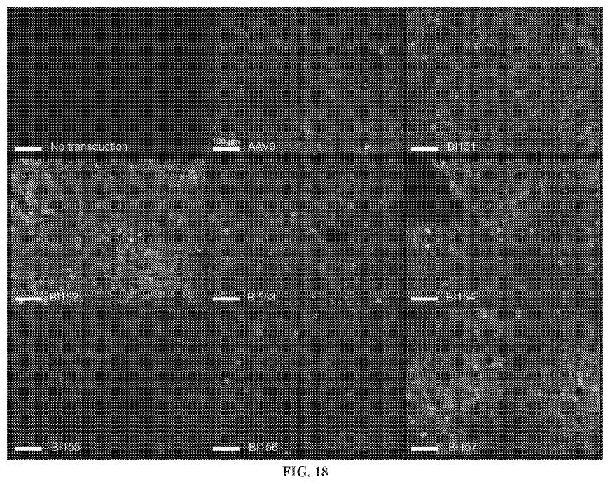

In various embodiments, the methods of the present disclosure involve selecting from a library of capsid polypeptides those capsid polypeptides having a trait(s) of interest (e.g., binding, biodistribution, or transduction capabilities). The selection can be carried out in silico or in vivo using a selection criterion or selective pressure. In embodiments, the methods of the present disclosure allow for the simultaneous optimization of multiple capsid functions, such as production, biodistribution to a target organ in a particular species, enhanced biodistribution to a target organ in a particular species, and enhanced target cell type (e.g., a human target cell type) transduction. In embodiments, machine learning (ML) models are used to deeply sample sequence space for capsids that have traits of interest. Capsids identified as having traits of interest can then be selected to populate a multi-function library containing the in silico predicted sequences (see, e.g., FIG. 17). Libraries of capsid sequences prepared in this manner can be referred to as “Fit4Function” libraries or “MultiFunction” libraries. In various instances, Fit4Function libraries contain only AAV capsids that have a trait of interest, such as production fitness (FIG. 17). The Fit4Function libraries and individual capsids thereof can be characterized, and information gained through such characterization can be used to further optimize the machine learning models to more accurately identify capsids with traits of interest. The Fit4Function libraries can be screened in vivo or in silico for capsid variants having enhanced functions (FIG. 17). Such screens can be carried out using any methods available in the art and/or those methods described herein.

In embodiments, a Fit4Function library contains high production capsids. It can be advantageous for the Fit4Function libraries to contain less amino acid bias than libraries constructed using alternative approaches (e.g., random selection of sequences, such as traditional NNN/NNK libraries). All sequences contained within a Fit4Function library are known and, accordingly, each library is accompanied by a member list providing a comprehensive list of all capsid sequences contained within the library. In some cases, the Fit4Function libraries facilitate more accurate machine learning (ML) models that can learn a theoretical sequence-to-function mapping. The Fit4Function libraries can enable efficient exploration of the multi-functional fitness space and/or enable data accumulation across species and experiments.

In some cases, the present disclosure provides libraries of capsid polypeptides, or polynucleotides encoding the same, where the libraries contain capsid polypeptides that satisfy a detargeting trait. Non-limiting examples of detargeting traits include reduced transduction of a target cell type or organ (e.g., reduced liver transduction) and reduced biodistribution in a particular organ (e.g., spleen biodistribution) or species.

In various embodiments, a library of capsids of the present disclosure contains a higher proportion of capsids with a trait(s) of interest than a reference library of randomly selected capsid sequences (e.g., capsids containing a random selection of 7-mer peptides inserted at a particular amino acid position within a reference capsid polypeptide sequence). In embodiments, a library of capsids contains capsids or contains only capsids having one or more (e.g., 1, 2, 3, 4, 5, or all) of the following traits: 1) high binding affinity to HepG2 cells, 2) high binding affinity to THLE cells, 3) high transduction of HepG2 wells, 4) high transduction of THLE cells, 5) high biodistribution to C57 mice liver, and 6) high production fitness.

In embodiments, viral particles containing capsids polypeptides of the present disclosure can transduce muscle, liver, brain, retina, and/or lung cells in vivo and/or in vitro. The efficiency of rAAV transduction is dependent on the efficiency at each step of AAV infection, i.e., virus binding, entry, trafficking, nuclear entry, uncoating, and second-strand synthesis.

### Adeno-Associated Virus (AAV)

Adeno-associated viruses (AA-V) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV vector genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes) The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms Chiorini et al, J. Vir. 71: 6823-33(1997) Srivastava et al., J. Vir. 45:555-64 (1983.) Chiorini et al., J. Vir. 73:1309-1319 (1999); Rutledge et al., J. Vir. 72:309-319 (1998), and Wu et al., J. Vir 74. 8635-47 (2000), the contents of each of which are incorporated herein by reference in their entirety.

In certain embodiments, AAV particles of the present disclosure are recombinant AAV viral vectors which are replication defective and lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ, or an organism.

In certain embodiments, the viral genome of the AAV particles of the present disclosure comprises at least one control element which provides for the replication, transcription, and translation of a coding sequence encoded therein. Not all of the control elements need always be present as long as the coding sequence is capable of being replicated, transcribed, and/or translated in an appropriate host cell Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present invention also provides for self-complementary AAV (scAAVs) viral genomes, scAAV vector genomes contain DNA strands which anneal together to form double stranded DNA. By skipping second strand synthesis, scAAVs allow for rapid expression in the transduced cell.

In certain embodiments, the AAV particle of the present disclosure is an scAAV.

In certain embodiments, the AAV particle of the present disclosure is an ssAAV.

Methods for producing and/or modifying AAV particles are provided herein and are disclosed in the art such as pseudotyped AAV vectors (PCT Patent Publication Nos. WO200028004, WO200123001; WO2004112727; WO2005005610; and WO2005072364, the content of each of which is incorporated herein by reference in its entirety).

AAV particles may be modified by methods such as those provided herein to enhance the efficiency of delivery. Such modified AAV particles can be packaged efficiently and be used to successfully infect the target cells at high frequency and with minimal toxicity. In some embodiments, the capsids of the AAV particles are engineered according to the methods provided herein and/or those described in US Publication Number US20130195801, the contents of which are incorporated herein by reference in their entirety.

AAVs are well suited for use as vectors and vehicles for gene transfer to cells. AAVs provide safe, long-term expression in a cell (e.g., a nerve cell). AAV vectors have been highly successful in fulfilling all of the features desired for a delivery vehicle, such as the ability to attach to and enter the target cell, successful transfer to the nucleus, the ability to be expressed in the nucleus for a sustained period of time, and a general lack of pathogenicity and toxicity. Recombinant AAV (rAAV) is advantageous as a delivery vector, particularly for delivery to the central nervous system, as it is focally injectable; it exhibits stable expression over time; and it is both non-pathogenic and non-integrative into the genome of the cell into which it is transduced. Twelve human serotypes of AAV (AAV serotype 1 (AAV-1) to AAV-12) and more than 100 serotypes from nonhuman primates have been reported to date. (Daya, S. and Berns, K. I., 2008, Clin. Microbiol. Rev., 21(4):583-593). In addition, rAAV has been approved by the FDA for use as a vector in at least 38 protocols for several different human clinical trials. AAV's lack of pathogenicity, persistence and its many available serotypes have increased the potential of the virus as a delivery vehicle for a gene therapy application in accordance with the described compositions and methods.

### AAV Capsids

AAV particles of the present disclosure may comprise or be derived from any natural or recombinant AAV serotype. AAV serotypes may differ in traits such as, but not limited to, packaging, tropism, transduction, and immunogenic profiles. While not wishing to be bound by theory, the AAV capsid protein is often considered to be the driver of AAV particle tropism to a particular tissue.

In certain embodiments, an AAV particle may have a capsid protein and ITR sequences derived from the same parent serotype (e.g., AAV2 capsid and AAV2 ITRs). In another embodiment, the AAV particle may be a pseudo-typed AAV particle, wherein the capsid protein and ITR sequences are derived from different parent serotypes (e.g., AAV9 capsid and AAV2 ITRs; AAV2/9).

The AAV particles of the present disclosure may comprise an AAV capsid protein with a targeting peptide inserted into the parent sequence. The parent capsid or serotype may comprise or be derived from any natural or recombinant AAV serotype. As used herein, a “parent” sequence is a nucleotide or amino acid sequence into which a targeting sequence is inserted (i.e., nucleotide insertion into nucleic acid sequence or amino acid sequence insertion into amino acid sequence).

In another embodiment, the parent AAV capsid nucleotide sequence is a K449R variant, wherein the codon encoding a lysine (e.g., AAA or AAG) at position 449 in the amino acid sequence is exchanged for one encoding an arginine (CGT, CGC, CGA, CGG, AGA, AGG). The K449R variant has the same function as wild-type AAV9.

The parent AAV serotype and associated capsid sequence may be any of those known in the art. Non-limiting examples of such AAV serotypes include. AAV9, AAV9 K449R (or K449R AAV9), AAV1, AAVrh10, AAV-DJ, AAV-DJ8, AAV5, AAVPHP.B (PHP.B), AAVPHP.A (PHP A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.13-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/(G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2. AAV8, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3. AAV24.1, AAV27.3, AAV42.12, AAV42-1b, AAV42-2, AAV42-3a, AAV42-3b, AAV42-4, AAV42-Sa, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49. AAV2-15/rh.62, AAV2-3/rh.61, AAV2-4/rh 50, AAV2-5/rh.51, AAV3.1/hu.6. AAV3.1/hu 9, AAV3-9/rh.52, AAV3-11/rh.53, AAV4-8/r11.64, AAV4-9/rh.54. AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb 2, AAV106.1/hu.37, AAV114.3/hu.40, AAV127.2/hu.41. AAV127.5/hu.42, AAV128.3/hu.44, AAV130.4/hu.48, AAV145.1/hu.53, AAV145.5/hu 54, AAV145.6/hu.55, AAV161.10/hu 60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrb.68, AAVrh.70, AAVpi.1 AAVpi.3, AAVpi.2, AAVrh.60, AAVrh.44, AAVrh.65, AAVrh.55. AAVrh.47, AAVrh.69, AAVrh.45, AAVrh.59, AAVhu.12, AAVH16, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2. AAVcy.3. AAVcy.4. AAVcy 5, AAVCy.5R1, AAVCy.5R2, AAVCy.5R3, AAVCy.5R4, AAVcy 6, AAVhu.1, AAVhu.2, AAVhu.3, AAVhu.4, AAVhu.5, AAVhu.6, AAVhu.7, AAVhu.9, AAVhu.10, AAVhu.11, AAVhu 13, AAVhu.15, AAVbu.16, AAVhu.17, AAVhu.18, AAVhu.20, AAVhu.21. AAVhu.22, AAVhu.23.2, AAVhu.24, AAVhu.25, AAVhu.27, AAVhu 28, AAVhu.29. AAVhu.29R. AAVbu.31. AAVhu 32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVbu.46, AAVhu 47, AAVhu.48, AAVhu.48R1, AAVhu.48R2. AAVhu.48R3, AAVhu.49, AAVbu.51, AAVhu 52, AAVhu.54, AAVhu.55, AAVhu.56, AAVhu.57, AAVhu.58, AAVhu.60, AAVhu.61, AAVhu.63, AAVhu 64, AAVhu.66, AAVhu.67, AAVhu.14/9, AAVhu.t 19, AAVrh.2, AAVrh.2R. AAVrb.8, AAVrh.8R, AAVrh.10, AAVrh.12, AAVrh.13. AAVrh.13R. AAVrh.14. AAVrh.17, AAVrh.18, AAVrh.19, AAVrh.20, AAVrh.21 AAVrh.22, AAVrh.23, AAVrh.24, AAVrh.25, AAVrh.31, AAVrh.32, AAVrh.33, AAVrh.34, AAVrh.35, AAVrb.36, AAVrb.37, AAVrh.37R2, AAVrh.38, AAVrh.39, AAVrh.40, AAVrh.46, AAVrb.48, AAVrh.48.1, AAVrh.48.1.2, AAVrh.48.2, AAVrh.49, AAVrh.51. AAVrh.52, AAVrh.53, AAVrh.54, AAVrh.56, AAVrh.57, AAVrb.58, AAVrb.61, AAVrh.64, AAVrh.64R1, AAVrh.64R2, AAVrb.67, AAVrh.73, AAVrb 74, AAVrh8R, AAVrh8R A586R mutant, AAVrh8R R533A mutant, AAAV.BAAV, caprine AAV, bovine AAV, AAVhE1.1. AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.ST, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2. AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu 27, AAV46.2/hu.28, AAV46.6/hu.29, AAV128.1/hu.43, true type AAV (ttAAV), UPENN AAV10, Japanese AAV10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8. AAV CBr-137.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV C-It-P1, AAV CHt-P2, AAV CHt-P5, AAV CHt-P6, AAV CHt-P8, AAV CHt-P9, AAV CKd-1, AAV CKd-10. AAV CKd-2, AAV CKd-3, AAV CKd-4, AAV CKd-6, AAV CKd-7, AAV CKd-8, AAV CKd-BI, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV CLv1-10, AAV CLv1-2, AAV CLv-12, AAV CLv1-3, AAV CLv-13, AAV CLv1-4, AAV Clv1-7, AAV CLv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-KL, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV hu.48R3, AAV.VR-355, AAV3B, AAV4, AAV5, AAVF1/HSC1, AAVF11/HSC11. AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In embodiments, a capsid or capsid library of the present disclosure is derived from AAV-PHP.B (see, e.g., Deverman, et al. “Cre-dependent selection yields AAV variants for widespread gene transfer to the adult brain,” Nat Biotechnol. 2016 February; 34(2) 204-209. PMCID. PMC5088052, the disclosure of which is incorporated herein by reference in its entirety for all purposes), AAV-PHP.eB (described in Deverman B E, Pravdo P L, Simpson B P, Kumar S R, Chan K Y, Banerjee A, Wu W-L, Yang B, Huber N, Pasca S P, Gradinaru V. Cre-dependent selection yields AAV variants for widespread gene transfer to the adult brain. Nat Biotechnol. 2016 February; 34(2):204-209. PMCID: PMC5088052; and Chan K Y, Jang M J, Yoo B B, Greenbaum A, Ravi N, Wu W-L, Sinchez-Guardado L. Lois C, Mazmanian S K, Deverman B E, Gradinaru V. Engineered AAVs for efficient noninvasive gene delivery to the central and peripheral nervous systems. Nat Neurosci. 2017 August; 20(8):1172-1179. PMCID: PMC5529245), AAVF (described in Hanlon K S, Meltzer J C, Buzhdygan T, Cheng M J, Sena-Esteves M, Bennett R E, Sullivan T P, Razmpour R, Gong Y, Ng C. Nammour J, Maiz D, Dujardin S. Ramirez S H, Hudry E, Maguire C A. Selection of an Efficient AAV Vector for Robust CNS Transgene Expression. Mol Ther Methods Clin Dev. 2019 Dec. 13; 15:320-332. PMCID: PMC6881693, the disclosure of which is incorporated herein by reference in its entirety for all purposes), AAV-PHP.B4-B8, AAV-PHP.C1-C3 (Kumar, S. R. et al. Multiplexed Cre-dependent selection yields systemic AAVs for targeting distinct brain cell types. Nat Methods 17, 541-550 (2020), 9P31) or other capsids with similar properties (Nonnenmacher, M. et al. Rapid Evolution of Blood-Brain Barrier-Penetrating AAV Capsids by RNA-Driven Biopanning. Mol Ther—Methods Clin Dev (2020) doi:10.1016/j.omtm.2020.12.006), or CAP-B10 or CAP-B22 (Goertsen, D. et al. AAV capsid variants with brain-wide transgene expression and decreased liver targeting after intravenous delivery in mouse and marmoset. Nat Neurosci 1-10 (2021) doi:10.1038/s41593-021-00969-4). Further non-limiting examples of AAV capsids suitable for encapsidation of polynucleotides include those described in PCT/US2019/044796, PCT/US2020/027708, PCT/US2020/044487, or PCT/US2020/015972, the disclosures of each of which are incorporated herein by reference in their entireties for all purposes.

In some embodiments, the serotype may be AAVDJ or a variant thereof, such as AAVDJ8 (or AAV-DJ8), as described by Grimm et al. (Journal of Virology 82(12): 5887-5911 (2008), US Publication US20140359799 and U.S. Pat. No. 7,588,772, each of which is herein incorporated by reference in its entirety). The amino acid sequence of AAVDJ8 may comprise two or more mutations in order to remove the heparin binding domain (1-BD). As a non-limiting example, the AAV-DJ sequence is as described by SEQ ID NO: 1 in U.S. Pat. No. 7,588,772, the contents of which are herein incorporated by reference in their entirety, and the AAVDJ8 sequence may comprise two mutations: (1) R587Q where arginine (R, Arg) at amino acid 587 is changed to glutamine (Q; Gln) and (2) R590T where arginine (R; Arg) at amino acid 590 is changed to threonine (T: Thr). As another non-limiting example, the AAVDJ8 sequence may comprise three mutations: (1) K406R where lysine (K; Lys) at amino acid 406 is changed to arginine (R: Arg), (2) R587Q where arginine (R Arg) at amino acid 587 is changed to glutamine (Q; Gln) and (3) R590T where arginine (R: Arg) at amino acid 590 is changed to threonine (T; Thr).

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

In certain embodiments, the initiation codon for translation of the AAV VP1 capsid protein may be CTG, TTG, or GTG as described in U.S. Pat. No. 8,163,543, the contents of which are herein incorporated by reference in their entirety.

The present disclosure refers to structural capsid proteins (including VP1. VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19, 327(5968) 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence.

As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1 sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1−), VP3 (Met−/AAV1−) or a combination of VP3 (Met+/AA1+) and VP3 (Met−/AA1−); and can also include similar optional combinations of VP2 (Met+/AA1) and VP2 (Met−/AA1−).

In certain embodiments, the parent AAV capsid sequence may comprise an amino acid sequence with 50%, 51%, 52%, 53%, 54%, 55%, 56%, 57%, 58%, 59%, 0, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those amino acid sequences (e.g., 7-mer peptide sequences) provided in the Sequence Listing.

In certain embodiments, the parent AAV capsid sequence may be encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those nucleotide sequences provided in the Sequence Listing.

Recombinant or engineered AAV vectors have shown promise for use in therapy for the treatment of human disease. However, a need still exists for AAV particles with more specific and/or enhanced tropism for target tissues. Capsid engineering methods, including those provided herein, have been used to try to identify capsids with enhanced transduction of target tissues (e.g., brain, spinal cord, DRG). A variety of methods have been used, including mutational methods, DNA barcoding, directed evolution, random peptide insertions, and capsid shuffling and/or chimeras.

One method used to generate AAV particles with desirable traits is through the use of insertion of peptides, such as those provided herein, into a parent AAV capsid sequence according to the methods provided herein.

Rational engineering and mutational methods have been used to direct AAV to a target tissue. In rational design, structure-function relationships are used to determine regions in which changes to the capsid sequence may be made. As non-limiting examples, surface loop structures, receptor binding sites, and/or heparin binding sites may be mutated, or otherwise altered, for rational design of recombinant AAV capsids for enhanced targeting to a target tissue. In one example of rational design, AAV capsids were modified by mutation of surface exposed tyrosines to phenylalanine, in order to evade ubiquitination, reduce proteasomal degradation and allow for increased AAV particle and viral genome expression (Lochrie M A, et al, J Virol 2006 January; 80(2):821-34; Santiago-Ortiz J L and Schaffer D V, J Control Release, 2016 Oct. 28; 240:287-301, the contents of each of which are incorporated by reference in their entirety). Rational design also encompasses the addition of targeting peptides to a parent AAV capsid sequence, wherein the targeting peptide may have an affinity for a receptor of interest within a target tissue.

In certain embodiments, rational engineering and/or mutational methods are used to identify AAV capsids and/or targeting peptides having enhanced transduction of a target tissue (e.g., CNS or PNS).

Capsid shuffling, and/or chimeras describe a method in which fragments of at least two parent AAV capsids are combined to generate a new recombinant capsid protein, the number of parent AAV capsids used may be 2-20, or more than 20.

In certain embodiments, capsid shuffling is used to identify AAV capsids and/or targeting peptides having enhanced transduction of a target tissue (e.g., CNS or PNS).

Directed evolution involves the generation of AAV capsid libraries (^{˜}10⁴-10⁸) by any of a variety of mutagenesis techniques and selection of lead candidates based on response to selective pressure by properties of interest (e.g., tropism). Directed evolution of AAV capsids allows for positive selection from a pool of diverse mutants without necessitating extensive prior characterization of the mutant library. Directed evolution libraries may be generated by any molecular biology technique known in the art, and may include, DNA shuffling, random point mutagenesis, insertional mutagenesis (e.g., targeting peptides), random peptide insertions, or ancestral reconstructions. AAV capsid libraries may be subjected to more than one round of selection using directed evolution for further optimization. Directed evolution methods are most commonly used to identify AAV capsid proteins with enhanced transduction of a target tissue. Capsids with enhanced transduction of a target tissue have been identified for the targeting human airway epithelium, neural stem cells, human pluripotent stem cells, retinal cells, and other in vivo and in vivo cells.

In certain embodiments directed evolution methods are used to identify AAV capsids and/or targeting peptides having enhanced transduction of a target tissue (e.g., CNS or PNS).

One method described for high-throughput characterization of the phenotypes of a large number of AAV serotypes is known as AAV Barcode-Seq (Adachi K et al, Nature Communications 5:3075 (2014), the contents of which are herein incorporated by reference in their entirety) In this next-generation sequence (NGS) based method, AAV libraries are created comprising DNA barcode tags, which can be assessed by multi-plexed Illumina barcode sequencing. This method can be used to identify AAV variants with altered receptor binding, tropism, neutralization and or blood clearance as compared to wild-type or non-variant sequences. Amino acids of the AAV capsid that are important to these functions can also be identified in this manner.

As described in Adachi et al 2014, AAV capsid libraries were generated, wherein each mutant carried a wild-type AAV2 rep gene and an AAV cap gene derived from a series of variants or mutants, and a pair of left and right 12-nucleotide long DNA bar-codes downstream of an AAV2 polyadenylation signal (pA). In this manner, 7 different DNA barcode AAV capsid libraries were generated. Capsid libraries were then provided to mice. At a pre-set timepoint, samples were collected, DNA extracted and PCR-amplified using AAV-clone specific virus bar codes and sample-specific bar code attached PCR primers. All the virus barcode PCR amplicons were Illumina sequenced and converted to raw sequence read number data by a computational algorithm. The core of the Barcode-Seq approach is a 96-nucleotide cassette comprising the DNA bar-codes (left and right) described above, three PCR primer binding sites and two restriction enzyme sites. As an exemplar, an AAV rep-cap genome was used, but the system can be applied to any AAV viral genome, including one devoid of rep and cap genes. The advantage of the Barcode Seq method is the collection of a large data set and correlation to desirable phenotype with few replicates and in a short period of time.

The DNA Barcode Seq method can be similarly applied to RNA In certain embodiments, the Barcode Seq method is used to identify AAV capsids and/or targeting peptides having enhanced transduction of a target tissue (e.g., CNS or PNS)

### Capsid Engineering

The rational design of AAV vectors that display selective tissue/organ targeting has broadened the applications of AAV as vector/vehicle for polynucleotide delivery to cells. Both direct and indirect targeting approaches have been used to enhance AAV vector cell targeting specificity and retargeting. By way of example, in direct targeting, AAV vector targeting to certain cell types is mediated by small peptides or ligands that have been directly inserted into the viral capsid sequence. This approach has been successfully employed to target endothelial cells. Direct targeting requires detailed knowledge of the capsid structure such that peptides or ligands are positioned at sites that are exposed to the capsid surface; the insertion does not significantly affect capsid structure and assembly; and the native tropism is ablated to maximize targeting to a specific cell type. In indirect targeting, AAV vector targeting is mediated by an associating molecule that interacts with both the viral surface and the specific cell surface receptor. Such associating molecules for AAV vectors may include bispecific antibodies and biotin. The advantages of indirect targeting are that different adaptors can be coupled to the capsid without resulting in significant changes in the capsid structure, and the native tropism can be easily ablated. A disadvantage of using adaptors for targeting involves a potential for decreased stability of the capsid-adaptor complex in vivo.

In addition, AAV vectors may be produced that comprise capsids that allow for the increased transduction of cells and gene transfer to the central nervous system and the brain via the vasculature (Chan, K. Y. et al., 2017, Nat. Neurosci., 20(8):1172-1179). Such vectors facilitate robust transduction of neuronal cells, including interneurons. In embodiments, AAV vectors contain an AAVF, AAV-PHP.B4, AAV-PHP.B5, AAV-PHP.C1, 9P31, or an AAV-PHP.eB capsid.

### Viral Genome of an AAV Particle

AA V particles of the disclosure, comprising targeting peptides, may be used for the delivery of any viral genome to a target tissue. The viral genome may encode any payload, such as but not limited to a polypeptide, an antibody, an enzyme, an RNAi agent and/or components of a gene editing system. In certain embodiments, the AAV particles of the disclosure are used to deliver a payload to cells of the CNS, after intravenous delivery. In some embodiments, the AAV particles of the disclosure are used to deliver a payload to cells of the liver, kidney, spleen, brain, spinal cord, serum, heart, or lungs. In some cases, the AAV particles of the disclosure are used to deliver a payload to a cell (e.g., HEK293, primary mouse brain microvascular endothelial cell, primary human BMVEC, and human brain endothelial cell line hCMEC/D3, human liver epithelial cells, hepatocytes, or human hepatocellular carcinoma cells (HepG2)). In embodiments, a viral particle comprising a capsid of the present disclosure has one or more traits selected from the following. 1) high binding affinity to HepG2 cells, 2) high binding affinity to THLE cells, 3) high transduction of HepG2 cells, 4) high transduction of THLE cells, 5) high biodistribution to C57 mice liver, and 6) high production fitness.

A viral genome of an AAV particle of the disclosure, comprises a nucleic acid sequence with at least one payload region encoding a payload, and at least one ITR. A viral genome typically comprises two ITR sequences, one at each of the 5′ and 3′ ends. Further, a viral genome of the AAV particles of the disclosure may comprise nucleic acid sequences for additional components, such as, but not limited to, a regulatory element (e.g., promoter), untranslated regions (UTR), a polyadenylation sequence (polyA), a filler or stuffer sequence, an intron, and/or a linker sequence for enhanced expression.

These viral genome components can be selected and/or engineered to further tailor the specificity and efficiency of expression of a given payload in a target tissue (e.g., CNS or DRG).

### Inverted Terminal Repeals (ITRs)

The AAV particles of the present disclosure comprise a viral genome with at least one ITR and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged ITRs incorporated into viral genomes of the disclosure may be comprised of naturally occurring polynucleotide sequences or recombinantly derived polynucleotide sequences.

The ITRs may be derived from the same serotype as the capsid, selected from any of the known serotypes, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 105, 130, 140, 141, 142, 145 nucleotides in length. ITRs encompassed by the present disclosure include those with at least 90% identity, at least 95% identity, at least 98% identity, or at least 99% identity to a known AAV serotype ITR sequence.

### Promoters

In certain embodiments, the payload region of the viral genome comprises at least one element to enhance the payload target specificity and expression (See e.g., Powell et al. Viral Expression Cassette Elements to Enhance Transgene Target Specificity and Expression in Gene Therapy, 2015: the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of a payload in a target cell may require a specific promoter, including but not limited to, a promoter that is species specific, inducible, tissue-specific, or cell cycle-specific (Parr et al., Nat. Med 3.1145-9 (1997): the contents of which are herein incorporated by reference in their entirety).

In certain embodiments, the promoter is deemed to be efficient when it drives expression of the payload encoded by the viral genome of the AAV particle.

In certain embodiments, the promoter is a promoter deemed to be efficient when it drives expression in a cell being targeted.

In certain embodiments, the promoter is a promoter having a tropism for a cell being targeted.

In certain embodiments, the promoter drives expression of the payload for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years. As a non-limiting example, the promoter is a selected for sustained expression of a payload in tissues and/or cells of the central or peripheral nervous system.

Promoters may be naturally occurring or non-naturally occurring. Non-limiting examples of promoters include those derived from viruses, plants, mammals, or humans. In some embodiments, the promoters may be those derived from human cells or systems in some embodiments, the promoter may be truncated or mutated.

Promoters which drive or promote expression in most tissues include, but are not limited to, the human elongation factor 1α-subunit (EF1α) promoter, the cytomegalovirus (CMV) immediate-early enhancer and/or promoter, the chicken β-actin (CBA) promoter and its derivative CAG, β glucuronidase (GUSB) promoter, or ubiquitin C (UBC) promoter Tissue-specific promoters can be used to restrict expression to certain cell types such as, but not limited to, cells of the central or peripheral nervous systems, targeted regions within (e.g., frontal cortex), and/or sub-sets of cells therein (e.g., excitatory neurons). As non-limiting examples, cell-type specific promoters may be used to restrict expression of a payload to excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

Cell-type specific promoters also exist for other tissues of the body, with non-limiting examples including, liver promoters (e.g., hAAT, TBG), skeletal muscle specific promoters (e.g., desmin, MCK, C512), B cell promoters, monocyte promoters, leukocyte promoters, macrophage promoters, pancreatic acinar cell promoters, endothelial cell promoters, lung tissue promoters, and/or cardiac or cardiovascular promoters (e.g., αMHC, cTnT, and CMV-MLC2k).

Non-limiting examples of tissue-specific promoters for targeting payload expression to central nervous system tissues and cells include synapsin (Syn), glutamate vesicular transporter (VGLUT), vesicular GABA transporter (VGAT), parvalbumin (PV), sodium channel Nav 1.8, tyrosine hydroxylase (TH), choline acetyltransferase (ChaT), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), neuron-specific enolase (NSE), p-globin minigene np2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In certain embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. The promoter may have a length between 200-300, 200400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 nucleotides.

In certain embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. Each component may have a length between 200-300, 200400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 nucleotides. In certain embodiments, the promoter is a combination of a 382 nucleotide CMV-enhancer sequence and a 260 nucleotide CBA-promoter sequence.

In certain embodiments, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2BI-CBX3).

Yu et al (Molecular Pam 2011, 7:63; the contents of which are herein incorporated by reference in their entirety) evaluated the expression of eGFP under the CAG, EF1α, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015, 2(2): ENEURO.0001-15; the contents of which are herein incorporated by reference in their entirety) evaluated the expression of eGFP in AAV8 with CMV and UBC promoters and AAV2 with the CMV promoter after injection in the motor cortex. Intranasal administration of a plasmid containing a UBC or EF1a promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009, 16(7) 927-932; the contents of which are herein incorporated by reference in their entirety) evaluated an NOH construct with a hGUSB promoter, a HSV-ILAT promoter and an NSE promoter and found that the HpH construct showed weaker expression than NSE in mouse brain. Passini and Wolfe (J. Virol. 2001, 12382-12392, the contents of which are herein incorporated by reference in their entirety) evaluated the long-term effects of the HpH vector following an intraventricular injection in neonatal mice and found that there was sustained expression for at least 1 year Low expression in all brain regions was found by Xu et al. (Gene Therapy 2001, 8, 1323-1332, the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650-nucleotide promoter and NFH is a 920-nucleotide promoter which are both absent in the liver but NFH is abundant in the sensory proprioceptive neurons, brain, and spinal cord and NFH is present in the heart. SCN8A (Nav 1.6) is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A. Mamm Genome (2007) 18:723-731; and Raymond et al. Expression of Alternatively Spliced Sodium Channel α-subunit genes Journal of Biological Chemistry (2004) 279(44) 46234-46241; the contents of each of which are herein incorporated by reference in their entireties).

Any of the promoters taught by the aforementioned Yu, Soderblom, Gill, Husain, Passini, Xu, Drews or Raymond may be used herein.

In certain embodiments, the promoter is not cell specific.

In certain embodiments, the promoter is a RNA pol III promoter. As a non-limiting example, the RNA pol III promoter is U6. As a non-limiting example, the RNA pol III promoter is H1.

In certain embodiments, the viral genome comprises an enhancer element.

In certain embodiments, the viral genome comprises an engineered promoter.

In another embodiment, the viral genome comprises a promoter from a naturally expressed protein.

### Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue or DRG) may be engineered into UTRs to enhance stability and protein production. As a non-limiting example, a 5′ UTR from mRNA normally expressed in the brain (e.g., huntingtin) may be used in the viral genomes of the AAV particles of the disclosure to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCRCCAUGG, where R is a purine (adenine or guanine) three bases upstream of the start codon (ATG), which is followed by another ‘G’.

In certain embodiments, the 5′UTR in the viral genome includes a Kozak sequence.

In certain embodiments, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class II ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal, or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise. AREs can be identified and removed or mutated to increase the intracellular stability and thus increase translation and production of the resultant protein.

In certain embodiments, the 3′ UTR of the viral genome may include an oligo(dT) sequence for templated addition of a poly-A tail.

In certain embodiments, the viral genome may include at least one miRNA seed, binding site or full sequence, microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which has perfect Watson-Crick sequence complementarity to the miRNA target sequence of the nucleic acid.

In certain embodiments, the viral genome may be engineered to include, alter, or remove at least one miRNA binding site, full sequence, or seed region.

Any UTR from any gene known in the art may be incorporated into the viral genome of the AAV particle. These UTRs, or portions thereof, may be placed in the same orientation as in the gene from which they were selected, or they may be altered in orientation or location. In certain embodiments, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In certain embodiments, the viral genome of the AAV particle comprises at least one artificial UTR which is not a variant of a wild type UTR.

In certain embodiments, the viral genome of the AAV particle comprises UTRs which have been selected from a family of transcripts whose proteins share a common function, structure, feature, or property.

### Polyadenylation Sequence

The viral genome of the AAV particles of the present disclosure may comprise at least one polyadenylation sequence. In certain embodiments, the viral genome of the AAV particle comprises a polyadenylation sequence between the 3′ end of the payload encoding region and the 5′ end of the 3′ITR.

In certain embodiments, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 nucleotides in length.

### Introns

In certain embodiments, the viral genome of the AAV particles of the present disclosure comprises at least one element to enhance the payload target specificity and expression (See e.g., Powell et al. Viral Expression Cassette Elements to Enhance Transgene Target Specificity and Expression in Gene Therapy, Discov. Med, 2015, 19(102): 49-57; the contents of which are herein incorporated by reference in their entirety) such as an intron. Non-limiting examples of introns include. MVM (67-97 bps), FIX truncated intron 1 (300 bps), pi-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In certain embodiments, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

### Stuffer Sequences

In certain embodiments, the viral genome of the AAV particles of the present disclosure comprises at least one element to improve packaging efficiency and expression, such as a stuffer or tiller sequence. Non-limiting examples of stuffer sequences include albumin and/or alpha-1 antitrypsin. Any known viral, mammalian, or plant sequence may be manipulated for use as a stuffer sequence.

In certain embodiments, the stuffer or tiller sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

miRNA

In certain embodiments, the viral genome comprises at least one sequence encoding a miRNA to reduce the expression of the payload in an “off-target” tissue. As used herein, “off-target” indicates a tissue or cell-type unintentionally targeted by the AAV particles of the disclosure. As an example, an “off-target” tissue or cell when targeting the DRG, may be neurons of other ganglia, such as those of the sympathetic or parasympathetic nervous system, miRNAs and their targeted tissues are well known in the art. As a non-limiting example, a miR-122 miRNA may be encoded in the viral genome to reduce the expression of the viral genome in the liver.

### Selectable Marker

In some embodiments, the viral genome of the AAV particles of the disclosure optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are described in International Publication Nos. WO 1996023810 and WO 1996030540; Heim et al., Current Biology 2:178-182 (1996); Heim et al., Proc. Natl. Acad. Sci. USA (1995); or Heim et al., Science 373:663-664 (1995), the contents of each of which are incorporated herein by reference in their entirety.

### Genome Size

In certain embodiments, the AAV particles of the disclosure may comprise a single-stranded or double-stranded viral genome. The size of the viral genome may be small, medium, large or the maximum size. As described above, the viral genome may comprise a promoter and a polyA tail.

In certain embodiments, the viral genome may be a small single stranded viral genome. A small single stranded viral genome may be 2.1 to 3.5 kb in size such as, but not limited to, about 2.1, 2.2, 2.3, 2.4, 2.5, 2.6, 2.7, 2.8, 2.9, 3.0, 3.1, 3.2, 3.3, 3.4, and 3.5 kb in size.

In certain embodiments, the viral genome may be a small double stranded viral genome A small double stranded viral genome may be 1.3 to 1.7 kb in size such as, but not limited to, about 1.3, 1.4, 1.5, 1.6, and 1.7 kb in size.

In certain embodiments, the viral genome may be a medium single stranded viral genome. A medium single stranded viral genome may be 3.6 to 4.3 kb in size such as, but not limited to, about 3.6, 3.7, 3.8, 3.9, 4.0, 4.1, 4.2 and 4.3 kb in size.

In certain embodiments, the viral genome may be a medium double stranded viral genome. A medium double stranded viral genome may be 1.8 to 2.1 kb in size such as, but not limited to, about 1.8, 1.9, 2.0, and 2.1 kb in size.

In certain embodiments, the viral genome may be a large single stranded viral genome. A large single stranded viral genome may be 4.4 to 6.0 kb in size such as, but not limited to, about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size.

In certain embodiments, the viral genome may be a large double stranded viral genome A large double stranded viral genome may be 2.2 to 3.0 kb in size such as, but not limited to, about 2.2, 2.3, 2.4, 2.5, 2.6, 2.7, 2.8, 2.9 and 3.0 kb in size.

### Payloads

The AAV particles of the present disclosure comprise a viral genome with at least one payload region. As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is a polynucleotide or polypeptide. Payloads of the present disclosure may be, but are not limited to, peptides, polypeptides, proteins, antibodies, polynucleotides, etc.

The payload region can contain a combination of coding and non-coding nucleic acid sequences.

In certain embodiments, the AAV particle comprises a viral genome with a payload region encoding more than one payload of interest. In such an embodiment, a viral genome encoding more than one payload may be replicated and packaged into a viral particle. A target cell transduced with a viral particle comprising more than one payload may express each of the payloads in a single cell.

### Modified Polynucleotides

In some embodiments of any of the aspects, a nucleic acid sequence as described herein is chemically modified to enhance stability or other beneficial characteristics. The nucleic acids described herein may be synthesized and/or modified by methods such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include but are not limited to nucleic acids containing modified backbones or no natural internucleoside linkages nucleic acids having modified backbones include, among others, those that do not have a phosphorus atom in the backbone.

Modified nucleic acids that do not have a phosphorus atom in their internucleoside backbone can also be considered to be oligonucleosides. In some embodiments, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms, and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH₂ component parts, and oligonucleosides with heteroatom backbones, and in particular —CH2-NH—CH2-, —CH2-N(CH3)-O—CH2- [known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et ah, (2005) Nucleic Acids Research 33(1):439-447; Mook, O R. et al., (2007) Mol. Cane. Ther. 6(3):833-843; Grunweller, A. et ah, (2003) Nucleic Acids Research 31(12):3185-3193).

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, where the alkyl, alkenyl and alkynyl may be substituted or unsubstituted C1 to CIO alkyl or C2 to CIO alkenyl and alkynyl. Exemplary suitable modifications include O[(CH2)nO]mCH₃, O(CH2)nOCH₃, O(CH2)nNH2, O(CH2) nCH3, O(CH2)nONH2, and O(CH2)nON[(CH2)nCH3)]2, where n and m are from 1 to about 10. In some embodiments, nucleic acids include one of the following at the 2′ position: C1 to CIO lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments, the modification includes a 2′ methoxyethoxy (2′-O—CH2CH2OCH3, also known as 2′-O-(2-methoxyethyl) or 2′-MOE) (Martin et al, Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2).

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. “Unmodified” or “natural” nucleobases include the purine bases adenine (A) and guanine (G), and the pyrimidine bases thymine (T), cytosine (C) and uracil (U). Modified nucleobases can include other synthetic and natural nucleobases including but not limited to as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments, oligonucleotide tags (e.g., Oligopaint) comprise any modified nucleobases known in the art, i.e., any nucleobase that is modified from an unmodified and/or natural nucleobase.

The preparation of the modified nucleic acids, backbones, and nucleobases described above are known in the art.

Another modification of a nucleic acid featured in the disclosure involves chemically linking to a polynucleotide one or more ligands, moieties or conjugates that enhance the activity, cellular distribution, pharmacokinetic properties, or cellular uptake of the polynucleotide. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4: 1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et ak, EMBO J, 1991, 10: 1111-1118; Kabanov et al., LEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

### AAV Production

Viral production disclosed herein describes processes and methods for producing AAV particles may be used to contact a target cell to deliver a payload.

The present disclosure provides methods for the generation of AAV particles containing capsids with improved traits. In certain embodiments, the AAV particles are prepared by viral genome replication in a viral replication cell. Any method known in the art may be used for the preparation of AAV particles. In certain embodiments, AAV particles are produced in mammalian cells (e.g., HEK293). In another embodiment, AAV particles are produced in insect cells (e.g., Sf9)

Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191 6,566,118, 8,137,948; or International Publication Nos WO1996039530, WO1998010088, WO1999014354, WO1999015685, WO1999047691, WO2000055342, WO2000075353 and WO2001023597; Methods In Molecular Biology, ed. Richard, Humana Press, NJ (1995); O'Reilly et al., Baculovirus Expression Vectors, A Laboratory Manual. Oxford Univ. Press (1994); Samulski et al., J. Vir. 63:3822-8 (1989), Kajigaya et al., Proc. Nat'l. Acad. Sci. USA 88. 4646-50 (1991): Ruffing et al, J. Vir. 66:6922-30 (1992); Kimbauer et al, Vir., 219:37-44 (1996); Zhao et al., Vir. 272:382-93 (2000); the contents of each of which are herein incorporated by reference in their entirety. In certain embodiments, the AAV particles are made using the methods described in International Patent Publication WO2015191508, the contents of which are herein incorporated by reference in their entirety.

The viral replication cell may be selected from any biological organism, including prokaryotic (e.g., bacterial) cells, and eukaryotic cells, including, insect cells, yeast cells and mammalian cells. Viral replication cells commonly used for production of recombinant AAV viral particles include, but are not limited to, HEK293 cells, COS cells, HeLa cells, KB cells, and other mammalian cell lines as described in U.S. Pat. Nos. 6,156,303, 5,387,484, 5,741,683, 5,691,176, and 5,688,676; U.S. Patent Application Publication No. 2002/0081721, and International Patent Publication Nos. WO 2000047757, WO 2000024916, and WO 1996017947, the contents of each of which are herein incorporated by reference in their entirety. Viral replication cells may comprise other mammalian cells such as A549. WEH1, 3T3, 10T1/2, BHK, MDCK, COS 1, COS 7, BSC 1, BSC 40, BMT 10, VERO, W138, Saos, C2C12, L cells, HT1080, HepG2 and primary fibroblast, hepatocyte and myoblast cells derived from mammals. Viral replication cells may comprise cells derived from mammalian species including, but not limited to, human, monkey, mouse, rat, rabbit, and hamster. Viral replication cells may comprise cells derived from a cell type, including but not limited to fibroblast, hepatocyte, tumor cell, cell line transformed cell, etc.

In some embodiments, the present disclosure provides a method for producing an AAV particle in mammalian cells, comprising the steps of 1) simultaneously co-transfecting mammalian cells, such as, but not limited to HE-K293 cells, with a viral genome comprising a payload region (payload construct), a viral genome comprising polynucleotide sequences for rep and cap genes (rep/cap construct) and a viral genome comprising polynucleotide sequences encoding helper components (helper construct), 2) harvesting and purifying the AAV particles comprising a viral genome. This triple transfection method of AAV particle production may be utilized to produce small lots of virus.

In certain embodiments, the AAV particles may be produced in a viral replication cell that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995). O'Reilly et al., Baculovirus Expression Vectors, A Laboratory Manual, Oxford Univ Press (1994): Samulski et al., J. Vir. 63:3822-8 (1989) Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996), Zhao et al., Vir. 272. 82-93 (2000), and Samulski et al., U.S. Pat. No. 6,204,059, the contents of each of which is herein incorporated by reference in its entirety.

In some embodiments, the present disclosure provides a method for producing an AAV particle in a baculovirus/Sf9 system, comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, and 5) harvesting and purifying AAV particles comprising a viral genome.

Briefly, the viral construct vector and the AAV payload construct vector are each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses may be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M, et al., J Virol. 2006 February; 80 (4):1874-85, the contents of which are herein incorporated by reference in their entirety.

Production of AAV particles with baculovirus in an insect cell system may address known baculovirus genetic and physical instability. In certain embodiments, the production system addresses baculovirus instability over multiple passages by utilizing a titerless infected-cells preservation and scale-up system. Small scale seed cultures of viral producing cells are transfected with viral expression constructs encoding the structural, non-structural, components of the viral particle. Baculovirus-infected viral producing cells are harvested into aliquots that may be cryopreserved in liquid nitrogen, the aliquots retain viability and infectivity for infection of large scale viral producing cell culture Wasilko D J et al., Protein Expr Purif, 2009 June; 65(2).122-32, the contents of which are herein incorporated by reference in their entirety.

A genetically stable baculovirus may be used as the source of one or more of the components for producing AAV particles in invertebrate cells in certain embodiments, defective baculovirus expression vectors may be maintained episomally in insect cells. In such an embodiment the bacmid vector is engineered with replication control elements, including but not limited to promoters, enhancers, and/or cell-cycle regulated replication elements.

In certain embodiments, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

AAV particles described herein may be produced by triple transfection or baculovirus mediated virus production, or any other method known in the art. Any suitable permissive or packaging cell known in the art may be employed to produce the particles. Mammalian cells are often preferred. Also preferred are trans-complementing packaging cell lines that provide functions deleted from a replication-defective helper virus, e.g., 293 cells or other E1a trans-complementing cells. A packaging cell line may be used that is stably transformed to express cap and/or rep genes. Alternatively, a packaging cell line may be used that is stably transformed to express helper constructs necessary for AAV particle assembly.

Recombinant AAV virus particles are, in some cases, produced and purified from culture supernatants according to the procedure as described in US20160032254, the contents of which are incorporated by reference.

In certain embodiments, AAV particles are produced wherein all three VP proteins are expressed at a stoichiometry around 1:1:10 (VP1:VP2:VP3). While not wishing to be bound by theory, the regulatory mechanisms that allow this controlled level of expression include the production of two mRNAs, one for VP1, and the other for VP2 and VP3, produced by differential splicing.

In certain embodiments, the viral construct vector(s) used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In certain embodiments, the viral construct vector(s) used for AAV production may contain a nucleotide sequence encoding the AAV rep proteins where the initiation codon of the AAV rep protein or proteins is a non-ATG. In certain embodiments, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rcp78 as compared to Rep52, which may be advantageous in that it promotes high vector yields Small-scale production

In some cases, 293T cells (adhesion/suspension) are transfected with polyethyleneimine (PEI) with plasmids required for production of AAV, i.e., AAV2 rep, an adenoviral helper construct and a ITR flanked payload cassette. The AAV2 rep plasmid also contains the cap sequence of the particular virus being studied. Twenty-four hours after transfection (no medium changes for suspension), which occurs in DMEM/F17 with/without serum, the medium is replaced with fresh medium with or without serum. Three (3) days after transfection, a sample is taken from the culture medium of the 293 adherent cells. Subsequently cells are scraped, or suspension cells are pelleted, and transferred into a receptacle. For adhesion cells, after centrifugation to remove cellular pellet, a second sample is taken from the supernatant after scraping Next, cell lysis is achieved by three consecutive freeze-thaw cycles (−80 C to 37 C) or adding detergent triton. Cellular debris is removed by centrifugation or depth filtration and sample 3 is taken from the medium. The samples are quantified for AAV particles by DNase resistant genome titration by DNA qPCR. The total production yield from such a transfection is equal to the particle concentration from sample 3.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278).

### Large-Scale Production

In some embodiments. AAV particle production may be modified to increase the scale of production. Large scale viral production methods according to the present disclosure may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, N.Y.) and NUNC™ CELL FACTORY™ (ThermoFisher Scientific, Waltham, Mass.). In some cases, large-scale adherent cell surfaces may comprise from about 1,000 cm to about 100,000 cm². In some cases, large-scale adherent cell cultures may comprise from about 10⁷ to about 10⁹ cells, from about 1 to about 10¹⁰ cells, from about 10⁹ to about 10¹² cells or at least 10¹² cells. In some cases, large-scale adherent cultures may produce from about 10⁹ to about 10¹², from about 10¹⁰ to about 10¹³, from about 10¹¹ to about 10¹⁴, from about 10¹² to about 10¹⁵ or at least 10¹⁵ viral particles.

In some embodiments, large-scale viral production methods of the present disclosure may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate), organic compounds [e.g. polyethyleneimine (PEI)] or the use of non-chemical methods (e.g. electroporation.) With cells grown in suspension, transfection methods may include, but are not limited to the use of calcium phosphate and the use of PEI. In some cases, transfection of large-scale suspension cultures may be carried out according to the section entitled “Transfection Procedure” described in Feng, L, et al., 2008 Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours in some cases, cell cultures may be shocked at a temperature of from about 0° C. to about 20° C.

In some cases, transfections may include one or more vectors for expression of an RNA effector molecule to reduce expression of nucleic acids from one or more AAV payload constructs. Such methods may enhance the production of viral particles by reducing cellular resources wasted on expressing payload constructs. In some cases, such methods may be carried out according to those methods taught in US Publication No. US2014/0099666, the contents of which are herein incorporated by reference in their entirety.

### Compositions

Provided herein are compositions containing AAV particles, AAV capsids, and/or polynucleotides encoding the same. The AAV particles may be contained in any appropriate amount in any suitable carrier substance and is/are generally present in an amount of 0.01-95% by weight of the total weight of the composition. The composition may be provided in a form that is suitable for a parenteral (e.g., subcutaneous, intravenous, intramuscular, or intraperitoneal) administration route, such that the agent, such as a viral particle described herein, is systemically delivered. In some instances, a reporter product is also encoded by the vector. The compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Compositions may be formulated to release the viral particles substantially immediately upon administration or at any predetermined time or time after administration. The latter types of compositions are generally known as controlled release formulations, which include (i) compositions that create a substantially constant concentration of the agent within the body over an extended period of time; (ii) compositions that after a predetermined lag time create a substantially constant concentration of the drug within the body over an extended period of time; (iii) compositions that sustain action during a predetermined time period by maintaining a relatively constant, effective level in the body with concomitant minimization of undesirable side effects associated with fluctuations in the plasma level of the active substance (sawtooth kinetic pattern); (iv) compositions that localize action by, e.g., spatial placement of a controlled release composition adjacent to or in contact with a target site or location, e.g., in a region of a tissue or organ; (v) compositions that allow for convenient dosing, such that doses are administered, for example, once every one, two, or several weeks; and (vi) compositions that target a specific tissue or cell type using carriers, chemical derivatives, or specifically designed viral particles (e.g., comprising a certain capsid composition) to deliver a payload to a cell.

The composition may be administered systemically, for example, in an acceptable buffer such as physiological saline. In an embodiment, systemic injection of an rAAV vector as described herein allows for the delivery of a payload (e.g., a polynucleotide) to a cell or organ.

Routes of administration include, for example, intracranial, parenteral, subcutaneous (s.c.), intravenous (i.v.), intraperitoneal (i.p.), intramuscular (i.m.), or intradermal administration. The amount of the vector to be administered can vary depending upon the requirements of a given screen. Generally, amounts will be in the range of those used for other viral vector-based agents employed in the delivery of polynucleotides to cells. In embodiments, about, at least about, and/or no more than about 1×10e5, 1×10e6, 1×10e7, 1×10e8, 1×10e9, 1×10e10, 1×10e11, 1×10e12, 1×10e13, 1×10e14, or 1×10e15 vector genomes are delivered to a subject (e.g., a mouse) to screen a library of enhancers. A composition is administered at a level that is effective in meeting the objectives of a screen.

The composition may be in the form of a solution, a suspension, an emulsion, an infusion device, or a delivery device for implantation, or it may be presented as a dry powder to be reconstituted with water or another suitable vehicle before use. The composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition is formulated for intravenous delivery. As noted above, the compositions according to the described embodiments may be in a form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl, or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Delivery of Recombinant Adeno-Associated Viral Vectors

For direct delivery to the brain, rAAV vectors may be administered by open neurosurgical procedure or by focal injection in order to bypass the blood-brain barrier, to temporally and spatially restrict transgene expression, and to target specific areas of the brain, e.g., interneuron cells and brain tissue comprising these cells.

In some cases, an rAAV vector is delivered to a subject intravenously. In some cases, the rAAV vector is delivered to the central nervous system using the vasculature.

Systemic rAAV delivery (by intravenous injection) provides a non-invasive alternative for broad gene delivery to the nervous system. Several groups have developed rAAV capsids that enhance gene transfer to the CNS and certain tissues and cell populations after intravenous delivery. By way of example, AAV-AS capsid18 utilizes a polyalanine N-terminal extension to the AAV9.4719 VP2 capsid protein to provide higher neuronal transduction, particularly in the striatum. The AAV-BR1 capsid20, based on AAV2, may be useful for more efficient and selective transduction of brain endothelial cells. Another AAV capsid, AAV-PHP.B, comprises a capsid that transduces the majority of neurons and astrocytes across many regions of the adult mouse brain and spinal cord after intravenous injection.

Other modes of rAAV vector administration may include lipid-mediated vector delivery, hydrodynamic delivery, and a gene gun.

The virus vectors and compositions thereof as described herein may be used to screen libraries of capsid polypeptides that have specificity or particular activity levels in particular cell types or tissues (e.g., an organ).

### Polynucleotide Sequencing

Preparation of a library for sequencing may involve an amplification step. Amplification may involve thermocycling (e.g., PCR) or isothermal amplification (such as through the methods NEAR, RNA-Seq, RPA or LAMP). Amplification can refer to any method employing a primer and a polymerase capable of replicating a target sequence with reasonable fidelity. Amplification may be carried out by natural or recombinant DNA polymerases, such as TaqGold™, T7 DNA polymerase, Klenow fragment of E. coli DNA polymerase, and reverse transcriptase. A preferred amplification method is PCR. In some embodiments, isolated RNA is contacted with a reverse transcriptase to produce cDNA for sequencing and/or PCR amplification.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

### Hardware and Software

A computer system (or digital device) may be used to receive, transmit, display and/or store results, analyze the results, and/or produce a report of the results and analysis. A computer system may be understood as a logical apparatus that can read instructions from media (e.g., software) and/or network port (e.g., from the internet), which can optionally be connected to a server having fixed media. A computer system may comprise one or more of a CPU, disk drives, input devices such as keyboard and/or mouse, and a display (e.g., a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g., one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine-readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active-matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high-capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Kits

Also provided are kits comprising engineered AAV capsids, and/or polynucleotides encoding the same. Typically, kits will comprise sufficient amounts and/or numbers of components to allow a user to perform multiple treatments of a subject(s) and/or to perform multiple experiments.

Any of the capsid polypeptides, or polynucleotides encoding the same, of the present disclosure may be contained in a kit. In some embodiments, kits may further include reagents and/or instructions for creating and/or synthesizing compounds and/or compositions of the present disclosure. In some embodiments, kits may also include one or more buffers.

In some embodiments, kit components may be packaged either in aqueous media or in lyophilized form. The container means of the kits will generally include at least one vial, test tube, flask, bottle, syringe, or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

The kit can include instructions for use of the compositions in a method provided herein (e.g., to deliver a payload to a cell). The instructions may be printed directly on the container (when present), or as a label applied to the container, or as a separate sheet, pamphlet, card, computer-readable medium, or folder supplied in or with the container.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: High Production Fitness Space Mapping

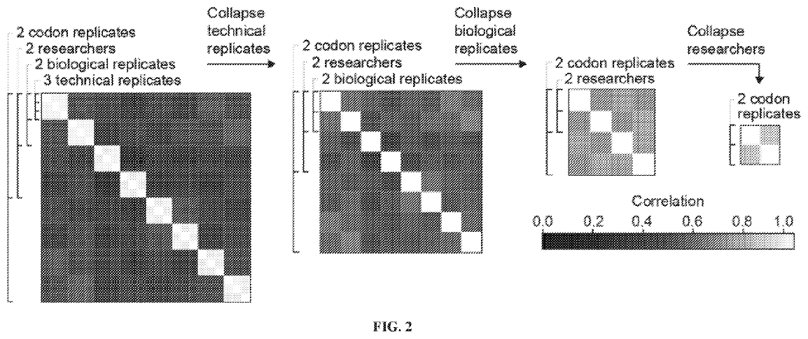

Experiments were undertaken to develop improved gene delivery vectors derived from AAV9, by inserting into the AAV9 capsid 7 amino acids (7-mer) between VP1 residues 588 and 589 (FIGS. 1A-1E). To create an accurate and generalizable sequence-to-production fitness ML model, synthetic training and assessment libraries were designed to each consist of 74.5K variants that evenly sample the sequence space (each amino acid was sampled with an equal probability at each position); 10K of the 74.5K were common to both libraries to assess reproducibility across libraries. This was distinct from conventional NNN or NNK (where N is any base and K is a G or T) libraries where millions of variants are synthesized stochastically by uniformly sampling the nucleotide space, which biases toward AAs represented by more codons. Both training and assessment libraries were also designed to assess whether codon usage impacted production fitness; each variant was represented by two maximally different nucleotide sequences (7-mer amino acid replicates). Both libraries were produced in triplicate, in two separate runs, by two different researchers, for a total of 12 replicates each. The reproducibility (measured by the agreement between replicates) of variant production fitness scores between preparations by different researchers improved as technical and biological replicates were aggregated (FIG. 2). Therefore, all subsequent analyses on production fitness were performed using scores aggregated across all replicates for each library.

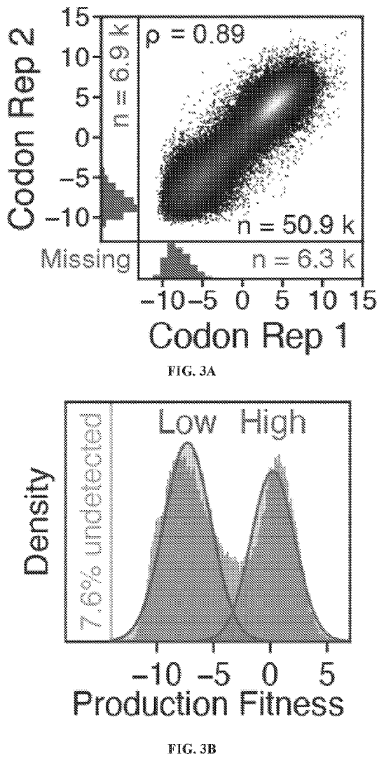

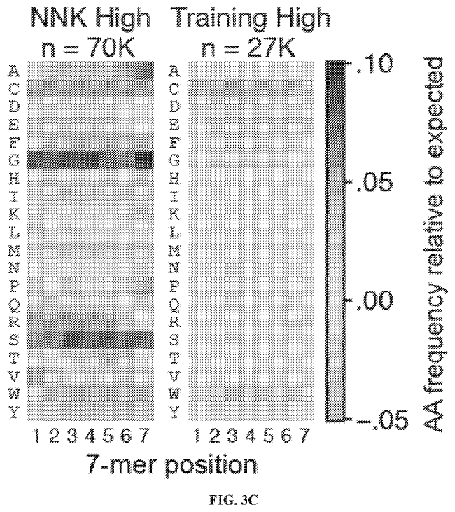

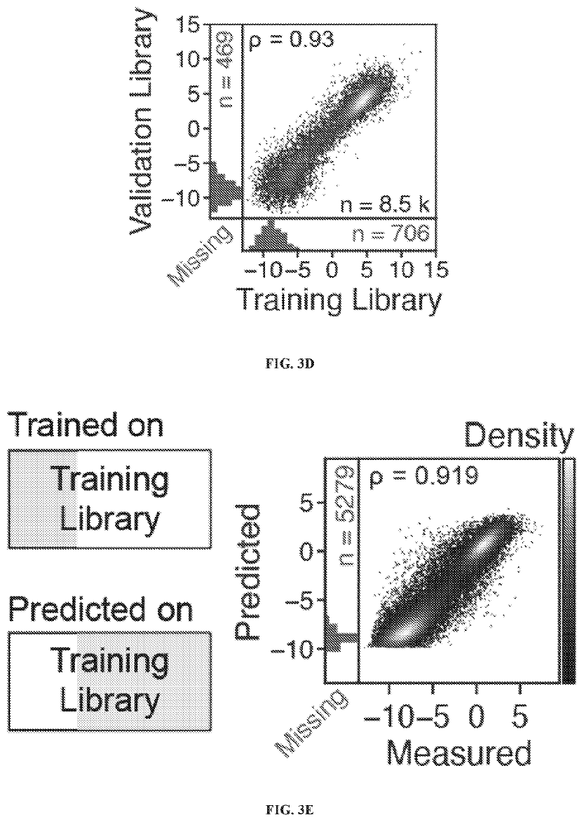

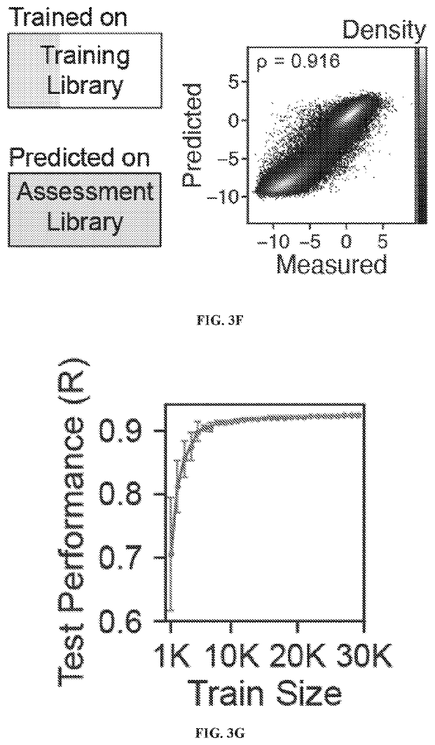

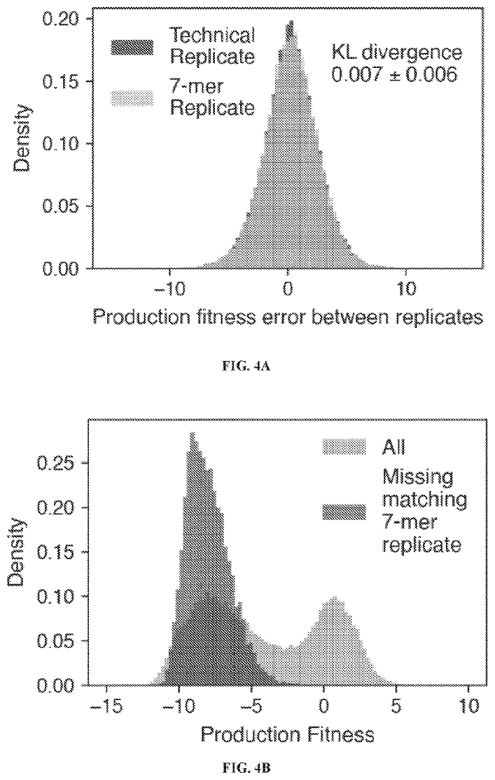

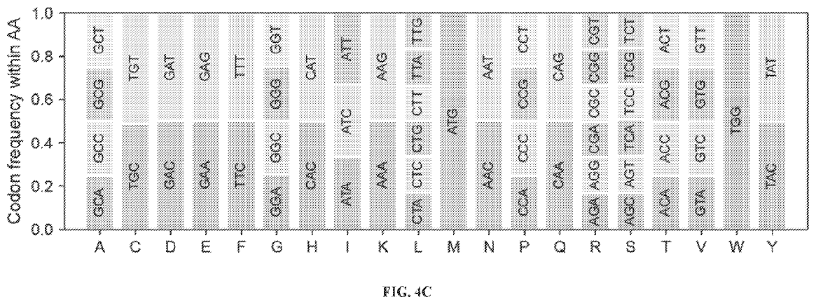

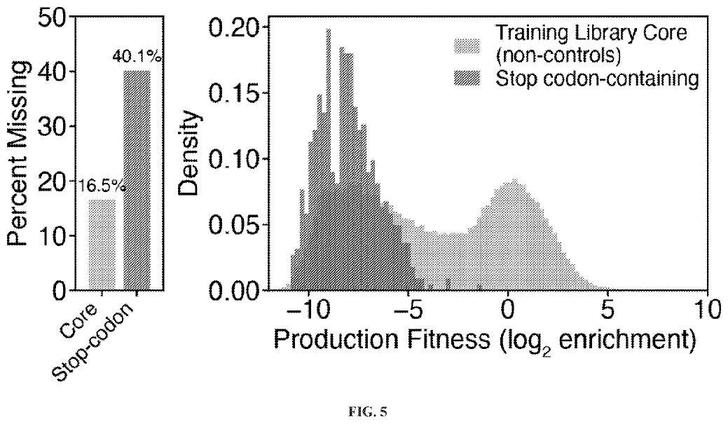

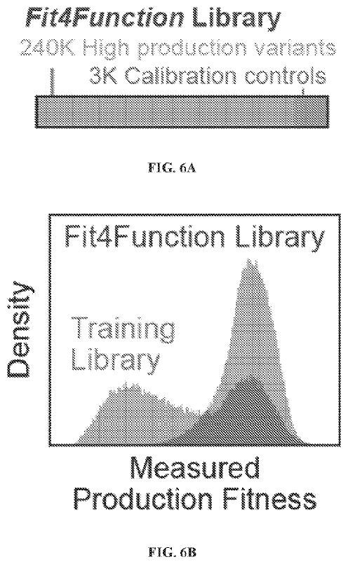

It was first assessed whether codon usage impacted the production fitness of identical amino acid variants. If so, it would be necessary to train on the nucleotide sequence space (617 for NNN, 31⁷ for NNK, where “N” represents any nucleotide and “K” represents G or T, and where 61 and 31 correspond to the number of amino-acid encoding codons with the sequence NNN or NNK, respectively), which is much larger than the amino acid sequence space (207). High correlation was observed between the fitness scores of 7-mer amino acid replicates (FIG. 3A, FIG. 5), and the distribution of measured differences between codon replicates did not exceed those observed between technical replicates (FIG. 4A). Of the 13,217 codon replicates where only one of the two codon sequences was detected in virus (20.5% of the 64,500 AA variants), >99% had fitness scores on the low end of the fitness distribution, suggesting that the missing replicates were not detected due to low abundance (FIGS. 3A and 4B). Furthermore, no codon usage bias was observed for individual AAs (FIG. 4C). Therefore, production fitness was averaged across 7-mer replicates for all downstream modeling.

The production fitness distribution of the training library was modeled by a mixture of two Gaussian distributions: a “low fitness” versus a “high fitness” distribution (FIG. 3B). The low fitness distribution overlapped with the production fitness distribution of the stop codon containing variants which were presumably detected in the virus library due to cross-packaging (FIG. 5). The variants in the high fitness distribution exhibited distinguishing amino acid sequence characteristics, such as a general enrichment of negatively charged residues and depletion of cysteine and tryptophan (FIG. 3C). Nonetheless, this high production fitness distribution had less bias than an analogous set of the most abundant 70K variants from an NNK library (FIG. 3C). The fitness scores for the 10K variants common to both libraries were consistent across the training and assessment libraries, suggesting that variant fitness is not noticeably impacted by the other variants in the library (FIG. 3D).

### Example 2: A Generalizable Production Fitness Model

A regression model was used to capture the large variation in relative production fitness scores (±5-fold; log 2 enrichment) within the high fitness and low fitness distributions (FIG. 3B). The model was first trained using the sequence and production fitness measurements of 24K variants unique to the training library. The accuracy of each model in this study was assessed by the agreement (Pearson correlation) between the measured fitness scores and the model's predicted scores. Remarkably, the sequence-to-production-fitness model achieved high accuracy the remaining subset of the library not used in the training process (FIG. 3E), as well as on the independent assessment library (FIG. 3F). In addition, the model did not require large amounts of training data to obtain high accuracy, reducing the training from 24K to 5K variants only slightly reduced performance (r=0.924±0.001 vs r=0.899±0.015, FIG. 3G). These data demonstrated that the model was generalizable across libraries and to unseen variants and required relatively small training datasets.

### Example 3: Fit4Function Enables Reproducible Data and Accurate Prediction Models

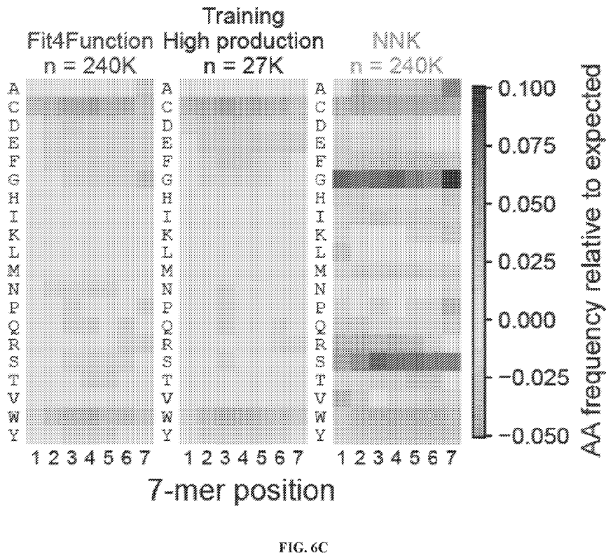

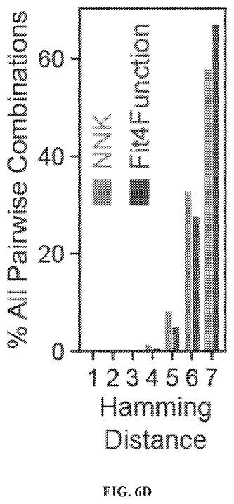

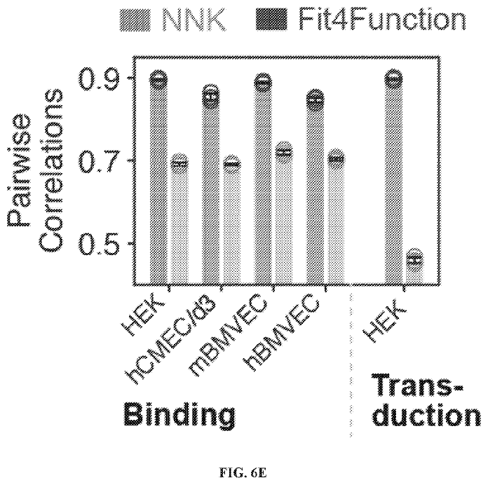

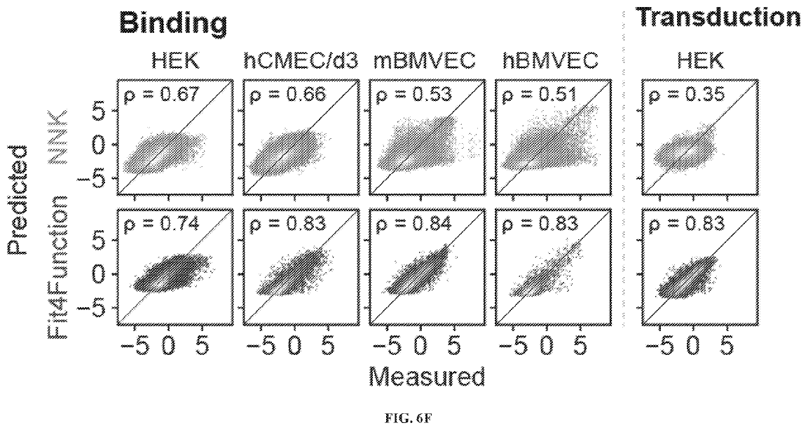

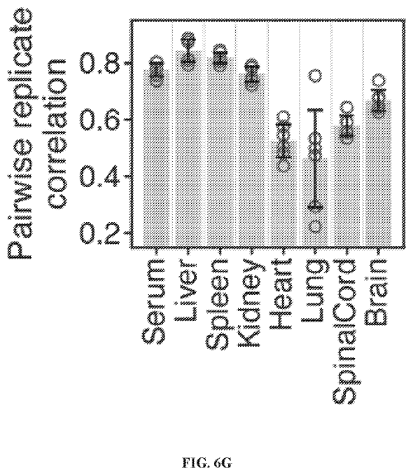

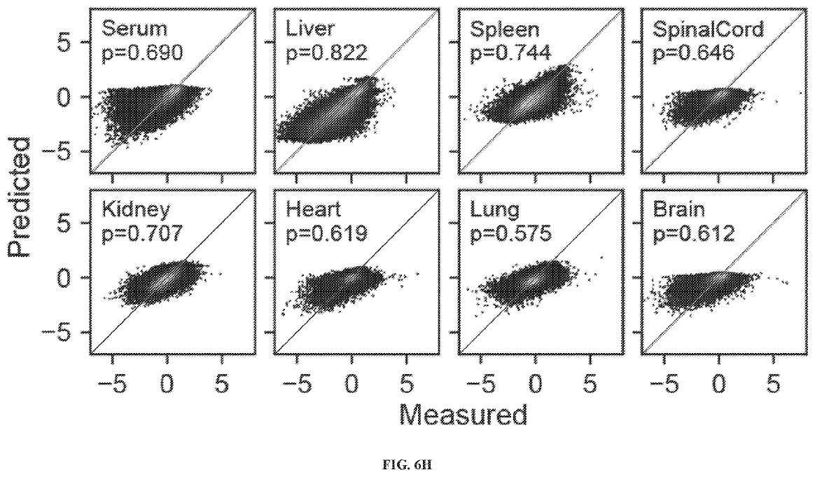

Using the production fitness model, the fitness of 24M AA variants was randomly generated and predicted in silico. The predicted high production fitness sequence space was then evenly sampled for 240K variants to create a “Fit4Function” library that evenly sampled only the high fit sequence space (FIG. 6A). As expected, the measured fitness scores for the Fit4Function variants, when synthesized, mapped to a single distribution that closely followed the production fitness distribution after calibration (FIG. 6B). The amino acid distribution in the Fit4Function library was similar to that of the production fitness distribution from the training library and was similarly less biased when compared to that of the 240K most abundant variants in an NNK library (FIG. 6C). To assess library diversity, the pairwise Hamming distance (how many residues differ) was computed between all variant pairs; 67% of the Fit4Function pairs had a distance of seven (all positions) compared to 57.8% of the pairs from the 240K most abundant sequences in an NNK library (FIG. 6D). It is important to note that the criterion for high production fitness in populating Fit4Function libraries was not so stringent as to eliminate potentially promising functional candidates for downstream optimization; only variants with poor production (i.e., those whose production fitness was comparable to stop-codon containing control sequences) were considered low in production fitness and not sampled for Fit4Function libraries (FIG. 5).

Fit4Function libraries were designed to enable the generation of reproducible and ML-compatible functional screening data. Specifically, the library was limited to a moderate size that enabled deeper sequencing depth and sampled only variants with high production fitness, which enabled more quantitative and reliable detection of each variant in the library. In addition, the library evenly sampled the high production fitness amino acid sequence space, which resulted in less biased ML models that generalized well across the sequence space.

The outcomes of the Fit4Function library screening strategy were compared versus an NNK library across five functional assays: (1) HEK293 cell binding, (2) primary mouse brain microvascular endothelial cell (BMVEC) binding, (3) primary human BMVEC binding, (4) human brain endothelial cell line (hCMEC/D3) binding, and (5) HEK293 transduction. Binding and transduction were measured by quantitative sequencing capsid variant abundance at the DNA and mRNA levels, respectively. The Fit4Function library consistently yielded higher replication quality data than the NNK library (one-tailed paired t-test, n=5 assays; p=0.0074; FIG. 6E). Models trained on functional data derived from the Fit4Function library were built and compared versus an NNK library (only data from the most abundant 240K variants in the NNK virus library was used). The Fit4Function-based models consistently achieved higher prediction accuracy (FIG. 6F).

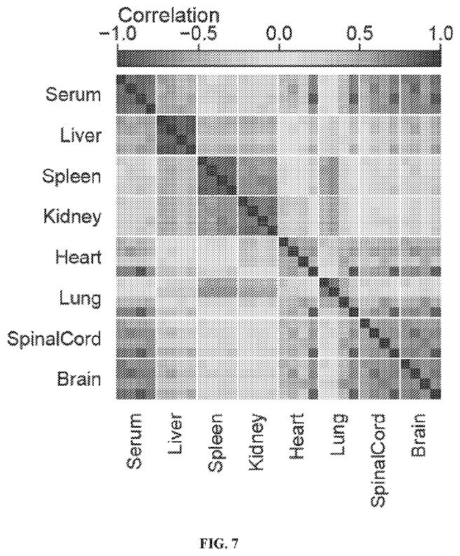

It was next sought to examine the use of the Fit4Function library to train prediction models of in vivo AAV biodistribution after systemic administration in adult C57BL/6J mice. The replication quality was high in liver, kidney, and spleen, and moderate in the brain, spinal cord, serum, heart, and lungs (FIGS. 6G and 7). Independent models were trained to predict the variant tropism for each organ. The training data measurements were aggregated across three animals, and the data from the fourth animal was held out for independent testing. The models performed reasonably well when trained on assays with more reproducible data (FIG. 6H; model performance correlated with the data replication quality FIG. 6G), demonstrating the applicability of the approach to in vivo data.

### Example 4: Multi-Trait Capsid Identification

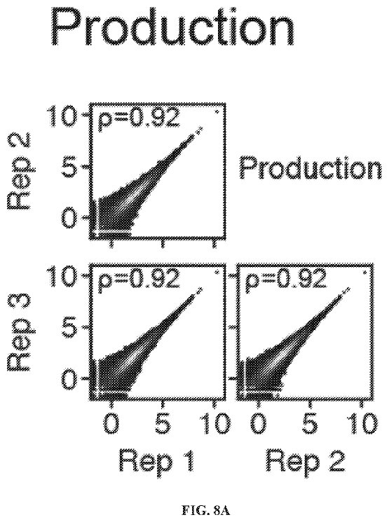

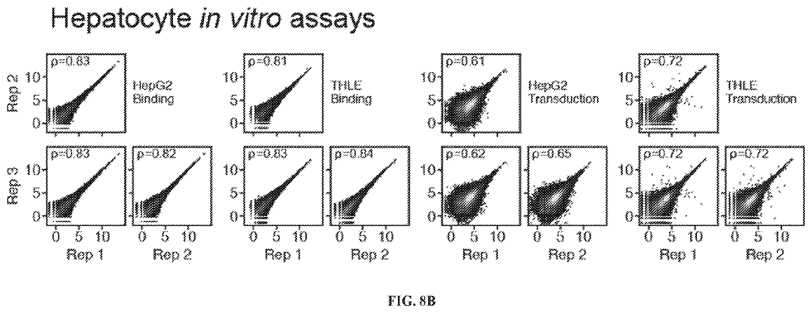

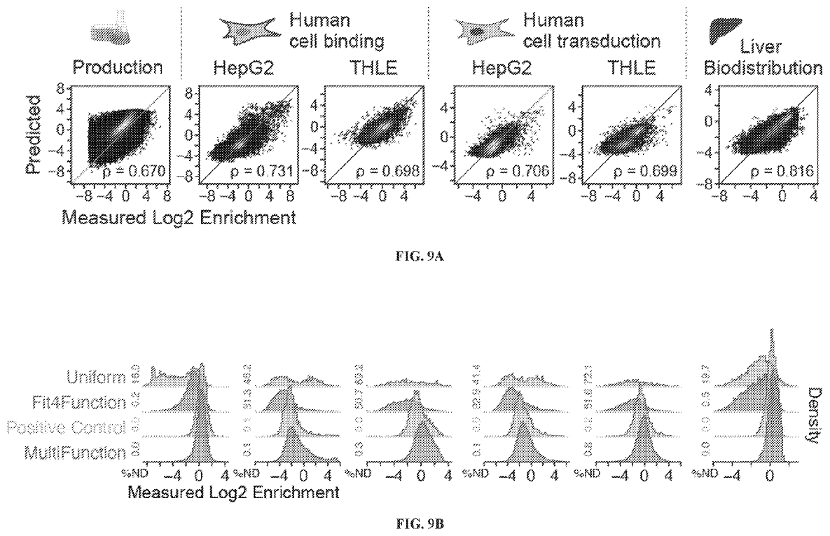

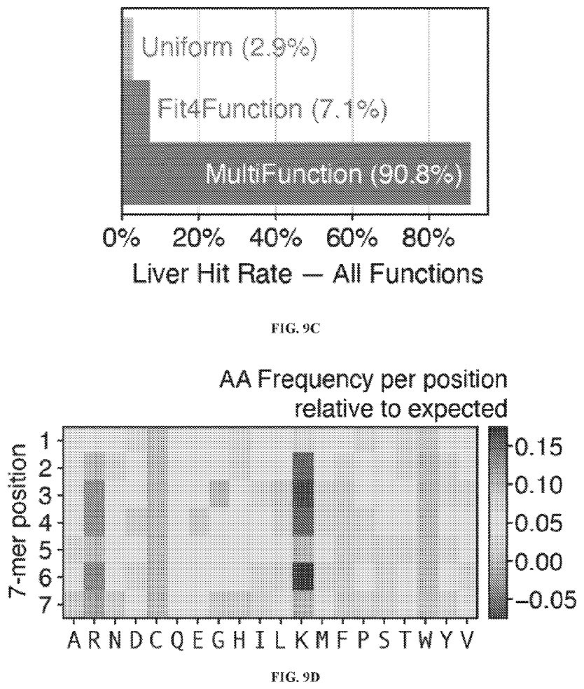

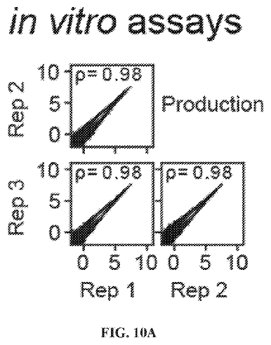

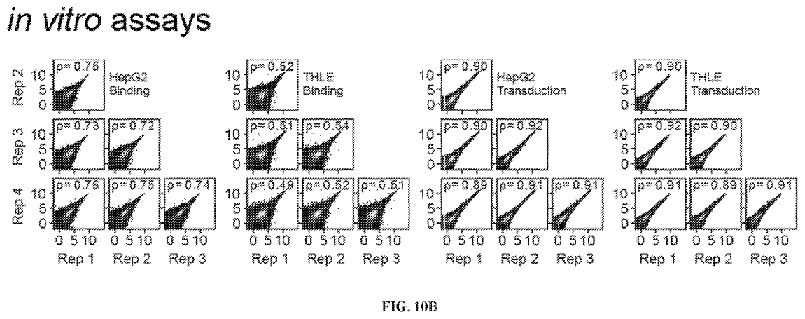

Efficient and durable gene delivery to the liver remains challenging due to capsid antigen presentation and T cell-mediated immunity. Liver-directed therapies should benefit from the development of potent AAV vectors that can be administered at lower doses to reduce the exposure to capsid antigens. There is a need for capsids that are compatible with preclinical efficacy and safety testing. The objective was to design a ‘MultiFunction’ library consisting only of variants that were each predicted to possess multiple enhanced functions related to cross-species hepatocyte gene delivery. Toward this goal, five separate functional screens of the Fit4Function library were performed for capsids capable of cross-species hepatocyte gene delivery: (1) binding (2) transduction of the human hepatocellular carcinoma cell line (HepG2), (3) binding or (4) transduction of the human liver epithelial cell line (THLE), and (5) efficient liver biodistribution in C57BL/6J mice (FIGS. 8A and 8B). This high-quality data from these functional screens was used to train and assess the performance of five independent sequence-to-function models (FIG. 9A). With the and production fitness model and these five functional fitness models, 10M randomly generated capsid variants were screened in silico and 30K liver-targeted MultiFunction candidate variants predicted to have enhanced phenotypes across all five functions and production fitness were selected. “Enhanced phenotype” was arbitrarily defined as any variant above the 50th percentile of measured enrichment scores. In the MultiFunction library, each variant was encoded by two nucleotide sequences serving as biological replicates. In addition, 3K variants were included from the training library (high and low production fitness; Uniform Control), 10K from the Fit4Function library (Fit4Function Control), and 3K from the known hits in Fit4Function library, i.e., variants from the Fit4Function library that had been experimentally confirmed to exhibit enhanced phenotypes for the five hepatocyte-related traits and production fitness (Positive Control).

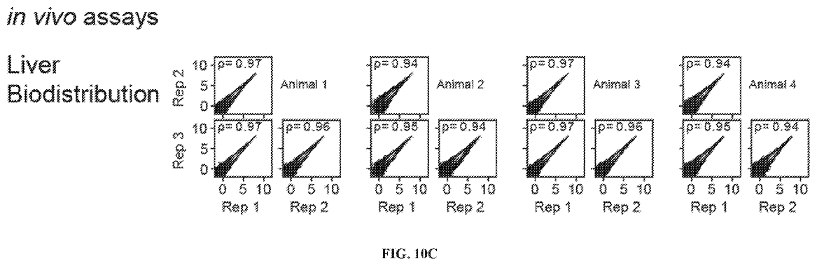

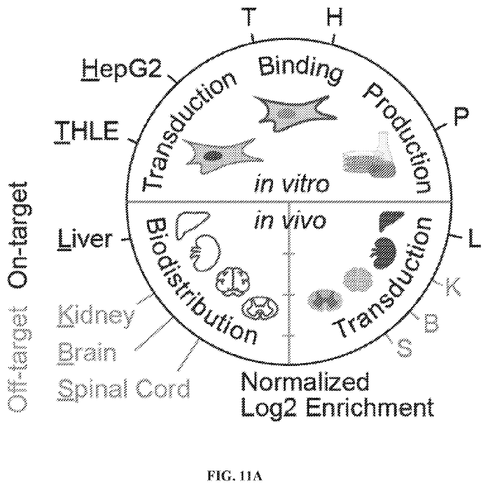

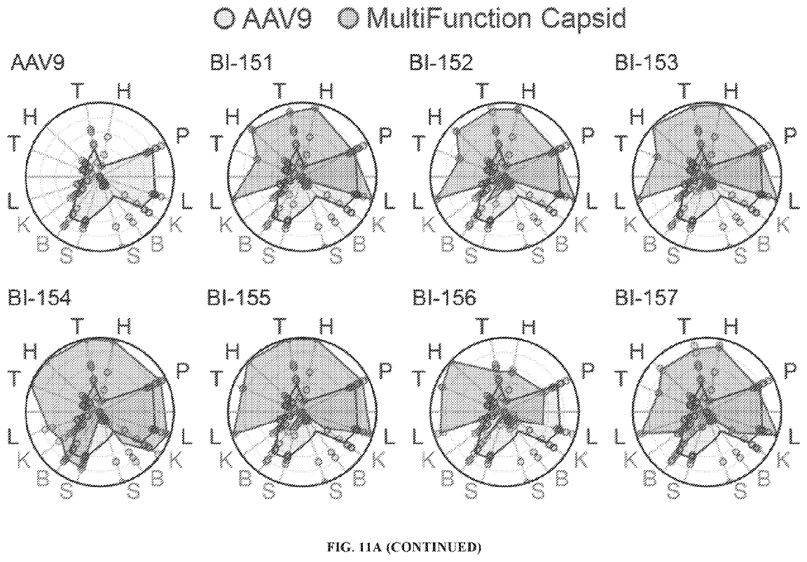

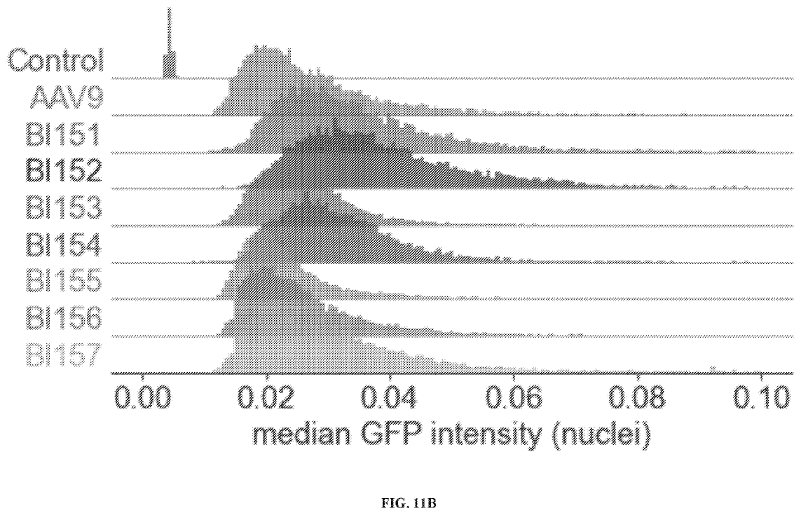

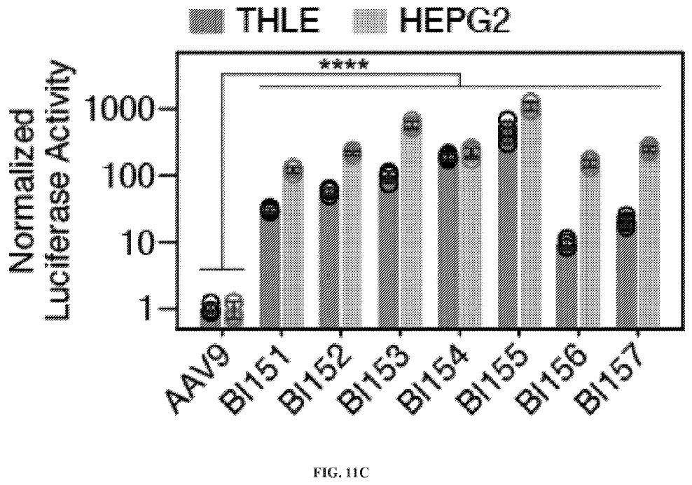

To assess the accuracy of researchers' predictions and identify the top-performing variants, the MultiFunction library was screened on the same five assays related to hepatocyte targeting and on production fitness (see replicate correlations in FIGS. 10A-10C).). The MultiFunction variants either matched or surpassed the performance of the positive controls from the Fit4Function library (FIG. 9B); >88.5% of the MultiFunction library variants satisfied the enhanced phenotype definition as compared to 2.9% of sequences in the uniform space or 7.1% of the Fit4Function library control (FIG. 9C). Although the 7-mer sequences in the MultiFunction library have an increased frequency of arginine and lysine, the library diversity remained high (FIG. 9D).

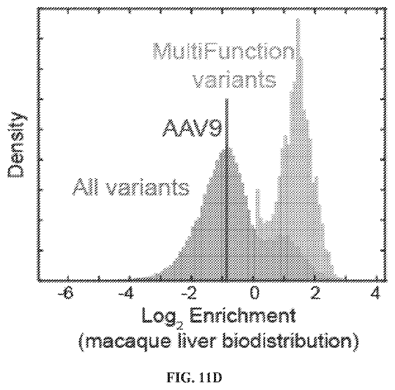

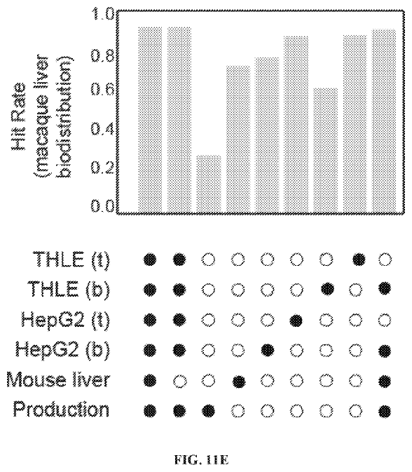

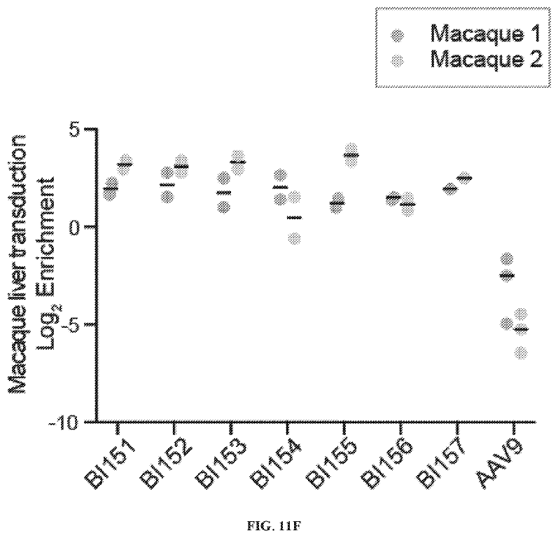

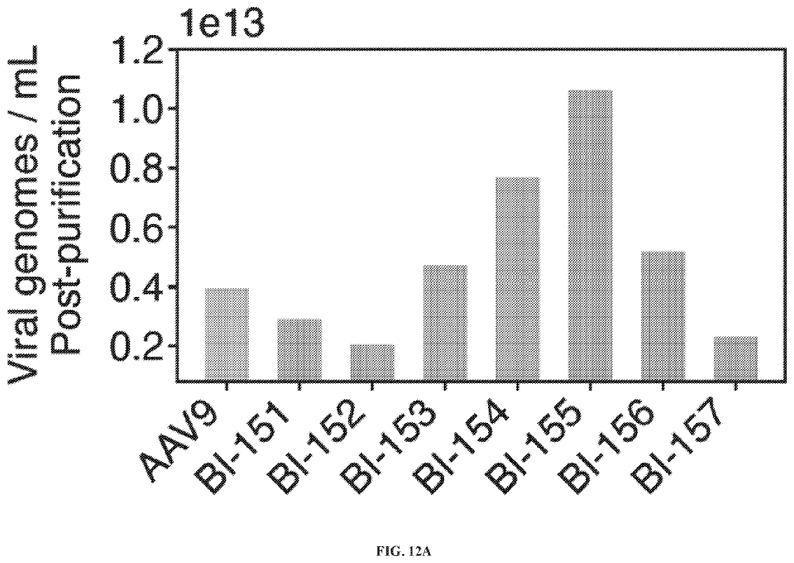

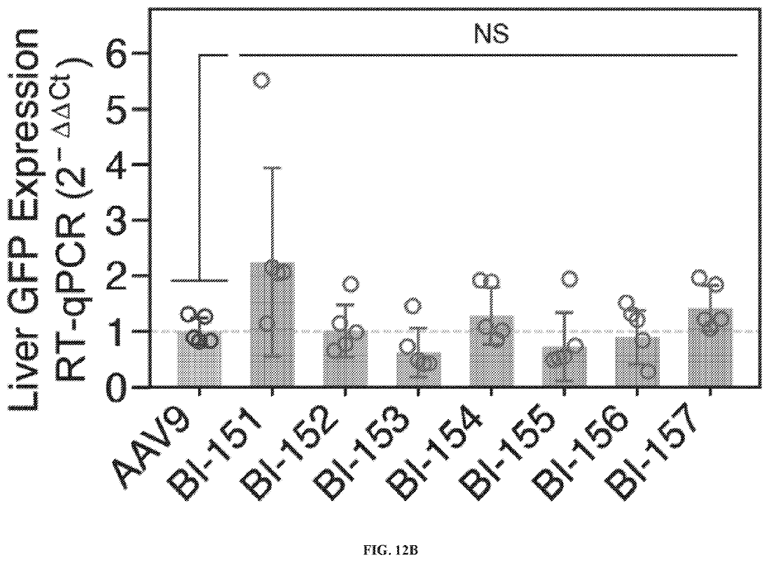

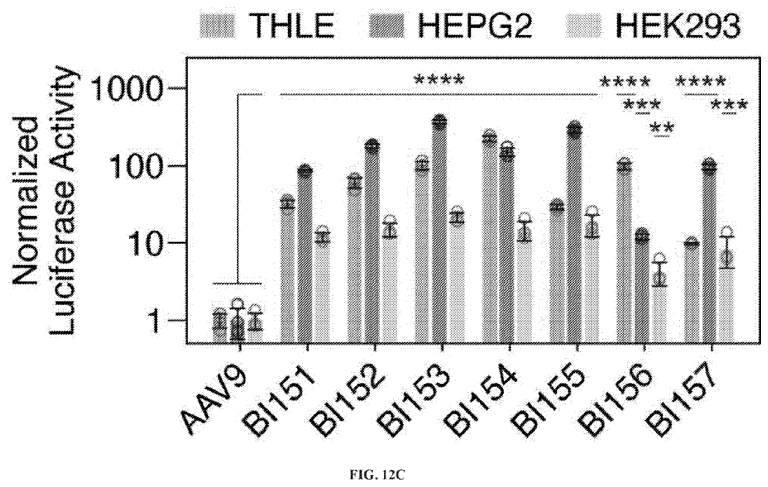

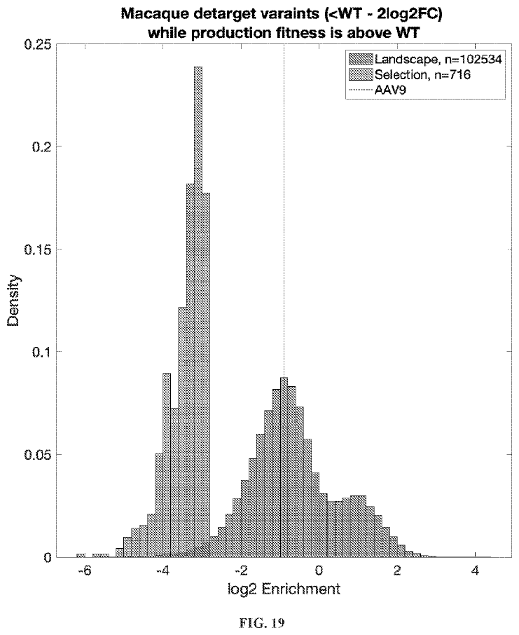

The performance of seven variants that were selected from the MultiFunction library were individually assessed based on their measured production fitness, liver biodistribution and transduction in mice, and their enhanced ability to bind and transduce human HEPG2 and THLE cells (FIG. 11A). Each capsid and AAV9, as a control, were used to package a single-stranded GFP and Luciferase dual reporter AAV2 genome. Production yields were comparable to that of AAV9 (FIG. 12A). When administered to mice at 1×10¹⁰ vg/mouse and assessed for GFP expression three weeks later, each capsid and AAV9 efficiently transduced hepatocytes as assessed by the native GFP fluorescence in DAPI⁺ liver nuclei (FIGS. 11B, 12B, and 18). All novel AAVs were more effective than AAV9 at transducing the HEPG2 and THLE cell lines (FIGS. 11C and 12C).

### Example 5: Fit4Function Translates Across Species to Macaques

A 100K member Fit4Function library was administered intravenously to an adult cynomolgus macaque and assessed biodistribution. Liver-targeted MultiFunction capsids, predicted with the six prior models that were trained only on human cell and mouse data and production fitness, were highly enriched in terms of macaque liver biodistribution (FIG. 11D). The combination of multiple functional predictors was more effective at identifying variants with increased biodistribution to the macaque liver than any single predictor used in isolation (FIG. 11E). The five liver models exhibited redundancy, which is unsurprising given that they are readouts of related functions (FIG. 11E). Surprisingly, the in vivo human hepatocyte transduction models translated better to cynomolgus macaque liver biodistribution compared to the in vivo mouse liver biodistribution model, which was neither necessary nor sufficient to demonstrate transferability to macaque liver biodistribution; the hit rate did not decrease when the mouse liver model was not included in the combination of models (FIG. 11E). The hit rate decreased only modestly when both human hepatocyte transduction models were excluded, demonstrating the utility of using models in combination (FIG. 11E). All seven of the liver MultiFunction capsids individually validated in mice and human cells (FIG. 11A, 12C) were more efficient than AAV9 at transducing the macaque liver (FIG. 11F; n=2 rhesus macaques) when administered as a library.

### Example 6: Production Fitness

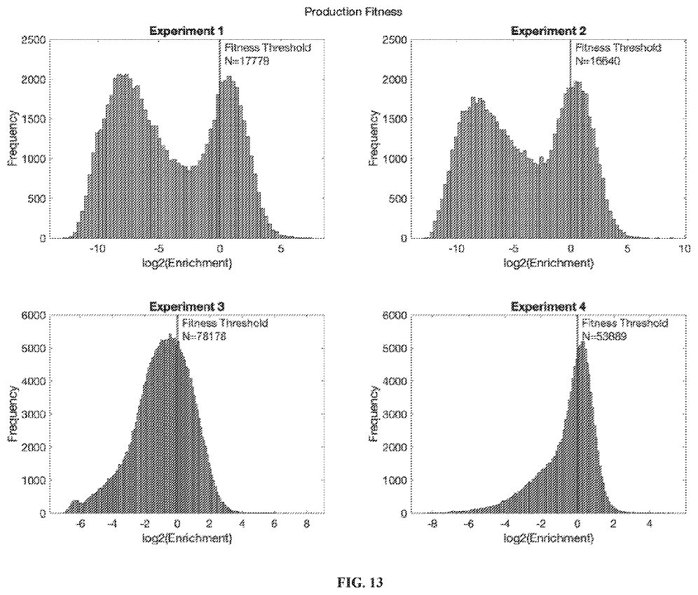

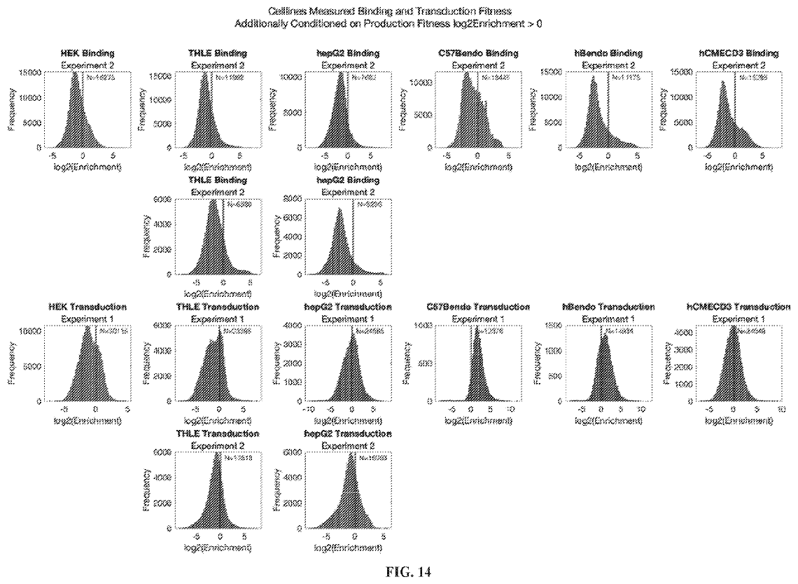

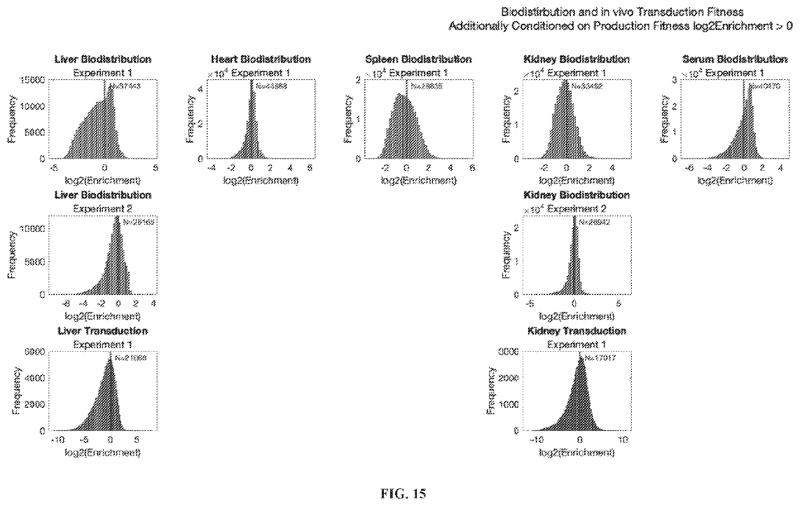

Production fitness is a bottleneck for manufacturability of viral vectors. Screening randomly synthesized libraries can result in the identification of capsids optimized for function, but that are challenging to manufacture. Four experiments were, therefore, undertaken (i.e., Experiments 1-4) to assess the “manufacturability” or production fitness under defined conditions for capsid variants in a library (FIG. 13). The process was compatible with low bias purification processes as well as more scalable customized manufacturing processes. Capsid production fitness was measured in a library format by measuring nuclease resistant (packaged) AAV genomes using next generation sequencing (NGS). Each genome was packaged by the capsid that it encodes, which made it possible to quantitatively measure the relative production fitness of individual variants within a capsid library. Production fitness was scored by measuring the log 2 enrichment (mean reads per million (RPM) for a capsid sequence in the packaged virus library vs the plasmid RPM used to generate the virus library). Variants with high production fitness were suitable to be utilized to generate a library suitable to be subsequently screened for different functions to obtain variants that would be manufacturable and carry enhanced function(s) of interest.

### Example 7: Functional Fitness In Vitro

AAV capsid variants had different attributes that could be assessed through in vitro and in vivo assays that measure the ability of specific capsids to bind or transduce relevant cell types including those derived from humans, mice, or other species commonly used for disease models. Accordingly, the data shown in FIG. 14 was generated using Fit4Function libraries to learn to map 7-mer sequence to in vivo cell binding and transduction. Tables 2-13 below list 7-mer motifs found to be enriched for the indicated trait, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position. In Tables 2-13, “enriched” means a log 2 enrichment value of greater than 0.

TABLE 2
7-mer motifs enriched for HEK293 binding.

| | Reported | Possible |
|---|---|---|
| Motifs | Variants | Variants |
| [RQMT][RNQP][RQPS][ANQGST]**G | 161 | 153600 |
| [QTV][ANQGKP][RNGSTY]*[ARGT]*[GP] | 180 | 345600 |
| [RKSV][FPS]**[RT][QGS][GS] | 23 | 57600 |
| [GST]*[QT][NQ]R*G | 19 | 4800 |
| [TV]**[NS]R[QGM]G | 23 | 4800 |
| [RNQT][ANPS][RNS]**[NQGS]G | 93 | 76800 |
| T[NGS]**R[AQS]G | 19 | 3600 |
| NR**[NG][QG]A | 9 | 1600 |
| R**QGGG (SEQ ID NO: 199501) | 4 | 400 |
| [QF][KS][RN]**[NP][AP] | 11 | 12800 |
| QR**S[TV]A (SEQ ID NO: 199502) | 9 | 800 |
| [QKM]*G[RSV][KT]*G | 14 | 7200 |
| [QMTV][RN]*[ANV][RQS]*[GS] | 45 | 57600 |
| [NQ]R[NGP][NQS]**A | 34 | 7200 |
| [QT]*[RT][RT]*[NQG][AG] | 32 | 19200 |
| NTR**SA (SEQ ID NO: 199503) | 4 | 400 |
| QRP*[AS]*[AS] | 12 | 1600 |
| RQ**TNA (SEQ ID NO: 199504) | 4 | 400 |
| QRP**[MV][AS] | 11 | 1600 |
| [RN]*N[RS]*[QG]G | 13 | 3200 |
| [MTV]R[PS]*[QT]*G | 19 | 4800 |
| [RY]S*[QK]*Q[GS] | 8 | 3200 |
| MRG**MG (SEQ ID NO: 199505) | 5 | 400 |
| [GV]*[NT]*R[QS]G | 11 | 3200 |
| [ST][RQ][RS]T**A | 12 | 3200 |
| R*S*STP (SEQ ID NO: 199506) | 4 | 400 |
| QR**TNG (SEQ ID NO: 199507) | 4 | 400 |
| Q*RQT*P (SEQ ID NO: 199508) | 4 | 400 |
| [NQ]RQ*[GS]*A | 13 | 1600 |
| TR**NNA (SEQ ID NO: 199509) | 4 | 400 |
| [RT]S*[RQ][QS]*A | 8 | 3200 |
| GQ*RV*G (SEQ ID NO: 199510) | 5 | 400 |
| T*TSR*G (SEQ ID NO: 199511) | 6 | 400 |
| TRG**TG (SEQ ID NO: 199512) | 4 | 400 |
| NR*[GT]*[TV]G | 10 | 1600 |
| T*RT*SA (SEQ ID NO: 199513) | 4 | 400 |
| MG*R*GA (SEQ ID NO: 199514) | 4 | 400 |
| [NQ]R*[NQ]S*A | 9 | 1600 |
| RQ*PT*A (SEQ ID NO: 199515) | 4 | 400 |
| T*T*RSG (SEQ ID NO: 199516) | 4 | 400 |
| T*RGS*P (SEQ ID NO: 199517) | 4 | 400 |
| TR**TMG (SEQ ID NO: 199518) | 4 | 400 |
| R*TS*SP (SEQ ID NO: 199519) | 5 | 400 |
| N**QRSA (SEQ ID NO: 199520) | 4 | 400 |
| [QT][RK]*S*[TY]A | 12 | 3200 |
| QR*PA*G (SEQ ID NO: 199521) | 4 | 400 |
| RS*S*GG (SEQ ID NO: 199522) | 4 | 400 |
| RTS*S*P (SEQ ID NO: 199523) | 4 | 400 |
| TRQ*T*G (SEQ ID NO: 199524) | 4 | 400 |
| QR*S*TG (SEQ ID NO: 199525) | 4 | 400 |
| R*NS*SP (SEQ ID NO: 199526) | 5 | 400 |
| MR*G*QS (SEQ ID NO: 199527) | 4 | 400 |
| N**SRQG (SEQ ID NO: 199528) | 4 | 400 |
| NR*ST*A (SEQ ID NO: 199529) | 4 | 400 |
| RSQ*G*G (SEQ ID NO: 199530) | 4 | 400 |
| T*RTN*A (SEQ ID NO: 199531) | 4 | 400 |
| T*S*RMG (SEQ ID NO: 199532) | 4 | 400 |
| TR**TQA (SEQ ID NO: 199533) | 4 | 400 |
| TRT**SG (SEQ ID NO: 199534) | 4 | 400 |
| YSGK**G (SEQ ID NO: 199535) | 4 | 400 |

TABLE 3
7-mer motifs enriched for
HEK293 transduction.

| | Reported | Possible |
|---|---|---|
| Motifs | Variants | Variants |
| [RQGKST][ARNQGFPST]** | 1130 | 1360800 |
| [ARNQGKMTV][ANQGMSV]G | | |
| [\hat{ }ADCEHILKPW][ARNQGKPS] | 3535 | 11520000 |
| [ARNQGKST][ARNQKPSTV]**[NDGPS] | | |
| [NQKMFSTY][RNGKT][?CEHILKFSWY] | 1872 | 8640000 |
| **[RNQKMPSTV][ANDEPS] | | |
| [\hat{ }ARDCEHILW][RGLKPSY]* | 1289 | 1724800 |
| [NQGKPSTV]*[ANQGFST]G | | |
| [\hat{ }ARDCEHILPW][ARQGKPT] | 1139 | 1176000 |
| [RQGKPST]**[ANGMST]G | | |
| [\hat{ }ADCQEHILW][NQGLKMPST]* | 2306 | 6336000 |
| [\hat{ }NCEHILMFWY][ARNQGMTV]*[GS] | | |
| [RGMY][ANQGPS][NQKPST]** | 503 | 403200 |
| [ANGKMST]G | | |
| [NQGKFSTV][RNQGKS]**[ARNGKT] | 1614 | 4608000 |
| [NQGKFSYV][ANDGS] | | |
| [\hat{ }ARDCEHILFW]*[ARNQGKMPT] | 1297 | 1440000 |
| [RQGKPSTV]*[AQGST]G | | |
| [\hat{ }ARDCEHILPW]*[RNGKPST]* | 1236 | 1372000 |
| [ARNGKSV][NQGKMST]G | | |
| [RNKT][RNQPS]*[RQGPS][RQGS]* | 330 | 480000 |
| [APS] | | |
| [RNMPY][RNQKPS]*[ANQTV]* | 733 | 1680000 |
| [ANQGKTV][DGPS] | | |
| [NQGKMFTV][ARQGKMPTV] | 1056 | 1612800 |
| [RNQGKPTY]*[ARQKMST]*G | | |
| [RNQMFST][RNQGKT]*[ARNQPST]* | 1150 | 2116800 |
| [AQGSTY][APS] | | |
| [QGKMSTV]*[ARNQGKMPT][RNQGKT] | 770 | 907200 |
| [ARNSTV]*G | | |
| [RMT]*[NQKT]*[ARQGT][ANQS][GP] | 222 | 192000 |
| [RKMSV]**[ANQGKPS][NGMSTV] | 910 | 1764000 |
| [ANGKSTV][DGS] | | |
| [NQKMPTYV]*[ARGKPTV][NGST] | 960 | 1612800 |
| [RNQKST]*[GPS] | | |
| [RNQKMSY][RQKST][ADQKPST]* | 837 | 1960000 |
| [AKSTV]*[ADPS] | | |
| [RK]**[NG][AS][GT]A | 29 | 6400 |
| [RQ]*[RNPST][AQGST][AQGSTV]* | 411 | 360000 |
| [GPS] | | |
| R[QPS]*S*[QGV]G | 19 | 3600 |
| [RKV][NGKP]*N[ANGT]*G | 41 | 19200 |
| [NQGLKFT][NGKS][QKMS]** | 507 | 1344000 |
| [ARQKSV][ANDEG] | | |
| [RQMS]*[RNQKST][NPST]* | 478 | 806400 |
| [ARNQGMS][DGP] | | |
| [RKT]**[PST][QT][ANG]G | 54 | 21600 |
| [NQKMT][ARDKPST][RNGKP]*G*G | 117 | 70000 |
| [RNQKMFT][RDGKPST][RNQGKPT]* | 819 | 1372000 |
| [NQGST]*[AS] | | |
| [RNQMST][RNDQKST][RGKPST] | 1093 | 2822400 |
| [AQGKPST]**[APSV] | | |
| [QKT]*[KS]*[AQGS][NQS][AS] | 69 | 57600 |
| [KFV]*[ANK]*[ST][KS][AD] | 31 | 28800 |
| [RQKMTYV][RNDKPS][RQGPS] | 749 | 1176000 |
| [NDQGPST]**[GP] | | |
| [NQMT]R*[NGPTV][AQGS]*[GS] | 114 | 64000 |
| [QMSY][RKP]**S[AQGKV][ADGS] | 116 | 96000 |
| R[NS]**[ANQ][AQT]A | 21 | 7200 |
| R[QS]**T[NQ][AS] | 13 | 3200 |
| [RQ]*[KPT][GST]*[ANQGT]G | 85 | 36000 |
| [RN][RQP]*[GPS]T*A | 20 | 7200 |
| [RNMPT]*[RNGKST][ANDQKPST] | 355 | 480000 |
| [RNGST]*A | | |
| K[QPS]*[NMS]*[QV][GS] | 27 | 14400 |
| K*[NQG]*[NT][AST]G | 33 | 7200 |
| RN*Q*SG (SEQ ID NO: 199536) | 4 | 400 |
| K*[ANGST][NST]*[AMS][GS] | 68 | 36000 |
| KT*S*GA (SEQ ID NO: 199537) | 4 | 400 |
| [RKMT][RQKPST]S*[ARSTV]*G | 86 | 48000 |
| [KM]*N*GNA (SEQ ID NO: 199538) | 8 | 800 |
| N**S[GT][GM]A | 9 | 1600 |
| K*[GPS][GT][AT]*A | 20 | 4800 |
| [QG]K*[GTV][AMS]*[AN] | 23 | 14400 |
| [RQT]*[RNQPT][RNST]*[QGS]A | 102 | 72000 |
| [NQ][RK]**[GS]TA | 12 | 3200 |
| [TV]**NRQG (SEQ ID NO: 199539) | 8 | 800 |
| R[AGPS][NQPT]*[NGST]*G | 94 | 25600 |
| R[MST]N**[QT][GP] | 21 | 4800 |
| [RNQ]*[PT][GT][RS]*A | 24 | 9600 |
| RS**NTG (SEQ ID NO: 199540) | 4 | 400 |
| [RK]*[PS]G*[NQGT]A | 31 | 6400 |
| Q*KSA*A (SEQ ID NO: 199541) | 4 | 400 |
| KN*G*TA (SEQ ID NO: 199542) | 4 | 400 |
| K*S*[AS]GA (SEQ ID NO: 199543) | 8 | 800 |
| [NG]K*[AS][GT]*A | 14 | 3200 |
| [GV]NS*[RK]*G | 8 | 1600 |
| KT*S*SA (SEQ ID NO: 199544) | 4 | 400 |
| [RM][KP]*[AS]S*G | 15 | 3200 |
| VK**STG (SEQ ID NO: 199545) | 4 | 400 |
| [QKT][RQGMSTV][AGKP][NGST]**A | 138 | 134400 |
| PR*AT*G (SEQ ID NO: 199546) | 4 | 400 |
| TRS*T*M (SEQ ID NO: 199547) | 4 | 400 |
| T**RQQA (SEQ ID NO: 199548) | 4 | 400 |
| KN*S*SG (SEQ ID NO: 199549) | 4 | 400 |
| RNS*[AG]*G (SEQ ID NO: 199550) | 8 | 800 |
| R*S*[AS]T[PS] | 9 | 1600 |
| [RG][KS]S*[GT]*[AG] | 13 | 6400 |
| QR*NS*A (SEQ ID NO: 199551) | 4 | 400 |
| R*SN*TG (SEQ ID NO: 199552) | 4 | 400 |
| R*[NS]*[GT]GG | 11 | 1600 |
| RAP**NS (SEQ ID NO: 199553) | 4 | 400 |
| R*NNS*G (SEQ ID NO: 199554) | 4 | 400 |
| [NK]*[GKP][GST]*[GS]A | 37 | 14400 |
| QK*GT*G (SEQ ID NO: 199555) | 4 | 400 |
| T**NRGG (SEQ ID NO: 199556) | 4 | 400 |
| VK*AS*A (SEQ ID NO: 199557) | 5 | 400 |
| K*P*TGG (SEQ ID NO: 199558) | 4 | 400 |
| K**QNQG (SEQ ID NO: 199559) | 4 | 400 |
| R*S*TAP (SEQ ID NO: 199560) | 4 | 400 |
| K*S[NG]*[GS][GS] | 13 | 3200 |
| R*SN*NA (SEQ ID NO: 199561) | 4 | 400 |
| RMP**GA (SEQ ID NO: 199562) | 4 | 400 |
| [RW][ANP]N[AKS]**A | 14 | 7200 |
| KT**SGG (SEQ ID NO: 199563) | 4 | 400 |
| R*P*TGA (SEQ ID NO: 199564) | 4 | 400 |
| N**QRSA (SEQ ID NO: 199520) | 4 | 400 |
| K*[NQ]ST*A (SEQ ID NO: 199565) | 8 | 800 |
| MKN[TV]**A (SEQ ID NO: 199566) | 9 | 800 |
| KGNN**G (SEQ ID NO: 199567) | 5 | 400 |
| TKP**AA (SEQ ID NO: 199568) | 4 | 400 |
| TR*GT*G (SEQ ID NO: 199569) | 4 | 400 |
| KN*G*SA (SEQ ID NO: 199570) | 5 | 400 |
| K*ASS*A (SEQ ID NO: 199571) | 4 | 400 |
| KLNS**G (SEQ ID NO: 199572) | 4 | 400 |
| N**SRQG (SEQ ID NO: 199528) | 4 | 400 |
| QNR*A*P (SEQ ID NO: 199573) | 4 | 400 |
| R*P*AGA (SEQ ID NO: 199574) | 4 | 400 |
| RT**STP (SEQ ID NO: 199575) | 4 | 400 |
| SRTT*NG (SEQ ID NO: 199576) | 4 | 20 |
| T*RT*NA (SEQ ID NO: 199577) | 4 | 400 |
| TK*NS*G (SEQ ID NO: 199578) | 4 | 400 |
| TRP**AA (SEQ ID NO: 199579) | 4 | 400 |

TABLE 4
7-mer motifs enriched for THLE binding.

| | Reported | Possible |
|---|---|---|
| Motifs | Variants | Variants |
| QSRT**P (SEQ ID NO: 199580) | 4 | 400 |
| K[HP][NT]*P*[NS] | 12 | 3200 |
| RN*P*TS (SEQ ID NO: 199581) | 4 | 400 |
| K**GPKD (SEQ ID NO: 199582) | 5 | 400 |
| NRGQ**A (SEQ ID NO: 199583) | 4 | 400 |
| A**NEKR (SEQ ID NO: 199584) | 4 | 400 |
| TG**RSG (SEQ ID NO: 199585) | 4 | 400 |
| TAN*R*G (SEQ ID NO: 199586) | 4 | 400 |
| T*TNR*G (SEQ ID NO: 199587) | 4 | 400 |
| QSR**NP (SEQ ID NO: 199588) | 4 | 400 |
| T*T*RSG (SEQ ID NO: 199516) | 4 | 400 |
| K**NPAN (SEQ ID NO: 199589) | 4 | 400 |
| KM**PKD (SEQ ID NO: 199590) | 4 | 400 |
| MSRN**A (SEQ ID NO: 199591) | 4 | 400 |
| NDA**KK (SEQ ID NO: 199592) | 4 | 400 |
| QR*GP*M (SEQ ID NO: 199593) | 4 | 400 |
| RS*P*NA (SEQ ID NO: 199594) | 4 | 400 |
| T*S*RMG (SEQ ID NO: 199532) | 4 | 400 |
| T*TSR*G (SEQ ID NO: 199511) | 4 | 400 |
| VAR*H*G (SEQ ID NO: 199595) | 4 | 400 |

TABLE 5
7-mer motifs enriched for THLE transduction.

| | Reported | Possible |
|---|---|---|
| Motifs | Variants | Variants |
| [GKMFSTYV][NGKST]**[ARNGKSV] | 613 | 672000 |
| [AQMFST]G | | |
| [RNQKTY]*[NQGKPST][ANGPST]* | 603 | 504000 |
| [NQGTV]G | | |
| [QLMFPSTYV]K*[GPSTV]*[QGST]G | 202 | 72000 |
| [RNQGKMPTY][ARNQGPST] | 1338 | 1612800 |
| [ARNQGKPT]**[AGKMSTV]G | | |
| [RQGMTV]*[RQGKPST][QGPST]* | 812 | 2016000 |
| [ARQMPS][DGPS] | | |
| [RNQGKMPST]*[AQGKMPSTV] | 1315 | 1555200 |
| [RNQGST][ARNQGKST]*G | | |
| [NGKTV]**[RNQGPSTV][ARQGKMST] | 2062 | 4096000 |
| [ANQGKFST][AGPS] | | |
| [RNGKMFSTY][ANQLKMPT]* | 1289 | 3225600 |
| [ARDQGSTV][ARQGKTV]*[GS] | | |
| [RQGKMFTV][ARNQGPST][RGKPST]* | 1343 | 1689600 |
| [^DCEILFPWY]*G | | |
| [RNQGIKMST][RDLKMFST][ANDQHMPST] | 2136 | 14515200 |
| **[ARNGKMSV][ANDEKPS] | | |
| [RNQGIKFV]*[ARNQPST]*[ARGKMTV] | 1151 | 3763200 |
| [ARNQGT][DEGS] | | |
| [^ARNCEHILP][ANQGKMPST] | 3823 | 12830400 |
| [RNQGHKMST][ARNQGKPTV]**[ARGS] | | |
| [RNQGIMFT][RKPSTV][AQGHKPST]* | 1492 | 4300800 |
| A[RNIKST]*[EGPS] | | |
| [NQKMFSTV][RKS]**[AGST][ARNDKSTV] | 483 | 1228800 |
| [ADEK] | | |
| [RNQKMSTY][RNGKST]*[ARNQMPSTV]* | 2032 | 8640000 |
| [^CEHILKFPWV][ADPST] | | |
| K*[GMP][QST]*[AQM][GS] | 40 | 21600 |
| [RQKTY]*[ARQKPST][QGST][ANGST]* | 617 | 1120000 |
| [ADPS] | | |
| [NQFTY]*[KST]*[ARGS][AQGKMTV]G | 203 | 168000 |
| [RT][RPT]**[ANST][NQT][APS] | 88 | 86400 |
| [RNKMPY][ARNGLKS]*[ANQKST]* | 505 | 504000 |
| [AQGSV]G | | |
| [RNQSY][KPST]**[RNGMS][QG]G | 126 | 80000 |
| [RKT][PS]*[RPST][QGS]*A | 60 | 28800 |
| [RNQEKMS][RNQT][RGIPST][NQGKPT] | 606 | 2016000 |
| **[AEGFP] | | |
| [NQT]K*[AGPST][GMS]*[AS] | 85 | 36000 |
| [RNK][RDLS][RNPT]S**[GP] | 32 | 38400 |
| [RK]*[NP][GV]*[GT]A | 18 | 6400 |
| [RQGMFSY][KS][NQGMPS]**[NQGS] | 299 | 268800 |
| [GS] | | |
| [MFPV]K[ANPS]S**[GS] | 42 | 12800 |
| [MF]K[NT]**[QPT]A | 17 | 4800 |
| [GFT]*[GKT]*[RNQK][KSTV][GS] | 92 | 115200 |
| [QGM][RKS][NGPS]*[AST]*A | 94 | 43200 |
| [RT]*[KP]*[QT][QG]A | 12 | 6400 |
| [GM][HP]K**[AP]A | 10 | 3200 |
| [TV]*[NK]*S[NS][AS] | 23 | 6400 |
| [QGMT][RK]*[GPTV][AS]*G | 63 | 25600 |
| [RV][KP]*NT*G | 9 | 1600 |
| M*SKS*A (SEQ ID NO: 199596) | 4 | 400 |
| A**NEKR (SEQ ID NO: 199584) | 4 | 400 |
| PR*AT*G (SEQ ID NO: 199546) | 4 | 400 |
| NK[AGP][AQV]**[DGS] | 28 | 10800 |
| [MS]**K[ST][GT]G | 10 | 3200 |
| [RQK][ARF]**T[AGS]G | 26 | 10800 |
| [QF]K**[AN][QS]s | 10 | 3200 |
| [NMT]K[ANP]*G*G | 19 | 3600 |
| R**KEEK (SEQ ID NO: 199597) | 4 | 400 |
| [RGV][KP]*[AS][ST]*[AS] | 35 | 19200 |
| NK[NS]*G*A (SEQ ID NO: 200021) | 8 | 800 |
| QK*GT*G (SEQ ID NO: 199555) | 5 | 400 |
| TK*NS*G (SEQ ID NO: 199578) | 4 | 400 |
| TGK**[AT]A (SEQ ID NO: 199598) | 8 | 800 |
| R**AGVG (SEQ ID NO: 199599) | 4 | 400 |
| [NT]K*[TV]*KD | 9 | 1600 |
| MKS**TG (SEQ ID NO: 199600) | 4 | 400 |
| G*KSV*G (SEQ ID NO: 199601) | 4 | 400 |
| [KM]*[NS]*[GS][NG]A | 25 | 6400 |
| TNK**QG (SEQ ID NO: 199602) | 4 | 400 |
| K[DKPS][RNDQ]*[GK]*[AD] | 22 | 25600 |
| NAR*T*G (SEQ ID NO: 199603) | 4 | 400 |
| MR*NQ*G (SEQ ID NO: 199604) | 4 | 400 |
| [FT]*K[AT]*QA | 10 | 1600 |
| KT**GGA (SEQ ID NO: 199605) | 4 | 400 |
| V*NKV*G (SEQ ID NO: 199606) | 4 | 400 |
| FKG**SA (SEQ ID NO: 199607) | 4 | 400 |
| FNK**QG (SEQ ID NO: 199608) | 4 | 400 |
| GPK*T*A (SEQ ID NO: 199609) | 4 | 400 |
| KQS*S*P (SEQ ID NO: 199610) | 4 | 400 |
| [NS]*KG*[ST]A | 9 | 1600 |
| GP*G*KG (SEQ ID NO: 199611) | 4 | 400 |
| RDKS**A (SEQ ID NO: 199612) | 4 | 400 |
| R*S*STP (SEQ ID NO: 199506) | 4 | 400 |
| KT**AGG (SEQ ID NO: 199613) | 4 | 400 |
| FK**TQG (SEQ ID NO: 199614) | 4 | 400 |
| FGK**TG (SEQ ID NO: 199615) | 5 | 400 |
| NKTG**A (SEQ ID NO: 199616) | 4 | 400 |
| TK**TYG (SEQ ID NO: 199617) | 5 | 400 |
| TKPG**G (SEQ ID NO: 199618) | 4 | 400 |
| KP*T*GG (SEQ ID NO: 199619) | 4 | 400 |
| TGK**SA (SEQ ID NO: 199620) | 4 | 400 |
| KPN*S*A (SEQ ID NO: 199621) | 7 | 400 |
| TGKS**A (SEQ ID NO: 199622) | 5 | 400 |
| T*RT*SA (SEQ ID NO: 199513) | 4 | 400 |
| V*KS*TG (SEQ ID NO: 199623) | 4 | 400 |
| F*K*TSA (SEQ ID NO: 199624) | 4 | 400 |
| FGK**SG (SEQ ID NO: 199625) | 4 | 400 |
| K*GG*AG (SEQ ID NO: 199626) | 4 | 400 |
| KPS*N*A (SEQ ID NO: 199627) | 4 | 400 |
| QR*NS*A (SEQ ID NO: 199551) | 4 | 400 |
| R**QGGG (SEQ ID NO: 199501) | 4 | 400 |
| RP*N*GG (SEQ ID NO: 199628) | 4 | 400 |
| TK**TQG (SEQ ID NO: 199629) | 4 | 400 |
| TKSS**A (SEQ ID NO: 199630) | 4 | 400 |
| V*KSQ*G (SEQ ID NO: 199631) | 4 | 400 |

TABLE 6
7-mer motifs enriched for HepG2 binding.

| | Reported | Possible |
|---|---|---|
| Motifs | Variants | Variants |
| [ARDTV]**[NKP][REG][QEGK][RGK] | 42 | 216000 |
| NAR**GG (SEQ ID NO: 199632) | 4 | 400 |
| N[AR][RG]Q**[AG] | 9 | 3200 |
| VR**SSA (SEQ ID NO: 199633) | 4 | 400 |
| TG**RSG (SEQ ID NO: 199585) | 5 | 400 |
| R*KDS*A (SEQ ID NO: 199634) | 4 | 400 |
| T*T*RSG (SEQ ID NO: 199516) | 4 | 400 |
| T*TNR*G (SEQ ID NO: 199587) | 4 | 400 |
| T*TSR*G (SEQ ID NO: 199511) | 5 | 400 |
| G**SIRS (SEQ ID NO: 199635) | 4 | 400 |
| GQSS**R (SEQ ID NO: 199636) | 4 | 400 |
| M*KP*RD (SEQ ID NO: 199637) | 4 | 400 |
| NDA**KK (SEQ ID NO: 199592) | 4 | 400 |
| NK*DR*G (SEQ ID NO: 199638) | 4 | 400 |
| QRP*A*A (SEQ ID NO: 199639) | 4 | 400 |
| SP**RGG (SEQ ID NO: 199640) | 4 | 400 |
| V*N*SSA (SEQ ID NO: 199641) | 4 | 400 |

TABLE 7
7-mer motifs enriched for HepG2 transduction.

| | Reported | Possible |
|---|---|---|
| Motifs | Variants | Variants |
| [NDGILST]K*[RNGHKPSTV]*[ANDGKFSY] | 442 | 1008000 |
| [ADQST] | | |
| [RQGKMFPS][RNGHPST][RQGKPST]** | 1786 | 5644800 |
| [ARQGMPSTV][AEGS] | | |
| [MFTYV]K**[ANST][AQSTY]G | 133 | 40000 |
| [NQGKMFSYV][RQGLKS]*[ANDQKPS] | 866 | 1360800 |
| [ARNQGMSTV]*G | | |
| [RDQEK][ARGLMST]*[ANGKPT]*[RNKMS] | 278 | 1680000 |
| [AELM] | | |
| [\hat{ }ARDCEHFPWV]K[ANDQMPST]**[RQGKMT] | 684 | 768000 |
| [NDEG] | | |
| [\hat{ }ACGHILPW][RNDQGLKS][\hat{ }DCELKFWYV] | 5154 | 38016000 |
| [\hat{ }RNCEILMFWY]**[RNDEGKFPS] | | |
| [NKMSTW][ARKSTV][NQGPS][AQGKSV]**A | 293 | 432000 |
| [RNQGKTYV]*[ANQGMPST][ANQGKST]* | 1991 | 4300800 |
| [AQGKMS][AEGS] | | |
| [NQGIKFSTY]K[NDQHPS]*[ARNGKT]* | 442 | 648000 |
| [ADEPS] | | |
| [NMFTYV][KPS]*[AQGKPTV]*[AQGKS] | 551 | 1008000 |
| [DEGS] | | |
| [\hat{ }ADCEHILPW][ARNQGKFPT]** | 4152 | 14968800 |
| [ARNQKMSTY][ARNQGMS][ADEGPS] | | |
| [NQIKFY][ADQLK][ANGHM]**[ARQK] | 255 | 960000 |
| [NDGK] | | |
| [ARNDQGSTV]**[RNQGKP][RQEGKMT] | 822 | 3628800 |
| [NEGKFS][ARGK] | | |
| [NQGMT]K*[AQGPSTV][AGMST]*[ANGS] | 540 | 280000 |
| [DQKS][QKMST]**[AGST]K[DEK] | 73 | 96000 |
| [NQWV]K[GST]**SG | 27 | 4800 |
| K**[ANQS][ANQT][ADGK][ARDEG] | 105 | 128000 |
| K**[NQSTY]S[AQGKT][EGPS] | 71 | 40000 |
| [RNQGT]*[AGST][QGKT][RGKT]*G | 179 | 128000 |
| [RNQGMST][RNQGKPSV][ANGPST]* | 1668 | 3225600 |
| [ARQGIKST]*[AGS] | | |
| [RNQGKTV][RNQGKPS]*[ANQGSTV] | 1885 | 3292800 |
| [NGKMST]*[AGPS] | | |
| [QGKMT][GFP]K*[NQEGTY]*[AQGY] | 137 | 144000 |
| [NT][AD]*K[RS]*[LP] | 8 | 6400 |
| [NQMFTV]*K*[AQGMS][AQGKTV][EG] | 200 | 144000 |
| [RGIKF]*[ARNQGT]*[AKMT][ARGKS] | 268 | 720000 |
| [DEG] | | |
| [QMFT][RNQGMPS][RKP][ANGST]**G | 232 | 168000 |
| [NGMFT][NGKS]K[DQT]**[GSW] | 89 | 72000 |
| [GKFTY]*[AKPT]*[RNQST][AGKST][GS] | 315 | 400000 |
| [QFS]K[GT]**[ST]A | 23 | 4800 |
| T[RNS][ARGK]**[RQGT][EG] | 39 | 38400 |
| T*N*SKG (SEQ ID NO: 199642) | 4 | 400 |
| [GSTYV]*K[QT][NGS]*[GS] | 62 | 24000 |
| [KMFPSY][LK]*S*[ANQIST]G | 58 | 28800 |
| K[KS][DST]*S*G | 14 | 2400 |
| [RNQMPTV]*[GKT]S[ARQKS]*[AGS] | 134 | 126000 |
| G*K*TAA (SEQ ID NO: 199643) | 4 | 400 |
| K*[ANS][GS][GT]*[ADP] | 36 | 14400 |
| [RQGKP]*[GKP][NDPT][AQMS]*A | 92 | 96000 |
| [MFT]*K[APT]*[RQ][ADS] | 32 | 21600 |
| M*SKS*A (SEQ ID NO: 199596) | 6 | 400 |
| TK[NMP]**[ANQ]A | 21 | 3600 |
| [NQGS]K**G[QGF]G | 29 | 4800 |
| [NQF][QS]K*[AS]*G | 21 | 4800 |
| GK[GT][QT]**G | 11 | 1600 |
| [RGT][DGP]K[NQS]**A | 26 | 10800 |
| TGK**[AST]A (SEQ ID NO: 199644) | 12 | 1200 |
| K[DS][RN]*G*[AG] | 11 | 3200 |
| [GMPTV]*K[ASTV]*[QST]G | 74 | 24000 |
| [ST]K*[AQ]*[QS]A | 18 | 3200 |
| K*A[TV]*[RK]D | 11 | 1600 |
| [MF]KN**[QP]A | 8 | 1600 |
| Q[RQY][KP][ST]**A | 16 | 4800 |
| [NT]*KS*[ST][AP] | 16 | 3200 |
| TG**MKG (SEQ ID NO: 199645) | 4 | 400 |
| [QT][RG]*S*[KT][AP] | 12 | 6400 |
| [FV]*K*TSA (SEQ ID NO: 199646) | 8 | 800 |
| QK[GT][NS]**A | 8 | 1600 |
| T**KS[GS]G (SEQ ID NO: 200022) | 8 | 800 |
| [NP]K*S*GG (SEQ ID NO: 199647) | 10 | 800 |
| NK**G[ST]A (SEQ ID NO: 199648) | 8 | 800 |
| [QG]KN**SA (SEQ ID NO: 199649) | 10 | 800 |
| TKS**SN (SEQ ID NO: 199650) | 4 | 400 |
| MKNT**A (SEQ ID NO: 199651) | 4 | 400 |
| Q*K[GS]*[NV]G | 11 | 1600 |
| Q**KSNA (SEQ ID NO: 199652) | 4 | 400 |
| NN**SKG (SEQ ID NO: 199653) | 5 | 400 |
| M*N*GNA (SEQ ID NO: 199654) | 4 | 400 |
| KTQ*S*S (SEQ ID NO: 199655) | 4 | 400 |
| T**SGTP (SEQ ID NO: 199656) | 4 | 400 |
| RS*S*GG (SEQ ID NO: 199522) | 4 | 400 |
| MG*R*GA (SEQ ID NO: 199514) | 4 | 400 |
| [QT]*K[NS][ST]*G | 15 | 3200 |
| Q*KP*QG (SEQ ID NO: 199657) | 4 | 400 |
| KA**GDR (SEQ ID NO: 199658) | 4 | 400 |
| V*N*SSA (SEQ ID NO: 199641) | 4 | 400 |
| K*TG*RE (SEQ ID NO: 199659) | 4 | 400 |
| M*K*TSG (SEQ ID NO: 199660) | 4 | 400 |
| T*K*SSA (SEQ ID NO: 199661) | 4 | 400 |
| YQK*S*S (SEQ ID NO: 199662) | 4 | 400 |
| MK*T*TA (SEQ ID NO: 199663) | 4 | 400 |
| KPN*S*A (SEQ ID NO: 199621) | 5 | 400 |
| TK**GMG (SEQ ID NO: 199664) | 5 | 400 |
| MPK*S*S (SEQ ID NO: 199665) | 5 | 400 |
| DL*KP*K (SEQ ID NO: 199666) | 4 | 400 |
| KGNT**A (SEQ ID NO: 199667) | 4 | 400 |
| MK*G*TG (SEQ ID NO: 199668) | 4 | 400 |
| N**STMA (SEQ ID NO: 199669) | 4 | 400 |
| NK**SDK (SEQ ID NO: 199670) | 4 | 400 |
| T*K*SNS (SEQ ID NO: 199671) | 4 | 400 |
| T*KG*VG (SEQ ID NO: 199672) | 4 | 400 |
| TKQ**SA (SEQ ID NO: 199673) | 4 | 400 |
| V*NKV*G (SEQ ID NO: 199606) | 4 | 400 |

TABLE 8
7-mer motifs enriched for C57 brain
endothelia binding.

| | Reported | Possible |
|---|---|---|
| Motifs | Variants | Variants |
| KQ**AKD (SEQ ID NO: 199674) | 4 | 400 |
| NR**GGA (SEQ ID NO: 199675) | 5 | 400 |
| KKD**RD (SEQ ID NO: 199676) | 4 | 400 |
| QRNS**A (SEQ ID NO: 199677) | 4 | 400 |
| NRGQ**A (SEQ ID NO: 199583) | 5 | 400 |
| KKD**KD (SEQ ID NO: 199678) | 4 | 400 |
| R*KDS*A (SEQ ID NO: 199634) | 4 | 400 |
| RN**SGA (SEQ ID NO: 199679) | 4 | 400 |
| RQ*PT*A (SEQ ID NO: 199515) | 4 | 400 |

TABLE 9
7-mer motifs enriched for C57 brain endothelia transduction.

| | Reported | Possible |
|---|---|---|
| Motifs | Variants | Variants |
| [QKMPT][NKMPT][ARGKS][AQGT]**[AG] | 230 | 400000 |
| [TV]KPS**G (SEQ ID NO: 199680) | 8 | 800 |
| [QT][KS]*G[NKT]*G | 22 | 4800 |
| [KMT][RT][GS]**[NMT]G | 24 | 14400 |
| [RQMT][RQGK][QPST]*[RGTV]*G | 84 | 102400 |
| [GM]K[PS]**[NT]G | 13 | 3200 |
| KPT**MA (SEQ ID NO: 199681) | 4 | 400 |
| [QGKT]*[GPT][NQGT][RKT]*G | 65 | 57600 |
| [KT][KS]SS**[AG] | 8 | 3200 |
| [NGS][AK]*[GKS][NST]*[AP] | 35 | 43200 |
| [GK]*T*[RT][QS]G | 8 | 3200 |
| [QKT][GKF]**[RGT][GSY]G | 38 | 32400 |
| [QKS][RGKT]*[NT][ANGT]*G | 44 | 38400 |
| T**NRGG (SEQ ID NO: 199556) | 4 | 400 |
| [NK][RY]*T*[TV]G | 8 | 3200 |
| Q*PTS*A (SEQ ID NO: 199682) | 4 | 400 |
| [FS][NG]K*[GS]*G | 10 | 3200 |
| QR**STA (SEQ ID NO: 199683) | 4 | 400 |
| [QMS]K*G*[NT]G | 14 | 2400 |
| K*ST*NG (SEQ ID NO: 199684) | 5 | 400 |
| QRSS**G (SEQ ID NO: 199685) | 4 | 400 |
| [KV][NK]*[GV]*S[AG] | 12 | 6400 |
| N[RT][AR]*[NT]*A | 10 | 3200 |
| KN*GQ*G (SEQ ID NO: 199686) | 5 | 400 |
| K*S*QSA (SEQ ID NO: 199687) | 5 | 400 |
| [RN][ST][KS]*S*[GP] | 12 | 6400 |
| QKN*A*A (SEQ ID NO: 199688) | 4 | 400 |
| RP**MAG (SEQ ID NO: 199689) | 4 | 400 |
| N**STMA (SEQ ID NO: 199669) | 4 | 400 |
| N*K*GGG (SEQ ID NO: 199690) | 4 | 400 |
| KTT**GG (SEQ ID NO: 199691) | 5 | 400 |
| YKQ**GG (SEQ ID NO: 199692) | 4 | 400 |
| N*K*SNP (SEQ ID NO: 199693) | 4 | 400 |
| NKN*G*A (SEQ ID NO: 199694) | 5 | 400 |
| S**KTGG (SEQ ID NO: 199695) | 5 | 400 |
| GN*VK*G (SEQ ID NO: 199696) | 4 | 400 |
| M*SKS*A (SEQ ID NO: 199596) | 4 | 400 |
| MK**SAG (SEQ ID NO: 199697) | 4 | 400 |
| N*K*SQG (SEQ ID NO: 199698) | 4 | 400 |
| NRPS**P (SEQ ID NO: 199699) | 4 | 400 |
| QKT**GG (SEQ ID NO: 199700) | 4 | 400 |
| RS*Q*QS (SEQ ID NO: 199701) | 4 | 400 |
| T*RT*GG (SEQ ID NO: 199702) | 4 | 400 |

TABLE 10
7-mer motifs enriched for human brain endothelia binding.

| | Reported | Possible |
|---|---|---|
| Motifs | Variants | Variants |
| QSRT**P (SEQ ID NO: 199580) | 4 | 400 |
| MSRN**A (SEQ ID NO: 199591) | 4 | 400 |
| KKD**RD (SEQ ID NO: 199676) | 4 | 400 |
| NRGQ**A (SEQ ID NO: 199583) | 5 | 400 |
| KKD**KD (SEQ ID NO: 199678) | 4 | 400 |
| KKD*K*D (SEQ ID NO: 199703) | 4 | 400 |
| NR**GGA (SEQ ID NO: 199675) | 4 | 400 |
| R*KDS*A (SEQ ID NO: 199634) | 4 | 400 |

TABLE 11
7-mer motifs enriched for human brain endothelia transduction.

| | Reported | Possible |
|---|---|---|
| Motifs | Variants | Variants |
| NR**GGA (SEQ ID NO: 199675) | 4 | 400 |
| [STY][KP]*[QS][GSV]*G | 20 | 14400 |
| [GM]**[GT][GK][NF] G | 11 | 6400 |
| QRPN**A (SEQ ID NO: 199704) | 5 | 400 |
| [QS]K[GT]*S*G | 10 | 1600 |
| Q*K*AQG (SEQ ID NO: 199705) | 4 | 400 |
| Q*[GK][NS][KS]*G | 12 | 3200 |
| [QY][RK]P*[AT]*[AP] | 12 | 6400 |
| [QM]K*G*TG (SEQ ID NO: 199706) | 8 | 800 |
| TK*N*QG (SEQ ID NO: 199707) | 5 | 400 |
| K*ST*SG (SEQ ID NO: 199708) | 4 | 400 |
| KN*G*SA (SEQ ID NO: 199570) | 4 | 400 |
| KN*GQ*G (SEQ ID NO: 199686) | 5 | 400 |
| MK**SQG (SEQ ID NO: 199709) | 4 | 400 |
| TGT*R*G (SEQ ID NO: 199710) | 5 | 400 |
| GK*ST*A (SEQ ID NO: 199711) | 4 | 400 |
| MK*GS*G (SEQ ID NO: 199712) | 5 | 400 |
| MSK**AG (SEQ ID NO: 199713) | 5 | 400 |
| K*PTT*G (SEQ ID NO: 199714) | 4 | 400 |
| KP*T*GG (SEQ ID NO: 199619) | 4 | 400 |
| MP**SGS (SEQ ID NO: 199715) | 4 | 400 |
| Q*K*SNG (SEQ ID NO: 199716) | 4 | 400 |
| QKY*T*G (SEQ ID NO: 199717) | 4 | 400 |
| R*PS*QG (SEQ ID NO: 199718) | 4 | 400 |
| T**PTAG (SEQ ID NO: 199719) | 4 | 400 |
| TGKS**A (SEQ ID NO: 199622) | 4 | 400 |

TABLE 12
7-mer motifs enriched for human CMECD3 binding.

| | Reported | Possible |
|---|---|---|
| Motifs | Variants | Variants |
| N**SRQG (SEQ ID NO: 199528) | 4 | 400 |
| SP**RGG (SEQ ID NO: 199640) | 4 | 400 |
| NQ**RSA (SEQ ID NO: 199720) | 4 | 400 |
| QKY*T*G (SEQ ID NO: 199717) | 4 | 400 |
| S*QQR*G (SEQ ID NO: 199721) | 4 | 400 |
| MRG**MG (SEQ ID NO: 199505) | 5 | 400 |
| NR**GGA (SEQ ID NO: 199675) | 5 | 400 |
| NRGQ**A (SEQ ID NO: 199583) | 4 | 400 |
| T**NRGG (SEQ ID NO: 199556) | 4 | 400 |
| T*S*RMG (SEQ ID NO: 199532) | 4 | 400 |
| T*TNR*G (SEQ ID NO: 199587) | 4 | 400 |
| TAN*R*G (SEQ ID NO: 199586) | 4 | 400 |

TABLE 13
7-mer motifs enriched for human CMED3 transduction.

| | Reported | Possible |
|---|---|---|
| Motifs | Variants | Variants |
| [QKMFT][RNQP][RQKPS][ARDG]**G | 87 | 160000 |
| [RQGMSTY][ARNKP][RNGKPST]**[RNQGKMST][AEG] | 910 | 2352000 |
| [QGF][GPT][RK]*[NMS]*G | 26 | 21600 |
| [RK][NQ][PT]**[AT]G | 18 | 6400 |
| [RMT][RQGT][NPST]*[RQV]*G | 60 | 57600 |
| [KMST]*[NQGT][RQGS][RQT]*G | 69 | 76800 |
| [RNY][RKP]Q**[QGT][AG] | 28 | 21600 |
| [NQKT][RKP]**[NGS][NQGTV]A | 89 | 72000 |
| [RKMST][NGKPS]*[ARQS]*[ANQGTY][AS] | 178 | 480000 |
| [RKMP][NKS]*[ANQS]*[AGSV]G | 95 | 76800 |
| [RGMFT][NK]*[AGPV][NGKTV]*G | 71 | 80000 |
| [RKMTY]*[RKPST][AGST][GST]*[GPS] | 273 | 360000 |
| SK*GN*A (SEQ ID NO: 199722) | 4 | 400 |
| KP[AP]**G[AG] | 11 | 1600 |
| [NST][RK]*T*[NGV]G | 21 | 7200 |
| [RNT]*[GKT]*[ARGK][QMST]G | 62 | 57600 |
| [GK][NP][KS]*[NS]*[AS] | 15 | 12800 |
| QK*GT*G (SEQ ID NO: 199555) | 4 | 400 |
| [RQK]**[ANT][RT][GT]G | 30 | 14400 |
| [RK][NF]**[GTV]SG | 15 | 4800 |
| [GK][KP]*[SV]S*[NP] | 8 | 6400 |
| TG*NK*A (SEQ ID NO: 199723) | 5 | 400 |
| [NT][RT][PS][KS]**[AP] | 14 | 12800 |
| QKY*T*G (SEQ ID NO: 199717) | 4 | 400 |
| RS**TQS (SEQ ID NO: 199724) | 4 | 400 |
| [RN]*[NT][RS]*GG | 11 | 3200 |
| KTQ**AS (SEQ ID NO: 199725) | 4 | 400 |
| RS**GGG (SEQ ID NO: 199726) | 4 | 400 |
| KPP*T*G (SEQ ID NO: 199727) | 4 | 400 |
| [KY]*[QP]T*[GT]G | 12 | 3200 |
| S*TT*NG (SEQ ID NO: 199728) | 4 | 400 |
| KSPT**A (SEQ ID NO: 199729) | 4 | 400 |
| SRTT**G (SEQ ID NO: 199730) | 4 | 400 |
| [GY]K[QT][QT]**G | 14 | 3200 |
| Q**KSNA (SEQ ID NO: 199652) | 4 | 400 |
| K*NST*A (SEQ ID NO: 199731) | 4 | 400 |
| TRS*T*G (SEQ ID NO: 199732) | 4 | 400 |
| K*S*SGA (SEQ ID NO: 199733) | 4 | 400 |
| N**SRQG (SEQ ID NO: 199528) | 4 | 400 |
| VRP*T*G (SEQ ID NO: 199734) | 4 | 400 |
| Y*T*SKG (SEQ ID NO: 199735) | 4 | 400 |
| TR*VS*G (SEQ ID NO: 199736) | 5 | 400 |
| TG**RSG (SEQ ID NO: 199585) | 7 | 400 |
| K*P*SGG (SEQ ID NO: 199737) | 4 | 400 |
| KTS**GG (SEQ ID NO: 199738) | 4 | 400 |
| N**GQKG (SEQ ID NO: 199739) | 4 | 400 |
| N*GNR*A (SEQ ID NO: 199740) | 4 | 400 |
| QR*NS*A (SEQ ID NO: 199551) | 4 | 400 |
| QRP*S*A (SEQ ID NO: 199741) | 4 | 400 |
| R*QN*TG (SEQ ID NO: 199742) | 4 | 400 |
| RP*T*AP (SEQ ID NO: 199743) | 4 | 400 |
| SRTT*NG (SEQ ID NO: 199576) | 4 | 20 |
| TKSS**A (SEQ ID NO: 199630) | 4 | 400 |

### Example 8: Functional In Vivo Fitness

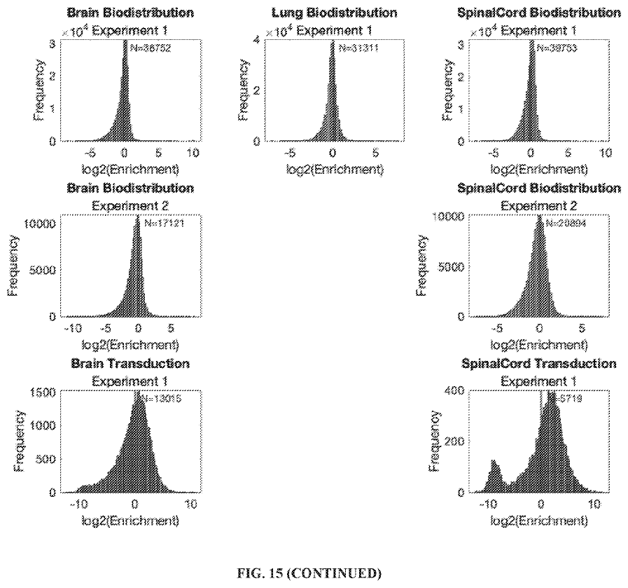

AAV libraries were screened to assess their in vivo biodistribution in mice (FIG. 15). Variants of AAV9 capsids modified at 588 site loop VIII with 7mer insertions were positively enriched for biodistribution or transduction of the indicated C57BL/6J mouse organ. Plotted sequences were also positively enriched for production fitness. Biodistribution/transduction fitness enrichment was measured by the fold change increase in abundance after screening in the indicated assay relative to its amount in the unscreened virus library. Enrichment was averaged across technical and biological replicates for each experiment.

Tables 14-24 below list 7-mer motifs found to be enriched for the indicated trait. In Tables 14-24 provided herein, “*” represents any amino acid, amino acids listed between brackets (“[ ]”) without the symbol “\hat{ }” represent the amino acids observed at a position, and amino acids listed between brackets (“[ ]”) and preceded by the symbol “\hat{ }” indicate amino acids not observed at the position. In Tables 14-24, “enriched” means a log 2 enrichment value of greater than 0.

TABLE 14
7-mer motifs enriched for liver biodistribution.

| | Reported | Possible |
|---|---|---|
| Motifs | Variants | Variants |
| [^{∧}ADCEGHILWY]*[^{∧}DCEGHILFWY] | 3195 | 3240000 |
| [ARNGKMPST\|[ARNQGISTV]*G | | |
| [^{∧}ADCEHILMWY][^{∧}DCEHILFPWY] | 3688 | 4400000 |
| [^{∧}RCEHILMFW]*[^{∧}NDCEHLFPWY]*G | | |
| [^{∧}ADCEHILMW][^{∧}CEGHIFWYV][^{∧}CEHILFPW] | 3883 | 5808000 |
| [^{∧}CEHILKMFWY]**G | | |
| [RNDQKMPV][ARNQMPST]**[ARNQGKST] | 3311 | 12902400 |
| [ARNDQGKTV][ARDIKPS] | | |
| [^{∧}ARDCEHILW][ARQGKPST]** | 2792 | 2851200 |
| [ARNQKMSTVI[ANQGKMTYVIG | | |
| [^{∧}ACEHILMFSW][^{∧}CEHIKFWYV]* | 8148 | 31944000 |
| [^{∧}DCQEHLFWY][^{∧}DCEHLFTWY]*[AEGLKP] | | |
| [RQKMSTV]**[ARNQKPSTV][ARNQGMSTV] | 2256 | 1814400 |
| [ARNQGSTVIG | | |
| [^{∧}RDCEHLPW][^{∧}CEHILMPSWV][^{∧}ANCEILMFWV] | 8875 | 47520000 |
| *[^{∧}DCQILMFWV]*[NDQEGMPSY] | | |
| [RNQGKMS][RQKST][^{∧}RDCEHILFWY]* | 2047 | 2940000 |
| [ARNMSTV]*[APS] | | |
| [^{∧}ADCEHLFPW]*[ARNQGPST][RQGKSTY]* | 2293 | 1971200 |
| [ANGKMSTV]G | | |
| [^{∧}ADCEHIK][^{∧}CQEHILFWY][ARNQGKMPS]** | 3429 | 4118400 |
| [ANQGKMSV]G | | |
| [^{∧}ADCEHPTWV][^{∧}ARCEIFWYV][^{∧}CILFTWY]* | 11347 | 50965200 |
| *[ARQGKPSTV][ANDQEGKMS] | | |
| [RNQGSTV]**[RNGKSTV][^{∧}ADCHLMFPWY] | 5260 | 12936000 |
| [^{∧}CHILKPWYV][AGKPSV] | | |
| [RNGKFTYV]*[ARNQGPST]*[ARNQKPSTV] | 2196 | 2073600 |
| [ADQGKMSTV]G | | |
| [ANQMFSTV]*K*[AQGST][ANQGMST]G | 421 | 112000 |
| [^{∧}ADCEHILW]*[?ACEHILFWYV] | 12565 | 30240000 |
| [ANQGKPSTV]*[^{∧}DCEHILKFWY][ANDGPSY] | | |
| [^{∧}ADCEHITW][^{∧}DCEGHIMFWY]* | 3249 | 4320000 |
| [ANQGMPSTV]*[^{∧}RDCEHLPTWY]G | | |
| [RNQILKFSV][QGKPST]T**[ARQGKMST] | 522 | 864000 |
| [ADEGS] | | |
| [^{∧}ACHILKW][^{∧}CEHILMFPWY] | 11213 | 57200000 |
| [^{∧}NDCELFWYV][^{∧}RNCEILMFWY]**[^{∧}CQHILKMPTY] | | |
| [RNQGKMSY][^{∧}RDCGHILMWY]** | 4166 | 10240000 |
| [ARGKMSTV][^{∧}NDCEHLPWYV][DGPS] | | |
| [^{∧}ARCEHILWY][ARQGKST]*[^{∧}CQEHILMFWY] | 4433 | 10780000 |
| [ARNGPST]*[NDGPS] | | |
| TK[ANQMST]**[QKS][NDS] | 50 | 21600 |
| [NQGKMST]*[ARNQGKPS][ARNQGPTV] | 2571 | 5017600 |
| [RNQHKMT]*[NGPS] | | |
| [NGFST]K**[ANGSTY][ANGMPT]A | 191 | 72000 |
| [RQMFS][RKPT]**[NQGT][NQS][NGS] | 324 | 288000 |
| [^{∧}ADCEHPTW][RNQLKMPST]*[^{∧}CEILKFWY] | 9173 | 45619200 |
| *[^{∧}CEHILPSWY][ANDQEKPS] | | |
| [QT]*[QT]R*QA | 9 | 1600 |
| [RNGT]*[RNGKST][ARNQGT][RNQGKMT]* | 1092 | 806400 |
| [AG] | | |
| [DQEMPST][ARGK]*[ARNQGKS]* | 979 | 3292800 |
| [ADQGKSY][ADLMPT] | | |
| [NGIKFTV]*[ARNGKTY]*[NQKMSTV] | 1746 | 4802000 |
| [ARNIKSV][ADEPS] | | |
| [RNQKMFST][RDQKS][RNQPST]*[AGS]*[AS] | 622 | 576000 |
| [RQGKS][QKST][RNQST]**[NT][GPS] | 281 | 240000 |
| K**[ANGSTY][QGMST][ANGKMT][ADEGP] | 457 | 360000 |
| [RKM][RNQGPS]*[ANQST][NQST]*[GS] | 477 | 288000 |
| [RQGIKT][PS][RKPS]*[ANGMT]*G | 168 | 96000 |
| [RQKM]*[NPS]*[AQGT][ANQGST][APS] | 461 | 345600 |
| [ADK]**[NGPST][AEGMST][ANQGKT][ARS] | 290 | 648000 |
| [QKMPTV]*[NKPS][QGKPT]S*A | 105 | 48000 |
| [RNQGST]*[RNQGKSV]*[RNQGK][AGKTV]G | 515 | 420000 |
| [NQGKT][RNK]*[ANGST][ANGMT]*A | 277 | 150000 |
| [RNQGKMY][RNQGS]*[NQGKS]*[AQGST] | 1049 | 700000 |
| [AG] | | |
| [NK]**G[QT][KS]G | 20 | 3200 |
| [RKMTY][RP][RNGKPS]*[ANQST]*[GPST] | 388 | 480000 |
| [QKMFPYV][RNGKPST][RNGPST] | 2021 | 2822400 |
| [ARDQGKST]**[GPS] | | |
| [RNQGKWV][ANQKPSTV][ARNQGKST] | 1476 | 2150400 |
| [AQGKST]**[AP] | | |
| TK*[GSTV]*[QGV]G | 30 | 4800 |
| T[RGS][RGKT]**[RNST][EGP] | 65 | 57600 |
| [RGV]P[RNK]*[QST]*A | 25 | 10800 |
| [RKMV]*[ANPST]S[GKST]*[DGPS] | 194 | 128000 |
| [NQMSTV][RNQGHK]**[ARGIKMST] | 1003 | 2073600 |
| [ARQKSV][ADE] | | |
| [NQK]*[NKT]*S[NV]G | 27 | 7200 |
| [RNQFT][RQK][QGKST]N**[APS] | 105 | 90000 |
| [NT][GK][NKMS]**[ANQT]A | 61 | 25600 |
| [RQK]*[ANQGPST][NGS][ANGKPT]*[AGP] | 759 | 453600 |
| [TYV]*[RK][QGT]S*[GPS] | 52 | 21600 |
| [NK][RNP][PT][NS]**[GP] | 34 | 19200 |
| K*[NG][AY]*KE | 11 | 1600 |
| [NQ]R[NQGT]**[NQT]A | 33 | 9600 |
| MKN*[GS]*G (SEQ ID NO: 199744) | 9 | 800 |
| [QGKM]*[GS]*[KS][NG][AG] | 56 | 25600 |
| [RNGMTYV][RGKS]*[AQKPS][NGS]*A | 227 | 168000 |
| [QMTYV][GK]*[NGKS]*TG | 58 | 16000 |
| [QKM][RKMS][NDP][GSTV]**A | 87 | 57600 |
| [NQSY]K*[QS][ANQSV]*G | 65 | 16000 |
| [QMT]RP**[AMS]A | 20 | 3600 |
| K*T[GI]*[RK][DE] | 12 | 3200 |
| [RQP]*[KPT][DQGS][AS]*A | 55 | 28800 |
| TKP**[AQ]A (SEQ ID NO: 199745) | 9 | 800 |
| T[RK]**[NGL][NMS][AG] | 39 | 14400 |
| K**Q[AS][GK][DS] | 11 | 3200 |
| RQ*[NP]T*A (SEQ ID NO: 199746) | 8 | 800 |
| [KS][GY]*T*[TV]G | 9 | 3200 |
| K*[QT][GT][GS]*[AS] | 27 | 6400 |
| RT*T*SA (SEQ ID NO: 199747) | 5 | 400 |
| K*A[NV]*[RS][DG] | 10 | 3200 |
| [NK]**Q[AR]S[AS] | 9 | 3200 |
| [QG][RP][KP]N**A | 10 | 3200 |
| [RT][GS]**[RN][ST]G | 21 | 6400 |
| MRPN**G (SEQ ID NO: 199748) | 4 | 400 |
| G*KSV*G (SEQ ID NO: 199601) | 4 | 400 |
| NK*T*SA (SEQ ID NO: 199749) | 5 | 400 |
| [NF]*[NK]*[NG]T[AS] | 14 | 6400 |
| [GK][KP][NQT]*[GS]*A | 27 | 9600 |
| MRT**SP (SEQ ID NO: 199750) | 4 | 400 |
| [NM]K*QT*[GS] | 10 | 1600 |
| GN**KNG (SEQ ID NO: 199751) | 4 | 400 |
| [NT][GT][RK]*N*A | 11 | 3200 |
| K*ASS*A (SEQ ID NO: 199571) | 4 | 400 |
| RT*GT*G (SEQ ID NO: 199752) | 5 | 400 |
| [RQS][AR][PT]**[NV][GS] | 26 | 19200 |
| RPT*S*[GS] (SEQ ID NO: 199753) | 8 | 800 |
| K**GKSA (SEQ ID NO: 199754) | 4 | 400 |
| [GV]KP**NA (SEQ ID NO: 199755) | 8 | 800 |
| TK*T*[KS][AD] | 11 | 1600 |
| TGK*G*A (SEQ ID NO: 199756) | 4 | 400 |
| KP*GT*G (SEQ ID NO: 199757) | 5 | 400 |
| RP*QQ*A (SEQ ID NO: 199758) | 4 | 400 |
| KPNN**P (SEQ ID NO: 199759) | 4 | 400 |
| TGK**SA (SEQ ID NO: 199620) | 4 | 400 |
| G**QKSG (SEQ ID NO: 199760) | 6 | 400 |
| TG**KTA (SEQ ID NO: 199761) | 4 | 400 |
| TN**RQG (SEQ ID NO: 199762) | 4 | 400 |
| TG*K*SG (SEQ ID NO: 199763) | 4 | 400 |
| I*AR*KE (SEQ ID NO: 199764) | 4 | 400 |
| N*K*NNG (SEQ ID NO: 199765) | 5 | 400 |
| R*S*STP (SEQ ID NO: 199506) | 5 | 400 |
| KGNN**G (SEQ ID NO: 199567) | 5 | 400 |
| TK*N*QG (SEQ ID NO: 199707) | 5 | 400 |
| G**QKGG (SEQ ID NO: 199766) | 4 | 400 |
| K*AT*KD (SEQ ID NO: 199767) | 4 | 400 |
| K*NQS*G (SEQ ID NO: 199768) | 4 | 400 |
| KPS*N*A (SEQ ID NO: 199627) | 4 | 400 |
| R*S*NVA (SEQ ID NO: 199769) | 4 | 400 |
| RP*GT*A (SEQ ID NO: 199770) | 4 | 400 |
| SRTT*NG (SEQ ID NO: 199576) | 4 | 20 |
| TKQ**SA (SEQ ID NO: 199673) | 4 | 400 |
| TS**RTP (SEQ ID NO: 199771) | 4 | 400 |

TABLE 15
7-mer motifs enriched for liver transduction.

| | Reported | Possible |
|---|---|---|
| Motifs | Variants | Variants |
| RQSA**T (SEQ ID NO: 199772) | 4 | 400 |
| RAHS**A (SEQ ID NO: 199773) | 4 | 400 |
| DG*K*KL (SEQ ID NO: 199774) | 4 | 400 |
| NR*A*DK (SEQ ID NO: 199775) | 4 | 400 |
| G*N*ANR (SEQ ID NO: 199776) | 4 | 400 |
| RQ**NST (SEQ ID NO: 199777) | 4 | 400 |
| [RKT][STV][RQE]**R[ADE] | 18 | 32400 |
| T*GG*RN (SEQ ID NO: 199778) | 4 | 400 |
| Q*G*VRG (SEQ ID NO: 199779) | 4 | 400 |
| MDK**QR (SEQ ID NO: 199780) | 4 | 400 |
| NGG**GR (SEQ ID NO: 199781) | 4 | 400 |
| V*RQ*AG (SEQ ID NO: 199782) | 4 | 400 |
| L*REG*R (SEQ ID NO: 199783) | 4 | 400 |
| QAG**RG (SEQ ID NO: 199784) | 4 | 400 |
| QR*VV*A (SEQ ID NO: 199785) | 4 | 400 |
| RE**ARG (SEQ ID NO: 199786) | 4 | 400 |
| RQMA**A (SEQ ID NO: 199787) | 4 | 400 |
| S**REIR (SEQ ID NO: 199788) | 4 | 400 |
| TK*R*DT (SEQ ID NO: 199789) | 4 | 400 |
| V*R*SAG (SEQ ID NO: 199790) | 4 | 400 |
| V*RS*GG (SEQ ID NO: 199791) | 4 | 400 |
| VQR*S*G (SEQ ID NO: 199792) | 4 | 400 |

TABLE 16
7-mer motifs enriched for heart biodistribution.

| | Reported | Possible |
|---|---|---|
| Motifs | Variants | Variants |
| [RNGPS]*[GKSTY][RNGKST]*[ANGMSTV]A | 347 | 420000 |
| [RNGKST][NQGKP]*[NQGKPS][NQKMSTV]*A | 505 | 504000 |
| K**[AS][AS][AK][EPS] | 18 | 9600 |
| [RNQGKPSTV][ARNGKPSTV][ANDQGPSTY][NQGKPST]** | 3694 | 10206000 |
| [AMPSV] | | |
| [RQGKPST][RQLKPT][NDQST]*[ANQGSV]*[AGSV] | 1057 | 2016000 |
| [RGKMST][NKPST]**[AQGKSTV][ANDQG][RDGPS] | 670 | 2100000 |
| [RKMPST][ARGKPST][ANQPSTYV]**[ARNGKMST][ADMPS] | 1643 | 5376000 |
| [NDGPT]K*[NQGKPT]*[NQG]A | 84 | 36000 |
| [KP][RNGPT]*[QGST][ANQT]*[GPS] | 159 | 192000 |
| [QGPS][QK]**[ANST][GMST]A | 96 | 51200 |
| [NGPT][NQGK][NKV]**[ANGST]A | 106 | 96000 |
| [NPST]*[NK][AQKT]*[QS][PS] | 54 | 51200 |
| [RNGK]*[NQST][ANQGKST][ARNGPT]*A | 335 | 268800 |
| [RNQPST][QGK]*S*[ANQGKS][GP] | 88 | 86400 |
| K[NPT]*[ANGS]*[QTV]A | 40 | 14400 |
| [RQPT][NDGKT][KS][AGST]**A | 72 | 64000 |
| [RNQK][PST][NQKT]**[NQT][APS] | 214 | 172800 |
| [KP][RQP]*T[GT]*A | 14 | 4800 |
| [RKP][QS][NGKPS]*[GT]*A | 48 | 24000 |
| K*[NS][AQGT]*[QGS]A | 34 | 9600 |
| [RN][ANSV][NGK]**[QMV]G | 24 | 28800 |
| [NK][KST]**[NGP][AQGT]A | 60 | 28800 |
| [NGK]*[QKT]*[NQGP][QGMTV][AGS] | 199 | 216000 |
| [QPST]K*[NGPS][AMST]*[AG] | 105 | 51200 |
| [KT][RT]*SG*[PV] | 9 | 3200 |
| KQ[GT][QT]**A | 10 | 1600 |
| [NKMT]*[RNQPS][NGK][ARGT]*[GMPV] | 130 | 384000 |
| K*N*[NG][NGV]A | 14 | 2400 |
| R[PS]*[PS]G*A | 9 | 1600 |
| [GST][QGS]K*N*A | 14 | 3600 |
| [KS]*[NT]*[GS][IS][AP] | 23 | 12800 |
| NKPA**S (SEQ ID NO: 199793) | 5 | 400 |
| [GKPS][RKST]*[AQS]*[NS][AGS] | 109 | 115200 |
| [NK]**[NPST][GPT][ANM]A | 51 | 28800 |
| [NQP]*K[PST][ST]*A | 25 | 7200 |
| [PT]*KG[AG]*A | 9 | 1600 |
| SK[MT]**Q[AP] | 12 | 1600 |
| NTR**SA (SEQ ID NO: 199503) | 4 | 400 |
| PKS**SG (SEQ ID NO: 199794) | 4 | 400 |
| TK*PS*S (SEQ ID NO: 199795) | 5 | 400 |
| [NG]*K[ST][QT]*[GP] | 21 | 6400 |
| FGKQ**S (SEQ ID NO: 199796) | 4 | 400 |
| [NQ][RKPSTV][ARNKT]*[NGT]*[AGM] | 152 | 216000 |
| [RK]*[NT][NS]*[NS]P | 26 | 6400 |
| K*N[QT]S*A (SEQ ID NO: 199797) | 9 | 800 |
| [KMS]**[GS][AN][NQT][AG] | 45 | 28800 |
| [NQ]K*[AT][AG]*A | 12 | 3200 |
| KQ**AKD (SEQ ID NO: 199674) | 4 | 400 |
| K*[PT]S*[AQG]A | 18 | 2400 |
| [NMS]*K[PS]*[NG]G | 19 | 4800 |
| NAKS**G (SEQ ID NO: 199798) | 5 | 400 |
| KQ**TQA (SEQ ID NO: 199799) | 5 | 400 |
| [NK][ST][NK]*[AT]*P | 12 | 6400 |
| K*TQ*GA (SEQ ID NO: 199800) | 4 | 400 |
| T**QSGF (SEQ ID NO: 199801) | 4 | 400 |
| KTQ*T*G (SEQ ID NO: 199802) | 4 | 400 |
| QK**GAP (SEQ ID NO: 199803) | 4 | 400 |
| SK**NAA (SEQ ID NO: 199804) | 4 | 400 |
| K*AT*KD (SEQ ID NO: 199767) | 4 | 400 |
| R*N*ANP (SEQ ID NO: 199805) | 4 | 400 |
| GKQ*S*P (SEQ ID NO: 199806) | 4 | 400 |
| PQKS**A (SEQ ID NO: 199807) | 4 | 400 |
| N[GK]*[KT]*SA | 9 | 1600 |
| SQ**TNP (SEQ ID NO: 199808) | 4 | 400 |
| PR*Q*SP (SEQ ID NO: 199809) | 5 | 400 |
| NG*R*TP (SEQ ID NO: 199810) | 4 | 400 |
| TKM**QA (SEQ ID NO: 199811) | 4 | 400 |
| NK*TN*S (SEQ ID NO: 199812) | 5 | 400 |
| N*K*NNG (SEQ ID NO: 199765) | 5 | 400 |
| PK*GN*A (SEQ ID NO: 199813) | 5 | 400 |
| FQKA**G (SEQ ID NO: 199814) | 4 | 400 |
| G*KT*QA (SEQ ID NO: 199815) | 4 | 400 |
| K**TSQS (SEQ ID NO: 199816) | 4 | 400 |
| K*ASS*A (SEQ ID NO: 199571) | 4 | 400 |
| MNQ*R*P (SEQ ID NO: 199817) | 4 | 400 |
| RQ*A*TS (SEQ ID NO: 199818) | 4 | 400 |
| SK**NQP (SEQ ID NO: 199819) | 4 | 400 |
| TKN**QS (SEQ ID NO: 199820) | 4 | 400 |

TABLE 17
7-mer motifs enriched for spleen biodistribution.

| | Reported | Possible |
|---|---|---|
| Motifs | Variants | Variants |
| [GFTW][AKM][NQKPT][QGKP]**[AG] | 70 | 192000 |
| [MT][RGP][RQP][KS]**G | 28 | 14400 |
| [QMT][RGP][RQGP]*[QKMT]*G | 56 | 57600 |
| [QG]*[AGP][GT]K*G | 17 | 4800 |
| [RF][KP]**[AQ][NQ]G | 9 | 6400 |
| KT[AS]**[GK][AEP] | 15 | 4800 |
| G**TKMG (SEQ ID NO: 199821) | 4 | 400 |
| [GK]*[PT][QG]*[QS]G | 14 | 6400 |
| [GT][NG]**[RK]SG | 13 | 3200 |
| [QT]*[RG]S[KT]*G | 9 | 3200 |
| [QK]*[GP]*[KST][GSV]G | 25 | 14400 |
| [RM]SS[KT]**[AS] | 11 | 3200 |
| [RM][NQG]S*[GKV]*G | 17 | 7200 |
| [MT]*STK*G (SEQ ID NO: 200023) | 8 | 800 |
| K[GT]S[QT]**G | 14 | 1600 |
| K*P[NS]*GA (SEQ ID NO: 199822) | 8 | 800 |
| K[MST][PS][GT]**A | 21 | 4800 |
| K[DT][AR]S**[AG] | 14 | 3200 |
| [IY]KP**[KS][ND] | 9 | 3200 |
| R[NQ]S[AS]**G | 9 | 1600 |
| M*SKS*A (SEQ ID NO: 199596) | 4 | 400 |
| K*S[AN]*[AGS][AS] | 20 | 4800 |
| [QGF][NKS]*[GPV][NGK]*G | 29 | 32400 |
| [RK][DST][RS]*[GS]*[AGP] | 47 | 28800 |
| [KT][GY]*[KT]*TG | 8 | 3200 |
| R*SN*TG (SEQ ID NO: 199552) | 5 | 400 |
| AKY*K*E (SEQ ID NO: 199823) | 4 | 400 |
| [KT][QG][AT]**[ST]G | 11 | 6400 |
| MR*NQ*G (SEQ ID NO: 199604) | 4 | 400 |
| K*PT*TG (SEQ ID NO: 199824) | 4 | 400 |
| NRPS**P (SEQ ID NO: 199699) | 5 | 400 |
| KRPD**G (SEQ ID NO: 199825) | 4 | 400 |
| R*SSV*G (SEQ ID NO: 199826) | 4 | 400 |
| KSSS**G (SEQ ID NO: 199827) | 5 | 400 |
| TK*S*YA (SEQ ID NO: 199828) | 4 | 400 |
| FK*S*QG (SEQ ID NO: 199829) | 4 | 400 |
| K**NTTG (SEQ ID NO: 199830) | 5 | 400 |
| FK*P*QG (SEQ ID NO: 199831) | 4 | 400 |
| FKM**QG (SEQ ID NO: 199832) | 4 | 400 |
| GK*VS*N (SEQ ID NO: 199833) | 4 | 400 |
| K*SSV*G (SEQ ID NO: 199834) | 4 | 400 |
| K*ST*NG (SEQ ID NO: 199684) | 4 | 400 |
| KF**TSG (SEQ ID NO: 199835) | 4 | 400 |
| KGSS**P (SEQ ID NO: 199836) | 4 | 400 |
| N*K*GMG (SEQ ID NO: 199837) | 4 | 400 |
| YKP*T*P (SEQ ID NO: 199838) | 4 | 400 |

TABLE 18
7-mer motifs enriched for kidney biodistribution.

| | Reported | Possible |
|---|---|---|
| Motifs | Variants | Variants |
| [NEGKMFTWY][ARDKPST][ARNQGST] | 1237 | 4939200 |
| [ANQKPST]**[AGFS] | | |
| [ARQLKM][ANGHIT]*[RQGHP]* | 329 | 1800000 |
| [AGKST][ARDKS] | | |
| [GKFTY][GKT]**[ARGV][AQFS]G | 79 | 96000 |
| [NGKT]**[NGKMPT][LKPST] | 328 | 1680000 |
| [NGHKMFT][RDGSY] | | |
| [RNQKMT][RNGIL]P[NDHKST]** | 172 | 432000 |
| [ANGKPV] | | |
| [DEGKMFT][GKMFP][ARHMF]** | 287 | 1680000 |
| [NQGKST][DGKP] | | |
| [RQGFYV]*[RGLKPS][RNGS]* | 382 | 1728000 |
| [ANDQPT][RGKPY] | | |
| [RQKM][RGT]*[NGP][AQGPV]*[GMY] | 86 | 216000 |
| [RQGIKMTYV][RNDQGHPST] | 5044 | 24948000 |
| [^{∧}DCEILMFWYV]*[^{∧}DCEHLKFWY]*[ANGKFPS] | | |
| [RGKMSTV]*[ARNHMST][NQKST] | 587 | 1764000 |
| [ARGKTV]*[ARG] | | |
| K*PG*QG (SEQ ID NO: 199839) | 4 | 400 |
| [QGFT][ANK]*[APTV][ARNGKP]* | 89 | 230400 |
| [GV] | | |
| [TY]KP*T*[PY] | 8 | 1600 |
| [QGTY][RK]P**[AGMS][ANG] | 71 | 38400 |
| [GLMTY]K*[HPS]*[NFSY][AQG] | 67 | 72000 |
| [NKT][RGY]*[KT]*[TV]G | 20 | 14400 |
| [RM][HP][KP]**[HP][AM] | 10 | 12800 |
| [RKMTY]*[GPT]*[ARNST][QGKS]G | 96 | 120000 |
| Q*[RP][QG][KT]*[GP] | 9 | 6400 |
| S*NAH*R (SEQ ID NO: 199840) | 4 | 400 |
| R**VRDV (SEQ ID NO: 199841) | | |
| KM**PKD (SEQ ID NO: 199590) | 4 | 400 |
| RP**[QG][NG]G | 10 | 1600 |
| SP**RGG (SEQ ID NO: 199640) | 4 | 400 |
| [MST][HKY][GP][AQ]**[KY] | 17 | 28800 |
| [QFV][QP][RNG]H**[KV] | 16 | 14400 |
| [RY]P*[KS]S*[AGS] | 18 | 4800 |
| FK*[PS]*QG (SEQ ID NO: 199842) | 8 | 800 |
| MGS*K*G (SEQ ID NO: 199843) | 4 | 400 |
| [NK]*[RS]*[TV][TY][GM] | 17 | 12800 |
| G*TQ*SG (SEQ ID NO: 199844) | 4 | 400 |
| TR*VS*G (SEQ ID NO: 199736) | 4 | 400 |
| YKPP**G (SEQ ID NO: 199845) | 4 | 400 |
| QR*S*TG (SEQ ID NO: 199525) | 4 | 400 |
| SK**YGA (SEQ ID NO: 199846) | 4 | 400 |
| TK*VS*N (SEQ ID NO: 199847) | 4 | 400 |
| QK*PS*A (SEQ ID NO: 199848) | 4 | 400 |
| GK*ST*A (SEQ ID NO: 199711) | 4 | 400 |
| QKS*S*G (SEQ ID NO: 199849) | 4 | 400 |
| K*TI*KD (SEQ ID NO: 199850) | 4 | 400 |
| GK*PS*A (SEQ ID NO: 199851) | 4 | 400 |
| AKY*K*E (SEQ ID NO: 199823) | 4 | 400 |
| FK*T*MS (SEQ ID NO: 199852) | 4 | 400 |
| GK*VS*N (SEQ ID NO: 199833) | 4 | 400 |
| K*MQ*QG (SEQ ID NO: 199853) | 4 | 400 |
| Q*KPH*N (SEQ ID NO: 199854) | 4 | 400 |
| QKT**GG (SEQ ID NO: 199700) | 4 | 400 |
| QPRG**G (SEQ ID NO: 199855) | 4 | 400 |
| R**SVAG (SEQ ID NO: 199856) | 4 | 400 |
| R*Q*APF (SEQ ID NO: 199857) | 4 | 400 |
| SK**NQP (SEQ ID NO: 199819) | 4 | 400 |

TABLE 19
7-mer motifs enriched for kidney transduction.

| | Reported | Possible |
|---|---|---|
| Motifs | Variants | Variants |
| YMNN**K (SEQ ID NO: 199858) | 4 | 400 |
| I*RS*TG (SEQ ID NO: 199859) | 4 | 400 |
| NGG**GR (SEQ ID NO: 199781) | 4 | 400 |
| RQMA**A (SEQ ID NO: 199787) | 4 | 400 |

TABLE 20
7-mer motifs enriched for serum biodistribution.

| | Reported | Possible |
|---|---|---|
| Motifs | Variants | Variants |
| GK**[ST][NMT][AP] | 14 | 4800 |
| [NK]*[NS][KT][NS]*A | 14 | 6400 |
| [NGP][QKS][KT]*[NGS]*[AG] | 54 | 43200 |
| K*N[AN]*[NQG][AP] | 22 | 4800 |
| [NQKPS][NQKT][AQGKST][AQGST]**[AGS] | 459 | 720000 |
| [KP][KPST]**[ANK][ANDG][ADPS] | 88 | 153600 |
| [NKV][NG]*[GK]*[KST][AG] | 24 | 28800 |
| [GP][RNK][NKPT]**[GS][AM] | 43 | 38400 |
| K*TS*[AS][AV] | 8 | 1600 |
| [NGPST][GK]*[NQGKS][NKT]*A | 77 | 60000 |
| PR*Q*SP (SEQ ID NO: 199809) | 4 | 400 |
| [NP][RKS][NK]*[GT]*[APV] | 31 | 28800 |
| [NG][QK][KT]N**A | 11 | 3200 |
| [RPST][KT][NMS]**[AQT][PS] | 46 | 57600 |
| [NT]*KS*[ST][AP] | 16 | 3200 |
| [KM][NPT]**G[QG][ARS] | 21 | 14400 |
| K*N*[GS][NI]A | 9 | 1600 |
| [RNPS][ARNK][ANG][QGKS]**[AGP] | 118 | 230400 |
| K[LKPT][ND]*[AGS]*A | 25 | 9600 |
| KT[NQ]**[AQGMS][AS] | 42 | 8000 |
| K[NQT]*[GPS][AQGV]*[APV] | 68 | 43200 |
| [PS]*K[AQ]*QS | 9 | 1600 |
| [NP]*KS[ST]*A | 9 | 1600 |
| KQ[GT]*[TV]*A | 12 | 1600 |
| K*TS*QA (SEQ ID NO: 199860) | 4 | 400 |
| [KT]*[NKP][NG][AG]*[AMP] | 33 | 28800 |
| P*KG*GV (SEQ ID NO: 199861) | 4 | 400 |
| [NP][RG]*[QS][QK]*P | 9 | 6400 |
| KP[QS][NT]**[AM] | 9 | 3200 |
| K**PGMA (SEQ ID NO: 199862) | 4 | 400 |
| N*K*NNG (SEQ ID NO: 199765) | 4 | 400 |
| RPNN**P (SEQ ID NO: 199863) | 4 | 400 |
| K*T*QQA (SEQ ID NO: 199864) | 4 | 400 |
| [PS]K[PT]**[QG][AS] | 15 | 6400 |
| PK*PS*A (SEQ ID NO: 199865) | 4 | 400 |
| KTV**KD (SEQ ID NO: 199866) | 4 | 400 |
| KA*TN*A (SEQ ID NO: 199867) | 4 | 400 |
| KN*A*QA (SEQ ID NO: 199868) | 4 | 400 |
| K*QTG*A (SEQ ID NO: 199869) | 4 | 400 |
| KTNG**P (SEQ ID NO: 199870) | 4 | 400 |
| K*T*NTS (SEQ ID NO: 199871) | 4 | 400 |
| KTN*A*P (SEQ ID NO: 199872) | 4 | 400 |
| KST**TA (SEQ ID NO: 199873) | 4 | 400 |
| DK*K*GA (SEQ ID NO: 199874) | 4 | 400 |
| KPNN**P (SEQ ID NO: 199759) | 5 | 400 |
| KTQ*S*S (SEQ ID NO: 199655) | 5 | 400 |
| N*QKT*G (SEQ ID NO: 199875) | 5 | 400 |
| K**TGNA (SEQ ID NO: 199876) | 4 | 400 |
| K*N*NVA (SEQ ID NO: 199877) | 4 | 400 |
| KP*TN*S (SEQ ID NO: 199878) | 4 | 400 |
| KPA**GA (SEQ ID NO: 199879) | 4 | 400 |
| P*KG*SA (SEQ ID NO: 199880) | 4 | 400 |
| QK**GAP (SEQ ID NO: 199803) | 4 | 400 |
| SK*S*QP (SEQ ID NO: 199881) | 4 | 400 |

TABLE 21
7-mer motifs enriched for brain biodistribution.

| | Reported | Possible |
|---|---|---|
| Motifs | Variants | Variants |
| [RQKP][RQPT]**[ANGT][ANGS][AS] | 192 | 204800 |
| [RNQKP][RGST][RNQKPV]**[ARNGKMST][RDGMPS] | 535 | 2304000 |
| K*[NQT]*[QGS][NQIM]A | 34 | 14400 |
| [EGKMPST][RGKPST][ANQPSTYV]**[ANGST][AKPS] | 1106 | 2688000 |
| [RNQGKPW][ARNQGPST][ANDQGKS][NQGKT]**[AKMPS] | 1453 | 3920000 |
| [NGT]*[KS][NGKS][ANGMT]*A | 71 | 48000 |
| [RNGHKMP]*[NQGPSTYV][ARNQGK]*[ANQGKS][ARDG] | 1039 | 3225600 |
| [NKT]*[NQPS][NGK][ARGT]*[GMP] | 92 | 172800 |
| [NGPS]*[NQK][AQKT]*[NQST][GPS] | 202 | 230400 |
| [NQGMPSV][KS][ANKSTY][ANQGT]**[AP] | 324 | 336000 |
| [RGKMPSV][QKPST][NDQKST]*[ANQGSV]*A | 465 | 504000 |
| [KMPT][RPT][NK]*[GS]*[GPSV] | 67 | 76800 |
| [QGPSTV]K*[NQGPSV][ANMST]*[AGPS] | 412 | 288000 |
| [QK]*[NG][NS]*[NGV][GP] | 34 | 19200 |
| [RGTV]**[NQS][QGTY][NMT][AKP] | 96 | 172800 |
| [NPS]*K[GS]*[ST]A | 26 | 4800 |
| K**[QG]A[QS][AS] | 11 | 3200 |
| [NQPT][KV][NQKT]*[AGS]*[GMS] | 111 | 115200 |
| [RNGKT][QGPST]*[NQGKS][ANQGKTV]*[AGV] | 606 | 1050000 |
| [QGT][GT]K**[AT]A | 17 | 4800 |
| [NDKS]*[RKT]*[NGMS][NPST][KPS] | 85 | 230400 |
| [NK][AGT]*[GKS][AKS]*P | 33 | 21600 |
| [GK]*[QHK]*[NP][LTV][AQ] | 24 | 28800 |
| [QKP]*[ANK][QST]S*A | 27 | 10800 |
| [NV][RS][AKP]*[NT]*A | 17 | 9600 |
| [NST]K[MPT]**[QG][AP] | 39 | 14400 |
| [NQKST][RNK][ANDQST]S**A | 55 | 36000 |
| T**SSNR (SEQ ID NO: 199882) | 4 | 400 |
| [KP][ARQ]*T[NGT]*A | 18 | 7200 |
| [NQG][QK]*[KST][ANGS]*[AM] | 69 | 57600 |
| [SV][RK]TS**[GP] | 14 | 3200 |
| K**GTNA (SEQ ID NO: 199883) | 4 | 400 |
| NK[NQ]*[AG]*A | 9 | 1600 |
| K*[PT]S*[QGS][AV] | 17 | 4800 |
| [KTW][NKT][QGS]**Q[ANKS] | 37 | 43200 |
| [RNKP][RNGT]*[QGKPS]*[GST][APS] | 225 | 288000 |
| PKS**SG (SEQ ID NO: 199794) | 4 | 400 |
| RDKS**A (SEQ ID NO: 199612) | 5 | 400 |
| [RY]*[ST][KS]*[ST][AP] | 17 | 12800 |
| [GF]*K[QT]T*[PS] | 9 | 3200 |
| [RN]*[AP][GK]S*[AG] | 11 | 6400 |
| RNGS**G (SEQ ID NO: 199884) | 4 | 400 |
| KT[NQG]*[AS]*[GPS] | 26 | 7200 |
| K*NSS*[PT] (SEQ ID NO: 199885) | 8 | 800 |
| Q*GSK*G (SEQ ID NO: 199886) | 4 | 400 |
| KVS*T*A (SEQ ID NO: 199887) | 4 | 400 |
| NSK*T*P (SEQ ID NO: 199888) | 4 | 400 |
| [NGP]K*[NGPST]*[ANQG]A | 88 | 24000 |
| K[NT]*[AG]*QA | 9 | 1600 |
| [GKP][QKT]**[ANST][NGMT][AP] | 156 | 115200 |
| QK*GAP (SEQ ID NO: 199803) | 4 | 400 |
| FQK*A*G (SEQ ID NO: 199889) | 4 | 400 |
| K*QTG*A (SEQ ID NO: 199869) | 4 | 400 |
| [KS][QK]**[QT]Q[AP] | 11 | 6400 |
| KQTQ**A (SEQ ID NO: 199890) | 4 | 400 |
| [GP]*K[GT]*[QG][AV] | 11 | 6400 |
| [SV]*[RK]*SAG | 8 | 1600 |
| MN**GQR (SEQ ID NO: 199891) | 4 | 400 |
| [NS]K*A*S[AG] | 10 | 1600 |
| K**TGNA (SEQ ID NO: 199876) | 4 | 400 |
| N*K*GQG (SEQ ID NO: 199892) | 4 | 400 |
| TK*V*QG (SEQ ID NO: 199893) | 4 | 400 |
| QK*TS*P (SEQ ID NO: 199894) | 4 | 400 |
| QK*S[QS][PS] | 8 | 1600 |
| NKV**SA (SEQ ID NO: 199895) | 4 | 400 |
| K*SGT*A (SEQ ID NO: 199896) | 4 | 400 |
| PK*S*AG (SEQ ID NO: 199897) | 5 | 400 |
| G*Q*TQG (SEQ ID NO: 199898) | 4 | 400 |
| K**TSQS (SEQ ID NO: 199816) | 4 | 400 |
| K*TST*A (SEQ ID NO: 199899) | 4 | 400 |
| KNGS**G (SEQ ID NO: 199900) | 4 | 400 |
| KQG*T*A (SEQ ID NO: 199901) | 4 | 400 |
| NVK**QG (SEQ ID NO: 199902) | 4 | 400 |
| PR*QQ*P (SEQ ID NO: 199903) | 4 | 400 |
| SN**KSA (SEQ ID NO: 199904) | 4 | 400 |
| T*KS*TP (SEQ ID NO: 199905) | 4 | 400 |

TABLE 22
7-mer motifs enriched for lung biodistribution.

| | Reported | Possible |
|---|---|---|
| Motifs | Variants | Variants |
| [NPT]K[PY][GP]**A | 15 | 4800 |
| KT**PQA (SEQ ID NO: 199906) | 4 | 400 |
| KT**AKE (SEQ ID NO: 199907) | 4 | 400 |
| [KPTY][RQKT][APSY]**[AGKS][ANE] | 97 | 307200 |
| [RQK][QPST][KS][MST]**G | 38 | 28800 |
| K*[AGS][NS][AST]*[AP] | 36 | 14400 |
| [RK][GST][AS][GST]**[APS] | 67 | 43200 |
| Q*GSK*G (SEQ ID NO: 199886) | 6 | 400 |
| [RKMT][RQKS][PS]*[QKSTV]*G | 67 | 80000 |
| [RKY][QKT][PST]*[STV]*[AP] | 66 | 64800 |
| [NK][RMS]P[GST]**[AP] | 22 | 14400 |
| [MT]PK*[AT]*G | 9 | 1600 |
| K*TTG*G (SEQ ID NO: 199908) | 4 | 400 |
| [TV]KP[GS]**G | 13 | 1600 |
| PK**NGA (SEQ ID NO: 199909) | 4 | 400 |
| PK*PS*A (SEQ ID NO: 199865) | 4 | 400 |
| TPR*G*G (SEQ ID NO: 199910) | 4 | 400 |
| KTQ**QA (SEQ ID NO: 199911) | 5 | 400 |
| QPK*G*G (SEQ ID NO: 199912) | 4 | 400 |
| RNS**NG (SEQ ID NO: 199913) | 4 | 400 |
| K**NTTG (SEQ ID NO: 199830) | 4 | 400 |
| K*ST*[NS]G (SEQ ID NO: 199914) | 8 | 800 |
| YKPP**G (SEQ ID NO: 199845) | 4 | 400 |
| K*SSV*G (SEQ ID NO: 199834) | 4 | 400 |
| [RNQ]*[GKS]*[GK][MTV]G | 25 | 21600 |
| KQ*SV*A (SEQ ID NO: 199915) | 4 | 400 |
| PR*Q*SP (SEQ ID NO: 199809) | 4 | 400 |
| R*SN*[NT][AG] | 13 | 1600 |
| K*PS*QA (SEQ ID NO: 199916) | 4 | 400 |
| FK*TA*G (SEQ ID NO: 199917) | 4 | 400 |
| GK*PS*A (SEQ ID NO: 199851) | 5 | 400 |
| K*GS*MS (SEQ ID NO: 199918) | 4 | 400 |
| K*SN*GS (SEQ ID NO: 199919) | 4 | 400 |
| FK**AQG (SEQ ID NO: 199920) | 4 | 400 |
| GQ*RV*G (SEQ ID NO: 199510) | 4 | 400 |
| K*ASG*A (SEQ ID NO: 199921) | 4 | 400 |
| K*SN*AA (SEQ ID NO: 199922) | 4 | 400 |
| KY*T*TG (SEQ ID NO: 199923) | 4 | 400 |
| P*R*NGA (SEQ ID NO: 199924) | 4 | 400 |
| QPR*M*G (SEQ ID NO: 199925) | 4 | 400 |
| RPQ**TG (SEQ ID NO: 199926) | 4 | 400 |
| T**VNRG (SEQ ID NO: 199927) | 4 | 400 |
| TPRS**G (SEQ ID NO: 199928) | 4 | 400 |

TABLE 23
7-mer motifs enriched for spinal cord biodistribution.

| | Reported | Possible |
|---|---|---|
| Motifs | Variants | Variants |
| [RNGKS][KPTV][NQGS]*[ANQST]*A | 199 | 160000 |
| K**[NST][QS][AQK][DS] | 22 | 14400 |
| [NGMPSTW][RNQGK][QGKMSTV]**[AQKST][ARNDGKP] | 1096 | 3430000 |
| [RGKP][QKPT]**[NQST][NQGMT]A | 189 | 128000 |
| [NMPS]*[NQKS][AQGHK]*[QGS][RIPSV] | 198 | 480000 |
| [RNLPS][RGHKS]*[AQGKPT]*[ANGS][ARGP] | 410 | 960000 |
| [RQGMS][NDQK]**[NGKS][NQGS][ARQKPT] | 310 | 768000 |
| [RNQGKPSW][ARNGKPT][ANQGST][ANQGKST]**[AGKMPS] | 2230 | 5644800 |
| K[NQT]*[AGS]*Q[AS] | 24 | 7200 |
| [RKT][KT][NQV]**[ARQKT][DPS] | 54 | 108000 |
| [RNGKMPS][RQST][NGHKT]*[NQGTV]*[AKYV] | 362 | 1120000 |
| [GT]**[QS][SY][NM][RK] | 16 | 12800 |
| [KFSY]*[ANHKP][NQS][AGST]*[RMST] | 161 | 384000 |
| [NKP][ARGK][GHPS]**[AG][RMST] | 61 | 153600 |
| [GP]K*[NS]*[AQ][AG] | 24 | 6400 |
| [RKP]**[QT][QGS][NG]A | 35 | 14400 |
| [QGKP]*[NKS][KPST][AS]*A | 54 | 38400 |
| [GK]*[NQHT]*[NGPV][QLMTV][AQS] | 126 | 192000 |
| [GK]*[NKPS][ANGTV]*[AQT][AM] | 95 | 96000 |
| K*TS*[AQ]A (SEQ ID NO: 199929) | 8 | 800 |
| N*[ST][GK][RN]*A | 10 | 3200 |
| [GK]Q[QGT][QGT]**[AM] | 19 | 14400 |
| K*NN*NP (SEQ ID NO: 199930) | 5 | 400 |
| K[PT][QS]**[AQS]A | 18 | 4800 |
| [QKP][NST][KT]**[GT]A | 27 | 14400 |
| [KV][QT][QK]*S*[AS] | 15 | 6400 |
| [RNQGKS][AQGKPT]*[NGKST][ANGKTV]*[AMSV] | 860 | 1728000 |
| KN*G*SA (SEQ ID NO: 199570) | 5 | 400 |
| T**GPGR (SEQ ID NO: 199931) | 4 | 400 |
| K*TG*KD (SEQ ID NO: 199932) | 4 | 400 |
| K**G[AT][NQ]A | 11 | 1600 |
| [QT][NQK]K[DT]**A | 14 | 4800 |
| [NK]K[ND]*G*A | 10 | 1600 |
| N*QKT*G (SEQ ID NO: 199875) | 5 | 400 |
| K[GT]N**[GT]A | 8 | 1600 |
| GKN**SA (SEQ ID NO: 199933) | 4 | 400 |
| K[ST]**[AK][ND][DP] | 10 | 6400 |
| PK*[NGP][ANS]*A | 16 | 3600 |
| TK*V*QG (SEQ ID NO: 199893) | 4 | 400 |
| K*SGT*A (SEQ ID NO: 199896) | 5 | 400 |
| N*K*[NS]N[GP] | 9 | 1600 |
| [NG]KT*G*[AM] | 9 | 1600 |
| [MP][GK]T*S*[RG] | 9 | 3200 |
| [NP]*KS*[GS]A | 13 | 1600 |
| KP**[AG][AG]S | 9 | 1600 |
| SK*S*QP (SEQ ID NO: 199881) | 4 | 400 |
| S*T*GSP (SEQ ID NO: 199934) | 4 | 400 |
| HQKP**L (SEQ ID NO: 199935) | 4 | 400 |
| N*N*GTA (SEQ ID NO: 199936) | 4 | 400 |
| SRTP**A (SEQ ID NO: 199937) | 4 | 400 |
| PR*QQ*P (SEQ ID NO: 199903) | 4 | 400 |
| P*KG*SA (SEQ ID NO: 199880) | 4 | 400 |
| VK*NN*P (SEQ ID NO: 199938) | 4 | 400 |
| K*N*SIA (SEQ ID NO: 199939) | 5 | 400 |
| KPT*P*S (SEQ ID NO: 199940) | 4 | 400 |
| T*KS*TP (SEQ ID NO: 199905) | 4 | 400 |
| N*KST*A (SEQ ID NO: 199941) | 5 | 400 |
| R*TS*SP (SEQ ID NO: 199519) | 5 | 400 |
| K*N*GNA (SEQ ID NO: 199942) | 4 | 400 |
| KTN**GS (SEQ ID NO: 199943) | 4 | 400 |
| N**GRTA (SEQ ID NO: 199944) | 4 | 400 |
| NKPP**A (SEQ ID NO: 199945) | 4 | 400 |
| SK**TNS (SEQ ID NO: 199946) | 4 | 400 |
| SK*V*TS (SEQ ID NO: 199947) | 4 | 400 |
| TK*QN*A (SEQ ID NO: 199948) | 4 | 400 |
| TKQ*S*S (SEQ ID NO: 199949) | 4 | 400 |

TABLE 24
7-mer motifs enriched for spinal cord transduction.

| | Reported | Possible |
|---|---|---|
| Motifs | Variants | Variants |
| GAG**MR (SEQ ID NO: 199950) | 3 | 400 |
| LQSN**R (SEQ ID NO: 199951) | 3 | 400 |
| NN*TT*R (SEQ ID NO: 199952) | 3 | 400 |
| NQ*Q*TK (SEQ ID NO: 199953) | 3 | 400 |
| RVG**DK (SEQ ID NO: 199954) | 3 | 400 |
| Y*AG*SR (SEQ ID NO: 199955) | 3 | 400 |

### Example 9: Functional In Vivo Detargeting

Recombinant AAVs made using the naturally occurring AAV9 capsid transduced the C57BL/6J mouse liver with high efficiency. In some applications, it is preferable to reduce the accumulation of AAV particles within the liver and reduce the transduction of liver cells. Therefore, experiments were undertaken to identify AAV capsids that produced well but had low transduction to the liver or biodistribution to the spleen. Tables 25 and 26 below list 7-mer motifs observed for 7-mer sequences that had low transduction to the liver or low biodistribution to the spleen. In Tables 25 and 26, 7-mers associated with a negative log 2 enrichment value were considered as having the indicated trait (i.e., reduced liver transduction or reduced spleen biodistribution). In Tables 25 and 26, “*” represents any amino acid, amino acids listed between brackets (“[ ]”) without the symbol “\hat{ }” represent the amino acids observed at a position, and amino acids listed between brackets (“[ ]”) and preceded by the symbol “\hat{ }” indicate amino acids not observed at the position

TABLE 25
7-mer motifs associated with reduced liver transduction.

| | Reported | Possible |
|---|---|---|
| Motifs | Variants | Variants |
| WNG**QK (SEQ ID NO: 199956) | 4 | 400 |
| [DY][KT][FS]**[QK][GK] | 9 | 12800 |
| [QSWY][NIKM][NGP][NQH]**[KY] | 32 | 115200 |
| IKH*R*E (SEQ ID NO: 199957) | 4 | 400 |
| [IT][LK]*[DH]*[KV][RN] | 9 | 12800 |
| [KF]*[KS]*[PT][NY][LM] | 9 | 12800 |
| YKD**PR (SEQ ID NO: 199958) | 4 | 400 |
| HN*N*GK (SEQ ID NO: 199959) | 4 | 400 |
| KFK*E*Y (SEQ ID NO: 199960) | 4 | 400 |
| NGQ**AR (SEQ ID NO: 199961) | 4 | 400 |
| QI*H*AK (SEQ ID NO: 199962) | 4 | 400 |
| R**NGIQ (SEQ ID NO: 199963) | 4 | 400 |
| REKP**M (SEQ ID NO: 199964) | 4 | 400 |
| Y*H*MKG (SEQ ID NO: 199965) | 4 | 400 |

TABLE 26
7-mer motifs associated with reduced spleen biodistribution.

| | Reported | Possible |
|---|---|---|
| Motifs | Variants | Variants |
| [^{∧}ADCEHILKW][ARNQGKST][ARNQGKPTV] | 4663 | 11404800 |
| [ARNQKPSTV]**[NGPS] | | |
| [RNQKSV]*[ANQGPST]*[ARNQKT][ANQGKT] | 1403 | 2419200 |
| [EGPS] | | |
| [^{∧}ANDCEHILFW]*[ANQGKPST][ARDQGKPST] | 4651 | 9216000 |
| [ARNQGKSV]*[ADGS] | | |
| [NQGFST]*[NGKT]*[ARNQKST][ANQGKSTV][GP] | 1185 | 1075200 |
| [NQGKMSTY][RQGKPST]*[ARDQGKSTV][^{∧}ADCEHILFWY] | 1565 | 2016000 |
| *G | | |
| [RNGKMPT]**[AQGKST][ARNQGSV][AQGKMST] | 2047 | 3292800 |
| [EGPS] | | |
| [NQGKFST][ARQGKPST][ADQGKPST]*[ARNQIKST] | 1211 | 1433600 |
| *G | | |
| [RNQGHKPTV]*[ARNQGMPST][ARNQKST]* | 3742 | 9525600 |
| [ANQGKMS][DEGPSV] | | |
| [^{∧}ARDCEHILKW]K*[ANQGST]*[ANQIKMSTV] | 986 | 864000 |
| [DGPS] | | |
| [RNGKTV][ANQGSV]**[ARGKSTV][AQGKMS]G | 666 | 604800 |
| [^{∧}ADCQEHFWY][ARNQGKMPS][^{∧}CEHILKFWYV]** | 5052 | 15840000 |
| [ANQGKMSV][NDGPS] | | |
| [RNQGMPSTV][ARDQGKPST][NQGKST][ANGKPST] | 2449 | 4082400 |
| **[APS] | | |
| [RNGKMST][RGKST][RNQMT]**[ARNQMST][ADPS] | 1041 | 1960000 |
| [RNQGMPSTV]*[AQGKST][AQGKPSTV]*[NQGKSTV] | 2830 | 4838400 |
| [GPSV] | | |
| [NQST]*[QKT]*[GKT][NQMS][GP] | 152 | 115200 |
| [RNQKSTY][RKPT][ANQGST]**[ANQGT]G | 510 | 336000 |
| [RNQMTYV][NQKP][RNQGS]G**G | 118 | 56000 |
| [RGKMT]**[NT][RNGKS][ANGT]G | 133 | 80000 |
| [RNGT]*[ANGKT][ARNKMST][RNQGKS]*G | 444 | 336000 |
| [RQGKMPST][NQKPST]**[ARNKST][^{∧}RCQEHILFPW] | 2211 | 4608000 |
| [DGPS] | | |
| [RKS][NQGPST][ANQGKT][ANQGT]**[APS] | 680 | 648000 |
| [RNMPSTY][ARNQGKSV]*[ANQKST]*[ANGKSTV] | 1949 | 2822400 |
| [GPS] | | |
| [NQGKPSTV][ARQGKST]*[AQGKPST][ANKSTV]* | 1765 | 2822400 |
| [APS] | | |
| [RNQGKTY][RNQGLPST][ARNQKPST]*[ANQSTV]* | 1890 | 3225600 |
| [APS] | | |
| [NQGFST][ANGSV][RK]**[NQSV][GP] | 260 | 192000 |
| K[NQGLKST][NDQGMPT][NQST]**G | 174 | 78400 |
| [NQGKMFPST][RQGKPS][RDQKPST]*G*[AM] | 201 | 302400 |
| [RQGKMST][QKP][NDQGS]*[ANKST]*[ADEPS] | 688 | 1050000 |
| K*[QT]*[QG][QMT]A | 19 | 4800 |
| [NKMST][ANGKP][NGKP]*[ARNMPST]*G | 367 | 280000 |
| [RNQPST][RNK]**[ARNGS][ANGSTV]A | 328 | 216000 |
| [QGS]K**[GT][AMT][APS] | 49 | 21600 |
| [NQGMST][NK]**[AQGSV][QKMFS][RDGPS] | 348 | 600000 |
| [RDKST][NQKP]*[AGKS]*[AGSTV]A | 183 | 160000 |
| [NQGKSTV][RNQGK]*[NQGKS][ANQKST]*[GPS] | 1192 | 1260000 |
| [QK][NKSV][NDGKT][ST]**A | 54 | 32000 |
| [RKMT]**[GPS][AT][ANGS]G | 72 | 38400 |
| [RKM]*[NPST][AGT]T*[GV] | 66 | 28800 |
| [RKV]**[NGT][AGST][NQG]A | 92 | 43200 |
| [QKST][QKT]*[NG]G*[DG] | 42 | 19200 |
| [RQGKT]*[RNQGPST][RQGST]*[QGMS]A | 339 | 280000 |
| [KV]*N*[NS][AISV]A | 25 | 6400 |
| [NQKPT][RKP]*[AGST]G*[AP] | 96 | 48000 |
| [RNQKS][ANMPSV][RNQK]*G*G | 89 | 48000 |
| [RNST]*[QKMST][RGS][ARST]*[AP] | 172 | 192000 |
| [NKS][KST][NK]*[NG]*[AS] | 62 | 28800 |
| [RNGT]*[RQGKST][NKST][RNMT]*[AG] | 375 | 307200 |
| [RKT][RQT]**[GT][NQG]A | 40 | 21600 |
| [RNIK][RNQV]*[AGS]*[NQGT][GS] | 178 | 153600 |
| [RGKP][NKT][NQTV]**G[AS] | 54 | 38400 |
| [RNQST][RQK]S[ANQKS]**G | 72 | 30000 |
| R[NST]*[ANQG][ANTV]*G | 48 | 19200 |
| [QK][QT]**N[GS]A | 14 | 3200 |
| [QPT][RGK]*N[AKS]*A | 31 | 10800 |
| [QGT][QGT]K**[AT]A | 27 | 7200 |
| RP*[NG][NT]*G | 11 | 1600 |
| [FT]K**NQ[NG] | 8 | 1600 |
| [NGS]*[GKS][RGKS]*[AGST]A | 86 | 57600 |
| [ANGS][RGK]*[NKPT]*[ANGS]A | 109 | 76800 |
| [MT]K*[QM]*QA | 10 | 1600 |
| [KP][NP][KS]**[AG]A | 13 | 6400 |
| [NM]*[NK][NK]*[QT]G | 20 | 6400 |
| [RK]**NT[GS]G | 10 | 1600 |
| RN*[AN][AN]*A | 9 | 1600 |
| N**[NQS][RGK][QGS]A | 29 | 10800 |
| [KT]*[NGT]*[GSV][ANST][GS] | 95 | 57600 |
| K*[NQM][NG][ANG]*[GP] | 39 | 14400 |
| [NQ]*[AGK]G[QT]*G | 19 | 4800 |
| QKN**SA (SEQ ID NO: 199966) | 4 | 400 |
| Q*[NK]G*[AN]G | 16 | 1600 |
| K[NT]*[AG]*QA | 9 | 1600 |
| F*KT*QA (SEQ ID NO: 199967) | 4 | 400 |
| [NST]K[GPT]*G*G | 27 | 3600 |
| RP*[ST]G*A (SEQ ID NO: 200024) | 8 | 800 |
| PTK**SG (SEQ ID NO: 199968) | 4 | 400 |
| R**QQNA (SEQ ID NO: 199969) | 4 | 400 |
| QPK**GG (SEQ ID NO: 199970) | 4 | 400 |
| KN*ST*A (SEQ ID NO: 199971) | 4 | 400 |
| RQ*PT*A (SEQ ID NO: 199515) | 4 | 400 |
| K[NQG]*N*[AT]G | 16 | 2400 |
| R[AN][QS]*[AT]*G | 16 | 3200 |
| [NQ][RN][GKP][NQS]**A | 29 | 14400 |
| KP[GP]G**G (SEQ ID NO: 199972) | 8 | 800 |
| KPS[NGS]**[AM] | 15 | 2400 |
| RS*PG*A (SEQ ID NO: 199973) | 4 | 400 |
| K*[NQT]ST*A (SEQ ID NO: 199974) | 12 | 1200 |
| [RK]*[NS][AG]T*A | 14 | 3200 |
| KPSQ**P (SEQ ID NO: 199975) | 4 | 400 |
| [NKM]*N*[GS][NT]A | 28 | 4800 |
| RSS*G*A (SEQ ID NO: 199976) | 4 | 400 |
| RN*PG*G (SEQ ID NO: 199977) | 4 | 400 |
| K[NQ]*[GS]*SG | 11 | 1600 |
| [NT]*K*[QPS][NGS]A | 26 | 7200 |
| N*RNT*P (SEQ ID NO: 199978) | 4 | 400 |
| K*[NG]A*[QG]A | 11 | 1600 |
| K*GNQ*A (SEQ ID NO: 199979) | 4 | 400 |
| S**STQS (SEQ ID NO: 199980) | 4 | 400 |
| NK**[GT]GG (SEQ ID NO: 199981) | 9 | 800 |
| RN**NQA (SEQ ID NO: 199982) | 4 | 400 |
| NR**GQA (SEQ ID NO: 199983) | 4 | 400 |
| K*[NT][NS]*AA | 11 | 1600 |
| [RK][NT]*N*QA | 11 | 1600 |
| NR*N*QG (SEQ ID NO: 199984) | 4 | 400 |
| N**STMA (SEQ ID NO: 199669) | 5 | 400 |
| N*KA*AG (SEQ ID NO: 199985) | 4 | 400 |
| NKQ*A*A (SEQ ID NO: 199986) | 4 | 400 |
| MKQ*G*G (SEQ ID NO: 199987) | 4 | 400 |
| RGN*T*G (SEQ ID NO: 199988) | 4 | 400 |
| K*NSS*P (SEQ ID NO: 199989) | 4 | 400 |
| K*NNP*A (SEQ ID NO: 199990) | 5 | 400 |
| MKN*G*G (SEQ ID NO: 199991) | 5 | 400 |
| RN*NG*G (SEQ ID NO: 199992) | 5 | 400 |
| G*K*NVA (SEQ ID NO: 199993) | 4 | 400 |
| K*GG*AG (SEQ ID NO: 199626) | 4 | 400 |
| N**GTKG (SEQ ID NO: 199994) | 4 | 400 |
| NN**NKG (SEQ ID NO: 199995) | 4 | 400 |
| RDK**GG (SEQ ID NO: 199996) | 4 | 400 |
| RQ*N*QG (SEQ ID NO: 199997) | 4 | 400 |
| SRTT*NG (SEQ ID NO: 199576) | 4 | 20 |
| TSKG**G (SEQ ID NO: 199998) | 4 | 400 |

### Example 10: MultiFunction Liver Targeting

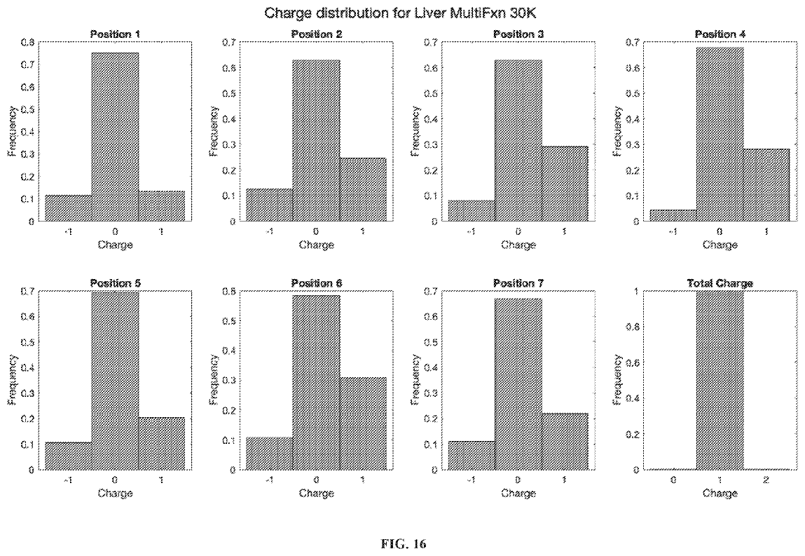

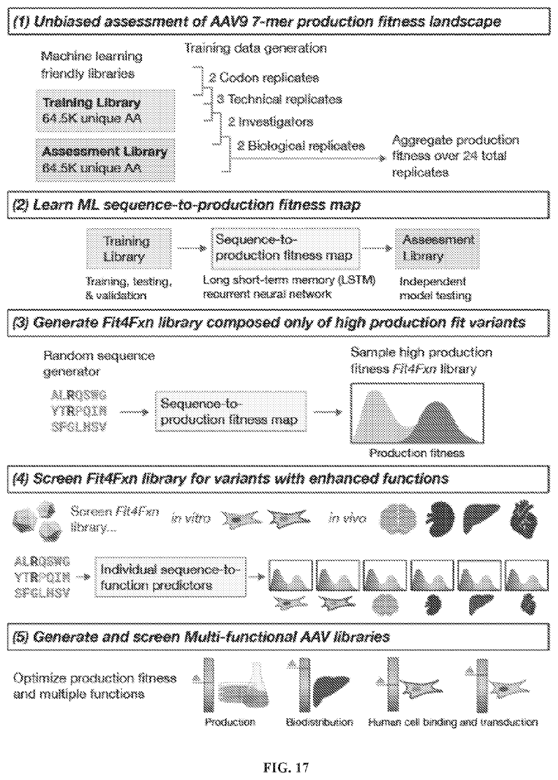

A positive control set of 3K variants was sampled from a pool of 240K variants such that each variant satisfied six traits relevant to cross-species hepatocyte targeting. Specifically, the traits were 1) high binding affinity to HepG2 cells, 2) high binding affinity to THLE cells, 3) high transduction of HepG2 cells, 4) high transduction of THLE cells, 5) high biodistribution to C57 mice liver, and 6) high production fitness. The positive set was then used in a different library of 240K along with other variants. After screening the new library for the six traits, the designed variants were considered as hits and selected only if they did not fall below the affinity/enrichment of the positive control distributions (threshold is the mean of each enrichment of each of the six traits minus 2 standard deviations) for the six traits simultaneously (see Table 27 below). Nearly all of the liver MultiFunction capsids (e.g., those containing the 7-mers listed in Table 27) had a 7-mer with an net charge of +1 (FIG. 16 and Table 28). In Table 27, “*” represents any amino acid, amino acids listed between brackets (“[ ]”) without the symbol “\hat{ }” represent the amino acids observed at a position, and amino acids listed between brackets (“[ ]”) and preceded by the symbol “\hat{ }” indicate amino acids not observed at the position

TABLE 27
7-mer motifs associated with vectors having the following six traits: 1) high
binding affinity to HepG2 cells, 2) high binding affinity to THLE cells, 3)
high transduction of HepG2 cells, 4) high transduction of THLE cells, 5) high
biodistribution to C57 mice liver, and 6) high production fitness.

| | Reported | Possible |
|---|---|---|
| Motifs | Variants | Varaints |
| [NQGIKT][DGKFPS][NEHKS]*[RDEHY]*[RQEKY] | 185 | 1800000 |
| [RDS][DQE]*[RPT][RNK]*[RKV] | 35 | 97200 |
| [RDKMSY]**[RNKSV][ARDEP][ARNDEIKS][ARNKMYV] | 581 | 3360000 |
| Q[RE]**[RK][DI][IK] | 9 | 6400 |
| [NMT][RDK]*[ARGTV]*[RDSY][ANKT] | 76 | 288000 |
| [GLFTY]*[HKY]*[NMPY][NKS][AQGL] | 70 | 288000 |
| [ANGST]*[ADEG][RNGH]*[RK][RNESV] | 140 | 320000 |
| [REKFY][ARLTYV][DGHKP][RDHIKS]**[ARNEIFT] | 250 | 2520000 |
| [ANQKT][ARKPS][RGKM]**[RSV][DEGY] | 121 | 480000 |
| [RMS]*[EK]*[RV][DPT][DKT] | 16 | 43200 |
| [QY]*G*V[RK]G | 9 | 1600 |
| [IPT][DEGP]R[AQHV]**[ANK] | 37 | 57600 |
| IRA**EK (SEQ ID NO: 199999) | 4 | 400 |
| [DK]*R[NE]*[KT][AQ] | 8 | 6400 |
| [QI][RD]*K[MP]*[RE] | 15 | 6400 |
| D*KPR*Q (SEQ ID NO: 200000) | 4 | 400 |
| [ST][QY][EK]*[RS]*[NK] | 12 | 12800 |
| [EY][ET]*K*RN | 8 | 1600 |
| ILH**KN (SEQ ID NO: 200001) | 4 | 400 |
| V*RSD*K (SEQ ID NO: 200002) | 4 | 400 |
| [ADE][ADG]*[GK]*K[LMY] | 22 | 21600 |
| [KV][QLT]R*[DS]*[GI] | 13 | 9600 |
| TD*KR*L (SEQ ID NO: 200003) | 5 | 400 |
| [RELF][EK]**[AQ][RD][DGKS] | 24 | 51200 |
| EK**TRQ (SEQ ID NO: 200004) | 4 | 400 |
| [MV]*R*[SV][AD][GK] | 10 | 6400 |
| IL*H*KN (SEQ ID NO: 200005) | 4 | 400 |
| V*KG*YN (SEQ ID NO: 200006) | 4 | 400 |
| G**HKQL (SEQ ID NO: 200007) | 4 | 400 |
| Y*SH*KG (SEQ ID NO: 200008) | 4 | 400 |
| D[NK][RH][KV]**[RV] | 10 | 6400 |
| I*RS*TG (SEQ ID NO: 199859) | 4 | 400 |
| [IK][GP]R*[DT]*[AG] | 14 | 6400 |
| QGR*L*A (SEQ ID NO: 200009) | 4 | 400 |
| YTS**KG (SEQ ID NO: 200010) | 4 | 400 |
| G**ETRK (SEQ ID NO: 200011) | 4 | 400 |
| V*RQ*AG (SEQ ID NO: 199782) | 4 | 400 |
| DL*K*RA (SEQ ID NO: 200012) | 5 | 400 |
| D*RPK*V (SEQ ID NO: 200013) | 4 | 400 |
| DR**VKQ (SEQ ID NO: 200014) | 4 | 400 |
| G**TEKK (SEQ ID NO: 200015) | 4 | 400 |
| I*R*MRE (SEQ ID NO: 200016) | 4 | 400 |
| NDMR**K (SEQ ID NO: 200017) | 4 | 400 |
| NE*KR*V (SEQ ID NO: 200018) | 4 | 400 |
| QAR*E*R (SEQ ID NO: 200019) | 4 | 400 |
| V*RS*GG (SEQ ID NO: 199791) | 4 | 400 |
| YTE**KK (SEQ ID NO: 200020) | 4 | 400 |

TABLE 28
The charge patterns in the selected 30K liver MultiFunction
capsid variants compared to their expected ratio in a random
sequence set. SumCharge indicates the 7-mer net charge, Pos
and Neg indicates the number of positive (Pos) and negative
(Neg) charges within the 7-mer, GroupCount indicates the number
of sequences with the defined charge characteristics, Percentage
provides the fraction of the liver MultiFunction library with
the indicated charge characteristics, and Expected provides
the frequency of sequences with the indicated charge characteristics
expected within a random set of 7-mers.

| Sum Charge | Pos | Neg | Group Count | Percentage | Expected |
|---|---|---|---|---|---|
| 1 | 2 | 1 | 18937 | 0.63123333 | 0.043092 |
| 1 | 1 | 0 | 10259 | 0.34196667 | 0.183788 |
| 1 | 3 | 2 | 775 | 0.02583333 | 0.001349 |
| 2 | 2 | 0 | 23 | 0.00076667 | 0.069128 |
| 0 | 2 | 2 | 3 | 0.0001 | 0.010598 |
| 1 | 4 | 3 | 3 | 0.0001 | 2.00E−06 |

### Example 11: MultiFunction Liver Targeting Individual Variants

Seven variants individually validated as described in the above Examples were found to transduce C57 mice livers and produce comparable to AAV9 WT while transducing two human hepatocyte cell lines 10×-1000× better than AAV9 WT (see Table 29). Full characterization of the individual variants and the selection process is described in the above Examples and the methods provided herein.

TABLE 29
Sequences.

| Variant Name | 7-mer | SEQ ID NO |
|---|---|---|
| AAV-BI151 | RPNRDTS | 144800 |
| AAV-BI152 | MDGQRRI | 132518 |
| AAV-BI153 | ETNRAGR | 116028 |
| AAV-BI154 | TGRVDSR | 149619 |
| AAV-BI155 | NMTRARD | 136472 |
| AAV-BI156 | GEKPKFT | 164722 |
| AAV-BI157 | MEPRQRT | 132640 |

The 7-mer sequences listed in Table 26 above were inserted between AAV9 K549R (SEQ ID NO: 199446) amino acid positions 588 and 589 and the amino acid and nucleotide sequences for the resulting capsids are provided below.

### Amino Acid Sequences:

>AAV-BI151
(SEQ ID NO: 199456)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQRPNRDTSAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*
>AAV-BI152
(SEQ ID NO: 199457)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQMDGQRRIAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*
>AAV-BI153
(SEQ ID NO: 199458)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQETNRAGRAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*
>AAV-BI154
(SEQ ID NO: 199459)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQTGRVDSRAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*
>AAV-BI155
(SEQ ID NO: 199460)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQNMTRARDAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*
>AAV-BI156
(SEQ ID NO: 199461)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQGEKPKFTAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*
>AAV-BI157
(SEQ ID NO: 199462)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQMEPRQRTAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*

### Nucleotide Sequences:

>AAV-BI151
(SEQ ID NO: 199463)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCT
AGAACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGTACCGGCAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCACAACGCCCGAATCGCGATACTTCCGCGCAGG
CTCAAACCGGTTGGGTTCAAAACCAAGGAATACTTCCGGGTATGGTTTGGCAGGACAGAGATGT
GTACCTGCAAGGACCCATTTGGGCCAAAATTCCTCACACGGACGGCAACTTTCACCCTTCTCCG
CTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCG
GAGATCCAGTACACTTCCAACTATTACAAGTCTAATAATGTTGAATTTGCTGTTAATACTGAAG
GTGTATATAGTGAACCCCGCCCCATTGGCACCAGATACCTGACTCGTAATCTGTAA
>AAV-BI152
(SEQ ID NO: 199464)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCT
AGAACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGTACCGGCAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCACAACGCCCGAATCGCGATACTTCCGCGCAGG
CTCAAACCGGTTGGGTTCAAAACCAAGGAATACTTCCGGGTATGGTTTGGCAGGACAGAGATGT
GTACCTGCAAGGACCCATTTGGGCCAAAATTCCTCACACGGACGGCAACTTTCACCCTTCTCCG
CTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCG
GAGATCCAGTACACTTCCAACTATTACAAGTCTAATAATGTTGAATTTGCTGTTAATACTGAAG
GTGTATATAGTGAACCCCGCCCCATTGGCACCAGATACCTGACTCGTAATCTGTAA
>AAV-BI153
(SEQ ID NO: 199465)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCT
AGAACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGTACCGGCAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCACAAGAAACCAACCGCGCGGGACGTGCGCAGG
CTCAAACCGGTTGGGTTCAAAACCAAGGAATACTTCCGGGTATGGTTTGGCAGGACAGAGATGT
GTACCTGCAAGGACCCATTTGGGCCAAAATTCCTCACACGGACGGCAACTTTCACCCTTCTCCG
CTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCG
GAGATCCAGTACACTTCCAACTATTACAAGTCTAATAATGTTGAATTTGCTGTTAATACTGAAG
GTGTATATAGTGAACCCCGCCCCATTGGCACCAGATACCTGACTCGTAATCTGTAA
>AAV-BI154
(SEQ ID NO: 199466)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCT
AGAACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGTACCGGCAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCACAAACTGGACGAGTCGATTCGCGTGCGCAGG
CTCAAACCGGTTGGGTTCAAAACCAAGGAATACTTCCGGGTATGGTTTGGCAGGACAGAGATGT
GTACCTGCAAGGACCCATTTGGGCCAAAATTCCTCACACGGACGGCAACTTTCACCCTTCTCCG
CTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCG
GAGATCCAGTACACTTCCAACTATTACAAGTCTAATAATGTTGAATTTGCTGTTAATACTGAAG
GTGTATATAGTGAACCCCGCCCCATTGGCACCAGATACCTGACTCGTAATCTGTAA
>AAV-BI155
(SEQ ID NO: 199467)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCT
AGAACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGTACCGGCAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCACAAAATATGACCAGGGCAAGGGACGCGCAGG
CTCAAACCGGTTGGGTTCAAAACCAAGGAATACTTCCGGGTATGGTTTGGCAGGACAGAGATGT
GTACCTGCAAGGACCCATTTGGGCCAAAATTCCTCACACGGACGGCAACTTTCACCCTTCTCCG
CTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCG
GAGATCCAGTACACTTCCAACTATTACAAGTCTAATAATGTTGAATTTGCTGTTAATACTGAAG
GTGTATATAGTGAACCCCGCCCCATTGGCACCAGATACCTGACTCGTAATCTGTAA
>AAV-BI156
(SEQ ID NO: 199468)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCT
AGAACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGTACCGGCAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCACAAGGTGAAAAGCCGAAGTTTACTGCGCAGG
CTCAAACCGGTTGGGTTCAAAACCAAGGAATACTTCCGGGTATGGTTTGGCAGGACAGAGATGT
GTACCTGCAAGGACCCATTTGGGCCAAAATTCCTCACACGGACGGCAACTTTCACCCTTCTCCG
CTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCG
GAGATCCAGTACACTTCCAACTATTACAAGTCTAATAATGTTGAATTTGCTGTTAATACTGAAG
GTGTATATAGTGAACCCCGCCCCATTGGCACCAGATACCTGACTCGTAATCTGTAA
>AAV-BI157
(SEQ ID NO: 199469)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCT
AGAACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGTACCGGCAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCACAAATGGAGCCGCGACAGAGGACTGCGCAGG
CTCAAACCGGTTGGGTTCAAAACCAAGGAATACTTCCGGGTATGGTTTGGCAGGACAGAGATGT
GTACCTGCAAGGACCCATTTGGGCCAAAATTCCTCACACGGACGGCAACTTTCACCCTTCTCCG
CTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCG
GAGATCCAGTACACTTCCAACTATTACAAGTCTAATAATGTTGAATTTGCTGTTAATACTGAAG
GTGTATATAGTGAACCCCGCCCCATTGGCACCAGATACCTGACTCGTAATCTGTAA

### Example 12: Saturation Mutagenesis

Triple site saturation mutagenesis was performed around each of the seven variants of Example 11 in silico using the same six trait prediction models as in Example 10 and filtration criteria to determine which variants were predicted to possess the six traits simultaneously. Because the 6-trait combined prediction hit rate was demonstrated to be ˜90%, those predicted variants were expected to have only a false discovery rate (FDR) of 0.1.

### Example 13: In Vivo Evaluation of Biodistribution of AAV Variants in Macaques

Experiments were undertaken to evaluate production fitness and enrichment in different organs of a macaque for AAV particles prepared as described above containing 7-mer inserts. In particular, a Fit4Function library was administered intravenously to an adult cynomolgus macaque and biodistribution of the AAV particles was assessed. The library administered had 100,000 unique amino acid variants designed according to Fit4Function criteria (uniformly sampled from a high production fitness sequence space) in addition to a calibration set (3K), control variants, and wild-type AAV9. Each capsid variant in the Fit4Function distribution was represented by either two or six 7-mer replicates (codon replicates; considered biological replicates in the same sample), and AAV9 was represented by two replicates. A purified virus library prepared based upon the Fit4Function distribution was injected at a dose of 4.6×10¹² viral genomes (vg)/kg into a female cynomolgus macaque that was pre-screened for neutralizing antibodies (NAbs) against AAV9 (CRL). Four hours after systemic delivery, the animal was perfused with cold PBS and organs were harvested and snap frozen using dry ice. DNA was extracted using a DNeasy kit in a Qiagen QIAcube Connect. Samples were then processed and analyzed as described herein. The data collected is provided below in Lists 1-14. The highly reproducible data obtained via the Fit4Function approach enabled the quantitative identification of top-performing capsids that localized to major organs in the macaque (i.e., accumulation of capsids in different organs after four hours of injection) in a reproducible manner across 7-mer replicates from a single round of screening (FIG. 20).

AAV particles containing the peptides listed in Lists 1-6 showed high levels of enrichment in macaque organs relative to AAV9 while maintaining good production fitness. For all organs except liver, the threshold for “high level of enrichment” was set as having an average log 2 enrichment that was 2-fold greater than AAV9 (mean abundance of replicates in organ/abundance in virus library administered). For the liver, the threshold was 3 log 2 fold changes. To avoid listing variants that were poorly produced/packaged and thus not manufacturable, all variants included in any of Lists 1-6 were determined to have good production fitness. “Good production fitness” was defined as being in a production-fit distribution determined using a control set of 3K variants, where the production fitness threshold was set as −2 on the log 2 scale.

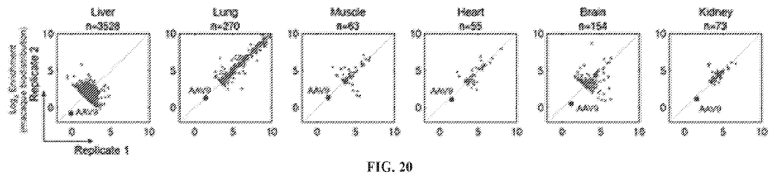

AAV particles containing the peptides listed in List 7 (see also FIG. 19) were poorly enriched (i.e., detargeted) in macaque liver relative to AAV9, while maintaining production fitness that was higher than AAV9. AAV particles having an average log 2 enrichment (abundance in liver/abundance in virus library administered to the macaque) that was more than 2-fold less than the log 2 enrichment of AAV9 in the liver were considered as being detargeted from the liver. By requiring that the listed peptides were associated with good production fitness, it was ensured that low levels of enrichment were not the result of low production fitness and a correspondingly low abundance in the library of AAV particles administered to the macaques.

AAV particles containing the peptides listed in Lists 8-12 showed low enrichment in the macaque liver relative to AAV9 while maintaining high biodistribution for other organs. AAV particles having an average log 2 enrichment (abundance in liver/abundance in virus library administered to the macaque) that was more than 2-fold less than the log 2 enrichment of AAV9 in the liver were considered as being detargeted from the liver. The targeting thresholds for the indicated organs were 1-fold (log 2 fold change) greater than AAV9, except for the kidney where the threshold was set as equal to or above AAV9.

In each of Lists 1-12, all enrichment scores include a SEQ ID NO for a peptide followed by one or two log 2 enrichment score values measured for the indicated organ(s) for AAV particles containing capsid polypeptides containing the peptide. An enrichment score for a peptide in an organ of a macaque was calculated as the log 2 of (the relative abundance in the organ of AAV particles containing capsid polypeptides containing the peptide)/(the relative abundance of AAV particles containing capsid polypeptides containing the peptide in a library of AAV particles administered to the macaque). Production fitness was calculated as log 2 of (abundance of an AAV capsid in a virus prep obtained from producer cells)/(abundance of an AAV capsid encoded by DNA used to transfect the producer cells). Abundance was calculated as “reads per million” (RPM).

List 1, which follows, provides a list of SEQ ID NOs and log 2 enrichment values for peptides inserted within the capsids of AAV particles that were found to be associated with good levels of enrichment of the AAV particles in the liver: 63377, 2.3; 72606, 2.4; 200042, 2.3; 63893, 2.3; 53936, 2.2; 43600, 2.2; 107077, 2.2; 52070, 2.2; 47760, 2.2; 83582, 2.1; 200087, 2.3; 200092, 2.2; 69722, 2.3; 53854, 2.3; 66599, 2.3; 73203, 2.3; 200160, 2.1; 68765, 2.3; 104947, 2.8; 200218, 2.4; 200219, 2.3; 74205, 2.1; 105895, 2.2; 83793, 2.3; 36564, 2.4; 76161, 2.3; 39205, 2.3; 78303, 2.2; 200316, 2.4; 101825, 2.7; 200335, 2.2; 200337, 2.3; 200340, 2.2; 78907, 2.2; 74944, 2.3; 77319, 2.5; 200344, 2.3; 200345, 2.3; 200346, 2.1; 50231, 2.1; 58710, 2.4; 115673, 2.1; 200386, 2.2; 43597, 2.3; 86219, 2.3; 109013, 2.3; 82151, 2.2; 67799, 2.4; 200403, 2.2; 200443, 2.2; 80572, 2.2; 200450, 2.5; 35715, 2.3; 39958, 2.1; 63448, 2.2; 50707, 2.3; 200467, 2.4; 48339, 2.3; 52382, 2.2; 200472, 2.3; 45011, 2.5; 67162, 2.4; 200479, 2.2; 102466, 2.1; 33589, 2.2; 78623, 2.1; 46938, 2.2; 33617, 2.1; 57932, 2.3; 81772, 2.4; 200499, 2.2; 49034, 2.4; 53890, 2.1; 91074, 2.2; 106483, 2.4; 100827, 2.3; 37681, 2.2; 77327, 2.2; 120765, 2.4; 74299, 2.2; 87527, 2.3; 49384, 2.2; 200624, 2.2; 50918, 2.2; 200626, 2.2; 41626, 2.1; 105940, 2.1; 88295, 2.2; 43046, 2.2; 35789, 2.5; 68794, 2.1; 200678, 2.1; 32616, 2.5; 64285, 2.1; 44345, 2.2; 57401, 2.3; 90989, 2.5; 42065, 2.1; 38223, 2.4; 55985, 2.2; 45001, 2.2; 65734, 2.1; 69593, 2.2; 83057, 2.2; 42050, 2.1; 56186, 2.3; 67135, 2.5; 62413, 2.1; 50995, 2.1; 45109, 2.3; 200772, 2.4; 94568, 2.1; 200776, 2.3; 108000, 2.2; 200777, 2.1; 200778, 2.1; 200783, 2.1; 200785, 2.2; 52400, 2.1; 200794, 2.2; 69533, 2.4; 105880, 2.5; 101506, 2.3; 63358, 2.1; 68670, 2.4; 39479, 2.1; 44338, 2.1; 36166, 2.2; 47387, 2.3; 97547, 2.1; 35833, 2.2; 43467, 2.6; 38169, 2.3; 200888, 2.2; 45708, 2.2; 47571, 2.1; 35892, 2.1; 99065, 2.2; 81255, 2.2; 200902, 2.1; 93082, 2.2; 89706, 2.3; 48974, 2.1; 200922, 2.1; 200923, 2.1; 64883, 2.2; 67094, 2.1; 200927, 2.2; 65933, 2.1; 38734, 2.3; 78793, 2.2; 55643, 2.2; 200937, 2.1; 98298, 2.3; 200968, 2.1; 106882, 2.1; 46939, 2.4; 60204, 2.2; 200999, 2.3; 201003, 2.3; 201005, 2.2; 70168, 2.6; 48238, 2.2; 201027, 2.2; 35219, 2.3; 102543, 2.5; 51915, 2.1; 201085, 2.2; 201094, 2.2; 95810, 2.2; 201102, 2.3; 108261, 2.3; 64635, 2.2; 49434, 2.5; 85369, 2.5; 201112, 2.5; 201115, 2.5; 52112, 2.1; 201137, 2.2; 43959, 2.2; 37155, 2.2; 201152, 2.2; 74640, 2.2; 201159, 2.2; 100982, 2.4; 75005, 2.1; 143060, 2.1; 88402, 2.3; 79669, 2.2; 80900, 2.1; 13607, 2.2; 75657, 2.2; 86415, 2.3; 201176, 2.1; 201177, 2.3; 163393, 2.1; 201179, 2.1; 85309, 2.1; 87337, 2.2; 88148, 2.3; 201197, 2.2; 94565, 2.5; 56622, 2.3; 91083, 2.1; 201207, 2.2; 201208, 2.4; 68541, 2.5; 69173, 2.4; 49742, 2.4; 62222, 2.1; 201276, 2.2; 201279, 2.1; 201280, 2.3; 72078, 2.2; 65825, 2.2; 201299, 2.2; 83935, 2.1; 201314, 2.5; 108279, 2.2; 97373, 2.2; 66461, 2.2; 201328, 2.2; 72227, 2.2; 38217, 2.2; 40082, 2.2; 52736, 2.1; 201341, 2.3; 201346, 2.1; 50977, 2.3; 201361, 2.3; 65895, 2.1; 201363, 2.5; 38419, 2.2; 101482, 2.4; 54528, 2.4; 64493, 2.1; 36563, 2.1; 201398, 2.3; 201408, 2.4; 60113, 2.1; 81262, 2.2; 201420, 2.1; 201421, 2.2; 201422, 2.1; 152992, 2.1; 50948, 2.2; 201442, 2.2; 37358, 2.2; 153995, 2.2; 201449, 2.1; 84004, 2.2; 51244, 2.2; 201458, 2.3; 41729, 2.1; 66907, 2.2; 61875, 2.1; 103566, 2.3; 59464, 2.4; 201482, 2.8; 64902, 2.6; 98751, 2.2; 50376, 2.2; 42538, 2.6; 155181, 2.1; 55980, 2.6; 71904, 2.1; 79516, 2.2; 201506, 2.5; 47079, 2.1; 82333, 2.3; 52416, 2.3; 163321, 2.3; 201524, 2.2; 201526, 2.2; 201527, 2.1; 98668, 2.3; 201534, 2.3; 96547, 2.3; 90806, 2.7; 94161, 2.2; 52119, 2.1; and 59395, 2.3.

List 2, which follows, provides a list of SEQ ID NOs and log 2 enrichment values for peptides inserted within the capsids of AAV particles that were found to be associated with good levels of enrichment of the AAV particles in the lungs: 200031, 4.1; 200048, 3.5; 200052, 4.9; 200059, 3.2; 200060, 3.3; 84763, 5.3; 200065, 9.3; 200102, 6.8; 200121, 6.9; 32538, 3.3; 200189, 5.4; 200191, 5.8; 200212, 3.8; 200216, 8.1; 200280, 3.8; 200327, 5.4; 200348, 3.1; 200355, 4.5; 200364, 4.4; 200371, 4.8; 7263, 3.9; 200398, 7.4; 200399, 3.4; 200406, 3.5; 200418, 8.9; 200427, 8.5; 97877, 5.1; 200471, 3.2; 61260, 3.5; 71754, 3.9; 200507, 8.0; 200521, 7.5; 200528, 3.5; 200540, 5.1; 200545, 7.7; 200546, 6.7; 200585, 3.7; 200611, 8.9; 200617, 4.5; 200618, 5.9; 200631, 3.1; 200646, 8.9; 101635, 7.7; 52625, 5.6; 200664, 3.5; 200711, 3.3; 94148, 3.4; 200723, 4.0; 109331, 3.1; 89312, 6.4; 200755, 3.6; 68196, 5.5; 92906, 7.5; 99892, 4.8; 200761, 6.0; 200763, 3.8; 66764, 3.5; 101214, 8.8; 200793, 6.3; 200796, 6.6; 200798, 4.7; 200799, 9.6; 200802, 8.7; 200809, 3.1; 200814, 6.3; 108890, 3.5; 200873, 3.4; 200874, 4.4; 200875, 8.6; 200891, 4.6; 103281, 3.2; 200893, 3.6; 71742, 3.1; 101302, 6.5; 200942, 5.4; 200973, 3.2; 200977, 8.4; 200979, 8.8; 200988, 4.2; 200991, 9.0; 201001, 4.2; 201002, 3.1; 201025, 4.9; 201028, 7.9; 33382, 4.3; 201035, 4.9; 201036, 8.8; 201049, 3.8; 201064, 7.8; 201067, 7.7; 201075, 6.6; 201077, 3.9; 201126, 8.1; 32565, 3.9; 44912, 4.2; 201139, 6.4; 201141, 3.1; 201158, 7.2; 201169, 5.1; 201171, 4.0; 32506, 4.5; 201172, 6.0; 75489, 3.1; 201209, 4.0; 201210, 4.8; 201225, 4.8; 201229, 5.3; 201236, 6.9; 107803, 6.2; 201256, 4.0; 201262, 3.5; 201268, 7.6; 201271, 5.2; 49119, 8.4; 201277, 9.8; 61210, 6.4; 201286, 4.0; 56844, 3.3; 201332, 3.5; 34471, 3.6; 201364, 3.3; 201381, 8.9; 201406, 3.4; 88680, 7.2; 110284, 6.2; 49578, 3.4; 201432, 9.1; 96654, 3.4; 201493, 4.1; 201517, 3.4; 48875, 5.1; 201523, 4.3; 201531, 8.6; and 201541, 3.4.

List 3, which follows, provides a list of SEQ ID NOs and log 2 enrichment values for peptides inserted within the capsids of AAV particles that were found to be associated with good levels of enrichment of the AAV particles in muscle: 200052, 4.3; 200056, 2.8; 105464, 2.9; 79382, 2.8; 200140, 5.8; 200189, 4.6; 200191, 6.4; 68765, 2.9; 200250, 5.2; 200280, 4.2; 200293, 3.5; 200327, 5.8; 53704, 3.3; 32763, 3.3; 200371, 4.8; 7263, 4.8; 200410, 3.3; 200534, 3.9; 200686, 3.7; 200723, 6.6; 200852, 3.1; 200943, 2.8; 44631, 3.0; 36404, 3.5; 53237, 3.7; 201040, 2.8; 201062, 3.9; 201075, 5.7; 33865, 3.3; 32998, 3.6; 59224, 3.0; 32565, 4.7; 201139, 5.8; 201154, 3.3; 201158, 6.8; 201178, 2.9; 32637, 3.4; 93298, 3.2; 55075, 2.9; 32712, 3.2; 32722, 3.6; 32709, 3.3; 46083, 3.6; 96654, 4.3; 32753, 3.7; 201493, 3.7; 156812, 3.3; and 36771, 3.6.

List 4, which follows, provides a list of SEQ ID NOs and log 2 enrichment values for peptides inserted within the capsids of AAV particles that were found to be associated with good levels of enrichment of the AAV particles in the heart: 200052, 3.0; 200140, 5.5; 200189, 3.7; 200191, 5.5; 200250, 3.5; 200327, 5.1; 32763, 2.9; 200371, 4.2; 7263, 4.4; 200534, 3.7; 200570, 8.0; 200723, 5.4; 36404, 2.9; 53237, 3.5; 201062, 3.7; 201075, 5.6; 33865, 3.3; 32998, 3.5; 59224, 3.0; 32565, 4.3; 201139, 5.7; 201154, 2.9; 201158, 6.7; 201169, 5.7; 201206, 4.4; 32637, 3.2; 93298, 3.4; 32712, 3.1; 32722, 3.4; 32709, 3.3; 46083, 3.4; 96654, 4.1; 32753, 3.5; 41456, 3.2; 201493, 4.0; 156812, 3.0; and 36771, 3.3.

List 5, which follows, provides a list of SEQ ID NOs and log 2 enrichment values for peptides inserted within the capsids of AAV particles that were found to be associated with good levels of enrichment of the AAV particles in the brain: 200028, 2.4; 200029, 2.2; 21985, 2.4; 200033, 3.0; 200045, 2.3; 53936, 2.4; 200055, 1.9; 200060, 2.4; 169361, 2.2; 200061, 2.2; 91886, 2.4; 200063, 2.2; 106074, 2.3; 84763, 2.2; 200064, 1.9; 86507, 2.1; 70796, 1.9; 200070, 3.7; 79598, 2.0; 109250, 2.1; 96433, 2.0; 96584, 2.0; 200080, 2.2; 200084, 2.2; 89468, 1.9; 77619, 1.9; 200088, 1.9; 200090, 2.0; 200099, 1.9; 71979, 2.3; 200101, 2.8; 200106, 2.0; 200107, 2.1; 200111, 2.0; 87751, 2.8; 46410, 2.5; 68765, 2.7; 200204, 1.9; 200208, 2.0; 200262, 2.3; 200268, 2.2; 200274, 3.7; 200284, 2.7; 200285, 2.7; 200294, 2.3; 200309, 2.3; 200323, 2.0; 200333, 2.6; 200346, 2.0; 200351, 2.4; 200363, 2.0; 53704, 2.2; 32763, 3.5; 33390, 2.1; 200373, 3.4; 70780, 2.3; 7263, 4.2; 200399, 1.9; 200400, 2.6; 200404, 1.9; 200406, 2.7; 200433, 2.8; 200456, 2.1; 200461, 1.9; 200462, 2.1; 200469, 2.2; 200473, 2.2; 61260, 2.8; 200476, 2.3; 200477, 2.0; 200478, 2.5; 64490, 2.1; 200480, 2.1; 106273, 2.7; 200481, 2.0; 200482, 2.0; 89826, 2.1; 200484, 2.2; 200485, 2.0; 10077, 2.1; 200488, 1.9; 78003, 2.0; 3124, 1.9; 200490, 2.1; 200491, 2.2; 117765, 2.4; 200500, 2.2; 167913, 2.3; 200501, 2.0; 200504, 2.5; 106483, 1.9; 63204, 2.2; 200516, 2.1; 200520, 1.9; 200525, 2.2; 42550, 2.1; 200526, 2.2; 76850, 2.1; 38739, 2.1; 101451, 2.2; 200532, 2.4; 200534, 3.4; 32519, 2.5; 57001, 3.0; 58157, 2.0; 103752, 2.2; 45722, 2.1; 86300, 1.9; 99128, 2.0; 50757, 1.9; 200556, 2.0; 42876, 2.5; 200561, 2.1; 40050, 2.3; 200565, 2.1; 200566, 2.0; 63907, 2.3; 200570, 10.2; 200572, 2.1; 200582, 3.0; 200590, 2.2; 200591, 2.6; 200594, 2.0; 72225, 2.0; 81977, 2.1; 91663, 2.2; 200601, 2.0; 200604, 2.6; 200607, 2.2; 200608, 2.1; 78915, 2.0; 200610, 2.0; 48213, 2.2; 200612, 2.4; 70453, 2.0; 48388, 2.1; 86682, 2.2; 102260, 1.9; 50918, 2.1; 99471, 2.1; 200626, 2.1; 200635, 2.0; 43046, 2.1; 200647, 2.0; 46259, 2.1; 77074, 1.9; 200650, 2.4; 13019, 2.2; 200661, 2.3; 200667, 2.5; 200668, 2.0; 88766, 2.7; 200677, 1.9; 200679, 3.3; 200680, 2.5; 200681, 2.0; 200682, 2.4; 200683, 1.9; 200684, 2.3; 200685, 2.4; 87904, 2.0; 108679, 1.9; 101370, 2.0; 34357, 2.2; 88923, 2.1; 200704, 2.1; 200706, 2.4; 200709, 2.0; 200711, 2.0; 200713, 2.0; 93829, 2.0; 108721, 2.1; 200718, 2.0; 65420, 2.1; 200720, 1.9; 200721, 2.2; 60848, 2.3; 200727, 2.1; 126497, 1.9; 200742, 2.2; 200743, 2.1; 200746, 1.9; 60287, 2.1; 75869, 2.4; 108841, 1.9; 200751, 2.3; 68056, 2.4; 94834, 2.0; 200755, 2.4; 72403, 1.9; 102461, 1.9; 45832, 2.5; 200757, 2.1; 72004, 2.0; 88621, 2.2; 68196, 2.0; 200758, 2.4; 76324, 2.1; 200764, 4.3; 200766, 2.6; 94599, 1.9; 13597, 2.1; 200767, 2.0; 94426, 1.9; 49869, 1.9; 200769, 2.0; 200770, 2.1; 200771, 2.4; 54060, 1.9; 67584, 1.9; 109588, 2.0; 91006, 2.0; 66764, 2.0; 70827, 3.7; 162412, 2.1; 200786, 2.1; 200787, 2.0; 52933, 1.9; 49962, 2.2; 200790, 2.2; 38653, 1.9; 58737, 2.3; 200797, 1.9; 200800, 2.0; 43137, 2.0; 200801, 2.6; 58042, 2.0; 94866, 2.0; 200805, 2.0; 200806, 2.2; 200810, 2.3; 38280, 1.9; 35108, 2.6; 34898, 1.9; 200823, 2.4; 200824, 2.2; 200825, 2.1; 200826, 2.2; 200828, 2.9; 200829, 2.0; 200830, 2.1; 200831, 2.1; 61975, 2.5; 84093, 2.1; 88723, 2.2; 200834, 1.9; 105687, 2.2; 94857, 2.3; 104076, 2.2; 40045, 2.0; 200843, 1.9; 78168, 2.3; 200844, 2.1; 200846, 2.3; 81592, 2.0; 100586, 2.6; 200847, 2.3; 70809, 2.1; 200848, 2.4; 200849, 2.0; 200853, 2.1; 48313, 2.0; 97547, 2.1; 200855, 2.3; 200858, 2.0; 200859, 1.9; 200860, 3.0; 105106, 2.1; 200861, 2.2; 35224, 2.0; 67019, 2.0; 200866, 2.1; 200867, 2.3; 200868, 1.9; 200869, 2.1; 68699, 1.9; 34718, 2.2; 200876, 1.9; 101351, 1.9; 200890, 2.1; 103281, 2.4; 65578, 2.0; 200892, 1.9; 133104, 2.2; 200895, 2.4; 200896, 2.3; 98413, 1.9; 200898, 2.0; 200899, 2.1; 200900, 2.1; 200901, 1.9; 89957, 1.9; 62586, 1.9; 200910, 3.0; 200911, 2.3; 200914, 2.7; 87020, 1.9; 200919, 2.3; 200921, 2.0; 200924, 2.4; 51909, 2.1; 200928, 2.1; 200929, 2.6; 200930, 2.0; 61587, 1.9; 200936, 1.9; 70504, 1.9; 109291, 2.9; 51953, 2.3; 200978, 2.1; 200983, 2.0; 200984, 2.0; 36404, 3.9; 53237, 3.6; 48216, 2.2; 60838, 2.0; 200994, 2.3; 200996, 2.2; 200997, 2.2; 200998, 2.2; 90107, 2.2; 201003, 2.2; 50151, 2.1; 201005, 1.9; 201007, 2.0; 201011, 2.9; 82422, 2.1; 201013, 2.1; 66952, 1.9; 79038, 2.0; 201025, 4.2; 33382, 2.5; 201030, 2.1; 201031, 2.2; 53701, 1.9; 55860, 2.0; 201045, 2.1; 201050, 2.4; 201051, 2.6; 201054, 2.1; 201056, 2.0; 201057, 2.0; 201062, 2.4; 201071, 2.0; 201076, 2.5; 201077, 2.4; 201078, 2.3; 201079, 1.9; 43755, 1.9; 201086, 1.9; 201094, 2.0; 201108, 2.4; 76196, 2.0; 51301, 2.0; 201113, 1.9; 95594, 2.3; 140888, 2.2; 101051, 2.1; 33865, 3.1; 32998, 2.7; 32565, 5.4; 102163, 2.1; 201135, 2.4; 103316, 1.9; 78067, 2.1; 63626, 1.9; 201140, 2.2; 91320, 1.9; 43796, 1.9; 74779, 2.0; 104866, 2.5; 201157, 1.9; 76252, 1.9; 201160, 2.5; 201161, 2.5; 2634, 2.1; 201169, 8.7; 32506, 2.5; 81155, 3.7; 99430, 1.9; 201175, 1.9; 89279, 2.0; 201178, 5.2; 201185, 2.1; 201187, 2.1; 201188, 2.4; 100364, 1.9; 201189, 2.0; 201190, 2.1; 84377, 2.1; 201194, 2.2; 201195, 2.6; 74793, 2.0; 201196, 2.2; 201199, 3.0; 75489, 2.4; 201203, 2.4; 76621, 2.1; 201205, 2.1; 75744, 2.0; 40943, 2.4; 201206, 6.5; 69429, 2.0; 76785, 2.2; 57077, 2.3; 201213, 2.1; 201217, 2.1; 201234, 1.9; 97177, 1.9; 100548, 2.5; 201251, 2.0; 201253, 2.2; 201254, 2.3; 106862, 1.9; 201260, 1.9; 94898, 3.9; 201264, 2.5; 72595, 2.1; 32637, 3.5; 32649, 2.4; 93298, 3.1; 8018, 2.0; 201273, 2.0; 109039, 2.3; 99240, 1.9; 35585, 1.9; 81812, 2.3; 61210, 3.0; 201281, 2.4; 201283, 2.0; 201290, 2.4; 201291, 2.3; 201298, 2.0; 51974, 2.2; 87296, 2.0; 201301, 2.5; 55075, 2.6; 201329, 1.9; 201334, 2.2; 201337, 2.7; 90405, 2.2; 105267, 1.9; 32958, 1.9; 38480, 1.9; 34471, 2.6; 49231, 1.9; 60450, 1.9; 201343, 2.0; 32712, 3.1; 201351, 3.5; 32722, 3.1; 201354, 2.1; 201359, 2.0; 109053, 2.0; 47063, 2.0; 201372, 2.4; 201373, 2.1; 104481, 2.0; 201375, 2.1; 201377, 2.5; 201380, 1.9; 99040, 2.1; 77969, 1.9; 109783, 2.5; 201382, 2.0; 201385, 2.0; 102859, 2.0; 201392, 1.9; 201405, 1.9; 201407, 2.2; 71829, 2.0; 91638, 2.1; 49578, 2.9; 201418, 2.6; 44640, 2.0; 80748, 1.9; 201423, 2.3; 201425, 2.1; 201428, 3.3; 94711, 2.0; 32709, 3.1; 46083, 3.9; 96654, 4.0; 32753, 3.7; 201436, 2.2; 201440, 2.3; 201446, 2.0; 81815, 2.3; 201464, 2.1; 201468, 2.3; 33824, 2.3; 201473, 1.9; 201476, 2.2; 201479, 2.4; 201480, 2.7; 201481, 2.1; 44517, 2.0; 201483, 2.0; 201485, 2.1; 2744, 2.2; 108782, 1.9; 43721, 2.4; 201488, 2.4; 97488, 1.9; 201490, 2.0; 108521, 4.5; 201491, 3.6; 49001, 2.1; 201492, 2.2; 201493, 3.4; 201505, 2.6; 201512, 2.0; 201513, 1.9; 201515, 2.2; 201518, 1.9; 156099, 2.0; 201519, 2.1; 101307, 2.2; 75166, 2.0; 63975, 2.6; 201522, 1.9; 76664, 2.5; 110032, 5.9; 201523, 2.3; 156812, 2.9; 66173, 1.9; 201529, 2.5; 36771, 2.7; 201530, 1.9; 99931, 1.9; 71471, 1.9; 77043, 2.2; 79041, 1.9; 70516, 1.9; 37561, 3.9; 48428, 2.1; 201542, 2.3; and 70659, 2.2.

List 6, which follows, provides a list of SEQ ID NOs and log 2 enrichment values for peptides inserted within the capsids of AAV particles that were found to be associated with good levels of enrichment of the AAV particles in the kidneys: 46026, 4.2; 50088, 4.5; 200098, 3.1; 38820, 3.2; 106273, 3.4; 200494, 3.5; 200495, 3.2; 200497, 3.9; 35576, 3.4; 38739, 4.6; 101451, 4.4; 58157, 4.5; 48089, 3.4; 62617, 4.0; 42876, 4.6; 85112, 3.7; 40050, 4.7; 67392, 3.5; 101176, 3.7; 200568, 3.8; 200570, 6.1; 48213, 4.9; 48388, 3.4; 200676, 3.1; 88766, 4.8; 103140, 3.2; 60287, 4.0; 103773, 4.3; 39984, 3.3; 61975, 3.4; 80013, 3.2; 41683, 3.5; 88943, 3.6; 200924, 4.8; 50855, 3.6; 51909, 4.7; 200929, 5.0; 200940, 3.1; 85694, 3.6; 50151, 3.3; 52650, 3.1; 201076, 3.6; 43755, 3.4; 201169, 4.9; 82993, 3.6; 40943, 3.5; 201206, 3.4; 69429, 3.7; 76853, 4.3; 57077, 3.5; 27565, 3.4; 49696, 3.2; 101305, 3.3; 38904, 3.4; 53231, 3.4; 100240, 3.8; 201428, 4.0; and 201521, 3.8.

List 7, which follows, provides a list of SEQ ID NOs and log 2 enrichment values for peptides inserted within the capsids of AAV particles that were found to be associated with low levels of enrichment (i.e., detargeting) of the AAV particles in the liver: 82853, −3.1; 200032, −3.9; 200034, −3.0; 99534, −3.2; 200036, −4.1; 107243, −3.3; 200037, −3.9; 200038, −3.3; 200039, −4.4; 200040, −3.2; 200041, −4.0; 200043, −2.9; 200044, −3.9; 200047, −3.0; 200050, −3.0; 200051, −3.4; 200053, −3.0; 200058, −3.8; 200068, −3.4; 159746, −2.9; 89132, −3.1; 200072, −4.6; 200073, −3.5; 200074, −3.2; 200075, −3.1; 200076, −3.9; 200077, −3.4; 97447, −3.2; 200082, −3.4; 200083, −3.4; 200086, −3.6; 200094, −2.9; 200095, −2.9; 200097, −3.0; 97276, −3.6; 200103, −2.9; 200105, −3.0; 200109, −3.0; 200110, −3.2; 200112, −3.0; 200114, −3.7; 200115, −3.6; 106717, −2.9; 276, −3.3; 200116, −3.5; 200118, −3.6; 79885, −2.9; 200119, −3.7; 200120, −3.3; 200122, −3.5; 200123, −3.6; 200125, −3.3; 200126, −3.1; 200127, −3.1; 200128, −2.9; 200129, −3.0; 200130, −4.0; 200131, −3.3; 200133, −3.0; 200134, −3.7; 200135, −3.2; 200136, −3.2; 200137, −3.0; 200141, −3.1; 200142, −3.0; 200143, −3.4; 200144, −3.6; 200145, −4.0; 200146, −3.1; 200148, −3.0; 200149, −3.4; 200150, −3.6; 9595, −3.3; 200151, −3.8; 200154, −3.0; 200155, −3.7; 200156, −3.2; 200157, −3.0; 200158, −3.3; 200159, −3.3; 200161, −3.1; 200163, −3.5; 200165, −3.5; 200166, −2.9; 200167, −3.4; 41434, −3.9; 200169, −3.5; 200170, −3.5; 200172, −3.0; 200174, −4.2; 200175, −3.1; 200178, −3.2; 200179, −4.5; 200180, −3.2; 200183, −3.6; 200185, −2.9; 200186, −4.5; 200187, −4.1; 200188, −3.0; 200190, −3.0; 200192, −5.0; 159634, −2.9; 200193, −3.9; 200194, −4.2; 104139, −3.3; 200195, −3.3; 200196, −3.7; 200197, −2.9; 200198, −3.5; 200199, −2.9; 200200, −3.5; 200201, −3.2; 200203, −5.1; 200205, −3.0; 200207, −3.4; 200209, −3.1; 200210, −3.9; 200211, −4.9; 86589, −3.0; 200213, −4.1; 200214, −3.0; 11809, −3.0; 200215, −3.4; 105416, −2.9; 200217, −3.7; 200221, −4.6; 200222, −3.7; 200223, −3.1; 200224, −4.0; 200226, −3.2; 200227, −3.2; 200229, −4.8; 200230, −3.9; 200231, −4.1; 200233, −3.1; 200235, −3.0; 200236, −3.2; 200237, −3.2; 200239, −3.2; 200241, −3.3; 200244, −3.2; 200245, −4.1; 200249, −3.2; 200252, −3.5; 200254, −2.9; 200256, −3.1; 200258, −3.2; 200259, −4.6; 200260, −3.1; 200265, −4.2; 200267, −3.3; 200269, −3.1; 200271, −2.9; 200272, −3.4; 74254, −3.2; 200273, −3.1; 200276, −3.1; 78462, −3.0; 200278, −3.0; 99977, −4.4; 200281, −2.9; 99543, −3.0; 12395, −4.9; 200289, −4.3; 200290, −4.2; 200291, −3.2; 200295, −3.3; 200296, −3.2; 200297, −3.9; 200298, −4.6; 200299, −3.6; 200300, −5.7; 200301, −4.9; 200302, −3.3; 200303, −3.0; 200306, −4.3; 200307, −3.1; 200314, −3.0; 7772, −2.9; 200315, −3.5; 200317, −3.7; 200319, −3.0; 200320, −3.4; 200322, −3.0; 109195, −3.1; 92765, −3.0; 200324, −4.0; 200325, −3.8; 200326, −4.1; 200328, −3.4; 200329, −3.5; 97581, −3.4; 200330, −3.3; 109197, −2.9; 200331, −3.1; 200332, −3.2; 95485, −4.3; 200339, −3.3; 200341, −3.3; 200342, −3.2; 200343, −3.5; 200347, −3.3; 200352, −3.1; 200354, −3.4; 200360, −3.7; 200362, −2.9; 63264, −3.0; 200366, −2.9; 200367, −3.8; 200369, −3.5; 200372, −3.1; 79466, −3.0; 200376, −3.8; 200377, −3.3; 200379, −3.4; 200380, −3.0; 200381, −2.9; 200382, −3.9; 200383, −3.3; 200384, −3.1; 200385, −3.0; 200387, −3.0; 200388, −2.9; 200392, −2.9; 200393, −3.4; 200394, −3.3; 200395, −3.1; 200397, −3.0; 200401, −3.3; 200407, −3.9; 200408, −4.2; 200409, −5.6; 200411, −3.5; 200412, −4.8; 200413, −3.4; 200417, −3.8; 200420, −3.9; 200421, −3.5; 200422, −3.0; 200423, −3.8; 200424, −4.1; 200425, −3.2; 200432, −3.2; 200434, −3.0; 200435, −4.1; 200436, −3.2; 200438, −3.2; 200440, −3.3; 200442, −3.1; 200451, −3.5; 200452, −3.2; 200454, −3.8; 200455, −4.9; 200458, −3.1; 109676, −3.0; 200463, −3.3; 200468, −3.0; 200474, −3.6; 200486, −3.6; 200487, −4.1; 200489, −4.3; 200496, −3.0; 200505, −3.1; 200508, −3.8; 100527, −2.9; 200509, −4.2; 200510, −3.3; 200511, −3.7; 200512, −3.6; 200514, −3.1; 200517, −3.2; 200518, −4.1; 200519, −3.3; 200522, −2.9; 200524, −3.1; 83308, −3.6; 83170, −3.7; 200530, −3.6; 200533, −3.2; 93048, −4.1; 200536, −3.1; 200538, −3.3; 200539, −3.9; 100632, −3.9; 200543, −3.8; 200544, −3.4; 200549, −3.5; 200552, −3.7; 200553, −3.6; 200554, −4.4; 200557, −3.5; 200558, −3.1; 200562, −3.7; 200564, −2.9; 200574, −3.3; 200575, −4.2; 200576, −3.8; 200578, −3.9; 200579, −3.8; 200580, −3.6; 200581, −3.0; 200586, −4.0; 200587, −3.0; 200589, −3.0; 200592, −3.1; 87987, −3.1; 200595, −3.0; 200596, −3.5; 100356, −3.9; 109414, −3.5; 67930, −2.9; 200598, −3.0; 200613, −3.6; 200614, −3.7; 200615, −3.0; 81196, −3.5; 200619, −3.6; 200620, −3.8; 200621, −2.9; 200623, −3.2; 200627, −3.9; 200629, −4.1; 200630, −3.2; 200633, −3.0; 200634, −3.6; 200636, −3.3; 200637, −3.3; 200638, −3.6; 200639, −3.6; 200640, −3.9; 200643, −3.1; 200651, −3.2; 200652, −3.6; 97034, −3.4; 200653, −3.3; 200654, −2.9; 107841, −3.2; 200655, −3.9; 200656, −4.3; 200657, −3.5; 200659, −3.1; 200660, −3.4; 200662, −3.3; 200663, −4.1; 200666, −3.5; 200670, −3.5; 200671, −2.9; 200687, −3.1; 200688, −4.3; 200690, −4.0; 200693, −3.5; 200694, −3.3; 200699, −3.2; 200703, −3.1; 107939, −3.5; 200707, −4.0; 200712, −3.0; 200715, −3.3; 200716, −3.0; 200717, −3.7; 200724, −4.1; 200725, −3.0; 200726, −3.1; 200729, −3.1; 100441, −3.2; 200731, −3.8; 200732, −3.9; 200739, −3.1; 99822, −3.2; 200745, −2.9; 200747, −3.1; 84445, −3.1; 200749, −3.4; 160029, −3.4; 90695, −3.9; 200759, −4.8; 200760, −4.2; 106405, −2.9; 200768, −3.9; 200774, −3.2; 200780, −3.0; 200781, −3.0; 200782, −3.0; 200788, −3.4; 65598, −3.3; 200803, −3.1; 200804, −3.7; 94066, −3.7; 200808, −3.7; 200816, −2.9; 200820, −3.5; 200821, −3.7; 110191, −3.1; 160068, −3.0; 200837, −3.2; 200839, −3.4; 200841, −3.3; 200842, −3.7; 100348, −3.3; 160076, −3.4; 200850, −3.2; 200851, −3.2; 200854, −3.2; 104536, −3.0; 200863, −3.0; 200870, −3.7; 200877, −3.3; 44930, −2.9; 200880, −2.9; 200881, −3.1; 200883, −3.0; 200884, −3.0; 200886, −4.1; 200889, −4.4; 104699, −3.4; 200903, −3.7; 200907, −3.9; 200915, −3.2; 200916, −3.1; 200917, −3.5; 200918, −3.0; 200925, −3.3; 200931, −2.9; 200934, −3.0; 200938, −3.5; 88971, −2.9; 200945, −4.0; 106064, −3.0; 200947, −3.3; 200949, −3.2; 200950, −3.1; 200951, −4.1; 200952, −2.9; 200953, −3.3; 200956, −3.3; 200957, −3.0; 200958, −3.4; 200961, −3.1; 54729, −3.5; 200962, −3.2; 200963, −3.9; 200964, −4.0; 104615, −2.9; 200965, −3.9; 200966, −3.4; 200969, −2.9; 93726, −3.0; 200972, −2.9; 200976, −3.7; 136333, −3.9; 200980, −4.0; 200982, −3.0; 200985, −3.1; 200986, −3.1; 93161, −4.5; 99024, −3.2; 201000, −4.6; 201009, −3.2; 201012, −3.1; 9212, −3.3; 201014, −4.4; 201015, −3.4; 201017, −3.3; 201018, −3.0; 201019, −2.9; 201020, −3.3; 201021, −3.5; 201023, −3.0; 201029, −4.1; 201033, −3.9; 96403, −3.5; 201042, −3.2; 201044, −3.3; 201046, −3.2; 52630, −3.0; 201053, −4.1; 201055, −3.9; 201058, −2.9; 201059, −3.8; 201060, −3.0; 201065, −2.9; 67035, −3.0; 201068, −4.1; 98522, −3.2; 201069, −3.5; 201070, −2.9; 201072, −3.3; 201073, −4.0; 201080, −3.0; 201082, −3.3; 201083, −2.9; 201087, −3.5; 9420, −3.4; 201088, −4.6; 201089, −3.1; 201090, −3.3; 201091, −3.0; 201092, −3.5; 201093, −3.1; 201096, −3.2; 160223, −3.0; 201097, −3.3; 201099, −3.3; 201100, −3.4; 201103, −3.6; 201104, −3.9; 201105, −3.0; 201106, −3.2; 201109, −3.2; 201114, −3.1; 201116, −3.1; 201117, −4.2; 201118, −2.9; 201119, −2.9; 201120, −3.1; 201121, −3.1; 60853, −3.1; 201122, −3.1; 201123, −3.4; 110378, −3.5; 201124, −3.3; 87362, −2.9; 201127, −3.8; 201130, −2.9; 201132, −3.5; 201133, −3.3; 201142, −4.7; 201144, −3.1; 201145, −4.4; 81605, −2.9; 201147, −3.2; 201148, −4.0; 201149, −3.1; 201150, −2.9; 201151, −3.3; 201156, −2.9; 201162, −3.0; 201163, −3.4; 201164, −2.9; 201165, −3.3; 201166, −4.0; 201180, −3.5; 201182, −3.1; 86814, −3.1; 201211, −3.1; 201212, −3.1; 201214, −4.8; 201215, −3.1; 86262, −3.1; 201216, −3.1; 201218, −3.3; 201219, −3.9; 201220, −3.7; 90505, −2.9; 201222, −3.2; 201223, −3.0; 201231, −3.9; 201232, −3.2; 201233, −4.1; 201239, −5.0; 201240, −3.7; 201241, −3.3; 201242, −3.3; 104571, −2.9; 201245, −2.9; 201246, −3.1; 201247, −3.7; 201248, −3.1; 105643, −3.2; 201255, −2.9; 201257, −3.1; 201261, −3.9; 201263, −3.2; 201265, −3.6; 201266, −3.1; 92723, −3.5; 201267, −3.9; 201269, −3.6; 201272, −3.0; 201274, −2.9; 201275, −3.9; 15689, −3.6; 97843, −3.0; 201278, −3.0; 201284, −4.6; 201285, −3.4; 201287, −6.1; 100846, −3.1; 201288, −3.3; 201289, −3.7; 201292, −3.3; 201293, −3.7; 201294, −4.0; 201295, −3.1; 201296, −4.1; 201297, −4.2; 201302, −3.1; 82168, −3.1; 201303, −4.1; 201306, −3.2; 201309, −3.6; 201310, −2.9; 201315, −3.9; 201317, −3.1; 201318, −2.9; 201319, −3.0; 201320, −3.1; 201322, −3.5; 68865, −3.5; 201323, −3.0; 201325, −3.3; 201326, −3.2; 201330, −3.0; 89460, −3.0; 201333, −3.8; 201335, −3.1; 85836, −3.0; 201338, −4.4; 81455, −3.2; 201344, −4.2; 201345, −3.6; 201348, −3.0; 201349, −3.3; 201350, −3.0; 201353, −2.9; 80573, −3.1; 201355, −3.0; 201357, −3.6; 201358, −3.4; 201360, −3.0; 68273, −3.5; 201367, −3.1; 201368, −3.0; 89891, −3.4; 201369, −3.4; 201370, −4.8; 201374, −3.0; 94764, −3.2; 201376, −4.0; 201379, −3.4; 88050, −3.3; 201383, −3.8; 106023, −3.3; 201386, −3.1; 201388, −3.0; 201389, −3.0; 201390, −3.3; 201393, −3.2; 201394, −3.0; 201395, −3.4; 201396, −3.3; 82139, −2.9; 201401, −3.6; 201402, −3.8; 201403, −3.8; 201409, −3.7; 201412, −3.9; 201413, −2.9; 201414, −3.7; 201415, −3.3; 96034, −3.2; 201416, −3.1; 201417, −3.2; 63568, −3.7; 93991, −3.4; 201424, −3.8; 160398, −3.1; 201433, −3.0; 201434, −3.2; 201435, −3.5; 90928, −3.1; 84923, −3.2; 201438, −4.1; 201439, −3.4; 201447, −4.5; 201448, −3.2; 10491, −3.0; 201451, −2.9; 201452, −3.0; 201454, −4.5; 201456, −3.4; 201457, −4.6; 201459, −3.1; 201460, −3.9; 201461, −4.1; 201465, −3.5; 201469, −3.9; 201470, −3.4; 201471, −3.1; 201484, −3.4; 82420, −2.9; 201489, −3.4; 201494, −4.2; 201495, −3.3; 201496, −3.2; 201497, −3.3; 201499, −4.0; 201502, −2.9; 201503, −3.8; 201504, −4.6; 201507, −2.9; 201508, −3.1; 201510, −3.5; 201514, −3.2; 201516, −3.0; 56014, −3.2; 201525, −3.1; 90100, −3.1; 91383, −3.0; 201532, −3.2; 201533, −3.2; 201535, −4.3; 201536, −4.0; 201537, −3.7; 201540, −2.9; and 201544, −2.9.

List 8, which follows, provides a list of SEQ ID NOs and log 2 enrichment values for peptides inserted within the capsids of AAV particles that were found to be associated with low levels of enrichment (i.e., detargeting) of the AAV particles in the liver (first log 2 enrichment value) and good levels of enrichment of the AAV particles in the lungs (second log 2 enrichment value): 200189, −4.0, 5.4; 200191, −4.4, 5.8; 200216, −4.3, 8.1; 200327, −3.4, 5.4; 200371, −2.9, 4.8; 200418, −3.7, 8.9; 200427, −5.2, 8.5; 200507, −3.2, 8.0; 200597, −4.0, 6.2; 200605, −3.5, 9.6; 200611, −4.6, 8.9; 200645, −3.1, 3.2; 200646, −3.7, 8.9; 101214, −3.6, 8.8; 200822, −3.1, 8.6; 200875, −3.4, 8.6; 200879, −3.9, 9.0; 200905, −3.3, 8.4; 200909, −5.2, 7.0; 200979, −5.0, 8.8; 200991, −3.5, 9.0; 201026, −3.2, 7.2; 201028, −3.5, 7.9; 201036, −3.7, 8.8; 201039, −3.1, 8.2; 201047, −4.6, 9.3; 201048, −3.0, 9.0; 201075, −3.1, 6.6; 201243, −3.9, 6.1; 201304, −4.0, 8.8; 201339, −3.2, 7.0; 201347, −2.9, 2.5; and 201381, −5.1, 8.9.

List 9, which follows, provides a list of SEQ ID NOs and log 2 enrichment values for peptides inserted within the capsids of AAV particles that were found to be associated with low levels of enrichment (i.e., detargeting) of the AAV particles in the liver (first log 2 enrichment value) and good levels of enrichment of the AAV particles in muscle (second log 2 enrichment value): 200066, −3.3, 1.7; 200068, −3.4, 1.8; 200096, −3.2, 1.9; 200100, −3.5, 1.9; 200113, −3.2, 1.9; 200138, −2.9, 1.8; 200140, −3.7, 5.8; 200164, −3.1, 2.0; 200165, −3.5, 1.8; 200173, −3.7, 1.9; 200181, −3.3, 1.7; 200184, −3.4, 1.8; 200188, −3.0, 1.8; 200189, −4.0, 4.6; 200191, −4.4, 6.4; 200202, −3.0, 2.0; 200206, −3.0, 1.8; 200235, −3.0, 1.8; 200246, −3.2, 2.0; 200250, −4.5, 5.2; 200263, −3.5, 1.8; 200275, −3.0, 1.7; 200327, −3.4, 5.8; 200336, −4.9, 2.0; 200356, −3.4, 1.9; 200357, −3.0, 1.8; 200358, −3.1, 1.8; 200361, −3.2, 1.8; 200365, −4.3, 2.1; 200368, −3.0, 1.8; 200371, −2.9, 4.8; 200390, −2.9, 1.8; 200415, −3.1, 2.0; 200416, −3.3, 1.8; 200426, −3.0, 2.0; 200431, −4.3, 1.8; 200445, −3.1, 1.8; 200446, −3.2, 2.0; 200447, −3.2, 1.9; 200448, −3.2, 1.8; 200460, −3.6, 1.9; 200466, −3.4, 2.3; 200483, −3.6, 1.9; 200493, −3.1, 1.8; 200560, −3.3, 1.8; 200597, −4.0, 6.5; 96355, −4.4, 1.9; 200632, −2.9, 1.9; 106643, −3.0, 1.7; 200642, −3.1, 1.7; 200644, −3.0, 1.7; 200648, −3.6, 1.8; 200649, −3.0, 1.8; 200665, −3.4, 1.8; 200691, −3.2, 1.8; 200695, −3.9, 1.8; 200700, −2.9, 1.8; 200701, −3.2, 2.0; 200719, −3.4, 1.8; 200807, −2.9, 2.1; 200812, −3.0, 2.0; 200836, −3.3, 1.8; 200840, −2.9, 1.9; 200878, −3.3, 1.8; 200882, −3.2, 1.8; 200904, −4.4, 1.8; 200909, −5.2, 5.1; 200932, −3.2, 1.8; 200967, −3.0, 2.1; 201010, −2.9, 1.7; 201024, −3.1, 1.9; 201026, −3.2, 6.5; 201066, −3.3, 1.8; 201075, −3.1, 5.7; 201084, −3.3, 1.9; 201095, −3.2, 2.3; 201098, −3.0, 1.8; 201145, −4.4, 1.8; 201146, −3.2, 1.7; 201221, −4.1, 1.9; 201226, −3.1, 1.8; 201243, −3.9, 4.1; 201259, −3.2, 1.9; 201269, −3.6, 1.7; 201316, −2.9, 1.8; 201321, −3.8, 1.8; 68865, −3.5, 1.8; 201378, −4.2, 2.0; 201397, −4.3, 1.8; 201404, −3.0, 2.3; 201431, −3.1, 2.0; 201453, −3.6, 2.0; 201455, −3.9, 1.9; 160413, −3.7, 1.9; 201461, −4.1, 1.8; 201462, −3.0, 1.8; 201475, −4.7, 1.9; 201477, −3.9, 1.8; 201500, −3.6, 1.8; and 56014, −3.2, 1.8.

List 10, which follows, provides a list of SEQ ID NOs and log 2 enrichment values for peptides inserted within the capsids of AAV particles that were found to be associated with low levels of enrichment (i.e., detargeting) of the AAV particles in the liver (first log 2 enrichment value) and good levels of enrichment of the AAV particles in the heart (second log 2 enrichment value): 200140, −3.7, 5.5; 200189, −4.0, 3.7; 200191, −4.4, 5.5; 200250, −4.5, 3.5; 200327, −3.4, 5.1; 200371, −2.9, 4.2; 200483, −3.6, 2.2; 200597, −4.0, 5.8; 200645, −3.1, 1.9; 200909, −5.2, 5.2; 201026, −3.2, 6.3; 201075, −3.1, 5.6; 201111, −3.2, 1.9; and 201243, −3.9, 3.9.

List 11, which follows, provides a list of SEQ ID NOs and log 2 enrichment values for peptides inserted within the capsids of AAV particles that were found to be associated with low levels of enrichment (i.e., detargeting) of the AAV particles in the liver (first log 2 enrichment value) and good levels of enrichment of the AAV particles in the brain (second log 2 enrichment value): 82853, −3.1, 1.2; 200030, −3.0, 1.1; 200035, −3.3, 1.0; 104779, −3.1, 1.0; 200071, −3.4, 2.4; 200079, −3.5, 1.0; 200104, −3.3, 2.4; 200108, −2.9, 1.0; 200132, −2.9, 0.9; 200152, −3.1, 2.4; 200182, −3.8, 1.1; 200202, −3.0, 1.1; 200204, −3.7, 1.9; 200228, −3.9, 0.9; 200232, −3.5, 1.3; 200237, −3.2, 1.3; 200238, −3.0, 1.1; 200243, −3.5, 0.9; 200248, −3.5, 1.7; 200257, −3.3, 0.9; 200261, −3.0, 2.2; 200286, −3.3, 1.2; 200287, −2.9, 1.4; 200292, −2.9, 1.0; 200304, −3.2, 1.0; 200318, −3.4, 1.5; 200319, −3.0, 1.3; 200349, −3.1, 1.0; 200360, −3.7, 0.9; 79466, −3.0, 1.1; 109926, −3.1, 1.5; 200436, −3.2, 1.0; 200437, −3.3, 1.1; 200439, −3.0, 1.0; 200459, −3.5, 1.8; 200465, −3.3, 1.0; 200466, −3.4, 1.2; 200483, −3.6, 1.5; 200503, −3.5, 1.6; 200523, −3.3, 1.8; 83170, −3.7, 1.1; 200531, −3.2, 1.0; 200542, −3.2, 1.1; 200555, −3.0, 1.0; 200560, −3.3, 0.9; 200569, −3.1, 1.0; 200598, −3.0, 1.1; 200599, −3.2, 1.3; 200622, −2.9, 3.2; 200632, −2.9, 1.0; 200637, −3.3, 1.0; 200665, −3.4, 1.1; 200673, −3.2, 1.9; 200675, −2.9, 1.4; 200689, −3.1, 0.9; 200695, −3.9, 0.9; 200708, −3.6, 1.8; 200768, −3.9, 1.0; 200815, −4.1, 1.0; 200818, −3.1, 1.0; 200819, −3.0, 1.0; 200827, −4.3, 1.5; 200832, −3.4, 1.0; 200835, −3.1, 0.9; 99435, −3.1, 0.9; 200845, −2.9, 1.1; 78297, −3.2, 1.3; 200871, −3.3, 1.2; 200879, −3.9, 1.0; 200887, −2.9, 2.7; 79765, −3.0, 1.0; 200935, −2.9, 2.3; 200941, −3.5, 1.2; 200944, −3.4, 1.0; 200946, −3.6, 1.0; 200955, −3.1, 2.1; 200967, −3.0, 1.6; 200970, −2.9, 1.7; 200995, −2.9, 1.5; 201004, −3.0, 1.2; 201006, −3.8, 1.6; 95858, −3.0, 1.3; 201024, −3.1, 1.2; 98522, −3.2, 1.1; 201084, −3.3, 1.5; 201091, −3.0, 1.2; 201098, −3.0, 1.3; 201111, −3.2, 2.6; 201125, −3.0, 1.3; 201134, −3.3, 0.9; 201146, −3.2, 1.2; 201221, −4.1, 1.1; 201224, −4.5, 1.2; 201227, −3.2, 1.4; 201228, −3.8, 1.6; 201230, −3.2, 0.9; 201270, −3.5, 1.2; 160330, −2.9, 1.0; 92858, −3.1, 1.5; 201305, −3.0, 1.1; 201312, −3.1, 1.4; 201324, −4.3, 0.9; 201339, −3.2, 1.0; 201378, −4.2, 1.3; 88050, −3.3, 0.9; 201399, −3.0, 1.5; 201404, −3.0, 1.7; 201431, −3.1, 2.1; 201437, −3.7, 1.0; 201441, −3.8, 1.5; 201466, −2.9, 1.5; 201477, −3.9, 1.0; 201507, −2.9, 1.2; 201509, −3.1, 1.5; 201539, −3.1, 1.1; and 201543, −4.1, 1.2.

List 12, which follows, provides a list of SEQ ID NOs and log 2 enrichment values for peptides inserted within the capsids of AAV particles that were found to be associated with low levels of enrichment (i.e., detargeting) of the AAV particles in the liver (first log 2 enrichment value) and good levels of enrichment of the AAV particles in the kidneys (second log 2 enrichment value): 82853, −3.1, 1.4; 200030, −3.0, 1.1; 200035, −3.3, 1.9; 104779, −3.1, 1.1; 200049, −3.4, 1.2; 200054, −3.2, 1.3; 200057, −3.3, 1.1; 200062, −3.1, 1.3; 200066, −3.3, 1.2; 200067, −3.4, 1.3; 200068, −3.4, 1.2; 200069, −3.0, 1.5; 200071, −3.4, 1.3; 200089, −3.1, 1.1; 200091, −3.4, 1.1; 200096, −3.2, 1.2; 200100, −3.5, 1.9; 200108, −2.9, 1.6; 200113, −3.2, 1.1; 106717, −2.9, 1.2; 200117, −3.7, 1.1; 200124, −3.1, 1.3; 200138, −2.9, 1.2; 200147, −3.0, 1.3; 200152, −3.1, 1.1; 13424, −3.0, 1.2; 200153, −3.1, 1.2; 200164, −3.1, 1.1; 200166, −2.9, 1.1; 200168, −3.1, 1.2; 200171, −3.3, 1.2; 200173, −3.7, 1.3; 200176, −3.1, 1.1; 200181, −3.3, 1.1; 200182, −3.8, 1.1; 200184, −3.4, 1.1; 200202, −3.0, 1.2; 200225, −3.1, 1.2; 200228, −3.9, 1.1; 200234, −3.1, 1.1; 200240, −4.1, 1.2; 200242, −3.1, 1.2; 200247, −3.2, 1.2; 200251, −3.7, 1.1; 200253, −3.0, 1.1; 200255, −3.6, 1.2; 200261, −3.0, 1.6; 200266, −4.4, 1.1; 200270, −3.0, 1.0; 200275, −3.0, 1.1; 200279, −3.8, 1.1; 200283, −3.0, 1.2; 200286, −3.3, 1.2; 200287, −2.9, 1.5; 200288, −3.0, 1.2; 200305, −3.4, 1.1; 200308, −3.5, 1.1; 200311, −3.0, 1.2; 200312, −3.4, 1.2; 200313, −2.9, 1.2; 200318, −3.4, 1.3; 200321, −3.0, 1.1; 109197, −2.9, 1.2; 200334, −3.8, 1.1; 200336, −4.9, 1.3; 200338, −3.0, 1.3; 200343, −3.5, 1.0; 200349, −3.1, 1.2; 200350, −3.5, 1.4; 200353, −2.9, 1.4; 200356, −3.4, 1.4; 200357, −3.0, 1.1; 200361, −3.2, 1.3; 200365, −4.3, 1.5; 200368, −3.0, 1.3; 200370, −2.9, 1.1; 200374, −3.1, 1.3; 79466, −3.0, 1.3; 200375, −2.9, 1.3; 200378, −3.4, 1.2; 17057, −3.1, 1.3; 200389, −3.2, 1.4; 200390, −2.9, 1.4; 200391, −4.0, 1.4; 109926, −3.1, 1.2; 200414, −3.2, 1.2; 200415, −3.1, 1.3; 200419, −2.9, 1.8; 200426, −3.0, 1.3; 200429, −2.9, 1.4; 200430, −3.4, 1.4; 200441, −3.7, 1.1; 159845, −3.9, 1.3; 200444, −3.1, 1.1; 200445, −3.1, 1.2; 200446, −3.2, 1.3; 200447, −3.2, 1.4; 200448, −3.2, 1.3; 200449, −3.4, 1.4; 200453, −2.9, 1.2; 200457, −3.1, 1.2; 200459, −3.5, 1.3; 200460, −3.6, 1.2; 200463, −3.3, 1.6; 200464, −3.1, 1.2; 200465, −3.3, 1.2; 200466, −3.4, 1.6; 200474, −3.6, 1.1; 200475, −3.1, 1.3; 200483, −3.6, 2.2; 200493, −3.1, 1.2; 200502, −3.0, 1.3; 200503, −3.5, 1.8; 200531, −3.2, 1.1; 200547, −3.0, 1.2; 200548, −3.6, 1.1; 200560, −3.3, 1.3; 200577, −3.2, 1.1; 200583, −3.4, 1.1; 200584, −3.0, 1.6; 200588, −2.9, 1.6; 200600, −3.6, 1.7; 200603, −3.1, 1.2; 200606, −3.2, 1.3; 96355, −4.4, 1.2; 200632, −2.9, 1.5; 106643, −3.0, 1.1; 83899, −2.9, 1.3; 200642, −3.1, 1.2; 200643, −3.1, 1.0; 200644, −3.0, 1.3; 200645, −3.1, 1.7; 200648, −3.6, 1.2; 200649, −3.0, 1.3; 200651, −3.2, 1.0; 159979, −3.2, 1.2; 97034, −3.4, 1.1; 200673, −3.2, 1.6; 200674, −3.2, 1.1; 200675, −2.9, 1.5; 200691, −3.2, 1.1; 200692, −3.0, 1.3; 200695, −3.9, 1.4; 200700, −2.9, 1.2; 200701, −3.2, 1.1; 200705, −3.1, 1.8; 200710, −3.0, 1.2; 200714, −2.9, 1.1; 200717, −3.7, 1.1; 200719, −3.4, 1.1; 200752, −3.1, 1.2; 200754, −3.1, 1.4; 200779, −3.1, 1.1; 65598, −3.3, 1.3; 200807, −2.9, 1.6; 108006, −2.9, 1.4; 200811, −3.1, 1.2; 200812, −3.0, 1.1; 200813, −3.1, 1.1; 200819, −3.0, 1.4; 200832, −3.4, 1.4; 200833, −3.4, 1.2; 200835, −3.1, 1.1; 200836, −3.3, 1.3; 200838, −3.0, 1.2; 200840, −2.9, 1.3; 99435, −3.1, 1.4; 200845, −2.9, 1.4; 78297, −3.2, 1.2; 75929, −3.0, 1.1; 200856, −3.1, 1.2; 97381, −3.2, 1.3; 200871, −3.3, 1.4; 200872, −3.8, 1.3; 200878, −3.3, 1.1; 200882, −3.2, 1.1; 200885, −3.3, 1.1; 200894, −3.1, 1.4; 200904, −4.4, 1.1; 200906, −4.4, 1.1; 107915, −4.0, 1.1; 200912, −3.7, 1.1; 79765, −3.0, 1.7; 200920, −3.0, 1.2; 134573, −2.9, 1.1; 200932, −3.2, 1.2; 200941, −3.5, 1.3; 107749, −3.4, 1.1; 200946, −3.6, 1.5; 200954, −3.3, 1.1; 200959, −3.1, 1.1; 200960, −3.2, 1.1; 54729, −3.5, 1.2; 200967, −3.0, 1.1; 200971, −3.1, 1.4; 200981, −3.2, 1.1; 200987, −3.0, 1.1; 200990, −3.2, 1.1; 200992, −3.6, 1.3; 201006, −3.8, 1.1; 201008, −3.2, 1.1; 95858, −3.0, 1.6; 201010, −2.9, 1.2; 109276, −3.3, 1.1; 201023, −3.0, 1.2; 201024, −3.1, 1.4; 201037, −3.4, 1.2; 91104, −3.5, 1.4; 201043, −3.5, 1.1; 201058, −2.9, 1.0; 201061, −4.0, 1.1; 201066, −3.3, 1.2; 201074, −4.4, 1.1; 201081, −3.2, 1.3; 201095, −3.2, 1.5; 201098, −3.0, 1.3; 201111, −3.2, 2.2; 201134, −3.3, 1.1; 201138, −3.2, 1.1; 201143, −3.2, 1.1; 201146, −3.2, 1.4; 201181, −3.5, 1.1; 201221, −4.1, 1.4; 9200, −2.9, 1.1; 201224, −4.5, 1.2; 201237, −3.3, 1.1; 201250, −3.7, 1.4; 201258, −2.9, 1.3; 201259, −3.2, 1.3; 160330, −2.9, 1.1; 92858, −3.1, 1.4; 201308, −3.0, 1.1; 201311, −3.1, 1.1; 201312, −3.1, 1.1; 201313, −3.1, 1.1; 201316, −2.9, 1.2; 201319, −3.0, 1.2; 201321, −3.8, 1.2; 68865, −3.5, 1.3; 105742, −3.1, 1.2; 201327, −2.9, 1.2; 201342, −3.2, 1.4; 77117, −3.3, 1.2; 201365, −4.6, 1.0; 89087, −3.0, 1.1; 201378, −4.2, 1.5; 201384, −3.2, 1.1; 201387, −3.2, 1.1; 201397, −4.3, 1.2; 201400, −3.0, 1.4; 201404, −3.0, 1.6; 201411, −3.9, 1.1; 201419, −3.2, 1.4; 160391, −2.9, 1.3; 103120, −3.1, 1.3; 201427, −4.2, 1.1; 201430, −3.0, 1.1; 201441, −3.8, 1.9; 201453, −3.6, 1.3; 201455, −3.9, 1.3; 160413, −3.7, 1.9; 201462, −3.0, 1.2; 201466, −2.9, 1.5; 201472, −2.9, 1.5; 201474, −3.5, 1.2; 201475, −4.7, 1.4; 201477, −3.9, 1.6; 201478, −3.6, 1.1; 201484, −3.4, 1.1; 201487, −3.0, 1.1; 105656, −3.2, 1.4; 201498, −3.7, 1.4; 201500, −3.6, 1.2; 201501, −3.3, 1.9; 56014, −3.2, 1.1; 201528, −3.8, 1.1; 201537, −3.7, 1.1; 201538, −3.3, 1.1; 201539, −3.1, 1.1; and 201543, −4.1, 1.1.

### Example 14: In Vivo Evaluation Macaque Retina Transduction Using AAV Variants

Experiments were undertaken to evaluate the ability of the AAV variants prepared as described in Example X1 to transduce macaque retinas. The purified virus library was injected at a dose of 4.6×10¹¹ viral genomes (vg)/eye into a male cynomolgus macaque. The injection was intravitreal to both eyes, posterior to the limbus in the superotemporal quadrant. After 28 days, the retina and retinal pigment epithelium (RPE) were harvested and RNA was recovered using a QIACube Connect. Afterwards next generation sequencing (NGS) samples were processed as detailed herein. Five biological samples were collected, two from one eye and three from the other. List 13 provided below lists consistently enriched variants detected in the retina and RPE. Variants were included in List 13 if two or more of their 7-mer amino acid (AA) replicates (considered biological replicates in the same sample) were detected in at least four biological retina samples. As a reference, AAV9 was represented by five 7-mer AA replicates but only two were detected in a single retina sample from one of the eyes.

List 13, which is provided below, provides a list of SEQ ID NOs for peptides inserted within capsid polypeptides of AAV particles, where each SEQ ID NO is followed by the mean log 2 enrichment measured for each peptide in retina samples from each eye (Eye 1 and Eye 2) calculated across all 7-mer AA and sample replicates. An enrichment score for a peptide in an eye was calculated as the log 2 of (the relative abundance in the eye of AAV particles containing capsid polypeptides containing the peptide)/(the relative abundance of AAV particles containing capsid polypeptides containing the peptide in a library of AAV particles administered to the eye).

List 13: 73517, 2.0, 0.1; 102905, 2.1, 1.4; 86093, 1.8, 2.7; 43154, 2.7, 1.4; 110065, 2.2, 3.1; 110846, 2.3, 1.2; 102312, 2.5, 3.3; 68125, 1.9, 0.1; 97033, 2.6, 3.1; 39026, 1.9, 2.0; 33918, −0.3, 0.2; 200046, 1.2, 2.2; 72754, 3.0, 2.1; 53356, 1.4, 1.7; 56089, −0.5, 1.1; 73794, 1.6, 1.0; 39836, 0.0, −1.2; 64279, 1.6, 1.4; 98530, −0.2, −1.5; 88519, 2.2, 2.2; 44039, 2.6, 2.5; 200078, 0.3, 2.2; 61178, 2.3, 3.5; 74818, 1.2, −0.7; 50470, 1.3, 0.8; 200081, 1.7, 1.7; 82949, 2.5, 2.0; 87162, 3.5, 2.6; 200085, 1.8, 3.0; 50938, 0.9, 0.9; 34455, 0.4, −1.5; 93376, 0.9, 1.3; 200092, 3.0, 2.5; 200093, 0.9, −0.2; 84029, 3.6, 2.1; 70403, 2.3, 1.2; 60798, 3.7, 2.3; 59901, 0.5, 0.9; 80016, 2.1, 1.4; 60788, 1.9, 1.3; 33040, 4.7, 3.8; 99238, 1.1, 3.0; 43949, 3.2, 2.4; 93919, 2.2, 1.8; 100299, 4.3, 2.4; 73203, 3.7, 5.1; 71893, 0.9, 1.7; 103728, 1.1, 1.5; 200139, 1.9, 2.8; 168044, 2.9, −0.4; 54005, 3.0, 3.2; 42888, 2.5, 2.2; 47866, 1.8, 2.2; 33859, 1.5, 0.9; 200162, 4.6, 3.8; 33221, −0.1, 1.7; 76972, 1.4, 2.4; 74799, 0.5, −0.2; 113512, 2.9, 1.6; 96628, 0.1, −0.1; 79110, 2.1, 1.5; 78211, 0.6, 2.7; 43507, 1.9, 1.2; 74449, 4.0, 4.0; 82504, 2.7, 2.6; 54762, 2.6, 1.5; 200177, 2.9, 0.7; 53964, 2.0, 2.1; 72330, 4.6, 0.3; 38237, 2.0, 1.9; 67477, 3.2, 2.2; 84086, 1.4, 0.3; 69990, 1.6, 0.6; 93953, 0.1, −0.7; 97026, 1.3, −0.7; 114200, 0.1, 1.3; 61964, 1.9, 2.0; 41658, 1.1, 1.7; 82767, 1.4, 1.2; 110475, 2.2, 5.0; 67500, 0.8, 1.6; 70906, 0.5, −0.8; 200219, 2.6, 3.0; 62885, 1.2, 0.8; 87199, 2.5, 2.1; 200220, 3.0, 2.5; 72056, 2.9, 3.3; 58691, 1.8, 1.4; 76565, 0.5, 0.5; 200264, 1.1, 0.5; 68453, 1.5, 0.9; 43792, 1.7, 1.5; 200277, 4.6, 2.4; 98273, 3.8, 2.6; 200282, 1.7, 3.8; 86588, 1.3, 1.3; 73905, 1.1, 0.5; 64528, 2.8, 2.6; 61920, 3.5, 3.2; 200310, 2.4, 1.4; 200337, 2.6, 1.0; 77319, 2.0, 0.9; 200344, 0.7, 0.4; 99123, 1.8, 0.4; 35141, 2.3, 0.9; 35926, 2.3, 2.2; 85434, 1.4, 0.8; 200359, 4.6, 1.1; 50231, 2.6, 2.3; 109198, 1.3, 0.6; 81680, 2.7, 2.2; 92616, 1.1, 0.5; 104149, 3.4, 2.5; 76646, 2.7, 3.2; 200396, 3.1, 0.9; 85023, 2.3, 0.0; 62873, 3.3, 1.3; 92233, 0.7, 1.1; 67799, 1.5, 2.5; 36877, 0.6, 0.5; 115884, 4.0, 2.5; 48846, 2.9, 0.8; 44096, 2.8, 1.6; 40908, 0.8, 1.5; 200402, 0.8, 0.9; 68188, 2.2, 0.3; 200405, 2.9, 2.2; 63427, 1.8, 1.1; 200428, 1.4, 0.7; 89046, 1.8, 1.0; 116062, 1.6, 1.8; 116127, 1.9, 0.4; 107386, 1.7, 0.7; 103349, 1.7, 3.3; 70917, 3.5, 0.4; 103466, 1.4, 0.1; 52024, 0.6, 0.2; 108790, 1.9, 1.2; 49866, 1.2, 0.3; 200470, 0.6, −0.1; 41720, 1.3, 0.7; 48552, 1.7, 1.0; 45851, 1.4, 1.4; 32677, 3.6, 2.2; 49467, 3.0, 1.0; 74263, 1.5, −1.6; 87124, 1.6, 1.2; 53182, 2.0, 0.4; 57386, 2.8, 0.2; 33898, 0.9, 2.9; 78499, 0.9, 2.0; 39861, −0.4, 0.8; 44814, 0.9, 0.4; 38154, 1.3, −0.4; 32901, 1.2, 0.1; 45011, 2.5, 4.0; 37743, 1.4, 0.1; 41440, 1.0, 0.9; 66963, 1.0, −0.1; 68903, 1.7, 0.2; 37761, 1.3, 1.4; 90884, 5.0, 5.7; 39060, 0.2, −0.1; 36480, 0.2, 0.5; 43537, 0.9, 0.7; 49567, 1.6, 1.6; 40690, −0.7, 0.2; 57111, 0.8, −0.1; 84947, 3.7, 1.3; 36092, 1.9, 1.1; 45410, 1.4, 2.2; 47455, 2.3, 2.5; 77101, 0.9, 1.3; 200492, 3.0, 2.3; 97526, 1.9, 1.2; 40438, 2.3, 0.5; 44330, 0.6, 0.1; 109139, 0.7, 0.5; 66146, 1.4, 0.5; 106814, 1.3, −0.1; 94060, 2.0, 2.1; 55451, 2.4, 2.5; 50473, 0.9, 1.7; 63119, 0.3, 1.0; 78830, 3.3, 2.3; 200498, 1.5, 2.3; 117961, 3.0, 3.7; 109407, 1.0, 1.4; 56459, 1.5, −0.2; 36671, 1.0, 1.6; 200506, 2.2, 1.5; 73412, 1.3, 1.6; 63367, 1.2, 2.4; 45617, 0.0, 0.4; 73252, 1.8, 1.4; 36030, 1.2, 0.8; 48997, 1.5, 3.1; 59612, 1.2, 1.4; 75238, 2.8, 2.8; 68239, 3.3, 3.2; 108683, 1.7, 1.5; 52611, 0.2, 1.2; 41962, 1.9, 0.6; 200513, 2.8, 1.3; 92025, 0.5, 2.1; 72079, 1.6, 2.6; 200515, 2.5, 2.1; 32900, −0.1, 1.9; 55750, −0.8, 0.4; 95649, 2.4, 5.0; 73387, 1.7, 1.2; 71316, 2.8, 2.8; 46856, 2.2, 3.7; 34601, 0.3, −0.1; 36084, −0.3, 1.4; 36195, 1.8, 1.9; 92732, 1.4, 1.7; 64799, 3.2, 1.8; 93636, −0.1, 0.3; 67071, 0.9, 0.2; 36926, 0.2, 2.8; 97230, 2.0, 0.8; 200527, −0.3, 1.1; 67118, 1.3, 0.9; 42117, 1.0, 0.2; 57296, 0.0, 1.4; 200529, 2.0, 1.7; 37451, 0.5, 2.0; 108834, 2.4, 1.6; 75911, 1.3, 0.7; 39822, 0.4, 1.0; 91538, 1.5, 2.1; 42712, 0.2, 0.6; 80117, 0.3, 1.0; 51697, 1.3, 1.6; 38775, 0.8, −0.5; 67655, 0.5, 1.3; 78594, 0.6, 0.0; 53130, 1.3, −0.7; 49721, 2.2, 1.4; 33856, −0.8, 2.6; 34758, 3.2, 2.4; 52668, 0.6, 0.7; 69283, 0.1, 1.1; 96281, 1.3, 3.3; 44450, 1.3, 0.1; 99883, 5.3, 1.4; 103488, 1.1, 0.6; 200535, 1.0, 1.0; 44271, 2.7, 2.6; 56461, 3.2, 1.5; 200537, 2.1, 0.8; 89469, 1.5, 0.0; 34187, 0.9, 2.9; 67005, 1.4, 1.2; 63552, 2.9, 2.4; 36848, 1.0, 1.1; 34779, 1.6, 2.0; 91480, 1.0, 0.5; 40308, 1.5, 2.9; 75776, 0.6, 1.5; 40514, 0.9, 1.6; 200541, 2.3, 0.0; 99979, 1.8, 0.7; 32885, 1.2, 1.4; 35922, 0.7, 1.9; 72381, 1.2, −0.1; 63096, 2.7, 2.4; 70173, 1.8, 2.6; 39206, 2.7, 0.7; 45353, 1.0, 2.7; 67958, −0.8, 1.1; 35605, 0.8, −0.3; 70129, 2.2, 0.7; 52666, 0.9, 1.4; 71060, 1.6, 2.4; 200550, 1.7, 0.5; 82781, 1.3, 1.9; 35814, 2.7, −0.2; 50817, 0.9, 1.8; 200551, 2.5, −0.7; 71473, 2.0, 1.4; 90539, 1.8, 0.8; 64295, 3.5, 2.1; 88396, 3.6, 1.9; 43814, 0.7, 1.2; 39858, 0.9, 1.3; 85632, 0.9, 1.8; 81087, 3.0, 3.4; 200559, 1.9, 2.3; 37131, 0.4, 1.7; 59144, 0.7, 0.7; 83476, 0.0, 1.1; 41856, 4.1, 3.2; 200563, 2.7, 2.0; 200567, 2.3, 1.7; 200571, 1.1, 2.5; 200573, 1.9, 2.1; 55147, 3.4, 1.2; 57255, 0.7, 1.9; 200593, 2.7, 3.0; 200602, 0.0, −0.2; 40372, 1.3, 1.3; 122102, 1.6, 0.9; 65439, 0.4, 1.8; 200609, 2.6, 1.5; 45398, 1.7, 2.0; 74334, 3.0, 1.3; 58607, 1.7, 2.4; 48213, 3.1, 2.9; 102349, 1.2, 1.9; 47059, 1.9, −0.2; 63889, 2.4, 2.3; 200616, 2.3, 2.0; 92794, 2.0, 1.8; 65688, 1.8, 1.7; 48005, 0.4, 1.2; 200625, 2.9, 3.1; 47252, 1.6, 0.9; 200628, 1.7, 2.1; 59733, 2.6, 2.9; 57816, 1.3, 0.3; 122945, 1.9, 1.4; 159968, 1.5, 1.0; 59752, 3.3, 0.8; 51005, −0.5, 0.3; 87008, 2.4, 2.7; 108800, 1.2, 3.1; 200641, 3.6, −0.1; 35960, 1.8, 1.6; 66069, 3.7, 0.0; 42506, 2.2, 1.4; 89879, −0.3, −0.2; 73137, 0.9, 2.0; 85556, 0.6, 1.5; 51655, 3.2, 6.1; 49779, 1.7, 0.6; 33875, 0.8, 0.4; 84657, 1.6, 1.4; 40535, 0.2, −0.2; 70934, 1.8, 1.2; 33287, 2.6, 1.8; 200658, 1.2, 1.7; 43449, 1.7, 2.3; 54532, 3.5, 0.5; 46052, 2.8, 2.0; 46251, 1.5, 1.1; 47397, 3.0, 1.1; 36194, 0.0, 1.3; 96327, 0.9, 0.5; 39643, 1.4, 1.9; 65064, 1.0, 1.6; 37207, 3.1, 1.3; 51950, 0.8, 1.1; 36952, 2.2, 2.5; 56887, 0.9, 0.7; 200669, 2.1, −1.0; 33378, 0.5, 1.7; 200672, 0.8, 1.9; 73035, 2.3, 1.1; 90264, 1.6, 3.6; 100678, 1.3, −0.6; 90935, 0.3, 1.6; 83695, 1.0, 1.6; 45265, 2.2, 2.7; 80803, −0.2, 0.3; 37012, 1.2, 1.6; 48623, 0.4, 1.7; 39350, 0.7, 0.8; 57596, 1.4, −0.4; 44009, 1.3, 2.2; 37175, −0.6, 1.1; 46186, 1.6, 0.2; 50270, 2.6, 1.6; 54346, 1.2, 2.8; 51104, 1.5, 1.5; 68392, 1.4, −0.5; 46418, 0.5, 1.7; 49787, 0.1, 3.1; 74182, 0.7, 1.7; 169151, 2.1, 1.6; 80731, 0.3, 2.0; 41532, 1.9, 2.1; 73595, 2.1, 3.5; 64285, 2.9, 3.7; 72505, 3.0, 5.5; 44058, 1.3, 0.7; 57286, 1.6, 1.8; 70297, −0.6, −2.1; 33331, 0.0, 2.8; 47634, 1.2, 2.7; 35918, 0.6, 0.7; 43189, 2.4, 2.9; 40454, 1.3, 2.4; 35490, 3.0, 1.9; 98280, 2.8, 2.6; 83016, 1.8, 5.5; 53503, 1.3, 2.1; 75368, 1.7, 4.9; 35146, 1.9, 1.4; 44345, 0.8, 2.0; 88518, 1.8, 1.2; 39754, 0.7, 1.2; 70667, 4.0, 2.9; 105818, 0.9, 1.9; 200696, 2.4, 0.2; 56666, 2.1, 3.1; 71601, 1.9, 0.8; 38126, 1.0, 3.2; 200697, 2.7, 1.9; 70288, 0.8, 1.5; 160004, 2.1, 2.2; 48442, 1.0, 0.9; 35197, −0.2, 0.4; 52244, 1.9, 1.9; 200698, 1.7, 2.4; 200702, 2.8, 0.8; 62300, 1.6, 2.2; 106694, 0.9, 1.5; 87726, 1.4, 0.0; 74567, 1.3, 1.4; 75165, 1.5, 0.9; 43220, 2.2, 3.0; 108721, 2.4, 2.9; 96641, 2.3, −2.0; 36284, 1.4, 1.3; 200722, 2.9, 2.7; 75845, 0.8, −0.4; 91982, 0.0, 1.3; 81409, 3.3, 0.8; 71378, 0.8, −0.4; 52394, 0.9, −0.3; 95809, 0.7, 0.8; 160014, 0.8, 0.9; 200728, 0.7, 1.3; 200730, −0.2, −1.2; 42336, −1.3, 2.2; 51402, 1.5, 1.4; 87101, 4.8, 3.9; 36411, 2.8, 0.9; 38753, 0.2, 0.3; 200733, 0.8, 1.6; 200734, 1.9, −0.4; 200735, 0.5, 1.4; 200736, 2.4, −1.2; 72344, 0.4, 1.8; 200737, 0.7, −0.4; 200738, 1.1, 2.1; 51122, 0.4, −0.9; 77576, −0.2, 0.2; 200740, 1.6, 4.1; 65453, 0.0, 0.3; 54343, −0.3, 2.7; 200741, 0.7, 0.9; 48643, 0.0, −0.1; 75977, 2.0, 1.2; 37314, 1.1, 0.8; 62413, 2.6, 2.1; 35171, 0.0, 0.0; 49605, 1.5, 1.5; 200744, 2.8, 2.5; 55249, 3.7, 1.2; 61075, 1.9, 2.7; 46035, 1.5, 0.9; 34782, 2.1, 0.7; 35756, 3.2, 2.1; 42183, 1.2, 1.2; 63476, 1.1, 2.4; 52049, 0.9, 0.8; 83620, 1.8, 0.3; 200748, 0.7, 1.2; 200750, 1.9, 0.7; 69667, 0.7, 0.6; 200753, −0.6, 2.3; 48894, 1.9, 2.5; 53236, 0.8, 3.1; 34065, 1.7, 1.1; 45921, −0.6, 0.6; 98404, 1.4, 0.5; 53091, −1.9, −0.6; 40016, 1.0, 2.1; 53052, 1.6, 0.7; 64333, 1.2, 0.2; 45332, 2.1, −0.1; 200756, 1.7, 0.3; 46450, 2.0, 2.1; 49984, 1.2, 1.9; 85416, 0.9, −0.4; 86920, 0.5, 1.9; 39392, 0.4, 1.9; 73796, 1.2, 0.6; 38991, 1.7, 1.5; 65240, 2.3, 1.6; 200762, 1.3, 1.2; 110324, 2.7, 3.0; 57267, −0.2, 0.0; 36086, 2.2, 0.3; 78994, 1.2, 0.9; 35683, 0.4, 1.3; 74388, 1.3, 1.6; 46556, 1.3, 2.2; 66199, 0.4, 0.6; 32636, 0.2, −0.3; 67901, 1.2, 1.5; 37249, −0.3, −0.4; 35616, 1.1, 1.6; 87511, 1.0, 0.8; 200765, 0.2, 2.5; 70826, −0.4, 0.4; 36173, 0.8, 0.6; 75949, 0.3, 1.5; 75033, 1.4, 1.5; 69455, 1.6, 1.9; 55777, 2.1, 2.4; 40606, 1.1, 1.4; 107691, 2.4, 2.9; 81252, 1.9, 2.1; 104066, 0.8, −1.7; 81163, 0.0, 2.1; 200773, 1.2, 0.0; 54589, 1.1, 0.0; 34012, −0.6, 0.1; 67916, 2.1, 3.1; 94568, 2.7, 1.2; 80125, 1.4, 0.9; 71094, 4.3, 2.4; 58647, 1.2, 0.7; 81471, 2.5, 3.5; 200775, 2.9, 0.7; 68211, 0.7, 1.9; 43921, 0.4, 0.8; 91671, 0.2, 0.7; 50911, 0.6, 1.7; 128753, 2.1, 1.7; 39017, −0.9, 0.2; 33821, 2.5, 2.7; 66336, 3.8, 3.7; 44035, 0.2, 1.3; 77459, 3.3, 2.1; 44884, 2.0, 1.3; 49098, 2.0, 2.0; 103645, 1.9, 0.4; 35487, 0.3, 0.9; 73221, 0.0, 2.2; 200784, 3.7, 3.2; 70827, 1.5, 1.1; 75612, 0.1, 2.3; 73482, 0.5, 0.1; 40942, 1.3, 1.2; 35523, 2.2, 0.2; 78673, 1.0, −0.1; 200789, 2.4, 1.4; 67525, −0.7, −0.1; 64415, 2.0, −1.2; 72665, 1.3, 1.9; 71052, −0.3, 3.3; 94335, 1.3, 2.8; 94502, 0.3, 0.6; 69362, 1.5, 3.3; 66912, 0.8, 2.0; 107367, 1.1, 0.7; 129246, 1.5, 2.4; 200791, 0.3, 1.7; 200792, 1.4, 2.2; 88305, 1.3, 1.8; 87297, 2.1, 1.8; 42628, −0.5, 0.8; 200794, 3.3, 2.8; 200795, 2.2, 0.8; 105536, 1.0, 1.1; 63741, 0.3, 1.1; 57624, 2.6, 2.2; 38873, −0.1, 2.3; 45200, 0.6, 0.8; 53998, 0.3, −0.3; 57159, 1.9, 1.5; 101506, 1.6, 1.8; 78313, 3.5, 2.3; 36988, 1.6, 1.5; 94593, 1.3, 1.6; 59548, 4.4, 2.7; 62716, 0.1, 0.8; 46500, 1.3, 1.1; 200817, 2.5, 2.8; 64229, 1.1, 1.9; 34338, 3.7, 2.2; 56966, 2.2, 1.2; 39608, 2.3, 1.6; 81475, 2.6, 0.5; 99794, 2.0, 0.2; 36542, 1.1, 2.2; 84093, −0.2, 0.4; 34117, 2.4, 1.0; 51450, 2.3, 1.7; 67408, 0.8, −0.7; 54913, 1.9, 1.6; 54592, 2.1, 0.3; 37767, 1.3, 1.3; 54392, 0.4, 2.5; 33508, 2.7, 1.9; 81258, 2.5, 1.4; 72923, 2.0, 2.2; 101868, 3.1, 1.9; 200857, 3.2, 1.3; 86753, 1.7, 2.7; 56470, 2.4, 2.5; 33854, 2.6, 2.5; 200862, −0.2, 0.9; 41298, 2.5, 0.2; 40645, 0.9, 1.5; 56142, −0.1, 1.0; 77578, 3.0, 1.3; 58389, 3.9, 1.4; 38160, 1.3, 1.5; 33761, −0.3, 0.6; 42679, 0.9, 2.4; 49356, 2.2, 1.0; 200864, 0.1, 0.0; 200865, 0.2, 0.4; 36341, 1.3, 2.5; 200867, 3.8, 6.3; 39074, 2.9, 1.7; 101320, 2.2, 0.7; 44224, 1.0, 0.1; 34718, 1.7, 1.0; 33486, 1.0, 2.6; 99551, 2.1, 1.2; 48557, 0.0, 1.6; 36465, 0.3, 1.2; 91640, 1.4, 2.1; 34349, 1.9, −0.9; 38494, 2.6, 2.2; 78835, 1.1, 0.4; 33773, 2.6, 1.5; 44626, 1.7, 3.2; 66711, 1.1, 1.2; 41078, 2.1, 1.9; 55262, 1.0, 0.0; 44125, 2.6, 1.5; 38169, 2.5, 3.4; 92961, 1.9, 0.6; 71463, 2.3, 1.0; 53136, 0.9, 3.0; 34132, 1.4, 1.8; 42959, 1.8, 0.2; 40087, 1.2, 2.2; 33005, 5.2, 4.1; 34109, −0.3, 1.7; 73993, 2.4, 2.6; 35998, 3.0, 2.5; 75423, 1.2, 2.1; 97268, 1.3, 2.8; 52385, 1.7, 0.1; 133004, 0.6, 1.0; 93206, 0.9, 0.0; 41154, −0.8, 1.9; 61116, 0.2, −0.2; 81377, 1.1, 0.6; 71981, 1.7, 1.7; 52058, 0.0, 2.8; 32731, −1.1, 1.8; 50642, 0.7, 0.9; 69989, 0.8, 3.2; 35569, −0.5, 1.3; 41850, 3.6, 2.9; 200897, 3.3, 2.3; 44042, 1.6, 2.6; 46122, 1.6, 0.5; 200908, −0.3, 1.4; 33882, 3.0, 0.8; 48787, 1.5, 1.9; 65619, 0.9, 1.8; 98151, 0.0, 2.6; 34907, −0.4, −0.3; 51609, 0.4, 0.6; 52786, 1.3, 1.3; 37133, −0.4, 0.7; 83752, 2.8, 0.9; 58140, 0.7, 1.9; 80974, 0.6, 2.1; 200913, 1.4, −0.5; 66872, 2.7, 0.4; 34622, 1.3, 0.3; 42100, 0.8, −2.2; 104516, 0.6, 0.9; 56062, 0.4, 1.1; 37864, 2.4, 1.6; 56696, 1.7, 3.6; 47102, 0.9, 1.6; 60835, 0.6, 0.5; 200926, 5.3, 3.1; 85051, 1.2, 1.5; 84551, 3.2, 3.3; 32743, 2.7, −1.0; 38828, 0.2, 1.1; 59782, 4.6, 4.1; 56954, 3.1, 1.7; 48938, 1.6, 0.8; 38579, 0.9, −0.6; 50581, 2.7, 1.4; 62719, 1.4, 1.3; 43708, 2.3, 1.7; 33059, −0.3, 1.5; 42478, 1.7, 2.1; 34512, 0.4, 1.9; 55733, 2.3, 0.8; 63923, 2.1, 2.3; 34586, 1.4, 2.0; 40001, 1.0, −0.1; 45448, 0.4, 0.3; 54500, 0.3, 0.3; 40237, 0.9, −0.1; 50166, 0.3, 0.8; 76921, 1.7, 1.6; 58444, 1.9, 1.2; 39975, 3.0, 2.5; 200933, 2.0, 3.3; 67867, 2.0, 1.1; 33117, 1.2, 1.9; 55643, 2.8, 2.0; 200937, 4.1, 4.2; 98298, 2.3, 1.6; 48130, 0.5, 2.1; 200939, 2.3, 4.2; 33753, 1.9, 0.1; 37498, 0.0, 1.1; 99584, 0.7, 1.1; 41986, 0.9, 1.0; 59714, 1.7, 3.2; 48911, 1.6, 0.4; 48908, 1.2, 0.7; 42234, 0.4, 0.5; 64324, 0.7, 0.8; 35768, 2.5, 2.0; 69612, 0.7, 0.2; 107478, 2.1, 1.0; 58436, 2.1, 1.4; 96992, 1.0, 3.2; 88797, 3.1, 1.7; 200948, 0.4, 2.1; 37202, 0.2, 0.0; 38974, 1.4, −0.9; 62313, 1.8, 1.2; 59718, 1.8, 1.0; 90369, 1.4, 1.3; 76521, 1.6, 1.0; 80552, 0.7, 1.5; 55056, 0.6, 3.5; 51287, 1.0, 1.7; 88215, 1.9, 1.1; 108833, 1.6, 2.9; 63423, 1.4, 1.2; 41512, 1.9, 0.3; 76971, 0.1, 0.0; 51883, 0.0, 1.6; 42428, 0.7, −0.2; 74145, 0.1, 0.4; 37566, −0.6, 0.6; 39358, 0.5, 0.4; 200974, 2.0, −0.2; 35140, 2.0, 1.0; 58555, 1.2, 1.5; 47926, 0.0, 1.9; 32702, 1.0, 1.0; 41041, 1.3, 0.5; 33085, −0.1, 1.0; 200975, 0.4, 0.2; 41638, 1.7, 0.6; 34466, 2.9, 1.4; 33541, 1.7, 0.3; 74517, 0.8, 2.3; 59658, 2.6, 2.0; 64356, 2.2, 1.7; 43000, 2.9, 1.8; 84931, 0.1, 1.3; 78865, 2.3, 3.4; 46705, 0.5, 1.6; 84158, 0.3, 0.8; 35579, 2.3, −0.2; 50499, 2.2, 1.6; 57462, 1.7, 2.3; 91234, 1.8, 1.1; 48618, 1.8, −0.1; 36352, 1.1, 0.4; 200989, 2.5, 3.2; 65386, 2.0, 2.8; 88894, 2.3, 2.3; 93362, 1.9, 0.6; 50386, 0.9, 1.2; 87979, 0.7, 1.2; 200993, 1.8, 1.5; 63840, 0.1, 0.4; 34381, 0.9, 1.6; 94794, 1.8, 2.0; 39478, 1.7, −0.3; 56577, 0.2, 1.7; 74610, 2.0, 1.5; 33498, 2.8, 1.6; 105191, 3.1, 1.7; 65878, 2.7, 1.6; 60332, 1.4, 0.5; 49403, 0.9, 2.7; 53861, 3.9, 1.6; 65671, −0.4, 4.6; 44381, 1.0, 2.0; 86860, 1.4, 4.6; 101663, 0.5, 2.5; 86075, 2.7, 2.6; 42562, 2.1, 0.5; 61037, 1.1, 2.2; 46606, 2.5, 1.6; 37186, −0.6, 0.3; 37552, 0.9, 1.4; 96556, 0.7, 2.9; 55234, 1.7, 0.5; 38591, 2.5, 2.7; 56712, 1.6, 1.9; 38932, −0.3, −0.3; 99856, 2.9, 4.3; 85972, 2.3, −0.3; 40485, 0.7, 1.1; 109307, 0.2, 0.5; 79844, 0.7, 0.1; 62209, 1.0, 1.5; 90870, 1.9, 1.3; 42418, 0.9, 0.2; 41108, 0.7, 1.1; 41488, 2.0, 2.7; 48185, 0.5, 1.0; 52202, 0.9, 2.8; 41810, 1.7, 1.6; 201016, 1.1, 2.1; 83301, 2.5, 1.6; 201022, 0.8, 1.4; 43275, 2.5, 2.2; 35431, 2.0, 1.0; 68101, 2.0, 2.1; 43125, 1.6, 1.5; 46285, 0.9, −1.0; 44198, 0.4, 0.2; 74939, 1.8, 3.5; 78356, 2.0, 2.2; 62739, 1.6, 2.8; 201032, 1.0, 0.7; 201034, 1.1, 1.7; 201038, 5.7, 3.0; 201041, 3.1, 4.3; 43011, 2.8, 0.4; 41464, 1.7, 2.3; 65215, 1.5, 2.3; 71623, 0.8, 0.7; 57969, 0.4, 1.1; 106666, 1.6, 1.8; 89243, 2.7, 4.2; 107156, 0.8, 1.2; 64976, 1.5, 1.9; 65457, 1.8, 1.1; 201052, 3.9, 2.9; 108863, 3.2, 2.4; 105586, 2.3, 1.7; 96203, 0.2, 1.2; 87762, 3.4, 2.0; 34527, 1.3, 1.0; 37109, 1.3, 1.8; 74665, 0.9, 2.6; 64376, 1.3, 2.3; 49011, 1.2, 0.5; 201063, 1.8, 2.3; 104011, 3.1, 2.8; 97366, 0.9, 0.5; 39184, 1.1, 1.0; 58578, −0.2, 1.2; 34742, 1.8, 2.7; 64143, 1.2, −0.3; 33747, −0.3, 2.5; 55955, 1.4, 0.9; 72183, 3.0, 0.6; 61653, 2.3, 1.3; 82307, 1.5, 1.4; 71991, 4.9, 2.2; 76786, 1.8, −0.2; 43864, −1.0, 1.5; 58384, 0.3, 1.4; 54813, 2.0, 1.8; 40705, 1.1, 2.3; 104159, 1.5, 1.1; 47900, 2.2, 1.6; 94509, 2.8, 2.5; 78735, 1.7, 0.6; 52869, 2.5, 1.1; 103117, 2.0, 2.7; 79297, 0.1, 3.0; 42243, 1.5, 1.6; 51915, 3.3, 3.3; 139469, 0.3, 1.8; 103193, 1.3, 1.9; 56374, 1.7, 1.4; 40391, 1.6, 2.8; 37003, 1.4, 1.5; 44832, −0.4, 0.7; 201101, 1.2, 1.5; 70343, 1.7, 0.5; 34030, 0.9, −0.6; 47826, 2.5, 1.7; 34558, 0.0, 0.9; 46582, 2.0, 1.1; 76758, 1.4, 1.0; 87783, 1.0, −1.1; 63431, 0.8, 2.4; 83366, 1.5, 1.2; 201107, 0.6, 0.0; 75928, 3.3, 3.8; 64635, 2.0, 4.3; 39257, 1.2, −0.4; 140306, 2.7, 2.7; 72889, 0.2, 0.6; 54003, −0.4, 0.0; 42762, 1.6, 1.6; 63146, 1.4, 1.1; 50632, −0.5, −0.4; 35157, 0.6, 0.3; 74632, 2.0, −0.5; 201110, 1.2, 0.0; 73547, 3.0, −1.2; 41631, 2.5, 2.5; 68456, 0.2, −0.1; 89180, 1.4, 0.0; 85369, 2.0, 3.1; 93096, 0.7, 2.2; 49577, 0.6, 0.7; 73358, 1.3, 1.5; 54065, 0.4, 1.7; 77776, 1.5, 1.3; 33012, 1.5, 0.5; 36630, 1.2, 0.6; 96315, 4.0, 2.8; 82574, 0.2, 0.8; 63328, 1.9, 3.7; 78479, −0.5, 1.3; 85529, 2.0, 1.2; 33792, 2.3, 1.2; 43888, 0.1, 2.7; 48565, 0.6, 1.2; 106963, 0.8, −0.5; 89249, 2.6, 1.0; 32565, −0.3, 0.4; 103825, 1.9, 3.0; 71602, 1.7, 1.9; 49853, −0.5, 1.3; 38972, 0.5, 2.6; 44787, 3.3, 2.6; 201128, 1.5, 1.4; 33404, 0.4, 2.2; 33011, 1.8, 1.0; 201129, 2.2, 4.3; 35612, 1.1, 1.5; 201131, −0.1, 0.9; 56367, 0.2, 0.5; 33137, 0.3, 2.0; 48840, 0.6, 1.5; 59864, 1.1, 1.2; 39542, 3.2, 1.9; 53134, 0.6, 0.3; 83458, 1.3, 3.8; 35306, 0.7, 0.8; 38502, 0.6, 2.6; 46447, 4.1, 1.3; 105608, 3.7, 3.1; 83139, 2.0, 1.1; 52063, 2.8, 1.4; 44967, −1.0, 1.3; 201136, 0.8, −0.5; 42613, 1.6, −0.2; 55881, 2.5, −0.5; 51830, 1.0, 3.3; 34607, 1.0, −0.4; 42444, −0.2, 2.1; 34386, 1.2, −1.5; 89824, 4.9, 1.9; 42988, 3.5, 1.9; 64024, 2.2, 3.0; 35096, 0.4, −0.5; 142074, 2.0, 1.3; 46614, 2.3, 4.0; 85654, 1.2, 2.2; 45405, 1.6, 0.1; 56109, 0.9, 1.6; 107146, 2.2, 1.2; 42429, 2.1, 1.8; 43959, 2.5, 3.4; 89217, 1.3, 1.2; 48805, 0.8, −0.2; 64467, 1.4, 2.9; 47772, 1.2, 1.5; 85367, 1.4, 2.7; 52205, 2.5, 1.7; 45774, 1.3, 1.5; 201153, 2.4, −1.5; 201155, 0.8, 1.5; 81931, 0.7, 1.5; 34474, 1.2, 0.4; 57587, 1.5, 2.6; 78884, 2.3, 2.4; 109299, 1.6, 0.6; 90978, 1.6, 1.6; 55422, 2.2, 2.7; 81476, 2.6, 1.9; 34254, 1.1, 0.6; 67873, 1.7, 0.9; 82785, 2.5, 2.9; 86018, 0.9, 0.3; 86652, 0.0, 1.8; 45161, 2.1, 4.0; 39757, −0.2, −1.3; 143060, 2.7, 2.8; 98209, 1.0, 2.4; 88402, 2.0, 2.0; 75703, 2.8, −0.2; 143210, −0.2, 1.2; 201167, 0.5, −0.1; 89310, 1.7, 1.9; 85485, 3.4, 1.8; 97350, 4.2, 4.2; 201168, 2.0, 2.6; 89064, 0.1, −0.4; 201170, 2.7, 1.0; 79669, 2.8, 2.3; 61644, 3.0, 2.3; 35326, 2.9, 2.6; 59258, 1.2, −1.2; 33025, −0.1, −0.7; 54829, 1.8, 1.0; 35526, 0.4, 0.9; 64085, 3.0, 2.7; 60141, 2.1, 2.6; 48832, 1.6, 0.7; 53428, 1.4, 2.8; 36881, 3.5, 1.4; 81187, 2.9, 3.5; 201173, 1.6, 0.9; 55544, 0.5, 0.4; 78574, 3.3, 2.8; 57786, 0.9, 0.7; 79478, 1.6, 3.8; 201174, 2.7, 1.7; 65561, 1.9, 2.3; 96541, −1.0, 0.3; 51702, 2.6, 0.9; 80211, 2.1, 1.9; 85309, 3.3, 3.4; 34981, 0.5, −0.7; 91894, 0.7, 1.4; 201183, 2.1, 1.6; 201184, 3.7, 3.3; 64124, 3.1, 4.0; 42098, 1.3, 1.5; 40723, −0.1, 2.0; 201186, 1.6, 1.4; 41602, 0.5, 0.7; 71342, 0.2, 0.8; 59170, 0.6, 1.4; 85387, 0.4, −0.3; 68339, 1.2, 1.6; 63595, 2.6, −0.1; 34691, 1.9, −0.2; 59893, 1.5, 2.8; 42700, 1.3, 1.9; 88908, 2.4, −0.6; 44856, 0.9, 0.4; 49656, 0.3, −2.1; 46820, 1.7, 1.9; 45119, 0.6, 1.7; 43059, 1.5, 2.0; 33430, −0.5, 1.6; 40658, 0.4, 1.1; 42794, 2.2, 1.6; 71897, 0.3, 2.5; 105556, 1.1, 0.2; 48078, 1.6, 1.7; 201191, 0.7, −1.0; 201192, 1.8, 0.3; 84339, 4.0, 0.8; 44362, 0.7, 0.5; 201193, −0.1, 0.8; 57191, 1.5, 1.9; 50831, 1.5, 1.0; 81071, 1.1, 2.6; 39151, 3.5, 2.4; 36860, 1.6, 0.4; 77047, 1.1, 4.2; 90473, 0.8, 2.5; 41859, 1.6, 1.3; 201197, 3.5, 4.5; 96033, 2.6, 1.8; 47490, 0.2, −0.6; 201198, −0.2, 0.7; 57081, 1.4, 2.0; 43228, 0.5, 1.9; 40262, 0.5, 1.5; 201200, 3.9, 2.6; 60358, 1.1, 2.6; 52439, 1.1, 1.7; 52894, 1.5, 1.2; 40989, 0.7, −0.9; 38625, −0.4, 0.6; 86688, 1.8, 2.0; 201201, 0.9, 2.3; 49238, 1.3, 3.8; 57689, 0.9, 2.4; 63694, 1.1, 0.5; 201202, 1.9, 2.4; 92009, 2.3, 1.8; 57258, 0.8, 1.6; 35865, 1.0, 2.0; 35137, 1.4, −0.5; 43472, 1.0, −1.0; 72472, 3.5, 2.9; 67117, 0.6, 0.6; 92545, 1.8, 2.3; 95134, 1.9, 2.6; 47973, 1.9, 1.8; 68975, 1.9, −1.0; 78937, 2.5, 0.6; 56916, 1.1, 1.3; 201204, 0.3, 2.0; 43510, 1.2, 0.9; 72396, 2.0, 0.3; 40078, −0.4, 1.5; 56648, 1.6, 1.0; 95248, 1.8, 3.8; 87636, 2.8, 1.7; 64670, 4.0, 1.4; 43731, 4.5, 4.5; 79563, 1.1, 2.3; 108191, 1.2, 2.8; 95223, 2.2, 1.4; 58602, 2.1, 0.4; 40278, 1.4, 0.9; 76402, 1.3, 2.0; 63206, 1.6, 0.8; 52437, 0.6, 0.2; 70477, 1.3, 2.3; 68494, 1.3, 2.5; 57904, 2.0, 1.7; 69406, 2.0, 3.0; 74986, 2.7, 2.8; 201235, 2.3, 4.5; 98167, 1.1, −0.7; 201238, 0.9, 2.4; 85366, 2.5, 1.4; 68696, 1.9, −1.1; 94789, 0.8, 1.7; 57301, 2.6, 1.2; 40799, 2.1, 2.5; 64077, 1.8, 2.0; 106158, 0.8, 0.7; 201244, 3.6, 3.3; 51971, 1.3, −0.3; 105328, 1.5, 2.7; 201249, 1.9, −0.5; 67789, 0.8, 0.7; 102809, 4.3, 3.7; 88106, 0.6, 2.1; 51895, 0.0, 0.5; 201252, 1.6, 1.0; 62898, 1.2, 1.5; 61457, 1.1, 1.8; 44525, 0.4, 1.4; 49948, 0.5, 3.0; 45543, −0.9, 1.7; 34503, 0.7, 1.4; 78808, 2.3, 1.3; 38709, 0.7, 0.9; 34952, 1.2, 1.4; 61576, 2.8, 1.2; 41732, 2.6, 1.8; 73746, 1.7, 0.4; 59015, 1.2, 1.9; 44913, 2.6, 1.9; 45659, 2.2, 2.8; 41575, 2.8, 2.8; 73227, 2.0, 3.0; 43828, 1.5, 1.2; 108839, 3.0, 1.3; 94535, 2.8, 3.3; 86380, 1.1, 1.2; 59050, 0.5, 1.6; 64137, 1.5, 4.0; 51671, 2.2, 0.5; 91711, 2.4, 1.6; 73148, 2.0, 1.9; 74289, 1.7, 2.4; 60490, 2.8, 2.2; 43242, 2.0, 2.3; 60099, 0.5, 1.7; 54490, 1.0, −0.1; 65503, 1.4, 0.5; 33518, 0.9, 1.7; 65242, 1.7, 1.4; 51000, 0.0, 1.9; 50526, 1.4, 0.6; 61681, 3.0, 2.9; 38476, 2.2, 1.5; 38935, 0.5, 0.5; 74159, 0.8, 1.2; 35780, 2.8, 0.1; 38981, −0.3, −1.7; 81645, 1.3, 1.5; 201282, 1.1, 0.5; 42508, 1.5, 0.8; 86750, 2.3, 3.1; 60549, 0.9, 0.4; 51052, 2.9, 2.0; 64075, 0.8, 2.3; 96756, 0.5, 2.0; 65825, 2.1, 3.4; 98121, 3.1, 2.3; 54271, 0.8, 0.3; 97719, 1.2, 3.6; 43455, 2.9, 3.0; 201300, 2.1, 1.0; 48541, 1.1, 2.0; 46421, 3.0, 0.5; 55994, 1.9, 1.8; 39229, 1.4, 0.5; 77660, 1.3, 0.7; 35363, 0.5, 2.9; 35125, 2.0, 1.5; 201307, 2.3, 1.3; 57578, 2.2, 0.6; 37910, 0.4, 1.6; 36151, 0.9, 2.6; 42205, 2.2, 1.3; 33281, 1.2, 2.3; 47977, 0.6, 0.0; 66956, 0.8, 2.1; 57883, 3.3, 3.5; 40586, 1.6, 1.8; 47962, 1.9, 1.0; 201331, 0.1, 0.3; 56310, 2.5, 2.2; 38643, 0.4, 1.3; 42294, 1.9, 1.2; 36051, 1.0, 1.5; 56948, 1.9, 1.0; 201336, 0.7, 0.5; 54323, 2.6, −0.6; 37856, 2.9, 0.2; 36511, 0.4, 1.9; 35706, 2.6, 2.6; 39361, 1.3, 3.3; 72039, 0.9, 0.2; 34054, 1.9, 1.1; 61115, 1.6, 2.2; 45950, 2.4, 3.1; 68058, 0.5, −0.3; 63168, 0.9, 1.7; 52312, 1.2, 3.6; 91559, 1.0, 1.2; 38039, 1.6, 0.2; 32613, 1.7, 1.5; 43839, 1.3, 2.1; 38725, 1.1, 0.4; 56074, 0.4, 1.6; 78321, −0.4, 0.9; 201340, 1.0, 1.7; 50245, −0.1, −0.6; 81736, 1.3, 1.1; 81899, 2.3, 1.7; 58620, 1.8, 1.2; 55281, 0.3, 1.0; 150377, 1.1, 3.9; 54130, 0.6, 2.0; 71033, 2.2, 1.0; 53415, 2.1, 2.6; 47856, 0.1, −1.0; 38745, 1.4, −1.2; 40766, 1.2, 1.7; 62823, 3.0, 1.6; 36817, −0.4, 0.8; 52071, 1.7, 2.0; 201352, 0.0, −0.2; 77350, 1.5, 3.3; 34834, −0.3, −0.7; 66623, 2.1, 1.9; 75731, 3.6, 1.7; 201356, 0.1, −0.1; 46898, 2.3, 2.7; 94392, 2.2, 5.2; 36438, 1.1, 0.6; 37188, 2.4, 0.8; 50127, 0.6, −1.2; 51937, 0.4, 0.2; 79874, 0.8, −0.5; 75634, 3.4, 2.7; 35028, 1.6, 1.3; 47256, 0.5, 2.7; 37896, 0.3, 0.4; 56905, 2.3, 1.2; 201362, 2.4, 2.5; 49087, 2.2, 0.8; 52274, 0.4, 2.2; 42005, 1.0, 0.4; 37004, 2.0, 3.5; 41447, 2.5, 2.1; 47654, 1.0, 1.4; 55619, 3.2, 2.0; 87442, 2.1, 2.1; 50484, 1.5, 1.8; 43221, 0.6, 0.2; 63876, 3.9, 1.2; 38321, 1.1, 1.2; 42147, 1.9, 2.5; 69062, 1.9, 1.0; 75617, −0.3, −0.4; 201366, 1.8, 3.6; 33240, 1.6, 1.5; 36065, 3.7, 2.8; 86780, 0.3, 1.9; 39269, 1.2, 2.1; 44893, 3.4, 0.7; 35553, 3.1, 2.5; 36884, 0.2, −0.3; 50798, 3.5, 2.9; 201371, 2.7, 2.7; 37776, 0.5, 0.8; 47791, 1.3, 0.7; 34344, 1.8, 2.6; 110411, 2.2, 1.6; 68462, 1.9, 3.8; 37198, 2.4, 2.0; 41044, 2.0, 2.1; 42061, 1.0, 2.3; 33831, 1.5, 0.5; 87962, −0.5, 0.7; 48178, 1.9, 1.7; 35949, 0.7, 1.1; 67335, 2.4, 3.0; 57706, 1.6, 1.4; 110632, 2.8, 1.6; 201391, 3.3, 2.7; 97724, 1.7, −1.9; 94587, 1.5, 0.2; 38335, 0.4, −0.8; 91959, 0.7, 0.6; 75101, 0.0, −1.0; 77699, 0.5, 1.2; 57920, 1.0, 1.2; 201410, 1.0, 1.9; 71900, 2.2, 1.6; 37858, 2.5, 2.5; 37222, 1.4, 1.7; 88613, 2.4, 2.2; 52302, 2.1, −0.4; 79002, 2.0, 3.2; 46438, 0.1, 1.1; 102727, 0.5, 2.2; 77758, 1.2, 1.5; 80748, 2.8, 3.2; 47373, 1.9, −0.9; 32834, 2.5, 1.2; 45783, 1.8, 1.1; 58700, 2.6, 0.1; 50085, 0.1, 2.0; 53885, 0.8, 0.9; 48910, 0.6, 0.5; 53402, 1.1, 1.0; 58345, 1.7, 2.2; 58549, 0.9, 0.3; 92579, 2.2, 2.7; 69740, −0.7, 1.3; 201426, 2.2, 0.9; 106663, 2.9, 1.8; 48696, 1.7, 2.7; 201429, 1.4, 3.5; 32905, 0.5, 2.5; 38004, −0.6, 0.3; 54442, 0.7, −1.6; 38880, 1.6, 1.1; 48690, 1.7, 2.9; 47053, 1.9, 0.6; 93415, 0.7, 0.3; 32957, 1.8, 1.0; 99083, 1.4, 2.7; 71062, 1.7, 2.9; 62514, 1.3, 2.7; 44230, 1.7, 2.3; 201442, 2.8, 3.0; 41699, 1.2, 2.7; 201443, 2.6, 1.8; 67542, 3.2, 0.9; 201444, 0.2, 1.4; 37093, 2.9, 2.2; 201445, 1.0, 1.2; 201449, 2.1, 2.8; 84004, 4.3, 2.9; 33551, 1.1, 1.6; 37581, 1.5, 1.6; 201450, 2.5, 1.8; 73985, 0.3, 1.7; 71344, 1.6, 2.2; 85128, 0.9, 1.5; 110492, 0.6, 1.3; 36538, 1.9, 1.2; 201458, 2.7, 2.2; 46691, 1.1, 1.2; 107868, 2.1, 1.3; 37974, 0.5, 1.9; 84055, 0.2, 2.7; 110108, 1.2, 0.9; 95121, 2.5, 2.9; 201463, 4.2, 2.5; 40496, 1.6, 1.2; 201467, 2.0, 2.1; 41103, 2.5, 3.1; 37291, 2.8, 1.8; 74705, 2.9, 2.2; 89298, 4.1, 2.5; 33949, 0.8, 0.3; 46976, 2.6, 0.6; 60724, 0.4, 1.1; 79333, 3.1, 2.2; 37071, 2.1, 3.5; 35008, 1.9, −0.4; 46033, 2.8, 2.9; 35352, 2.4, 2.3; 201486, 1.3, 1.4; 35249, 1.3, 0.8; 67949, 1.2, 0.8; 86585, 1.5, 1.5; 55980, 3.3, 0.9; 71904, 2.8, 2.8; 38719, 1.4, 1.8; 59067, 2.7, 3.1; 36974, 1.3, 1.9; 48828, 1.2, 1.3; 36985, 1.5, 2.3; 46968, 1.5, 3.7; 49156, 1.0, 1.7; 201511, 0.6, 0.9; 36130, 0.9, 1.5; 35693, 2.1, −0.2; 86071, 3.0, 2.7; 82333, 2.4, 2.0; 64821, 1.1, 1.3; 201520, 2.1, 0.5; 57358, 1.5, 0.9; 37151, −0.1, 1.5; 41502, 0.9, 0.9; 40463, 0.5, 0.2; 32748, 0.0, 2.2; 47076, 1.1, 0.8; 67639, 0.1, 0.9; 59922, 1.1, 2.0; 39638, 1.7, 0.0; 201527, 1.9, 1.5; 102586, 3.2, 1.4; 34427, 1.4, 2.9; 59042, 2.3, 1.7; 47230, 0.9, 1.1; 47961, 2.4, 0.3; 44592, 2.2, 1.8; 49294, 1.5, 1.4; 58989, 1.1, 3.1; 38695, 0.4, 1.7; 40549, 2.1, 2.0; 34475, 2.2, 2.0; 52119, 2.4, 2.5; 37717, 1.4, 0.8; 36203, 1.1, 0.8; 37166, 0.1, 1.1; 46105, 2.5, 2.4; 50053, 2.1, 1.5; and 43726, 0.2, 0.5.

The following is a list of amino acid sequences for peptides used in the above examples, where each amino acid sequence is preceded by its corresponding SEQ ID NO: 200028, AAFHINA; 200029, AAIRGYA; 200030, AAITPEN; 200031, AASYKWE; 200032, ADDGPVK; 200033, ADHYVLG; 200034, ADNREDL; 200035, ADPPLQI; 200036, ADSGALE; 200037, ADTQDAA; 200038, ADYSGDT; 200039, AEDNVKA; 200040, AEDRTSE; 200041, AENSGGE; 200042, AFATRRE; 200043, AGAQQNE; 200044, AGHNEEN; 200045, AGLIISK; 200046, AGMPHLR; 200047, AGNHYDE; 200048, AGPLMGL; 200049, AGQHQYF; 200050, AGSEAWA; 200051, AHADNSV; 200052, AHDTYYL; 200053, AHGDVTS; 200054, AHHGAMW; 200055, AHHMVHH; 200056, AHLDMHI; 200057, AIEERVV; 200058, AIGSDPD; 200059, AILDIDW; 200060, AILGTVR; 200061, AIQQRTF; 200062, AISDISN; 200063, AKDVAWR; 200064, AKNYAYA; 200065, AKPFVQL; 200066, ALATVTD; 200067, ALLGTDT; 200068, ALTGNEL; 200069, AMKNDHW; 200070, AMLGTHR; 200071, AMQSEYN; 200072, ANDDNKD; 200073, ANDQDKW; 200074, ANDVHGE; 200075, ANEEAPQ; 200076, ANGDAYI; 200077, ANGHNEG; 200078, ANLNAAK; 200079, ANTEPTV; 200080, APIHHAI; 200081, APTHQRL; 200082, AQANSDL; 200083, AQHDTGD; 200084, AQLTMHS; 200085, AQPHPKF; 200086, ARDHLDE; 200087, ARYDINK; 200088, ARYPVTN; 200089, ASENFTV; 200090, ASFSKLP; 200091, ASIPGHW; 200092, ASLARRD; 200093, ASQMHNK; 200094, ASVTDKD; 200095, ATDSIAG; 200096, ATMHNDM; 200097, ATSPFQD; 200098, AVKFVPP; 200099, AVYGIDM; 200100, AYDPAPS; 200101, AYINQTA; 200102, AYQLQSL; 200103, AYTSDPE; 200104, CDHVKAQ; 200105, CEKSGCH; 200106, CGPQRVC; 200107, CMMKECR; 200108, CNLPMCK; 200109, CNTDKCI; 200110, CPTAECA; 200111, CSKSACL; 200112, CVNDPCT; 200113, DAALDNV; 200114, DAAQDKG; 200115, DAGERST; 200116, DAPPTSH; 200117, DAQNSEC; 200118, DASAEAG; 200119, DATQNNE; 200120, DATYAES; 200121, DAVLTSL; 200122, DDGANKV; 200123, DDKEREL; 200124, DDKISQI; 200125, DDKKDDF; 200126, DDQSSRA; 200127, DDSKQNI; 200128, DDSQGTW; 200129, DDTNAGK; 200130, DDTVDTT; 200131, DDWDGGN; 200132, DDYLSMH; 200133, DEDGYEK; 200134, DEHQQSA; 200135, DEKSLSS; 200136, DEPYQHK; 200137, DFDGKNS; 200138, DFGTPTT; 200139, DFKMSRN; 200140, DFVDLVD; 200141, DGAAQQP; 200142, DGDFEQE; 200143, DGDHSHD; 200144, DGDNSRQ; 200145, DGGQQVI; 200146, DGGSVNT; 200147, DGKAVAW; 200148, DGLGQEP; 200149, DGLNQDS; 200150, DGLQREE; 200151, DGPQNDK; 200152, DGPQSAW; 200153, DGYNEIH; 200154, DHDNAGR; 200155, DHDQASH; 200156, DHGNAYA; 200157, DHQRESE; 200158, DHSTETV; 200159, DIEDGTR; 200160, DIKRFAN; 200161, DINEQRA; 200162, DIRDGRR; 200163, DISTQND; 200164, DITAVNE; 200165, DKGTEQL; 200166, DKPPEIA; 200167, DKQPESI; 200168, DKTLDSF; 200169, DKTNDHE; 200170, DKWENGM; 200171, DLAMEKM; 200172, DLGGYNE; 200173, DLGLDST; 200174, DLSDAKA; 200175, DLTHDTH; 200176, DMEPKAF; 200177, DMGLMRK; 200178, DMGNDTN; 200179, DMGNKEI; 200180, DMNDNRV; 200181, DMQHQGI; 200182, DMSIQGT; 200183, DMSPNQD; 200184, DMSTAEM; 200185, DMTANTG; 200186, DMTNMAD; 200187, DMTSEST; 200188, DNAQNVH; 200189, DNDFDIW; 200190, DNDHRDI; 200191, DNDPYNM; 200192, DNDVTHA; 200193, DNGDKKI; 200194, DNGGGMD; 200195, DNGKEVP; 200196, DNGQFIE; 200197, DNGYDEK; 200198, DNHNESL; 200199, DNHNPHQ; 200200, DNPKPEN; 200201, DNQSEED; 200202, DNTPISQ; 200203, DNVPENQ; 200204, DPMYSEN; 200205, DPPNSST; 200206, DPPQATV; 200207, DPPTEGP; 200208, DPSKEKC; 200209, DPSLPGE; 200210, DQAEGTF; 200211, DQGNESN; 200212, DQGVTWI; 200213, DQPSMET; 200214, DQSFAAE; 200215, DRDNYTD; 200216, DRESFPW; 200217, DRGNEDE; 200218, DRNFTRQ; 200219, DRPMVNR; 200220, DRRTAIG; 200221, DRTDETS; 200222, DRTLEES; 200223, DRVNDDQ; 200224, DSAHGAA; 200225, DSAHLQN; 200226, DSDGISE; 200227, DSDQNNE; 200228, DSDVVGK; 200229, DSGGQEL; 200230, DSGQLVA; 200231, DSGQPQP; 200232, DSKEDMR; 200233, DSKNYSI; 200234, DSMQTDQ; 200235, DSNSVGH; 200236, DSRNEAQ; 200237, DSSTNDK; 200238, DSTAPEF; 200239, DSTNVSD; 200240, DSTPIQV; 200241, DSTPTAT; 200242, DSVRNEI; 200243, DSVRNET; 200244, DTAGTHN; 200245, DTANVDA; 200246, DTAQLVE; 200247, DTAVPAV; 200248, DTDHNKC; 200249, DTDHQLS; 200250, DTDPMNF; 200251, DTENRVF; 200252, DTFGGSE; 200253, DTGFQGI; 200254, DTGKEYH; 200255, DTGMVNV; 200256, DTGNHGS; 200257, DTGYEIT; 200258, DTHHQDA; 200259, DTKENSS; 200260, DTLGPEK; 200261, DTLPLTT; 200262, DTMVQSL; 200263, DTPMEGQ; 200264, DTTYLKK; 200265, DTYNDSE; 200266, DTYPAEI; 200267, DVGGSPA; 200268, DVGMSIL; 200269, DVHGTIE; 200270, DVINQDV; 200271, DVITPEE; 200272, DVKSEVE; 200273, DVPSQAH; 200274, DVVWVGE; 200275, DYADTHK; 200276, DYANDEN; 200277, DYKPIVK; 200278, EADNYDQ; 200279, EAGNLTV; 200280, EAGSEFW; 200281, EAIKEQG; 200282, EAKQLSK; 200283, EAPMINM; 200284, EAQVWTD; 200285, EATIIPT; 200286, EATLAQT; 200287, EAVHQFL; 200288, EAYQSPL; 200289, EDDGTMN; 200290, EDDSGGH; 200291, EDEVVWE; 200292, EDFGTAK; 200293, EDGLEEF; 200294, EDIILHS; 200295, EDKDYAI; 200296, EDNKDAA; 200297, EDQGLKN; 200298, EDSHSAY; 200299, EDTGMQG; 200300, EDYAGTE; 200301, EEADINK; 200302, EEAEDNV; 200303, EEDNNPR; 200304, EEHLRHD; 200305, EEHVMIN; 200306, EESGTAV; 200307, EETGHTL; 200308, EFGASSW; 200309, EFHGHFK; 200310, EFKTQLK; 200311, EFQDLPK; 200312, EFQNTSI; 200313, EFSYDLI; 200314, EGDEDRK; 200315, EGHQVGV; 200316, EGKMIRI; 200317, EGNMEKS; 200318, EGNTYLN; 200319, EGPTGNQ; 200320, EGQQERS; 200321, EGQTFNH; 200322, EGSMEGV; 200323, EGWNMEA; 200324, EHDKEGM; 200325, EHDNVAE; 200326, EHDQGSV; 200327, EHDVDWH; 200328, EHHNQHD; 200329, EHKEQQV; 200330, EHNHAEL; 200331, EHRDNER; 200332, EHTGPEV; 200333, EHYAIHS; 200334, EIKPQET; 200335, EIKVRDR; 200336, EILHEGK; 200337, EIRLMNK; 200338, EITPNTR; 200339, EITSGTK; 200340, EKALRIT; 200341, EKDTVIA; 200342, EKGEEIQ; 200343, EKGVEIK; 200344, EKKLIIS; 200345, EKRLNLA; 200346, EKSKIYF; 200347, EKTVENS; 200348, EKVPNYM; 200349, ELGPTIS; 200350, ELHLRFE; 200351, ELINLMP; 200352, ELKDNEY; 200353, ELPYTHN; 200354, ELQDSKG; 200355, ELQGNPW; 200356, ELSAALT; 200357, ELTAATV; 200358, EMADPKP; 200359, EMAFKKT; 200360, EMAGNQV; 200361, EMAQLKD; 200362, EMGASNM; 200363, EMHTDPQ; 200364, EMKRYGI; 200365, EMTSITT; 200366, ENADDGQ; 200367, ENDGMQA; 200368, ENHVPTI; 200369, ENISNDG; 200370, ENMQTVI; 200371, ENPGEFW; 200372, ENPNQDR; 200373, ENPVWTT; 200374, ENQQPIL; 200375, ENTSALW; 200376, ENTSTAL; 200377, ENVAEGP; 200378, ENVGTSF; 200379, ENYDQGI; 200380, EPFMKED; 200381, EPNSNYE; 200382, EPQDNHS; 200383, EPQGNTP; 200384, EPSKEQS; 200385, EQAGQGD; 200386, EQFKKMM; 200387, EQHVQAD; 200388, EQKNQVD; 200389, EQNIQIT; 200390, EQPVIVP; 200391, EQQLPLT; 200392, EQQSQYP; 200393, EQSSSHE; 200394, EQTAQHV; 200395, EQYTNDE; 200396, ERDAKVR; 200397, ERHDQEA; 200398, ERIPNWS; 200399, ERMLGMH; 200400, ERPEWLK; 200401, ERSDTNE; 200402, ERSLPNK; 200403, ERTKYAA; 200404, ERTLTHM; 200405, ERTPYKM; 200406, ERYGIQV; 200407, ESADFHD; 200408, ESAPTSE; 200409, ESAQKDE; 200410, ESDPFHY; 200411, ESHGHTD; 200412, ESHNDDA; 200413, ESHNTST; 200414, ESIQGVL; 200415, ESLTFDD; 200416, ESQLDNT; 200417, ESQQNEE; 200418, ESTFKPW; 200419, ESVLASV; 200420, ETDRGAT; 200421, ETDTYRE; 200422, ETEVDRM; 200423, ETGAMEH; 200424, ETGGEPK; 200425, ETHSPES; 200426, ETIGSIQ; 200427, ETIPDWV; 200428, ETMMIKK; 200429, ETMSIER; 200430, ETPLASV; 200431, ETQELDH; 200432, ETQEPQN; 200433, ETRNFTY; 200434, ETSNKES; 200435, ETTEQDS; 200436, ETTPQYA; 200437, ETVQHPY; 200438, ETYENKA; 200439, ETYQQHT; 200440, EVAGGGV; 200441, EVATFDN; 200442, EVATGHP; 200443, EVKITVR; 200444, EVLGKDV; 200445, EVMPPQH; 200446, EVQIEGI; 200447, EVQNLSV; 200448, EVQTHIQ; 200449, EVQTIVT; 200450, EVRKPPI; 200451, EVSDGGV; 200452, EVSEAAG; 200453, EVSPAMM; 200454, EVTGDEQ; 200455, EVYGNDS; 200456, EWNVTGT; 200457, EYNTMDK; 200458, EYNVDDR; 200459, EYQAVAI; 200460, EYTQQSL; 200461, FALNMMS; 200462, FALVTKS; 200463, FDPGNRE; 200464, FDSVPAT; 200465, FDSVPIM; 200466, FETQITV; 200467, FEVARKN; 200468, FGAQHVE; 200469, FGGNVVI; 200470, FGKSGQA; 200471, FGLHSVI; 200472, FGLRSGT; 200473, FGLSKPL; 200474, FGSNMEH; 200475, FIQTSQW; 200476, FITKQVH; 200477, FKENILG; 200478, FKPMHSH; 200479, FKRSAQD; 200480, FKTHLQN; 200481, FLSHPHI; 200482, FMAEKHR; 200483, FMGATGW; 200484, FMKPVGH; 200485, FMSNINK; 200486, FNDHEAQ; 200487, FNGENEK; 200488, FNSHLMK; 200489, FNSTDGE; 200490, FPVGIAV; 200491, FQALHAN; 200492, FQQKERL; 200493, FQVGEDI; 200494, FREKWPP; 200495, FRQQPPL; 200496, FSKEQIE; 200497, FSQPRVP; 200498, FSTKPDR; 200499, FTGSRVQ; 200500, FTKTHHP; 200501, FVDMNFA; 200502, FVDTGGF; 200503, FVIGAVN; 200504, FWLEQDP; 200505, GADKGNE; 200506, GADKVGR; 200507, GAHSHPW; 200508, GAMSDRE; 200509, GDGNDKS; 200510, GEHDQTP; 200511, GESENSA; 200512, GESGDSA; 200513, GFDTRKF; 200514, GFENSIS; 200515, GFQPQGR; 200516, GFTHDIR; 200517, GGDVVSG; 200518, GGGDDRK; 200519, GGGTDEK; 200520, GGKMFTI; 200521, GGLHAML; 200522, GGVWEEE; 200523, GHDQYVI; 200524, GHEREHQ; 200525, GHKPPFH; 200526, GIIMKPH; 200527, GIRGVNP; 200528, GKDLMPW; 200529, GKDSRFN; 200530, GKEASVD; 200531, GLADPPP; 200532, GMQYKNH; 200533, GNGQPET; 200534, GNIIRVH; 200535, GNMPSKH; 200536, GNPDGNH; 200537, GNQPVQK; 200538, GPDQGSA; 200539, GPGGDTQ; 200540, GPINSIF; 200541, GPVRNNG; 200542, GQDNCCD; 200543, GQDQTGE; 200544, GQHNNDP; 200545, GQITTPW; 200546, GQMSVPW; 200547, GSDLHGI; 200548, GSDTMIM; 200549, GSEHQDS; 200550, GSKAGHN; 200551, GSPLQSR; 200552, GSPPNID; 200553, GTDTSNI; 200554, GTSNELE; 200555, GTTMFTD; 200556, GVALLPR; 200557, GVDGAAN; 200558, GVGAGED; 200559, GVIKADR; 200560, GVMEVPN; 200561, GVMQHMR; 200562, GVNAQPD; 200563, GVRISEK; 200564, GWNEKGE; 200565, GWNQHHT; 200566, GWQINHP; 200567, GYMKQQH; 200568, GYMRNMG; 200569, HALGAQH; 200570, HALGQYK; 200571, HANISPR; 200572, HANMGRF; 200573, HDIKGKF; 200574, HDKPEIQ; 200575, HDQVFKE; 200576, HDSASEA; 200577, HDSDLRH; 200578, HDSDMKN; 200579, HEHATPE; 200580, HEKNDSP; 200581, HESNAAE; 200582, HFDNYNT; 200583, HGAGMDT; 200584, HGAGVNW; 200585, HGALDPL; 200586, HGDEVDN; 200587, HGDTGDA; 200588, HGDVLAF; 200589, HGGADEY; 200590, HGMPFGT; 200591, HGPFIKS; 200592, HGTHDQN; 200593, HGTLSVR; 200594, HGVIYEI; 200595, HGVPDQG; 200596, HHADTGE; 200597, HHDPLFQ; 200598, HHSDSMS; 200599, HHSGDQL; 200600, HIPNAYY; 200601, HITRPGY; 200602, HKERAVL; 200603, HKNYEGW; 200604, HKQFAHS; 200605, HKTFATL; 200606, HLADATN; 200607, HLALAVS; 200608, HLKSAML; 200609, HMGVNTR; 200610, HIMNLTAR; 200611, HNAFTTL; 200612, HNAYMTI; 200613, HNDSSTN; 200614, HNGDALD; 200615, HNGDNSM; 200616, HPIAMNR; 200617, HPTMMLP; 200618, HQAIERF; 200619, HQDASQG; 200620, HQEDVND; 200621, HQEHGPV; 200622, HQKLIGC; 200623, HQNDSEA; 200624, HQQPVKV; 200625, HQVKSTS; 200626, HQYQLKQ; 200627, HRDSDDH; 200628, HRIESSK; 200629, HSDSDDS; 200630, HSETKVE; 200631, HSLMGVL; 200632, HSLQDAI; 200633, HSNDIPQ; 200634, HSSDMQP; 200635, HSTEFAY; 200636, HSYDEEE; 200637, HTDHPQA; 200638, HTDSDIA; 200639, HTESPKT; 200640, HTGDLTE; 200641, HTGPMIK; 200642, HTLTASD; 200643, HTPSPEQ; 200644, HVAPAEH; 200645, HVVSSTW; 200646, HYNLESL; 200647, HYQYRDP; 200648, IALEQAT; 200649, IAVEQHI; 200650, IAYKMSQ; 200651, IDAQQIQ; 200652, IDHDAHE; 200653, IDKQDQT; 200654, IDNDLKA; 200655, IDSSASP; 200656, IDTEHAQ; 200657, IDTGQYD; 200658, IEIKMAK; 200659, IENNSET; 200660, IETSQPG; 200661, IFITPGG; 200662, IGAQQFE; 200663, IGDKPED; 200664, IGDRDFW; 200665, IGNEMTQ; 200666, IGSEKEF; 200667, IGYNRQS; 200668, IHAHILT; 200669, IHAISGR; 200670, IHDKPEE; 200671, IHENDPD; 200672, IHNKIPH; 200673, IHNQSHW; 200674, IHTAEIN; 200675, IIDSAGK; 200676, IIKPMIG; 200677, IIPSSTL; 200678, IISRNQT; 200679, IITQPMK; 200680, IKANAFI; 200681, IKTYPNV; 200682, ILGRNLM; 200683, ILIKNPA; 200684, ILLKVQP; 200685, ILNKPPI; 200686, ILNMEEW; 200687, IMNEYTD; 200688, INDAPVS; 200689, INDMSTF; 200690, INSDSPQ; 200691, INSEYTL; 200692, IQIDTGS; 200693, IQQDHKE; 200694, IQSDQES; 200695, IQTHLDD; 200696, IRDSVRH; 200697, IRMSLKD; 200698, IRTSTHN; 200699, ISAQVGD; 200700, ISDITPI; 200701, ISDYGDT; 200702, ISEPRAK; 200703, ISGMQDS; 200704, ISIQFKN; 200705, ISRDHFY; 200706, ISSFSTL; 200707, ISTDKSE; 200708, ISVAATC; 200709, ISVSMTV; 200710, ITAIETS; 200711, ITDMHMR; 200712, ITGTPGE; 200713, ITMQITK; 200714, ITQAEAV; 200715, ITSREDE; 200716, ITSTQGE; 200717, ITTDSHV; 200718, IVMAQSM; 200719, IVQPAEM; 200720, KAHNTLF; 200721, KAMHVQM; 200722, KAQWEAK; 200723, KDDPILM; 200724, KDDPTGV; 200725, KDDYSGA; 200726, KDGTHEE; 200727, KDMIMVM; 200728, KDNAQRY; 200729, KDNDSPK; 200730, KDNHPKV; 200731, KDSDEAH; 200732, KDTADGD; 200733, KDTTRVS; 200734, KEAHRNL; 200735, KEAMKDR; 200736, KEAQVMR; 200737, KEKFEAK; 200738, KEKQHNQ; 200739, KEKTDTL; 200740, KEPPSKF; 200741, KEVNPRS; 200742, KFYQSSD; 200743, KGLNAMY; 200744, KGMQEFR; 200745, KGNDIEM; 200746, KGVNYMT; 200747, KHDDVSD; 200748, KHTTQIQ; 200749, KIDGAEE; 200750, KIEKDVR; 200751, KIIHADW; 200752, KIKEDGV; 200753, KIMGSHI; 200754, KIVENTP; 200755, KLDIVVR; 200756, KLHAGMT; 200757, KLIVATG; 200758, KMVQIVN; 200759, KNDDAAT; 200760, KNDDAGD; 200761, KNDLPQL; 200762, KNEARQL; 200763, KNNFEVF; 200764, KPEGYFL; 200765, KPGKIFE; 200766, KPSFIQS; 200767, KPVNFVS; 200768, KQDTADL; 200769, KQFTQNP; 200770, KQHAVFV; 200771, KQIPMIS; 200772, KQIVRDY; 200773, KQPEQRS; 200774, KQSDEHD; 200775, KQYNEIK; 200776, KRANFDS; 200777, KRQFGEV; 200778, KRQPMME; 200779, KSDPAWT; 200780, KSDVIGE; 200781, KSHSDDA; 200782, KSQDEKI; 200783, KSRVPNE; 200784, KTDNYRP; 200785, KTGLKPP; 200786, KTHAMFP; 200787, KTHLPGI; 200788, KTMNEDI; 200789, KTMNTEK; 200790, KTYTTNS; 200791, KVMVKSE; 200792, KVQTQHA; 200793, KWHTAEL; 200794, KYDMGSR; 200795, KYDSGQK; 200796, KYEQMHF; 200797, KYMTAGL; 200798, KYNDVFL; 200799, KYPNQVF; 200800, LAITYTS; 200801, LAMSSFN; 200802, LAVIEQM; 200803, LDDTGNR; 200804, LDGPGAP; 200805, LDKLMVV; 200806, LDLSEKQ; 200807, LDTMIQH; 200808, LDVHNNE; 200809, LEAISNM; 200810, LEFEGDN; 200811, LELLAGP; 200812, LEQSVPK; 200813, LESHSQI; 200814, LGALSTF; 200815, LGDHDGV; 200816, LGESGGD; 200817, LGKIHQQ; 200818, LGLQDSS; 200819, LGMRDQW; 200820, LGNNEEN; 200821, LGNQDAP; 200822, LGTPEWI; 200823, LGTYLEG; 200824, LGVQPYS; 200825, LGVVPYK; 200826, LHALSDL; 200827, LHFAMPD; 200828, LHMNAPF; 200829, LHTIRPV; 200830, LILRSQP; 200831, LITQYSS; 200832, LLDGYER; 200833, LMDHQPT; 200834, LMGAMPR; 200835, LMSDVSQ; 200836, LNDLVEL; 200837, LNDQKET; 200838, LNDTPKH; 200839, LNGDTNL; 200840, LNGELSL; 200841, LNGGGVE; 200842, LNGQEST; 200843, LNHNFNM; 200844, LNVDLSF; 200845, LPAYSPY; 200846, LPGAVVR; 200847, LPIRMHG; 200848, LPMITKL; 200849, LPPNDYY; 200850, LQAGNEV; 200851, LQDDMPD; 200852, LQDPFNY; 200853, LQEAQIT; 200854, LQERGIE; 200855, LQTVTTT; 200856, LQVQEDI; 200857, LQYTQGK; 200858, LRIGNSM; 200859, LRISADY; 200860, LRITTTH; 200861, LRSEVMQ; 200862, LSAPHAK; 200863, LTGDPTQ; 200864, LTHGGQK; 200865, LTHGQNK; 200866, LTINQRI; 200867, LTMHVRS; 200868, LTMPIDQ; 200869, LTTGFMK; 200870, LVDNHQD; 200871, LVQSLND; 200872, LVYPHET; 200873, LWATPEL; 200874, LYQTPAL; 200875, LYRGFNE; 200876, LYVQAGI; 200877, MADQQPG; 200878, MADSIST; 200879, MAQAAWV; 200880, MASQNPE; 200881, MDHGAVD; 200882, MDNIQQM; 200883, MDNTGSE; 200884, MDYAAGD; 200885, MEDVSGV; 200886, MEHAQDS; 200887, MEYAITH; 200888, MGGFAQR; 200889, MGGSDDS; 200890, MGIMRPH; 200891, MGVLNHM; 200892, MIEIPHL; 200893, MIHDYNH; 200894, MISALVD; 200895, MKILTAQ; 200896, MKIMTHN; 200897, MLEQRQR; 200898, MLLHSKT; 200899, MLMQADI; 200900, MLPGTFS; 200901, MLQIQDI; 200902, MMRTHGN; 200903, MNDHGHD; 200904, MNDHVAV; 200905, MNEALWI; 200906, MNEHGGV; 200907, MNGADGN; 200908, MNKDAGK; 200909, MNTAEFW; 200910, MNTYHHT; 200911, MNVIHMK; 200912, MNVTGEN; 200913, MPAKDHK; 200914, MPAKFLQ; 200915, MPGQVPD; 200916, MPHNDAA; 200917, MPSDQNE; 200918, MQAAEQT; 200919, MQEQYSY; 200920, MQGVENI; 200921, MQHNIYS; 200922, MQHQRLT; 200923, MQHQRQN; 200924, MQMPRMP; 200925, MQTQGGD; 200926, MQVREIR; 200927, MRERIHQ; 200928, MRNMDMY; 200929, MRVMGGM; 200930, MRVMPNH; 200931, MSARVEE; 200932, MSGGEIL; 200933, MSLTRNN; 200934, MSSDHHE; 200935, MTAIKTC; 200936, MTARQTI; 200937, MTDTFKR; 200938, MTDTGAQ; 200939, MTGGTRL; 200940, MTGKLPP; 200941, MTSPMPD; 200942, MVSQQPW; 200943, MYDMAAM; 200944, NADGSFT; 200945, NADHVSE; 200946, NAISEIK; 200947, NAQHEQL; 200948, NAQLHNK; 200949, NATEPKS; 200950, NDAVDQS; 200951, NDDDYAE; 200952, NDDEYTG; 200953, NDFAQAE; 200954, NDFGYPV; 200955, NDGFAYI; 200956, NDHGDVA; 200957, NDKNSPE; 200958, NDKTDTI; 200959, NDNIAVK; 200960, NDQEVRC; 200961, NDVTAGG; 200962, NGDSHTP; 200963, NGGGEAL; 200964, NGGQEAM; 200965, NGTEVSS; 200966, NGTQEHN; 200967, NGVVEMQ; 200968, NGYRSIA; 200969, NHDDSSP; 200970, NIDSMRT; 200971, NIVGTYY; 200972, NKEYPQE; 200973, NKGVYPF; 200974, NKPKSGE; 200975, NKSQTTN; 200976, NLGGDQE; 200977, NLINYTG; 200978, NLLGDLA; 200979, NLTEAPW; 200980, NMDQANV; 200981, NMDSASI; 200982, NMGENTS; 200983, NMNIELN; 200984, NMRVTMM; 200985, NNAWHEG; 200986, NNGDKPF; 200987, NPNGNIY; 200988, NPNLHPY; 200989, NPPDNRR; 200990, NPQAEMV; 200991, NPVLPVL; 200992, NQDLAQI; 200993, NQGPGNR; 200994, NQNLRYT; 200995, NRDQMNC; 200996, NRGWPQE; 200997, NRMVSMT; 200998, NRNAPVY; 200999, NRQEMRY; 201000, NSDNEAV; 201001, NSHLQHL; 201002, NSMLRGL; 201003, NSMYRTS; 201004, NSQALEQ; 201005, NSQFSRM; 201006, NTAPEDC; 201007, NTAQKIW; 201008, NTASDLQ; 201009, NTDNPRH; 201010, NTDVYTH; 201011, NTIIMKN; 201012, NTKEDIT; 201013, NTNFHAK; 201014, NTSDNTV; 201015, NTYNDPE; 201016, NVAREIK; 201017, NVDSTPS; 201018, NVEQGDR; 201019, NVGDEPF; 201020, NVHDTPV; 201021, NVNHSDE; 201022, NVQVDRK; 201023, NVVEHHP; 201024, NVVLDSP; 201025, NVYSINV; 201026, NYDPLLI; 201027, NYQNLAR; 201028, PAESDWL; 201029, PAEWDKS; 201030, PALYNKI; 201031, PATQIKW; 201032, PDANKKF; 201033, PDHNKEE; 201034, PDTLSRK; 201035, PEIPDWM; 201036, PEMVKPW; 201037, PENSHPK; 201038, PENVKYR; 201039, PEPKHWI; 201040, PFDGLLL; 201041, PFKMHPG; 201042, PGDAQNS; 201043, PGLSNTD; 201044, PGSDNQT; 201045, PHNHGSW; 201046, PHSDGGN; 201047, PIAQSPW; 201048, PISQNPW; 201049, PISSDPW; 201050, PISVYKT; 201051, PKFDKGW; 201052, PLHGPTR; 201053, PLQDNTP; 201054, PLTAIPP; 201055, PMGEYKD; 201056, PMKVVPH; 201057, PMPIGKI; 201058, PMQDGKH; 201059, PNAQTSE; 201060, PNDPSDV; 201061, PNLTENQ; 201062, PNLTRGY; 201063, PQAQHNK; 201064, PQHMSHL; 201065, PQQGGGE; 201066, PQVGEHP; 201067, PRMfHFTE; 201068, PSADNRD; 201069, PSDQFHE; 201070, PSQDHQD; 201071, PSQKAHF; 201072, PTKDSEE; 201073, PVANQTE; 201074, PVPPADK; 201075, PVSSHIF; 201076, PWKSNIP; 201077, PYMDLPR; 201078, PYQNLRT; 201079, PYTYQSS; 201080, QADERSR; 201081, QAELPQP; 201082, QAGDVRE; 201083, QAHSADL; 201084, QAKNDAC; 201085, QASYTSR; 201086, QATGITL; 201087, QDDHEHA; 201088, QDDNVTQ; 201089, QDGHPGQ; 201090, QDGNAEV; 201091, QDGNGAM; 201092, QDHGMGE; 201093, QDKDNSI; 201094, QDKVRLY; 201095, QDLVLTV; 201096, QDQSNNN; 201097, QEHGMTA; 201098, QEHILEN; 201099, QETEHQV; 201100, QFSGAEE; 201101, QGDKQGR; 201102, QGFKSSI; 201103, QGGVEHE; 201104, QGNDPEN; 201105, QGQDPTD; 201106, QHDQENK; 201107, QHQVDKR; 201108, QIAIDRH; 201109, QKDADPN; 201110, QKPHQNG; 201111, QKVNEWY; 201112, QKYQSGF; 201113, QLDILTR; 201114, QLEQGVT; 201115, QMKKYFE; 201116, QMSEKER; 201117, QNADNEK; 201118, QNDHMGV; 201119, QNDKNEV; 201120, QNDPEKV; 201121, QNDWDEE; 201122, QNGQDTS; 201123, QNGQNDT; 201124, QNGVDES; 201125, QNTLDFI; 201126, QNVPDWV; 201127, QQDNSES; 201128, QQGEKPR; 201129, QQITPHR; 201130, QQKSDAD; 201131, QQNNVHK; 201132, QQSGDEV; 201133, QQTDPQE; 201134, QQTLTDN; 201135, QQTTILK; 201136, QRPASGG; 201137, QRQERIL; 201138, QSADQKW; 201139, QSDFLGM; 201140, QSINFVP; 201141, QSYNYVP; 201142, QTAEGMN; 201143, QTAMVDM; 201144, QTAYEGE; 201145, QTDQTNV; 201146, QTQNLDW; 201147, QTTATSD; 201148, QVEPRDV; 201149, QVGDTKT; 201150, QVGGDEA; 201151, QVQDDSE; 201152, QVRDYQR; 201153, QVTKQIH; 201154, QYDMMKL; 201155, QYEPTKK; 201156, QYTETPE; 201157, RAAGISF; 201158, RADPLRF; 201159, RAMRAEN; 201160, RDDGLFI; 201161, RDFSPPV; 201162, RDNERDH; 201163, RDQDDTV; 201164, RDQSETR; 201165, RDTHEEP; 201166, REDDGNT; 201167, REPNQMK; 201168, RESINRT; 201169, RESIPFH; 201170, REVPFKQ; 201171, RFELGSL; 201172, RGQAFGS; 201173, RGSTMQV; 201174, RHATVGT; 201175, RHTVPLT; 201176, RIETRVT; 201177, RINKEYQ; 201178, RKEQVYI; 201179, RKSQLPD; 201180, RLDDNAD; 201181, RLDSEAK; 201182, RLEDAEG; 201183, RMLESSR; 201184, RMLGATT; 201185, RMMAQQM; 201186, RMTENMR; 201187, RNEMYIH; 201188, RNWQAHE; 201189, RPGAIYN; 201190, RPTALMV; 201191, RQEQVAK; 201192, RQESIKF; 201193, RQGMTEK; 201194, RQGPPYM; 201195, RQIQYPP; 201196, RQTADMW; 201197, RRENSNF; 201198, RSDQRGG; 201199, RSGVYGQ; 201200, RSLGTER; 201201, RSPTTGS; 201202, RSSMDRA; 201203, RTGQQTW; 201204, RTLVGRE; 201205, RTMNIVN; 201206, RVEGYLH; 201207, RVISMER; 201208, RVKIPEM; 201209, RWNSDTL; 201210, RYKEMEW; 201211, SAANSEG; 201212, SAHDLKE; 201213, SAITPTL; 201214, SANEVDG; 201215, SANSGPE; 201216, SATEDKI; 201217, SAVSLLN; 201218, SDETDKL; 201219, SDGSDTR; 201220, SDGTIQP; 201221, SDIERHL; 201222, SDPPGDS; 201223, SDQGPVH; 201224, SDTAIQQ; 201225, SDTPEWM; 201226, SDVSQVY; 201227, SEHEPRT; 201228, SEHQVNQ; 201229, SEMMTRF; 201230, SEQADIC; 201231, SETAGTQ; 201232, SETSNDP; 201233, SEVSANA; 201234, SFAYGKV; 201235, SFVSEKK; 201236, SGALGNW; 201237, SGDYIQM; 201238, SGGVPRG; 201239, SGIQDDA; 201240, SGNAPEV; 201241, SGNYDED; 201242, SGSGDHI; 201243, SGTTDFW; 201244, SGVYPQR; 201245, SHDKHDE; 201246, SHGDASN; 201247, SHNSDEN; 201248, SHTDNLA; 201249, SIGDYKK; 201250, SIGEALI; 201251, SIVERVN; 201252, SKFDEKR; 201253, SKGMYVN; 201254, SKPLVYT; 201255, SKSDVSD; 201256, SKTMGLF; 201257, SLGETTE; 201258, SLLDQAA; 201259, SLQGEII; 201260, SLYGTTL; 201261, SMDQEKE; 201262, SMLHTAL; 201263, SMPNGEH; 201264, SMTRMMT; 201265, SNAGSEE; 201266, SNDEVMQ; 201267, SNDSVAE; 201268, SNEYKPW; 201269, SNGQDSM; 201270, SPANGDC; 201271, SPGMEWM; 201272, SPKEMED; 201273, SPRPPTY; 201274, SQDVPQS; 201275, SQEDDRP; 201276, SQPTYSR; 201277, SREMKPW; 201278, SRENNDM; 201279, SRFASGT; 201280, SRVREGL; 201281, SSIAAIL; 201282, SSPLHRV; 201283, SSVRGLY; 201284, SSYNEDE; 201285, STADGGL; 201286, STDAYPW; 201287, STDQEAM; 201288, STHDGGM; 201289, STHGDDD; 201290, STMPEKW; 201291, STVAIRQ; 201292, SVDNHNS; 201293, SVEHGSV; 201294, SVERDSP; 201295, SVGASVD; 201296, SVNAESD; 201297, SVSSQDE; 201298, SVTLLSK; 201299, SYEVKKY; 201300, SYGGKQM; 201301, TAAMDFT; 201302, TADDNRK; 201303, TADNAQI; 201304, TALHYLS; 201305, TAMQDNQ; 201306, TANENPQ; 201307, TAREIQK; 201308, TAVSETV; 201309, TDAGQTS; 201310, TDASQTA; 201311, TDAVPPI; 201312, TDHMVNV; 201313, TDIAQVT; 201314, TDKRQYI; 201315, TDNDTSA; 201316, TDNVTSL; 201317, TDQGQPH; 201318, TDQMNTS; 201319, TDQTYAK; 201320, TDQVPRE; 201321, TDSSVLY; 201322, TDTDVER; 201323, TEDKLTS; 201324, TEEKPWL; 201325, TEHGATQ; 201326, TESDSYY; 201327, TEVTSAK; 201328, TFRAQNM; 201329, TFTPNIR; 201330, TGDQFPS; 201331, TGEFKQK; 201332, TGFHVTL; 201333, TGHGEDP; 201334, TGLNPHT; 201335, TGNHDMH; 201336, TGPQIHK; 201337, TGSTKWI; 201338, TGTMGED; 201339, TGVHSIF; 201340, TKMTHGP; 201341, TKRVPPE; 201342, TLSDLGN; 201343, TMVTYPK; 201344, TNDVGGE; 201345, TNEHSNI; 201346, TNFRVQV; 201347, TNGDDWW; 201348, TNGGQTD; 201349, TNGHDPA; 201350, TNGIVSE; 201351, TNIGTLR; 201352, TNPNSKH; 201353, TNQDAGP; 201354, TNTALRF; 201355, TNTRESE; 201356, TPHGMTK; 201357, TQADEAV; 201358, TQGNEIE; 201359, TQIGTPY; 201360, TQQDVKI; 201361, TQRNYNQ; 201362, TRFDGPR; 201363, TRQFERT; 201364, TRQQDWL; 201365, TSDTPVL; 201366, TSGPVTR; 201367, TSTDNKH; 201368, TSTMAIE; 201369, TTDQYGA; 201370, TTINEGE; 201371, TTIRMNQ; 201372, TTMDMMM; 201373, TTPGFMI; 201374, TTQQTDG; 201375, TTVPVKL; 201376, TVDQGAP; 201377, TVGYKPQ; 201378, TVMEASI; 201379, TVNKEEV; 201380, TVTANMV; 201381, TYQLTQL; 201382, TYVPLEM; 201383, VADSKEA; 201384, VAKTDEC; 201385, VATHFTN; 201386, VDNYESS; 201387, VDPSPMR; 201388, VDSAGQQ; 201389, VDSEPNV; 201390, VDTGDAP; 201391, VDVRIRT; 201392, VDWDSRM; 201393, VEDQDHE; 201394, VEEGTWD; 201395, VEGSMEF; 201396, VEHGSSA; 201397, VEHQIEV; 201398, VEIYKNK; 201399, VENGYYT; 201400, VESPLFP; 201401, VESPQTS; 201402, VETSLDA; 201403, VETSSGD; 201404, VEYTVSL; 201405, VFIGVGT; 201406, VFSHQVL; 201407, VFSNPSF; 201408, VGDYRRV; 201409, VGGAADD; 201410, VGHKFPV; 201411, VGLTEQQ; 201412, VGNDDTS; 201413, VGNDQMS; 201414, VGNGQDE; 201415, VGQDQPS; 201416, VGTQTQD; 201417, VGVQQTD; 201418, VHLIRSP; 201419, VILDNSV; 201420, VINRAYN; 201421, VITRPVM; 201422, VKKAEWQ; 201423, VKNERWM; 201424, VLNKDDE; 201425, VLNPYGT; 201426, VLPDSRK; 201427, VMEKVEH; 201428, VMLNRIG; 201429, VMTNLHK; 201430, VMTSEPN; 201431, VNGQSQC; 201432, VNMLPQL; 201433, VNMNGQE; 201434, VNREETP; 201435, VNTQPED; 201436, VNVVKYP; 201437, VPDVHQP; 201438, VQGDLQE; 201439, VQGGSEG; 201440, VQISLAH; 201441, VQLDKWT; 201442, VRERSYQ; 201443, VRIKEDR; 201444, VRMTQNT; 201445, VRSNSNV; 201446, VRTMSPL; 201447, VSDSDAN; 201448, VSDYSDD; 201449, VSEMKRY; 201450, VSKTQVG; 201451, VSTQDAG; 201452, VTADTYA; 201453, VTDALVT; 201454, VTDGSQS; 201455, VTDPGLQ; 201456, VTGGELE; 201457, VTHDSGE; 201458, VTLRERT; 201459, VTSDNKM; 201460, VTSPDGE; 201461, VTSQETE; 201462, VVDSGGH; 201463, VVDSRVR; 201464, VVMYQTS; 201465, VVNSIED; 201466, VVSTLEK; 201467, VYDKRTL; 201468, WAGRTIN; 201469, WANSDPD; 201470, WDEETAD; 201471, WDGNDPV; 201472, WDPPVSD; 201473, WDVKFSV; 201474, WEDSYRI; 201475, WEPGDHI; 201476, WEQILDH; 201477, WGDILVT; 201478, WIDHGPY; 201479, WIRTDDF; 201480, WIVDTAP; 201481, WKDTLRQ; 201482, WKKLNIE; 201483, WNGNQNW; 201484, WNTSNVD; 201485, WPEVWTD; 201486, WPVEQKK; 201487, WQVSQPE; 201488, WSQQQPR; 201489, WTADTEV; 201490, WVQNSMK; 201491, YAGIPLI; 201492, YARPEWP; 201493, YDARARY; 201494, YDDVRAD; 201495, YDHSVEK; 201496, YDSGKQE; 201497, YDSKQPE; 201498, YDSMLAT; 201499, YDSQSEE; 201500, YDSVVQH; 201501, YDVTDRN; 201502, YEANVNT; 201503, YEDSATM; 201504, YEHAQEA; 201505, YEYDGSW; 201506, YFERVKS; 201507, YGAGDDM; 201508, YGAVTSE; 201509, YGDNIIK; 201510, YGEEKSV; 201511, YGELGKK; 201512, YGMKVSL; 201513, YGNQYNS; 201514, YGTIDHA; 201515, YHDALFH; 201516, YHSEYNE; 201517, YIDAQLR; 201518, YIQKSAI; 201519, YKAGSWV; 201520, YKEIQPR; 201521, YKLTPAL; 201522, YLAKSML; 201523, YMDQHPL; 201524, YMHKTSM; 201525, YNAFAEE; 201526, YNMPGNR; 201527, YNMSNRQ; 201528, YNRDNPD; 201529, YNTRAVH; 201530, YNYGMDA; 201531, YPNLHHL; 201532, YPTNDDK; 201533, YQGPDTD; 201534, YQMERGR; 201535, YQTGESE; 201536, YRNDDID; 201537, YSGVDNV; 201538, YSNEYIQ; 201539, YTDNTML; 201540, YTGDV™; 201541, YTIMGSP; 201542, YVPISQH; 201543, YYDVPSK; and 201544, YYQDETV.

The Fit4Function pipeline presents a significant conceptual and technological advance over prior AAV engineering studies, including those that leverage ML. Conventional in vivo selections use sequential rounds to narrow the focus of sequence exploration to a handful of top candidates, which may not have other traits required for translation to preclinical and clinical trials. Simultaneously engineering multiple traits into AAV capsids or other proteins of interest has become an important but challenging goal. To date, most protein engineering efforts, including those leveraging ML, have focused on optimizing a single function, e.g. generating more efficiently produced and diversified AAV capsid libraries but stopping short of multi-trait prediction. A few groups have gone beyond single trait engineering by combining multiple previously validated functional structures into a single protein, e.g., by recombining structurally independent segments from different channelrhodopsins possessing known functions, localizations, and photocurrent properties of interest, or by applying protein design tools to filter out variants that do not meet additional characteristics such as solubility and immunogenicity. A few groups have gone beyond single trait engineering by combining multiple previously validated functional structures into a single protein, e.g., by recombining structurally independent segments from different channelrhodopsins possessing known functions, localizations, and photocurrent properties of interest, or by applying protein design tools to filter out variants that do not meet additional characteristics such as solubility and immunogenicity. However, as these strategies rely on the recombination of multiple existing functional structures into a single protein or the use of third-party protein design tools, they cannot be broadly generalized to engineer multiple de novo functions. A key obstacle to combining multiple ML models that predict different traits is the aggregated error that increases with each added model. The Fit4Function approach directly tackles this problem by leveraging a moderately sized, all viable, low-bias (ML-designed) library to generate highly reproducible data for multi-trait learning with a low false positive rate. This allows the models to be applied in different combinations with a low risk of aggregating significant error. MultiFunction libraries can thus be generated to more efficiently explore the vast sequence space for multi-trait capsids.

The Fit4Function approach can help to reduce the need for extensive screening in macaques in two ways. Firstly, the unique features of Fit4Function libraries enable the quantitative assessment of capsid biodistribution and top candidate selection in multiple organs from just a single round of screening. It is only necessary to screen a Fit4Function library once for a given function to then predict the functionality of sequences that were not contained in the original library. In contrast, it typically requires 2-6 rounds of in vivo screening to reliably identify top candidates from conventional selections, and the data from these screens cannot be used to accurately predict the traits of variants not tested in that screen. This means that the Fit4Function approach can be used to design libraries full of diverse and promising candidates for more efficient screening in macaques or other animals or assays. Secondly, unlike existing screening strategies, our approach can systematically determine the functional assays or combinations thereof that drive cross-species transferability. As the Fit4Function approach is applied to more NHP functions of interest (e.g., BBB-crossing), it will become apparent whether it is worthwhile to continue screening in mice or other animals for those functions. This can inform the choice of cell or animal models to perform screens in and develop vectors that are more likely to translate preclinically and clinically.

As with other ML-guided approaches, Fit4Function can be more challenging to implement with assays that produce low quality data due to lower detection sensitivities. For example, data reproducibility and subsequent model performance can be bottlenecked by in vivo transduction assays in some organs due to the inherent tropism of the parental capsid, inter-animal variability, and technical challenges related to tissue sampling. One approach to improve data quality with low sensitivity assays may be to use smaller Fit4Function libraries, because reducing library diversity increases the sampling of each individual variant and therefore the quality of the screening data. A second limitation that affects any multi-objective engineering effort is that variants that are maximally optimized for multiple objectives may not exist, especially in cases where performance on functions are negatively correlated. While Fit4Function cannot overcome this fundamental problem, it provides the means to efficiently search the vast production fit sequence space for variants that are reasonably well optimized for multiple traits.

With continued application across experiments and laboratories, the Fit4Function approach should enable the assembly of a vast ML atlas that can accurately predict the performance of AAV capsid variants across dozens of traits and inform the design of screening pipelines. In addition, the Fit4Function approach should translate to engineering other proteins that are amenable to quantitative, high-throughput screening of libraries that are diversified at a defined set of residues.

The following materials and methods were employed in the above examples.

### Training and Assessment Library Design

The training and assessment libraries were designed to contain 150K nucleotide sequences each. The libraries were composed of 64.5K unique and 10K shared amino acid sequences generated by uniformly sampling all 20 amino acids at each position. The 74.5K variants were duplicated via 7-mer replication. 1K sequences containing stop codons were included to detect problems with cross packaging. In total, each library comprised a final set of 150K sequences.

### Capsid Library Synthesis

To produce synthetic library inserts, lyophilized DNA oligonucleotide libraries (Agilent G7223A) or NNK hand mixed primers (IDT) were spun down at 8000 RCF for 1 minute, resuspended in 10 μL UltraPure DNase/RNase-Free Distilled Water (Thermo Fisher Scientific, 10977015), and incubated at 37° C. for 20 minutes. For pooled synthetic oligonucleotide libraries, the following primer format was used: 5′-GTATTCCTTGGTTTTGAACCCAACCGGTCTGCGCCTGTGC-(NNN)₇-TTGGCACTCTGGTGGTTTGTGGCCAC. (SEQ ID NO: 199470, where the 7-mer contained 21 (7×3) nucleotides). To produce NNK inserts, the AAV9_K449R_Forward (CGGACTCAGACTATCAGCTCCC (SEQ ID NO: 199471)) and AAV9_K449R_NNK_Reverse (5′-GTATTCCTTGGTTTTGAACCCAACCGGTCTGCGCCTGTGC(MNN)₇TTGGGCACTCTGGTGGTTTG TG) (SEQ ID NO: 199472; where “N” represents A, C, G, or T and “M” represents A or C) primers were used.

To amplify the oligonucleotide libraries and incorporate them into an AAV9 (K449R) template, 2 μL of the resuspended pooled oligonucleotide library or NNK-based library was used as an initial reverse primer along with 0.5 μM AAV9_K449R_Forward primer in a 25 μL PCR amplification reaction using Q5 Hot Start High-Fidelity 2X Master Mix (NEB, M0494S). 50 ng of a plasmid containing only AAV9 (K449R) VP1 amino acids 347-586 was used as a PCR template. PCR was performed following the manufacturer's protocol with an annealing temperature of 65° C. for 20 seconds and an extension time of 90 seconds. After six PCR cycles, 0.5 μM AAV9_K449R_Reverse (GTATTCCTTGGTTTIGAACCCAACCG (SEQ ID NO: 199473)) was spiked into the reaction as a reverse primer to further amplify sequences containing the oligonucleotide library for an additional 25 cycles. To remove the PCR template, 1 μL of DpnI (NEB, R0176S) was added to the PCR reaction and incubated at 37° C. for one hour. Afterwards, the PCR products were cleaned using AMPure XP beads (Beckman, A63881) following the manufacturer's protocol.

The PCR insert was assembled into 1600 ng of a linearized mRNA selection vector (AAV9-CMV-Express) with NEBuilder HiFi DNA Assembly Master Mix (NEB, E2621L) at a 3:1 insert:vector Molar ratio in a 80 μL reaction volume, incubated at 50° C. for one hour, and then at 72° C. for 5 minutes. Afterwards, 4 μL of Quick CIP (NEB, M0508S) was spiked into the reaction and incubated at 37° C. for 30 minutes to dephosphorylate unincorporated dNTPs that may inhibit downstream processes. Finally, 4 μL of T5 Exonuclease (NEB M0663S) was added to the reaction and incubated at 37° C. for 30 minutes to remove unassembled products. The final assembled products were cleaned using AMPure XP beads (Beckman, A63881) following the manufacturer's protocol and their concentrations were quantified with a Qubit dsDNA HS Assay Kit (Thermo Fisher Scientific, Q32851) and a Qubit fluorometer.

mRNA Selection Vector

The mRNA selection vector (AAV9-CMV-Express) was designed to enrich for functional AAV capsid sequences by recovering capsid mRNA from transduced cells. AAV9-CMV-Express used a ubiquitous CMV enhancer and AAV5 p41 gene regulatory elements to drive AAV Cap expression. The AAV-Express plasmid was constructed by cloning the following elements into an AAV genome plasmid in the following order: a cytomegalovirus (CMV) enhancer-promoter, a synthetic intron and the AAV5 P41 promoter along with the 3′ end of the AAV2 Rep gene, which included the splice donor sequences for the capsid RNA. The capsid gene splice donor sequence in AAV2 Rep was modified from a non-consensus donor sequence CAGGTACCA to a consensus donor sequence CAGGTAAGT. The AAV9 capsid gene sequence was synthesized with nucleotide changes at S448 (TCA to TCT, silent mutation), K449R (AAG to AGA), and G594 (GGC to GGT, silent mutation) to introduce restriction enzyme recognition sites for oligonucleotide library fragment cloning. The AAV2 polyadenylation sequence was replaced with a simian virus 40 (SV40) late polyadenylation signal to terminate the capsid RNA transcript.

### Virus Production

For library production, HEK293T/17 cells (ATCC, CRL-11268) were seeded at 22 million cells per 15 cm plate the day before transfection and grown in DMEM with GlutaMAX (Gibco, 10569010) supplemented with 5% FBS and 1× non-essential amino acid solution (NEAA) (Gibco, 11140050). The next day, each plate was triple transfected with 39.93 g of total plasmid DNA encoding pHelper, RepStop encoding the AAV2 Rep genes, pUC19 at a ratio of 2:1:1, respectively, and with 10 ng of assembled library DNA. The media was exchanged for fresh DMEM with 5% FBS and 1×NEAA at 20 hours post transfection. At 60 hours, the media and cell lysates were harvested and purified following a protocol described in R. C. Challis, et al., “Systemic AAV vectors for widespread and targeted gene delivery in rodents,” Nat. Protoc. 14, 379-414 (2019).

Individual recombinant AAVs were produced in suspension HEK293T cells, using F17 media (ThermoFisher Scientific). Cell suspensions were incubated at 37° C., 8% C02, 125 RPM. 24 hours before transfection, cells were seeded in 200 mL at ˜1 million cells/mL. The day after, cells (˜2 million cells/mL) were transfected with pHelper, pRepCap and pTransgene (2:1:1 ratio, 2 ug DNA per million cells) using Transport 5 transfection reagent (Polysciences) with a 2:1 PEI:DNA ratio. Three days post-transfection, cells were pelleted at 2000 RPM for 10 minutes into Nalgene conical bottles. The supernatant was discarded, and cell pellets were stored at −20° C. until purification. Each pellet, corresponding to 200 mL of cell culture, was resuspended in 7 mL of 500 mM NaCl, 40 mM Tris-base, 10 mM MgCl₂, with Salt Active Nuclease (ArcticZymes, #70920-202) at 100 U/mL. Afterwards, the lysate was clarified at 2000 RCF for 10 minutes and loaded onto a density step gradient containing OptiPrep (Cosmo Bio, AXS-1114542) at 60%, 40%, 25%, and 15% at a volume of 5, 5, 6, and 6 mL respectively in OptiSeal tubes (Beckman, 361625). The step gradients were spun in a Beckman Type 70ti rotor (Beckman, 337922) in a Sorvall WX+ ultracentrifuge (ThermoFisher Scientific, 75000090) at 69,000 RPM for 1 hour at 18° C. Afterwards, ˜4.5 mL of the 40-60% interface was extracted using a 16-gauge needle, filtered through a 0.22 μm PES filter, buffer exchanged with 100K MWCO protein concentrators (Thermo Fisher Scientific, 88532) into PBS containing 0.001% Pluronic F-68, and concentrated down to a volume of 500 μL. The concentrated virus was filtered through a 0.22 μm PES filter and stored at 4° C. or −80° C.

### AAV Titering

To determine AAV titers, 5 μL of each purified virus library were incubated with 100 μL of an endonuclease cocktail consisting of 1000U/mL Turbonuclease (Sigma T4330-50KU) with 1× DNase I reaction buffer (NEB B0303S) in UltraPure DNase/RNase-Free distilled water at 37° C. for one hour. Next, the endonuclease solution was inactivated by adding 5 μL of 0.5M EDTA, pH 8.0 (Thermo Fisher Scientific, 15575020) and incubated at room temperature for 5 minutes and then at 70° C. for 10 minutes. To release the encapsidated AAV genomes, 120 μL of a Proteinase K cocktail consisting of 1M NaCl, 1% N-lauroylsarcosine, 100 g/mL Proteinase K (Qiagen, 19131) in UltraPure DNase/RNase-Free distilled water was added to the mixture and incubated at 56° C. for 2 to 16 hours. The Proteinase K-treated samples were then heat-inactivated at 95° C. for 10 minutes. The released AAV genomes were serial diluted between 460-460,000× in dilution buffer consisting of 1×PCR Buffer (Thermo Fisher Scientific, N8080129), 2 g/mL sheared salmon sperm DNA (Thermo Fisher Scientific, AM9680), and 0.05% Pluronic F68 (Thermo Fisher Scientific, 24040032) in UltraPure Water (Thermo Fisher Scientific). 2 μL of the diluted samples were used as input in a ddPCR supermix (Bio-Rad, 1863023). Primers and probes, targeting the ITR and CAG promoter region, were used for titration, at a final concentration of 900 nM and 250 nM, respectively (ITR2_Forward: GGAACCCCTAGTGATGGAGTT (SEQ ID NO: 199474); ITR2_Reverse: CGGCCTCAGTGAGCGA (SEQ ID NO: 199475); ITR2_Probe: CACTCCCTCTCTGCGCGCTCG (SEQ ID NO: 199476) [FAM/Iowa Black FQ Zen]; CAG Forward: TGTTCCCATAGTAACGCCAATAG (SEQ ID NO: 199477); CAG_Reverse: GTACTTGGCATATGATACACTTGATG (SEQ ID NO: 199478); CAG_Probe: TTACGGTAAACTGCCCACTTGGCA (SEQ ID NO: 199479) [FAM/Iowa Black FQ Zen]). Droplets were generated using a QX100 Droplet Generator following the manufacturer's protocol. The droplets were transferred to thermocycler and cycled according to the manufacturer's protocol with an annealing/extension of 58° C. for one minute. Finally, droplets were read on a QX100 Droplet Digital System to determine titers.

### Assessing Production Fitness

To recover only encapsidated AAV genomes for downstream analysis, 10¹¹ viral genomes were extracted using the endonuclease and Proteinase K steps outlined above (AAV Titering). After Proteinase K treatment, samples were column purified using a DNA Clean and Concentrator Kit (Zymo Research, D4033) and eluted in 25 μL elution buffer for NGS preparation.

### NGS Sample Preparation

To prepare AAV libraries for sequencing, qPCR was performed on extracted AAV genomes or cDNA to determine the cycle thresholds for each sample type to prevent overamplification. PCR amplification using equal primer pairs (1-8) (Table 30; Described in Huang et al., bioRxiv 2022.10.31.514553 (2022), the disclosure of which is incorporated herein by reference in its entirety for all purposes), was used to attach partial Illumina Read 1 and Read 2 sequences using Q5 Hot Start High-Fidelity 2× Master Mix with an annealing temperature of 65° C. for 20 seconds and an extension time of 60 seconds. Round one PCR products were purified using AMPure XP beads following the manufacturer's protocol and eluted in 25 μL UltraPure Water (Thermo Fisher Scientific). 2 μL was used as input in a second round of PCR to attach on Illumina adaptors and dual index primers (NEB, E7600S) for five PCR cycles using Q5 HotStart-High-Fidelity 2× Master Mix with an annealing temperature of 65° C. for 20 seconds and an extension time of 60 seconds. The round two PCR products were purified using AMPure XP beads following the manufacturer's protocol and eluted in 25 μL UltraPure DNase/RNase-Free distilled water (Thermo Fisher Scientific).

To quantify the amount of PCR products for NGS, an Agilent High Sensitivity DNA Kit (Agilent, 5067-4626) was used with an Agilent 2100 Bioanalyzer. PCR products were pooled and diluted to 2-4 nM in 10 mM Tris-HCl, pH 8.5 and sequenced on an Illumina NextSeq 550 following the manufacturer's instructions using a NextSeq 500/550 Mid or High Output Kit (Illumina, 20024904 or 20024907), or on an Illumina NextSeq 1000 following the manufacturer's instructions using NextSeq P2 v3 kits (Illumina, 20046812). Reads were allocated as follows: I1: 8, I2: 8, R1: 150, R2: 0.

TABLE 30
PCR1 primers

| | | | SEQ ID |
|---|---|---|---|
| Name | 5′ Handle | Sequence | NO |
| seq1_F | Read 1 | CTTTCCCTACACGACGCTCTTCCGATCTNNNNNNN | 199480 |
| | | NCCAACGAAGAAGAAATTAAAACTACTAACCCG | |
| seq2_F | Read 1 | CTTTCCCTACACGACGCTCTTCCGATCTNNNNNNN | 199481 |
| | | CCAACGAAGAAGAAATTAAAACTACTAACCCG | |
| seq3_F | Read 1 | CTTTCCCTACACGACGCTCTTCCGATCTNNNNNNC | 199482 |
| | | CAACGAAGAAGAAATTAAAACTACTAACCCG | |
| seq4_F | Read 1 | CTTTCCCTACACGACGCTCTTCCGATCTNNNNNCC | 199483 |
| | | AACGAAGAAGAAATTAAAACTACTAACCCG | |
| seq5_F | Read 1 | CTTTCCCTACACGACGCTCTTCCGATCTNNNNCCA | 199484 |
| | | ACGAAGAAGAAATTAAAACTACTAACCCG | |
| seq6_F | Read 1 | CTTTCCCTACACGACGCTCTTCCGATCTNNNCCAA | 199485 |
| | | CGAAGAAGAAATTAAAACTACTAACCCG | |
| seq7_F | Read 1 | CTTTCCCTACACGACGCTCTTCCGATCTNNCCAAC | 199486 |
| | | GAAGAAGAAATTAAAACTACTAACCCG | |
| seq8_F | Read 1 | CTTTCCCTACACGACGCTCTTCCGATCTNCCAACG | 199487 |
| | | AAGAAGAAATTAAAACTACTAACCCG | |
| seq1_R | Read 2 | GGAGTTCAGACGTGTGCTCTTCCGATCTCATCTCT | 199488 |
| | | GTCCTGCCAAACCATACC | |
| seq2_R | Read 2 | GGAGTTCAGACGTGTGCTCTTCCGATCTNCATCTC | 199489 |
| | | TGTCCTGCCAAACCATACC | |
| seq3_R | Read 2 | GGAGTTCAGACGTGTGCTCTTCCGATCTNNCATCT | 199490 |
| | | CTGTCCTGCCAAACCATACC | |
| seq4_R | Read 2 | GGAGTTCAGACGTGTGCTCTTCCGATCTNNNCATC | 199491 |
| | | TCTGTCCTGCCAAACCATACC | |
| seq5_R | Read 2 | GGAGTTCAGACGTGTGCTCTTCCGATCTNNNNCAT | 199492 |
| | | CTCTGTCCTGCCAAACCATACC | |
| seq6_R | Read 2 | GGAGTTCAGACGTGTGCTCTTCCGATCTNNNNNCA | 199493 |
| | | TCTCTGTCCTGCCAAACCATACC | |
| seq7_R | Read 2 | GGAGTTCAGACGTGTGCTCTTCCGATCTNNNNNNC | 199494 |
| | | ATCTCTGTCCTGCCAAACCATACC | |
| seq8_R | Read 2 | GGAGTTCAGACGTGTGCTCTTCCGATCTNNNNNNN | 199495 |
| | | CATCTCTGTCCTGCCAAACCATACC | |

### NGS Data Processing

Sequencing data was de-multiplexed with bcl2fastq (version v2.20.0.422) using the default parameters. The Read 1 sequence (excluding Illumina barcodes) was aligned to a short reference sequence of AAV9: reference sequence of AAV9: 5 CCAACGAAGAAGAAATTAAAACTACTAACCCGGTAGCAACGGAGTCCTATGGACAAGTGGCCAC AAACCACCAGAGTGCCCAANNNNNNNNNNNNNNNNNNNNNGCACAGGCGCAGACCGGTTGGGTT CAAAACCAAGGAATACTTCCG (SEQ ID NO: 199496). Alignment was performed with bowtie2 (version 2.4.1) (B. Langmead and S. L. Salzberg, “Fast gapped-read alignment with Bowtie 2,” Nat. Methods. 9, 357-359 (2012)) with the following parameters: −-end-to-end --very-sensitive --np 0 --n-ceil L,21,0.5 --xeq -N 1 --reorder --score-mmn L,−0.6,−0.6−5 8−3 8. Resulting sam files from bowtie2 were sorted by read and compressed to bam files with samtools (version 1.11-2-g26d7c73, htslib version 1.11-9-g2264113) (P. Danecek, et al, “Twelve years of SAMtools and BCFtools,” Gigascience. 10 (2021), doi:10.1093/gigascience/giab008; and H. Li, et al., “1000 Genome Project Data Processing Subgroup, The Sequence Alignment/Map format and SAMtools,” Bioinformatics. 25, 2078-2079 (2009)).

Python (version 3.8.3) scripts and pysam (version 0.15.4) were used to extract the 21 nucleotide insertion from each amplicon read. Each read was assigned to one of the following bins: Failed, Invalid, or Valid. Failed reads were defined as reads that did not align to the reference sequence, or that had an in/del in the insertion region (i.e., 20 bases instead of 21 bases). Invalid reads were defined as reads whose 21 bases were successfully extracted, but matched any of the following conditions: 1) Any one base of the 21 bases had a quality score (AKA Phred score, QScore) below 20, i.e., error probability >1/100, 2) Any one base was undetermined, i.e., “N”, 3) The 21 base sequence was not from the synthetic library (this case does not apply to NNK library). Valid reads were defined as reads that did not fit into either the Failed or Invalid bins. The Failed and Invalid reads were collected and analyzed for quality control purposes, and all subsequent analyses were performed on the Valid reads.

Count data for valid reads was aggregated per sequence, per sample, and was stored in a pivot table format, with nucleotide sequences on the rows, and samples (Illumina barcodes) on the columns. Sequences not detected in samples were assigned a count of 0.

### Data Normalization

Count data was read-per-million (RPM) normalized to the sequencing depth of each sample (Illumina barcode) with:

$${r}_{i , j} = \frac{{k}_{i , j}}{{\sum }_{l = 1}^{n} {k}_{i , j}} \times 1 , 000 , 000 ,$$

where r is the RPM-normalized count, k is the raw count, i=1 . . . n sequences, and j=1 . . . m samples.

As each biological sampi was run in triplicate data were aggregated for each sample by taking the mean of the RPMs:

$${μ}_{i , s} = \frac{{\sum }_{l = 1}^{p} {r}_{i , l}}{p} ,$$

across p replicates of sample s. Normalized variance was estimated across replicates by taking the coefficient of variaton (CV):

$$C {V}_{i , s} = \frac{{μ}_{i , s}}{{σ}_{i , s}} ,$$

where sigma{i,s} is the standard deviation for variant i in sample s over p replicates. Log 2 enrichment for each sequence was defined as:

$${e}_{i , s} = {log}_{2} ( \frac{{μ}_{i , s}}{{μ}_{corrected , i , t}} ) ,$$

where e is the log 2 enrichment, mu is the mean of the replicate RPMs, and t is the normalization sample. For production fitness, the sample s is the variant abundance after virus production, and the normalization sample t is the variant abundance in the plasmid pool. For functional screens, the sample s is the variant abundance of the screen, and the normalization factor t is the variant abundance after virus production. To avoid dividing by 0 in e (for NNK library processing), mu_corrected is defined as:

$${μ}_{corrected , i , t} = \{ \begin{matrix}{μ}_{i , t} , & for {μ}_{i , t} > 0 \\ 1 / {\sum }_{l = 1}^{n} {k}_{l , t} , & for {μ}_{i , t} = 0\end{matrix} \} ,$$

i.e., counts of 0 across all 3 replicates for the normalization sample were adjusted to a count of 1 across all 3 replicates.

### Production Fitness Training and Assessment

A robust ML framework was designed and used for the production fitness and Fit4Function functional mappings. A long short-term memory (LSTM) regression model with two hidden layers of 140 and 20 nodes was implemented in Keras (keras-team, GitHub-keras-team/keras: Deep Learning for humans. GitHub, (available at github.com/keras-team/keras)). RNNs, and LSTMs in particular, have been successfully applied for learning functions from biological sequence data as they are designed to capture local and distant relationships across different parts of the input sequences (D. H. Bryant, et al. “Deep diversification of an AAV capsid protein by machine learning.” Nat. Biotechnol. (2021), doi:10.1038/s41587-020-00793-4; and E. Alley, et al. “Unified rational protein engineering with sequence-based deep representation learning,” doi:10.21203/rs.2.13774/v1.). Model parameters and hyperparameters were subject to fine tuning processes but no significant performance was gained across all different functional models implemented in this study. Thus, the simplest model architecture was kept across all modeling throughout this study. The input layer was 7-mer amino acid sequences one-hot encoded into a 20×7 matrix. The target/output is the relative production (or functional) fitness score. Loss was optimized by mean-squared-error with Adam optimizer running on a learning rate of 0.001 (D. P. Kingma and J. Ba, “Adam: A Method for Stochastic Optimization” (2014), (available at arxiv.org/abs/1412.6980).). The batch size was set to 500 observations. To avoid overfitting, model training was controlled by a custom early stopping procedure where the training process was terminated if the ratio of training error to validation error dropped below 0.90.

For production fitness learning, the training size was optimized by training the framework on increments of 1K variants. Variants that were not detected (n=5,279) after virus production were filtered out from training. Model validation performance was reported at each training size, and a size of 24K variants was arbitrarily selected for final model training given that the model performance reached a plateau after a training size of ˜5K. The training library core variants (N ˜60K, after removing the non-detected sequences) were then randomly divided into training (24K), validation (12K) and testing subsets (24K), all from the training library. The model was trained on the training set (24K), validated during the training process on the validation set (12K), and tested on the testing set (24K). The model was further tested on the unique variants from the assessment library to assess its generalization across libraries.

### Fit4Function Library Sampling

The Fit4Function libraries were intended to be sampled from the high production fitness space. For the Fit4Function library utilized in the Examples of the present disclosure, a set of 7-mer amino acid sequences was first uniformly sampled 100 times the required library size (240K Fit4Function variants*100=24M variants), by equally sampling each amino acid at each of the 7 positions. Duplicates were removed and the remaining sequences were scored using the production fitness model. Then, the 240K Fit4Function library variants were probabilistically sampled from the parametrized high production fitness distribution. In addition to the 240K high production fitness variants, 1K stop codon-containing variants and 3K variants from the 10K shared variants between the training and assessment libraries were added as a control set.

### Fit4Function Library Validation

Fitness enrichment scores are relative across library variants due to normalization calculations; calibration is needed to make the fitness scores of two libraries of different compositions comparable for assessment or integration purposes. To calibrate the Fit4Function library production fitness, the 3K control set was used to fit an ordinary linear regression model of the measured production fitness scores between the Fit4Function library and the training library. These regression parameters were applied to the production fitness measured scores of the 240K Fit4Function variants to obtain calibrated production fitness scores. After synthesizing the Fit4Function library, the predicted fitness scores were compared to the calibrated measured fitness by means of correlation.

### Animals

All mouse procedures were performed as approved by the Broad Institute Institutional Animal Care and Use Committee (IACUC), approval number 0213-06-18-1. Female C57BL/6J (000664) mice were obtained from the Jackson Laboratory (JAX). Recombinant AAV vectors were administered intravenously via the retro-orbital sinus in young adult (7- to 8-week-old) animals. Mice were randomly assigned to groups based on predetermined sample sizes. No mice were excluded from the analyses. For all assays, mice were anesthetized with EUTHASOL™ (Virbac) and transcardially perfused with phosphate buffer saline, pH 7.4, at room temperature (RT). Experimenters were not blinded to the sample groups.

For the cynomolgus macaque experiments, the study plan involving the care and use of animals was reviewed and approved by the Charles River CR-LAV Institutional Animal Care and Use Committee (IACUC). During the study, the care and use of animals was conducted by CR-LAV with guidance from the USA National Research Council and the Canadian Council on Animal Care (CCAC). The Test Facility is accredited by the CCAC and AAALAC. Per the CCAC guidelines, this study was considered as a category of invasiveness C.

The rhesus macaque study (n=2) was conducted in the NIH Nonhuman Primate Testing Center for Evaluation of Somatic Cell Genome Editing Tools at the University of California, Davis. All procedures conformed to the requirements of the Animal Welfare Act, and protocols were approved prior to implementation by the UC Davis IACUC.

### AAV Mouse In Vivo Biodistribution Assays

Purified virus libraries were injected at a dose of 1×10¹² into C57BL/6J mice. Two hours post-injection serum was collected and organs were harvested using disposable 3 mm biopsy punches (Integra, 33-32-P/25) with a new biopsy punch used per organ per replicate. Harvested tissues were immediately frozen in dry ice. AAV genomes were recovered using a DNeasy kit (Qiagen, 69504) following the manufacturer's protocol and samples were eluted in 200 μL elution buffer for NGS preparation.

### AAV Cynomolgus Macaque In Vivo Biodistribution Assays

The library administered had 100K unique amino acid variants following the Fit4Function criteria (uniformly sampled from the high production fitness sequence space) in addition to a calibration set (3K), control variants, and AAV9. Each variant in the Fit4Function distribution was represented by either two or six 7-mer replicates; AAV9 was represented by two replicates. The purified virus library was injected at a dose of 4.6×1012 vg/kg into a female cynomolgus macaque that was pre-screened for NAbs against AAV9 (CRL). Six hours after systemic delivery, the animal was perfused with cold PBS and organs were harvested and snap frozen in dry ice. DNA was extracted using a DNeasy kit in a Qiagen QIAcube Connect. Samples were then processed as detailed in the NGS sample preparation section.

### AAV NHP In Vivo Transduction Assays

Approximately 3-month-old rhesus monkeys (˜1 kg; one male, one female) were screened then assigned to the project after confirming seronegative status for AAV9 antibodies. Sedation with Telazol (IM) was performed prior to IV administration of a purified virus library (1×1013 vg/kg) with blood samples collected (˜4 mL; hematology, clinical chemistry, serum, plasma; pre-administration then weekly post-administration). Animals were monitored closely during the study period and until endpoint (four weeks post-administration). They remained robust and healthy with no evidence of adverse findings (body weights, hematology and clinical chemistry panels were all in the normative range at all timepoints; data not shown). Four weeks after systemic delivery, tissues were collected and snap frozen over liquid nitrogen then placed on dry ice immediately prior to storage at ≤−80° C. RNA and DNA were extracted using TRIzol (Invitrogen, 15596026) following the manufacturer's instructions. Total RNA was cleaned up using a RNeasy kit (Qiagen, 74106) followed by on-column DNA digestion. RNA was converted to cDNA using Maxima H Minus Reverse Transcriptase (ThermoFisher Scientific, EP0751) according to the manufacturer's instructions. Samples were then processed as detailed in the NGS sample preparation section.

### NHP Serum Screening for Anti-AAV9 Neutralizing Antibodies

Neutralization assays were performed at two MOIs, 500 and 1000, in Perkin-Elmer white 96-well plates. Four-fold serial dilutions (1:4 to 1:16,384) of macaque serum samples were prepared in 96-well plates using DMEM supplemented with 5% FCS. Then, 40 μL of each dilution was transferred to a separate 96-well plate, mixed with an equal volume of AAV9.CAG-GFP-P2A-Luciferase-WPRE-SV40 vector (4-8E7 vg per 40 μL, diluted in DMEM-5% FCS), and incubated for one hour at 37° C. Following the incubation, AAV-serum samples were transferred into a new 96-well plate (20 uL triplicates) and a total of 80 μL of DMEM-5% FCS, containing 20,000 HEK293T cells, was added to each well (final volume of 100 μL). 96-well plates were incubated for 48 hours at 37° C., 5% C02. Luminescence levels were read using a Perkin Elner Victor Luminescence Plate Reader using the britelite plus Reporter Gene Assay System (Perkin-Elmer, #6066761). Data was analyzed using the neutcurve Python package developed by the Bloom lab. The neutralizing antibody titer was measured as the concentration that resulted in a 50% reduction in luciferase activity relative to the no-serum control. Animals used in the transduction study had NAb titers <1:12 in this set of antibody screens.

### In Vitro Binding and Transduction

HEK293T/17 (ATCC® CRL-11268™), HepG2 (ATCC® HB-8065™), THLE-2 (ATCC® CRL-2706™), hCMEC/D3 (Millipore, SCC066), and human and mouse BMVECs (Cell Biologics, H-6023 and C57-H6023) were grown in 100 mm dishes and exposed to the Fit4Function or (NNK) 7-mer library (MOI 1E4 for HEK293T/17, MOI3E4 for hCMEC/D3, MOI 6E4 for primary human and mouse BMVECs and MOI5E3 for HepG2 and THLE-2) diluted in 10 mL of growth media at 4° C. with gentle rocking for two hours. After that, cells were washed three times with DPBS, and total DNA was extracted with DNeasy kit (Qiagen) according to the manufacturer instructions. Half of the recovered DNA was used in PCR amplification for viral genome sequence recovery.

Transduction assays were performed as described above with the following exceptions. The cells were cultured in growth media containing virus for 60 hours and total RNA was then extracted with the RNeasy kit (Qiagen), 5 g of RNA was converted to cDNA using the Maxima H Minus Reverse Transcriptase according to the manufacturer's instructions.

### Sequence-to-Function Mapping

Functional scores were quantified as the log 2 of the fold-change enrichment of the variant reads-per-million (RPM) after the screen relative to its RPM in the virus library, i.e. log 2 (Assay RPM/Virus RPM). Fit4Function models utilized the same design of the ML framework utilized for production fitness mapping (two-layer LSTM, custom early stopping, batch size of 500 variants, MSE error and Adam optimizer). Out of the 240K variants in the Fit4Function library, 90K were allocated for training and testing the ML function models (model construction) and 150K variants were held-out for validation of the MultiFunction approach. The training size for each function model was optimized independently. As with the production fitness model, the function models were assessed by correlation between the predicted and measured functional scores.

### MultiFunction Library Design

Using the previously generated fitness models of the production fitness and the five functional models described in the Examples provided above, an in-silico screen of 10M randomly sampled 7-mer sequences was conducted to identify variants that are highly fit for all six traits. The threshold of high fitness for each function was arbitrarily set to the 50th percentile of each functional fitness distribution from the Fit4Function screening data. The percentiles were calculated on the detected variants of each functional assay from the 90K model construction data set. To reduce false positive predictions (variants predicted above the thresholds due to model errors), the filtration thresholds were increased slightly when applied to the predictions. For example, if the measured threshold is at fitness score of 2.5, variants predicted to have fitness >2.5+shift were considered. The shift in applied thresholds is arbitrarily set to be 5% of the fitness dynamic range of each function. The thresholds were then used to filter out the 10M variants that were run through the six functional prediction models. Out of the variants predicted to pass the six modified thresholds, 30K variants were sampled to be included in the MultiFunction library. The 30K variants were each represented by two 7-mer replicates.

The MultiFunction library also included (1) a positive control set (3K) that was drawn from the subset of the 150K Fit4Function validation set that met the six conditions on the actual measurements (without modifying the thresholds), (2) a set of 10K variants randomly sampled from the Fit4Function 240K core variants as background controls representing the high production fitness space, (3) a set of 3K calibration variants present in the Fit4Function library (and the training library) to be used as background controls representing the entire (unbiased) sequence space, and (4) 1K stop codon containing sequences.

### MultiFunction Library Validation

The MultiFunction library was synthesized, virus was produced, and the five liver-related functions were screened in the same way the Fit4Function library was processed. The success rate of the MultiFunction library was quantified in terms of hit rate, i.e. out of the 30K variants predicted to meet the six criteria, what percentage satisfied the six criteria when the MultiFunction library was screened on those functions (predicted positive versus measured positive). To determine whether a variant met specific functional criteria, the distribution of that function for the MultiFunction variants against was compared the positive control set. For a variant to be considered a hit for a specific function, its measured value should be above the mean-2SD (standard deviations) of the positive control set measured in the same experiment. A variant was considered a hit in calculating the MultiFunction hit rate only if it was a hit for all six functions; a variant that met five or fewer conditions was not considered a hit.

The hit rate of the Fit4Function space was the number of non-control variants from the Fit4Function library measured to pass the six thresholds (without the prediction marginal shifts used for MultiFunction variant design) divided by the number of non-control variants in the library. The hit rate for the uniform sequence space could be estimated as the hit rate in the Fit4Function library (representing the high production fitness space—all the low production fitness variants were filtered out from the selection), relative to the percentage of the space occupied by the high production fitness variants. Uniform hit rate=Fit4Function hit rate×High production fitness ratio=7.1%×40.8%=2.9%.

### Individual Capsid Characterization

Individual capsids were cloned into iCAP-AAV9 (K449R) backbone (GenScript), and administered to C57BL/6J (The Jackson Laboratory, 000664) mice at a dose of 1×10¹⁰ vg/mouse (n=5/group). Three weeks later, three separate lobes of the liver were collected for RNA extraction and a single lobe per mouse was dropped fixed into 4% PFA.

For microscopy, fixed liver tissues were sectioned at 100 μm using a Leica VT1200 vibratome. Sections were mounted with ProLong™ Gold Antifade Mountant with DAPI (ThermoFisher, P36931). Liver images were collected using the optical sectioning module on a Keyence BZ-X800 with a Plan Apochromat 20× objective (Keyence, BZ-PA20). 3 images were taken for each animal (n=5/group) and compared to a no injection control (n=3 animals). In CellProfiler, nuclei were segmented and DAPI+ nuclei were identified using a threshold on DAPI intensity determined from the no injection control. Each DAPI+ nuclei was then quantified with the median pixel intensity in the GFP channel.

For assessment of liver transduction by quantitative RT-PCR, total RNA was recovered using TRIzol (Invitrogen, 15596026) following the manufacturer's instructions. Afterwards, total RNA was cleaned up using a RNeasy kit (Qiagen, 74106) followed by on-column DNA digestion. RNA was converted to cDNA using Maxima H Minus Reverse Transcriptase (ThermoFisher Scientific, EP0751) according to manufacturer instructions. Afterwards, qPCR was used to detect AAV encoded RNA transcripts with the following primer pair (5′-GCACAAGCTGGAGTACAACTA-3′ (SEQ ID NO: 199497)) and (5′-TGTITGTGGCGGATCTTGAA-3′ (SEQ ID NO: 199498)) and the following primer pair for GAPDH (5′-ACCACAGTCCATGCCATCAC-3′ (SEQ ID NO: 199499)) and (5′-TCCACCACCCTGTTGCTGTA-3′ (SEQ ID NO: 199500)).

THLE and HepG2 cells were seeded in a 96 well plate the day before adding the AAVs at 5000 vg/cell. For binding assays, viruses were diluted in media and incubated with cells at 4° C. with gentle shaking for one hour. After incubation, cells were washed three times with PBS to remove unbound virus and treated with proteinase K to release viral genomes for qPCR quantification. For transduction assays, cells were incubated with the AAVs for 24 hours at 37° C. and assayed with Britelite plus (Perkin Elmer, cat #6066766) following the manufacturer's protocol.

7 Individual Variants when Tested in Macaque

The rhesus macaque study (n=2) was conducted in the NIH Nonhuman Primate Testing Center for Evaluation of Somatic Cell Genome Editing Tools at the University of California, Davis. All procedures conformed to the requirements of the Animal Welfare Act, and protocols were approved prior to implementation by the UC Davis IACUC.

Approximately 3-month-old rhesus monkeys (˜1 kg; one male, one female) were screened then assigned to the project after confirming seronegative status for AAV9 antibodies. Sedation with Telazol (IM) was performed prior to IV administration of a purified virus library (1×1013 vg/kg) with blood samples collected (˜4 mL; hematology, clinical chemistry, serum, plasma; pre-administration then weekly post-administration). Animals were monitored closely during the study period and until endpoint (four weeks post-administration). They remained robust and healthy with no evidence of adverse findings (body weights, hematology and clinical chemistry panels were all in the normative range at all timepoints; data not shown). Four weeks after systemic delivery, tissues were collected and snap frozen over liquid nitrogen then placed on dry ice immediately prior to storage at ≤−80° C. RNA and DNA were extracted using TRIzol (Invitrogen, 15596026) following the manufacturer's instructions. Total RNA was cleaned up using a RNeasy kit (Qiagen, 74106) followed by on-column DNA digestion. RNA was converted to cDNA using Maxima H Minus Reverse Transcriptase (ThermoFisher Scientific, EP0751) according to the manufacturer's instructions. Samples were then processed as detailed in the NGS sample preparation section.

Relative transduction efficiencies were assessed by measuring the enrichment of the capsid RNA for each variant in the liver relative to the starting virus. It was found that the macaque liver transduction efficiency for the seven individually characterized liver MultiFunction variants are significantly higher than that of AAV9 (FIG. 11F; n=2 rhesus macaques). In the virus library, each variant was represented by two 7-mer replicates while AAV9 was represented by three replicates.

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The disclosures of the following U.S. Provisional patent applications are incorporated herein by reference in their entireties for all purposes: 63/342,001, filed May 13, 2022 and 63/343,010, filed May 17, 2022.

1. An adeno-associated virus (AAV) capsid polypeptide comprising a peptide inserted within the capsid polypeptide, wherein the peptide comprises an amino acid sequence selected from the group consisting of RPNRDTS (SEQ ID NO: 144800); MDGQRRI (SEQ ID NO: 132518); ETNRAGR (SEQ ID NO: 116028); TGRVDSR (SEQ ID NO: 149619); NMTRARD (SEQ ID NO: 136472); GEKPKFT (SEQ ID NO: 164722); MEPRQRT (SEQ ID NO: 132640); and variants thereof comprising a substitution or deletion of one or two amino acids.

2. The capsid polypeptide of claim 1, wherein the capsid is an AAV1, AAV2, AAV3, AAV3b, AAV4, AAV5, AAV6, AAV7, AAV8, AAV9, AAV9 K449R, rh.10, rh.8, or LK03 capsid polypeptide.

3. The capsid polypeptide of claim 1,
wherein the peptide is inserted in Loop VIII of the capsid polypeptide;
wherein the peptide is inserted between amino acids 565 and 605 of an AAV9 K449R amino acid sequence, or at an equivalent insertion position in another AAV polypeptide;
wherein the peptide is inserted between amino acids 575 and 595 and 600 of an AAV9 K449R amino acid sequence, or at an equivalent insertion position in another AAV polypeptide;
and/or wherein the peptide is inserted between amino acids 588 and 589 of an AAV9 K449R amino acid sequence, or at an equivalent insertion position in another AAV polypeptide.

4-6. (canceled)

7. The capsid polypeptide of claim 1, wherein a viral particle comprising an AAV capsid comprising the peptide has increased transduction efficiency for a cell of interest relative to an AAV capsid lacking the peptide.

8. The capsid polypeptide of claim 7, wherein the cell of interest is a liver cell, brain cell, brain endothelial cell, kidney cell, spinal cord cell, spleen cell, nerve cell, or a cell of the spinal cord, heart, or lungs.

9. The capsid polypeptide of claim 7, wherein transduction efficiency is increased by at least about 10%, 25%, 50%, 100%, 200% or more relative to an AAV capsid lacking the peptide.

10. The capsid polypeptide of claim 1, wherein a viral particle comprising an AAV capsid comprising the peptide has increased production fitness relative to an AAV capsid lacking the peptide; and/or wherein a viral particle comprising an AAV capsid comprising the peptide has increased biodistribution in an organ of interest relative to an AAV capsid lacking the peptide.

11-13. (canceled)

14. An adeno-associated virus (AAV) capsid polypeptide comprising a peptide inserted within the capsid polypeptide, wherein the peptide comprises a motif selected from those listed in Tables 2-27.

15. The capsid polypeptide of claim 8, wherein the AAV capsid polypeptide is an AAV9 K449R capsid polypeptide and shares at least 85% sequence identity to an amino acid sequence selected from the group consisting of:
AAV-BI151
(SEQ ID NO: 199456)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDENRFHCHESPRDWQRLINNNWGFRPKRLNFKLFNI
QVKEVTDNNGVKTIANNLTSTVQVFTDSDYQLPYVLGSAHEGCLPPFPADVEMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQRPNRDTSAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI152
(SEQ ID NO: 199457)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHESPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQMDGQRRIAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI153
(SEQ ID NO: 199458)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDENRFHCHESPRDWQRLINNNWGFRPKRLNFKLFNI
QVKEVTDNNGVKTIANNLTSTVQVFTDSDYQLPYVLGSAHEGCLPPFPADVEMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQETNRAGRAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI154
(SEQ ID NO: 199459)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDENRFHCHESPRDWQRLINNNWGFRPKRLNFKLENI
QVKEVTDNNGVKTIANNLTSTVQVFTDSDYQLPYVLGSAHEGCLPPFPADVEMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQTGRVDSRAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI155
(SEQ ID NO: 199460)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDENRFHCHFSPRDWQRLINNNWGFRPKRLNFKLFNI
QVKEVTDNNGVKTIANNLTSTVQVFTDSDYQLPYVLGSAHEGCLPPFPADVEMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQNMTRARDAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
AAV-BI156
(SEQ ID NO: 199461)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLENI
QVKEVTDNNGVKTIANNLTSTVQVFTDSDYQLPYVLGSAHEGCLPPFPADVEMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQGEKPKFTAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL;
and
AAV-BI157
(SEQ ID NO: 199462)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLENI
QVKEVTDNNGVKTIANNLTSTVQVFTDSDYQLPYVLGSAHEGCLPPFPADVEMIPQYGYLTLND
GSQAVGRSSFYCLEYFPSQMLRTGNNFQFSYEFENVPFHSSYAHSQSLDRLMNPLIDQYLYYLS
RTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQMEPRQRTAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL*.

16. A viral particle comprising the AAV capsid polypeptide of claim 8.

17. A polynucleotide encoding the capsid polypeptide of claim 1.

18. A library of adeno-associated virus (AAV) capsid polypeptides or polynucleotides encoding the same, wherein the library comprises two or more capsid polypeptides of claim 1; or wherein the library comprises two or more capsid polypeptides each comprising a peptide with a sequence selected from the group consisting of SEQ ID NOs: 1-199427 and 200028-201544.

19-25. (canceled)

26. The library of claim 12, wherein the capsid is an AAV1, AAV2, AAV3, AAV3b, AAV4, AAV5, AAV6, AAV7, AAV8, AAV9, AAV9 K449R, rh.10, rh.8, or LK03 capsid polypeptide.

27. (canceled)

28. The library of claim 12, wherein the library comprises an amino acid sequence motif selected from the group consisting of:
[RQMT] [RNQP] [RQPS] [ANQGST] **G; [QTV] [ANQGKP] [RNGSTY] * [ARGT] * [GP];
[RKSV] [FPS] ** [RT] [QGS] [GS]; [GST] * [QT] [NQ]R*G; [TV] ** [NS]R[QGM]G;
[RNQT] [ANPS] [RNS] ** [NQGS]G; T [NGS] **R[AQS]G; NR** [NG] [QG]A;
R**QGGG (SEQ ID NO: 199501); [QF] [KS] [RN] ** [NP] [AP]; QR**S [TV]A (SEQ ID NO: 199502); [QKM] *G[RSV] [KT] *G;
[QMTV] [RN] * [ANV] [RQS] * [GS]; [NQ]R[NGP] [NQS] **A;
[QT] * [RT] [RT] * [NQG] [AG]; NTR**SA (SEQ ID NO: 199503);
QRP* [AS] * [AS]; RQ**TNA (SEQ ID NO: 199504); QRP** [MV] [AS];
[RN] *N[RS] *[QG]G; [MTV]R[PS] *[QT] *G; [RY]S*[QK] *Q[GS]; MRG**MG (SEQ ID NO: 199505); [GV] *[NT] *R[QS]G; [ST] [RQ] [RS]T**A; R*S*STP (SEQ ID NO: 199506); QR**TNG (SEQ ID NO: 199507); Q*RQT*P (SEQ ID NO: 199508); [NQ]RQ*[GS] *A; TR**NNA (SEQ ID NO: 199509);
[RT]S*[RQ] [QS] *A; GQ*RV*G (SEQ ID NO: 199510); T*TSR*G (SEQ ID NO: 199511); TRG**TG (SEQ ID NO: 199512); NR* [GT] * [TV]G; T*RT*SA (SEQ ID NO: 199513); MG*R*GA (SEQ ID NO: 199514); [NQ]R*[NQ]S*A;
RQ*PT*A (SEQ ID NO: 199515); T*T*RSG (SEQ ID NO: 199516); T*RGS*P (SEQ ID NO: 199517); TR**TMG (SEQ ID NO: 199518); R*TS*SP (SEQ ID NO: 199519); N**QRSA (SEQ ID NO: 199520); [QT] [RK] *S* [TY]A;
QR*PA*G (SEQ ID NO: 199521); RS*S*GG (SEQ ID NO: 199522); RTS*S*P (SEQ ID NO: 199523); TRQ*T*G (SEQ ID NO: 199524); QR*S*TG (SEQ ID NO: 199525); R*NS*SP (SEQ ID NO: 199526); MR*G*QS (SEQ ID NO: 199527); N**SRQG (SEQ ID NO: 199528); NR*ST*A (SEQ ID NO: 199529);
RSQ*G*G (SEQ ID NO: 199530); T*RTN*A (SEQ ID NO: 199531); T*S*RMG (SEQ ID NO: 199532); TR**TQA (SEQ ID NO: 199533); and TRT**SG (SEQ ID NO: 199534); YSGK**G (SEQ ID NO: 199535); or polynucleotides encoding the same, where “*” represents any amino acid and square brackets denote a list of amino acids that may occur at a position; and/or
wherein the library comprises an amino acid sequence motif selected from the group consisting of: [RQGKST] [ARNQGFPST] ** [ARNQGKMTV] [ANQGMSV]G;
[\hat{ }ADCEHILKPW] [ARNQGKPS] [ARNQGKST] [ARNQKPSTV] **[NDGPS];
[NQKMFSTY] [RNGKT] [\hat{ }CEHILKFSWY] ** [RNQKMPSTV] [ANDEPS];
[\hat{ }ARDCEHILW] [RGLKPSY] * [NQGKPSTV] * [ANQGFST]G;
[\hat{ }ARDCEHILPW] [ARQGKPT] [RQGKPST] ** [ANGMST]G;
[\hat{ }ADCQEHILW] [NQGLKMPST] * [\hat{ }NCEHILMFWY] [ARNQGMTV] * [GS];
[RGMY] [ANQGPS] [NQKPST] ** [ANGKMST]G;
[NQGKFSTV] [RNQGKS] ** [ARNGKT] [NQGKFSYV] [ANDGS];
[\hat{ }ARDCEHILFW] * [ARNQGKMPT] [RQGKPSTV] * [AQGST]G;
[\hat{ }ARDCEHILPW] * [RNGKPST] * [ARNGKSV] [NQGKMST]G;
[RNKT] [RNQPS] *[RQGPS] [RQGS] *[APS];
[RNMPY] [RNQKPS] * [ANQTV] * [ANQGKTV] [DGPS];
[NQGKMFTV] [ARQGKMPTV] [RNQGKPTY] * [ARQKMST] *G;
[RNQMFST] [RNQGKT] *[ARNQPST] *[AQGSTY] [APS];
[QGKMSTV] * [ARNQGKMPT] [RNQGKT] [ARNSTV] *G;
[RMT] * [NQKT] * [ARQGT] [ANQS] [GP];
[RKMSV] **L[ANQGKPS] [NGMSTV] [ANGKSTV] [DGS];
[NQKMPTYV] * [ARGKPTV] [NGST] [RNQKST] * [GPS];
[RNQKMSY] [RQKST] [ADQKPST] * [AKSTV] * [ADPS]; [RK] ** [NG] [AS] [GT]A;
[RQ] * [RNPST] [AQGST] [AQGSTV] * [GPS]; R[QPS] *S* [QGV]G;
[RKV] [NGKP] *N[ANGT] *G; [NQGLKFT] [NGKS] [QKMS] ** [ARQKSV] [ANDEG];
[RQMS] * [RNQKST] [NPST] * [ARNQGMS] [DGP]; [RKT] ** [PST] [QT] [ANG]G;
[NQKMT] [ARDKPST] [RNGKP] *G*G;
[RNQKMFT] [RDGKPST] [RNQGKPT] *[NQGST] *[AS];
[RNQMST] [RNDQKST] [RGKPST] [AQGKPST] **[APSV];
[QKT] * [KS] * [AQGS] [NQS] [AS]; [KFV] * [ANK] * [ST] [KS] [AD];
[RQKMTYV] [RNDKPS] [RQGPS] [NDQGPST] ** [GP];
[NQMT]R*[NGPTV] [AQGS] *[GS]; [QMSY] [RKP] **S[AQGKV] [ADGS];
R[NS] ** [ANQ] [AQT]A; R[QS] **T [NQ] [AS]; [RQ] * [KPT] [GST] * [ANQGT]G;
[RN] [RQP] * [GPS]T*A; [RNMPT] * [RNGKST] [ANDQKPST] [RNGST] *A;
K[QPS] *[NMS] *[QV] [GS]; K*[NQG] *[NT] [AST]G; RN*Q*SG (SEQ ID NO: 199536); K* [ANGST] [NST] * [AMS] [GS]; KT*S*GA (SEQ ID NO: 199537);
[RKMT] [RQKPST]S*[ARSTV] *G; [KM] *N*GNA (SEQ ID NO: 199538);
N**S [GT] [GM]A; K* [GPS] [GT] [AT] *A; [QG]K* [GTV] [AMS] * [AN];
[RQT] * [RNQPT] [RNST] * [QGS]A; [NQ] [RK] ** [GS]TA; [TV] **NRQG (SEQ ID NO: 199539); R[AGPS] [NQPT] *[NGST] *G; R[MST]N**[QT] [GP];
[RNQ] * [PT] [GT] [RS] *A; RS**NTG (SEQ ID NO: 199540);
[RK] *[PS]G*[NQGT]A; Q*KSA*A (SEQ ID NO: 199541); KN*G*TA (SEQ ID NO: 199542); K*S*[AS]GA (SEQ ID NO: 199543); [NG]K*[AS] [GT] *A;
[GV]NS*[RK] *G; KT*S*SA (SEQ ID NO: 199544); [RM] [KP] *[AS]S*G;
VK**STG (SEQ ID NO: 199545); [QKT] [RQGMSTV] [AGKP] [NGST] **A;
PR*AT*G (SEQ ID NO: 199546); TRS*T*M (SEQ ID NO: 199547); T**RQQA (SEQ ID NO: 199548); KN*S*SG (SEQ ID NO: 199549); RNS*[AG] *G (SEQ ID NO: 199550); R*S* [AS]T [PS]; [RG] [KS]S* [GT] * [AG]; QR*NS*A (SEQ ID NO: 199551); R*SN*TG (SEQ ID NO: 199552); R*[NS] *[GT]GG;
RAP**NS (SEQ ID NO: 199553); R*NNS*G (SEQ ID NO: 199554);
[NK] * [GKP] [GST] * [GS]A; QK*GT*G (SEQ ID NO: 199555); T**NRGG (SEQ ID NO: 199556); VK*AS*A (SEQ ID NO: 199557); K*P*TGG (SEQ ID NO: 199558); K**QNQG (SEQ ID NO: 199559); R*S*TAP (SEQ ID NO: 199560);
K*S[NG] *[GS] [GS]; R*SN*NA (SEQ ID NO: 199561); RMP**GA (SEQ ID NO: 199562); [RW] [ANP]N[AKS] **A; KT**SGG (SEQ ID NO: 199563); R*P*TGA (SEQ ID NO: 199564); N**QRSA (SEQ ID NO: 199520); K*[NQ]ST*A (SEQ ID NO: 199565); MKN[TV] **A (SEQ ID NO: 199566); KGNN**G (SEQ ID NO: 199567); TKP**AA (SEQ ID NO: 199568); TR*GT*G (SEQ ID NO: 199569); KN*G*SA (SEQ ID NO: 199570); K*ASS*A (SEQ ID NO: 199571);
KLNS**G (SEQ ID NO: 199572); N**SRQG (SEQ ID NO: 199528); and
QNR*A*P (SEQ ID NO: 199573); R*P*AGA (SEQ ID NO: 199574); RT**STP (SEQ ID NO: 199575); SRTT*NG (SEQ ID NO: 199576); T*RT*NA (SEQ ID NO: 199577); TK*NS*G (SEQ ID NO: 199578); TRP**AA (SEQ ID NO: 199579); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position.

29. (canceled)

30. The library 14, wherein a viral particle having a capsid comprising the motif has increased binding to a cell of interest relative to a control viral particle.

31. The library of claim 14, wherein the library comprises an amino acid sequence motif selected from the group consisting of: QSRT**P (SEQ ID NO: 199580); K[HP] [NT] *P* [NS]; RN*P*TS (SEQ ID NO: 199581); K**GPKD (SEQ ID NO: 199582); NRGQ**A (SEQ ID NO: 199583); A**NEKR (SEQ ID NO: 199584); TG**RSG (SEQ ID NO: 199585); TAN*R*G (SEQ ID NO: 199586); T*TNR*G (SEQ ID NO: 199587); QSR**NP (SEQ ID NO: 199588); T*T*RSG (SEQ ID NO: 199516); K**NPAN (SEQ ID NO: 199589); KM**PKD (SEQ ID NO: 199590); MSRN**A (SEQ ID NO: 199591); NDA**KK (SEQ ID NO: 199592); QR*GP*M (SEQ ID NO: 199593); RS*P*NA (SEQ ID NO: 199594); T*S*RMG (SEQ ID NO: 199532); T*TSR*G (SEQ ID NO: 199511); VAR* H*G (SEQ ID NO: 199595); or polynucleotides encoding the same, where “*” represents any amino acid and square brackets denote a list of amino acids that may occur at a position; and
wherein a viral particle having a capsid comprising the motif has increased binding to a liver cell relative to a control viral particle;
wherein the library comprises an amino acid sequence motif selected from the group consisting of: [GKMFSTYV] [NGKST] ** [ARNGKSV] [AQMFST]G;
[RNQKTY] *[NQGKPST] [ANGPST] *[NQGTV]G; [QLMFPSTYV]K* [GPSTV] * [QGST]G;
[RNQGKMPTY] [ARNQGPST] [ARNQGKPT] ** [AGKMSTV]G;
[RQGMTV] *[RQGKPST] [QGPST] *[ARQMPS] [DGPS];
[RNQGKMPST] * [AQGKMPSTV] [RNQGST] [ARNQGKST] *G;
[NGKTV] ** [RNQGPSTV] [ARQGKMST] [ANQGKFST] [AGPS];
[RNGKMFSTY] [ANQLKMPT] * [ARDQGSTV] [ARQGKTV] * [GS];
[RQGKMFTV] [ARNQGPST] [RGKPST] * [\hat{ }DCEILFPWY] *G;
[RNQGIKMST] [RDLKMFST] [ANDQHMPST] ** [ARNGKMSV] [ANDEKPS];
[RNQGIKFV] * [ARNQPST] * [ARGKMTV] [ARNQGT] [DEGS];
[\hat{ }ARNCEHILP] [ANQGKMPST] [RNQGHKMST] [ARNQGKPTV] ** [ARGS];
[RNQGIMFT] [RKPSTV] [AQGHKPST] *[ARNIKST] *[EGPS];
[NQKMFSTV] [RKS] **[AGST] [ARNDKSTV] [ADEK];
[RNQKMSTY] [RNGKST] * [ARNQMPSTV] * [\hat{ }CEHILKFPWV] [ADPST];
K* [GMP] [QST] * [AQM] [GS]; [RQKTY] * [ARQKPST] [QGST] [ANGST] * [ADPS];
[NQFTY] * [KST] * [ARGS] [AQGKMTV]G; [RT] [RPT] ** [ANST] [NQT] [APS];
[RNKMPY] [ARNGLKS] * [ANQKST] * [AQGSV]G; [RNQSY] [KPST] ** [RNGMS] [QG]G;
[RKT] [PS] * [RPST] [QGS] *A; [RNQEKMS] [RNQT] [RGIPST] [NQGKPT] ** [AEGFP];
[NQT]K* [AGPST] [GMS] * [AS]; [RNK] [RDLS] [RNPT]S** [GP];
[RK] * [NP] [GV] * [GT]A; [RQGMFSY] [KS] [NQGMPS] ** [NQGS] [GS];
[MFPV]K[ANPS]S**[GS]; [MF]K[NT] **[QPT]A;
[GFT] *[GKT] *[RNQK] [KSTV] [GS]; [QGM] [RKS] [NGPS] *[AST] *A;
[RT] * [KP] * [QT] [QG]A; [GM] [HP]K** [AP]A; [TV] * [NK] *S [NS] [AS];
[QGMT] [RK] * [GPTV] [AS] *G; [RV] [KP] *NT*G; M*SKS*A (SEQ ID NO: 199596); A**NEKR (SEQ ID NO: 199584); PR*AT*G (SEQ ID NO: 199546);
NK[AGP] [AQV] **[DGS]; [MS] **K[ST] [GT]G; [RQK] [ARF] **T[AGS]G;
[QF]K**[AN] [QS]S; [NMT]K[ANP] *G*G; R**KEEK (SEQ ID NO: 199597);
[RGV] [KP] *[AS] [ST] *[AS]; NK[NS] *G*A (SEQ ID NO: 200021); QK*GT*G (SEQ ID NO: 199555); TK*NS*G (SEQ ID NO: 199578); TGK**[AT]A (SEQ ID NO: 199598); R**AGVG (SEQ ID NO: 199599); [NT]K* [TV] *KD;
MKS**TG (SEQ ID NO: 199600); G*KSV*G (SEQ ID NO: 199601);
[KM] * [NS] * [GS] [NG]A; TNK**QG (SEQ ID NO: 199602);
K[DKPS] [RNDQ] *[GK] *[AD]; NAR*T*G (SEQ ID NO: 199603); MR*NQ*G (SEQ ID NO: 199604); [FT] *K[AT] *QA; KT**GGA (SEQ ID NO: 199605);
V*NKV*G (SEQ ID NO: 199606); FKG**SA (SEQ ID NO: 199607); FNK**QG (SEQ ID NO: 199608); GPK*T*A (SEQ ID NO: 199609); KQS*S*P (SEQ ID NO: 199610); [NS] *KG* [ST]A; GP*G*KG (SEQ ID NO: 199611); RDKS**A (SEQ ID NO: 199612); R*S*STP (SEQ ID NO: 199506); KT**AGG (SEQ ID NO: 199613); FK**TQG (SEQ ID NO: 199614); FGK**TG (SEQ ID NO: 199615); NKTG**A (SEQ ID NO: 199616); TK**TYG (SEQ ID NO: 199617);
TKPG**G (SEQ ID NO: 199618); KP*T*GG (SEQ ID NO: 199619); TGK**SA (SEQ ID NO: 199620); KPN*S*A (SEQ ID NO: 199621); TGKS**A (SEQ ID NO: 199622); T*RT*SA (SEQ ID NO: 199513); V*KS*TG (SEQ ID NO: 199623); F*K*TSA (SEQ ID NO: 199624); FGK**SG (SEQ ID NO: 199625);
K*GG*AG (SEQ ID NO: 199626); KPS*N*A (SEQ ID NO: 199627); QR*NS*A (SEQ ID NO: 199551); R**QGGG (SEQ ID NO: 199501); RP*N*GG (SEQ ID NO: 199628); TK**TQG (SEQ ID NO: 199629); TKSS**A (SEQ ID NO: 199630); and V*KSQ*G (SEQ ID NO: 199631); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position, wherein a viral particle having a capsid comprising the motif has increased transduction efficiency for a liver cell relative to a control viral particle;
wherein the library comprises an amino acid sequence motif selected from the group consisting of: [ARDTV] ** [NKP] [REG] [QEGK] [RGK]; NAR**GG (SEQ ID NO: 199632); N[AR] [RG]Q** [AG]; VR**SSA (SEQ ID NO: 199633); TG**RSG (SEQ ID NO: 199585); R*KDS*A (SEQ ID NO: 199634); T*T*RSG (SEQ ID NO: 199516); T*TNR*G (SEQ ID NO: 199587); T*TSR*G (SEQ ID NO: 199511); G**SIRS (SEQ ID NO: 199635); GQSS**R (SEQ ID NO: 199636); M*KP*RD (SEQ ID NO: 199637); NDA**KK (SEQ ID NO: 199592); NK*DR*G (SEQ ID NO: 199638); QRP*A*A (SEQ ID NO: 199639); SP**RGG (SEQ ID NO: 199640); V*N*SSA (SEQ ID NO: 199641); or polynucleotides encoding the same, where “*” represents any amino acid and square brackets denote a list of amino acids that may occur at a position, wherein a viral particle having a capsid comprising the motif has increased binding to a liver cell relative to a control viral particle;
wherein the library comprises an amino acid sequence motif selected from the group consisting of:
[NDGILST]K* [RNGHKPSTV] * [ANDGKFSY] [ADQST];
[RQGKMFPS] [RNGHPST] [RQGKPST] ** [ARQGMPSTV] [AEGS];
[MFTYV]K** [ANST] [AQSTY]G;
[NQGKMFSYV] [RQGLKS] * [ANDQKPS] [ARNQGMSTV] *G;
[RDQEK] [ARGLMST] * [ANGKPT] * [RNKMS] [AELM];
[\hat{ }ARDCEHFPWV]K [ANDQMPST] ** [RQGKMT] [NDEG];
[\hat{ }ACGHILPW] [RNDQGLKS] [\hat{ }DCELKFWYV] [\hat{ }RNCEILMFWY] **[RNDEGKFPS];
[NKMSTW] [ARKSTV] [NQGPS] [AQGKSV] **A;
[RNQGKTYV] * [ANQGMPST] [ANQGKST] * [AQGKMS] [AEGS];
[NQGIKFSTY]K[NDQHPS] *[ARNGKT] *[ADEPS];
[NMFTYV] [KPS] * [AQGKPTV] * [AQGKS] [DEGS];
[\hat{ }ADCEHILPW] [ARNQGKFPT] ** [ARNQKMSTY] [ARNQGMS] [ADEGPS];
[NQIKFY] [ADQLK] [ANGHM] **[ARQK] [NDGK];
[ARNDQGSTV] ** [RNQGKP] [RQEGKMT] [NEGKFS] [ARGK];
[NQGMT]K* [AQGPSTV] [AGMST] * [ANGS]; [DQKS] [QKMST] **[AGST]K[DEK];
[NQWV]K[GST] **SG; K** [ANQS] [ANQT] [ADGK] [ARDEG];
K**[NQSTY]S[AQGKT] [EGPS]; [RNQGT] *[AGST] [QGKT] [RGKT] *G;
[RNQGMST] [RNQGKPSV] [ANGPST] * [ARQGIKST] * [AGS];
[RNQGKTV] [RNQGKPS] * [ANQGSTV] [NGKMST] * [AGPS];
[QGKMT] [GFP]K* [NQEGTY] * [AQGY]; [NT] [AD] *K[RS] * [LP];
[NQMFTV] *K* [AQGMS] [AQGKTV] [EG];
[RGIKF] * [ARNQGT] * [AKMT] [ARGKS] [DEG];
[QMFT] [RNQGMPS] [RKP] [ANGST] **G; [NGMFT] [NGKS]K[DQT] **[GSW];
[GKFTY] *[AKPT] *[RNQST] [AGKST] [GS]; [QFS]K[GT] **[ST]A;
T [RNS] [ARGK] ** [RQGT] [EG]; T*N*SKG (SEQ ID NO: 199642);
[GSTYV] *K[QT] [NGS] * [GS]; [KMFPSY] [LK] *S* [ANQIST]G; K[KS] [DST] *S*G;
[RNQMPTV] *[GKT]S[ARQKS] *[AGS]; G*K*TAA (SEQ ID NO: 199643);
K* [ANS] [GS] [GT] * [ADP]; [RQGKP] * [GKP] [NDPT] [AQMS] *A;
[MFT] *K[APT] *[RQ] [ADS]; M*SKS*A (SEQ ID NO: 199596);
TK[NMP] **[ANQ]A; [NQGS]K**G[QGF]G; [NQF] [QS]K*[AS] *G;
GK[GT] [QT] **G; [RGT] [DGP]K[NQS] **A; TGK**[AST]A (SEQ ID NO: 199644); K[DS] [RN] *G* [AG]; [GMPTV] *K[ASTV] * [QST]G;
[ST]K* [AQ] * [QS]A; K*A[TV] * [RK]D; [MF]KN** [QP]A; Q [RQY] [KP] [ST] **A;
[NT] *KS* [ST] [AP]; TG**MKG (SEQ ID NO: 199645);
[QT] [RG] *S* [KT] [AP]; [FV] *K*TSA (SEQ ID NO: 199646);
QK[GT] [NS] **A; T**KS[GS]G (SEQ ID NO: 200022); [NP]K*S*GG (SEQ ID NO: 199647); NK**G[ST]A (SEQ ID NO: 199648); [QG]KN**SA (SEQ ID NO: 199649); TKS**SN (SEQ ID NO: 199650); MKNT**A (SEQ ID NO: 199651); Q*K[GS] *[NV]G; Q**KSNA (SEQ ID NO: 199652); NN**SKG (SEQ ID NO: 199653); M*N*GNA (SEQ ID NO: 199654); KTQ*S*S (SEQ ID NO: 199655); T**SGTP (SEQ ID NO: 199656); RS*S*GG (SEQ ID NO: 199522);
MG*R*GA (SEQ ID NO: 199514); [QT] *K[NS] [ST] *G; Q*KP*QG (SEQ ID NO: 199657); KA**GDR (SEQ ID NO: 199658); V*N*SSA (SEQ ID NO: 199641);
K*TG*RE (SEQ ID NO: 199659); M*K*TSG (SEQ ID NO: 199660); T*K*SSA (SEQ ID NO: 199661); YQK*S*S (SEQ ID NO: 199662); MK*T*TA (SEQ ID NO: 199663); KPN*S*A (SEQ ID NO: 199621); TK**GMG (SEQ ID NO: 199664); MPK*S*S (SEQ ID NO: 199665); DL*KP*K (SEQ ID NO: 199666);
KGNT**A (SEQ ID NO: 199667); MK*G*TG (SEQ ID NO: 199668); N**STMA (SEQ ID NO: 199669); NK**SDK (SEQ ID NO: 199670); T*K*SNS (SEQ ID NO: 199671); T*KG*VG (SEQ ID NO: 199672); TKQ**SA (SEQ ID NO: 199673); and V*NKV*G (SEQ ID NO: 199606); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position, wherein a viral particle having a capsid comprising the motif has increased binding to a liver cell relative to a control viral particle;
wherein the library comprises an amino acid sequence motif selected from the group consisting of: KQ**AKD (SEQ ID NO: 199674); NR**GGA (SEQ ID NO: 199675); KKD**RD (SEQ ID NO: 199676); QRNS**A (SEQ ID NO: 199677); NRGQ**A (SEQ ID NO: 199583); KKD**KD (SEQ ID NO: 199678); R*KDS*A (SEQ ID NO: 199634); RN**SGA (SEQ ID NO: 199679); RQ*PT*A (SEQ ID NO: 199515): or polynucleotides encoding the same, where “*” represents any amino acid and square brackets denote a list of amino acids that may occur at a position, wherein a viral particle having a capsid comprising the motif has increased binding to a brain endothelial cell relative to a control viral particle;
wherein the library comprises an amino acid sequence motif selected from the group consisting of: [QKMPT] [NKMPT] [ARGKS] [AQGT] ** [AG]; [TV]KPS**G (SEQ ID NO: 199680); [QT] [KS] *G [NKT] *G; [KMT] [RT] [GS] ** [NMT]G; [RQMT] [RQGK] [QPST] * [RGTV] *G; [GM]K[PS] ** [NT]G; KPT**MA (SEQ ID NO: 199681); [QGKT] * [GPT] [NQGT] [RKT] *G; [KT] [KS]SS** [AG]; [NGS] [AK] * [GKS] [NST] * [AP]; [GK] *T* [RT] [QS]G; [QKT] [GKF] ** [RGT] [GSY]G; [QKS] [RGKT] * [NT] [ANGT] *G; T**NRGG (SEQ ID NO: 199556); [NK] [RY] *T*[TV]G; Q*PTS*A (SEQ ID NO: 199682); [FS] [NG]K*[GS] *G; QR**STA (SEQ ID NO: 199683); [QMS]K*G*[NT]G; K*ST*NG (SEQ ID NO: 199684); QRSS**G (SEQ ID NO: 199685); [KV] [NK] *[GV] *S[AG]; N[RT] [AR] *[NT] *A; KN*GQ*G (SEQ ID NO: 199686); K*S*QSA (SEQ ID NO: 199687); [RN] [ST] [KS] *S* [GP]; QKN*A*A (SEQ ID NO: 199688); RP**MAG (SEQ ID NO: 199689); N**STMA (SEQ ID NO: 199669); N*K*GGG (SEQ ID NO: 199690); KTT**GG (SEQ ID NO: 199691); YKQ**GG (SEQ ID NO: 199692); N*K*SNP (SEQ ID NO: 199693); NKN*G*A (SEQ ID NO: 199694); S**KTGG (SEQ ID NO: 199695); GN*VK*G (SEQ ID NO: 199696); M*SKS*A (SEQ ID NO: 199596); MK**SAG (SEQ ID NO: 199697); N*K*SQG (SEQ ID NO: 199698); NRPS**P (SEQ ID NO: 199699); QKT**GG (SEQ ID NO: 199700); RS*Q*QS (SEQ ID NO: 199701); and T*RT*GG (SEQ ID NO: 199702); or polynucleotides encoding the same, where “*” represents any amino acid and square brackets denote a list of amino acids that may occur at a position, wherein a viral particle having a capsid comprising the motif has increased binding to a brain endothelial cell relative to a control viral particle;
wherein the library comprises an amino acid sequence motif selected from the group consisting of: QSRT**P (SEQ ID NO: 199580); MSRN**A (SEQ ID NO: 199591); KKD**RD (SEQ ID NO: 199676); NRGQ**A (SEQ ID NO: 199583); KKD**KD (SEQ ID NO: 199678); KKD*K*D (SEQ ID NO: 199703); NR**GGA (SEQ ID NO: 199675); and R*KDS*A (SEQ ID NO: 199634); or polynucleotides encoding the same, where “*” represents any amino acid and square brackets denote a list of amino acids that may occur at a position, wherein a viral particle having a capsid comprising the motif has increased binding to a brain endothelial cell relative to a control viral particle;
wherein the library an amino acid sequence motif selected from the group consisting of: NR**GGA (SEQ ID NO: 199675); [STY] [KP] * [QS] [GSV] *G; [GM] **[GT] [GK] [NF]G; QRPN**A (SEQ ID NO: 199704); [QS]K[GT] *S*G; Q*K*AQG (SEQ ID NO: 199705); Q* [GK] [NS] [KS] *G; [QY] [RK]P*[AT] *[AP]; [QM]K*G*TG (SEQ ID NO: 199706); TK*N*QG (SEQ ID NO: 199707); K*ST*SG (SEQ ID NO: 199708); KN*G*SA (SEQ ID NO: 199570); KN*GQ*G (SEQ ID NO: 199686); MK**SQG (SEQ ID NO: 199709); TGT*R*G (SEQ ID NO: 199710); GK*ST*A (SEQ ID NO: 199711); MK*GS*G (SEQ ID NO: 199712); MSK**AG (SEQ ID NO: 199713); K*PTT*G (SEQ ID NO: 199714); KP*T*GG (SEQ ID NO: 199619); MP**SGS (SEQ ID NO: 199715); Q*K*SNG (SEQ ID NO: 199716); QKY*T*G (SEQ ID NO: 199717); R*PS*QG (SEQ ID NO: 199718); T**PTAG (SEQ ID NO: 199719); and TGKS**A (SEQ ID NO: 199622); or polynucleotides encoding the same, where “*” represents any amino acid and square brackets denote a list of amino acids that may occur at a position, wherein a viral particle having a capsid comprising the motif has increased transduction efficiency for a brain endothelial cell relative to a control viral particle;
wherein the library comprises an amino acid sequence motif selected from the group consisting of: N**SRQG (SEQ ID NO: 199528); SP**RGG (SEQ ID NO: 199640); NQ**RSA (SEQ ID NO: 199720); QKY*T*G (SEQ ID NO: 199717); S*QQR*G (SEQ ID NO: 199721); MRG**MG (SEQ ID NO: 199505); NR**GGA (SEQ ID NO: 199675); NRGQ**A (SEQ ID NO: 199583); T**NRGG (SEQ ID NO: 199556); T*S*RMG (SEQ ID NO: 199532); T*TNR*G (SEQ ID NO: 199587); and TAN*R*G (SEQ ID NO: 199586); or polynucleotides encoding the same, where “*” represents any amino acid and square brackets denote a list of amino acids that may occur at a position, wherein a viral particle having a capsid comprising the motif has increased binding to a brain endothelial cell relative to a control viral particle;
wherein the library comprises an amino acid sequence motif selected from the group consisting of: [QKMFT] [RNQP] [RQKPS] [ARDG] **G;
[RQGMSTY] [ARNKP] [RNGKPST] **[RNQGKMST] [AEG];
[QGF] [GPT] [RK] * [NMS] *G; [RK] [NQ] [PT] ** [AT]G;
[RMT] [RQGT] [NPST] * [RQV] *G; [KMST] * [NQGT] [RQGS] [RQT] *G;
[RNY] [RKP]Q** [QGT] [AG]; [NQKT] [RKP] ** [NGS] [NQGTV]A;
[RKMST] [NGKPS] * [ARQS] * [ANQGTY] [AS]; [RKMP] [NKS] * [ANQS] * [AGSV]G;
[RGMFT] [NK] * [AGPV] [NGKTV] *G; [RKMTY] * [RKPST] [AGST] [GST] * [GPS];
SK*GN*A (SEQ ID NO: 199722); KP[AP] **G[AG]; [NST] [RK] *T* [NGV]G;
[RNT] * [GKT] * [ARGK] [QMST]G; [GK] [NP] [KS] * [NS] * [AS]; QK*GT*G (SEQ ID NO: 199555); [RQK] ** [ANT] [RT] [GT]G; [RK] [NF] ** [GTV]SG;
[GK] [KP] *[SV]S* [NP]; TG*NK*A (SEQ ID NO: 199723);
[NT] [RT] [PS] [KS] ** [AP]; QKY*T*G (SEQ ID NO: 199717); RS**TQS (SEQ ID NO: 199724); [RN] *[NT] [RS] *GG; KTQ**AS (SEQ ID NO: 199725);
RS**GGG (SEQ ID NO: 199726); KPP*T*G (SEQ ID NO: 199727);
[KY] * [QP]T* [GT]G; S*TT*NG (SEQ ID NO: 199728); KSPT**A (SEQ ID NO: 199729); SRTT**G (SEQ ID NO: 199730); [GY]K[QT] [QT] **G; Q**KSNA (SEQ ID NO: 199652); K*NST*A (SEQ ID NO: 199731); TRS*T*G (SEQ ID NO: 199732); K*S*SGA (SEQ ID NO: 199733); N**SRQG (SEQ ID NO: 199528); VRP*T*G (SEQ ID NO: 199734); Y*T*SKG (SEQ ID NO: 199735);
TR*VS*G (SEQ ID NO: 199736); TG**RSG (SEQ ID NO: 199585); K*P*SGG (SEQ ID NO: 199737); KTS**GG (SEQ ID NO: 199738); N**GQKG (SEQ ID NO: 199739); N*GNR*A (SEQ ID NO: 199740); QR*NS*A (SEQ ID NO: 199551); QRP*S*A (SEQ ID NO: 199741); R*QN*TG (SEQ ID NO: 199742);
RP*T*AP (SEQ ID NO: 199743); SRTT*NG (SEQ ID NO: 199576); and
TKSS**A (SEQ ID NO: 199630); or polynucleotides encoding the same, where “*” represents any amino acid and square brackets denote a list of amino acids that may occur at a position, wherein a viral particle having a capsid comprising the motif has increased transduction efficiency for a brain endothelial cell relative to a control viral particle;
wherein the library comprises an amino acid sequence motif selected from the group consisting of: [\hat{ }ADCEGHILWY] * [\hat{ }DCEGHILFWY] [ARNGKMPST] [ARNQGISTV] *G;
[\hat{ }ADCEHILMWY] [\hat{ }DCEHILFPWY] [\hat{ }RCEHILMFW] *[\hat{ }NDCEHLFPWY] *G;
[\hat{ }ADCEHILMW] [\hat{ }CEGHIFWYV] [\hat{ }CEHILFPW] [\hat{ }CEHILKMFWY] **G;
[RNDQKMPV] [ARNQMPST] ** [ARNQGKST] [ARNDQGKTV] [ARDIKPS];
[\hat{ }ARDCEHILW] [ARQGKPST] ** [ARNQKMSTV] [ANQGKMTYV]G;
[\hat{ }ACEHILMFSW] [\hat{ }CEHIKFWYV] *[\hat{ }DCQEHLFWY] [\hat{ }DCEHLFTWY] *[AEGLKP];
[RQKMSTV] ** [ARNQKPSTV] [ARNQGMSTV] [ARNQGSTV]G;
[\hat{ }RDCEHLPW] [\hat{ }CEHILMPSWV] [\hat{ }ANCEILMFWV] * [\hat{ }DCQILMFWV] * [NDQEGMPSY];
[RNQGKMS] [RQKST] [\hat{ }RDCEHILFWY] * [ARNMSTV] * [APS];
[\hat{ }ADCEHLFPW] * [ARNQGPST] [RQGKSTY] * [ANGKMSTV]G;
[\hat{ }ADCEHIK] [\hat{ }CQEHILFWY] [ARNQGKMPS] ** [ANQGKMSV]G;
[\hat{ }ADCEHPTWV] [\hat{ }ARCEIFWYV] [\hat{ }CILFTWY] ** [ARQGKPSTV] [ANDQEGKMS];
[RNQGSTV] **[RNGKSTV] [\hat{ }ADCHLMFPWY] [\hat{ }CHILKPWYV] [AGKPSV];
[RNGKFTYV] * [ARNQGPST] * [ARNQKPSTV] [ADQGKMSTV]G;
[ANQMFSTV] *K* [AQGST] [ANQGMST]G;
[\hat{ }ADCEHILW] * [\hat{ }ACEHILFWYV] [ANQGKPSTV] * [\hat{ }DCEHILKFWY] [ANDGPSY];
[\hat{ }ADCEHITW] [\hat{ }DCEGHIMFWY] * [ANQGMPSTV] * [\hat{ }RDCEHLPTWY]G;
[RNQILKFSV] [QGKPST]T** [ARQGKMST] [ADEGS];
[\hat{ }ACHILKW] [\hat{ }CEHILMFPWY] [\hat{ }NDCELFWYV] [\hat{ }RNCEILMFWY] ** [\hat{ }CQHILKMPTY];
[RNQGKMSY] [\hat{ }RDCGHILMWY] ** [ARGKMSTV] [\hat{ }NDCEHLPWYV] [DGPS];
[\hat{ }ARCEHILWY] [ARQGKST] *[\hat{ }CQEHILMFWY] [ARNGPST] *[NDGPS];
TK[ANQMST] ** [QKS] [NDS];
[NQGKMST] * [ARNQGKPS] [ARNQGPTV] [RNQHKMT] * [NGPS];
[NGFST]K** [ANGSTY] [ANGMPT]A; [RQMFS] [RKPT] ** [NQGT] [NQS] [NGS];
[\hat{ }ADCEHPTW] [RNQLKMPST] * [\hat{ }CEILKFWY] * [\hat{ }CEHILPSWY] [ANDQEKPS];
[QT] * [QT]R*QA; [RNGT] * [RNGKST] [ARNQGT] [RNQGKMT] * [AG];
[DQEMPST] [ARGK] *[ARNQGKS] *[ADQGKSY] [ADLMPT];
[NGIKFTV] * [ARNGKTY] * [NQKMSTV] [ARNIKSV] [ADEPS];
[RNQKMFST] [RDQKS] [RNQPST] *[AGS] *[AS];
[RQGKS] [QKST] [RNQST] ** [NT] [GPS];
K** [ANGSTY] [QGMST] [ANGKMT] [ADEGP];
[RKM] [RNQGPS] * [ANQST] [NQST] * [GS]; [RQGIKT] [PS] [RKPS] * [ANGMT] *G;
[RQKM] * [NPS] * [AQGT] [ANQGST] [APS];
[ADK] ** [NGPST] [AEGMST] [ANQGKT] [ARS]; [QKMPTV] * [NKPS] [QGKPT]S*A;
[RNQGST] * [RNQGKSV] * [RNQGK] [AGKTV]G; [NQGKT] [RNK] * [ANGST] [ANGMT] *A;
[RNQGKMY] [RNQGS] * [NQGKS] * [AQGST] [AG]; [NK] **G [QT] [KS]G;
[RKMTY] [RP] [RNGKPS] * [ANQST] * [GPST];
[QKMFPYV] [RNGKPST] [RNGPST] [ARDQGKST] ** [GPS];
[RNQGKWV] [ANQKPSTV] [ARNQGKST] [AQGKST] ** [AP]; TK* [GSTV] * [QGV]G;
T[RGS] [RGKT] **[RNST] [EGP]; [RGV]P[RNK] *[QST] *A;
[RKMV] * [ANPST]S [GKST] * [DGPS];
[NQMSTV] [RNQGHK] ** [ARGIKMST] [ARQKSV] [ADE]; [NQK] * [NKT] *S [NV]G;
[RNQFT] [RQK] [QGKST]N** [APS]; [NT] [GK] [NKMS] ** [ANQT]A;
[RQK] * [ANQGPST] [NGS] [ANGKPT] * [AGP]; [TYV] * [RK] [QGT]S* [GPS];
[NK] [RNP] [PT] [NS] ** [GP]; K* [NG] [AY] *KE; [NQ]R[NQGT] ** [NQT]A;
MKN* [GS] *G (SEQ ID NO: 199744); [QGKM] * [GS] * [KS] [NG] [AG];
[RNGMTYV] [RGKS] * [AQKPS] [NGS] *A; [QMTYV] [GK] * [NGKS] *TG;
[QKM] [RKMS] [NDP] [GSTV] **A; [NQSY]K* [QS] [ANQSV] *G; [QMT]RP** [AMS]A;
K*T [GI] * [RK] [DE]; [RQP] * [KPT] [DQGS] [AS] *A; TKP** [AQ]A (SEQ ID NO: 199745); T [RK] ** [NGL] [NMS] [AG]; K**Q[AS] [GK] [DS]; RQ* [NP]T*A (SEQ ID NO: 199746); [KS] [GY] *T* [TV]G; K* [QT] [GT] [GS] * [AS]; RT*T*SA (SEQ ID NO: 199747); K*A[NV] *[RS] [DG]; [NK] **Q[AR]S[AS];
[QG] [RP] [KP]N**A; [RT] [GS] ** [RN] [ST]G; MRPN**G (SEQ ID NO: 199748); G*KSV*G (SEQ ID NO: 199601); NK*T*SA (SEQ ID NO: 199749);
[NF] * [NK] * [NG]T [AS]; [GK] [KP] [NQT] * [GS] *A; MRT**SP (SEQ ID NO: 199750); [NM]K*QT* [GS]; GN**KNG (SEQ ID NO: 199751);
[NT] [GT] [RK] *N*A; K*ASS*A (SEQ ID NO: 199571); RT*GT*G (SEQ ID NO: 199752); [RQS] [AR] [PT] ** [NV] [GS]; RPT*S*[GS](SEQ ID NO: 199753);
K**GKSA (SEQ ID NO: 199754); [GV]KP**NA (SEQ ID NO: 199755);
TK*T* [KS] [AD]; TGK*G*A (SEQ ID NO: 199756); KP*GT*G (SEQ ID NO: 199757); RP*QQ*A (SEQ ID NO: 199758); KPNN**P (SEQ ID NO: 199759);
TGK**SA (SEQ ID NO: 199620); G**QKSG (SEQ ID NO: 199760); TG**KTA (SEQ ID NO: 199761); TN**RQG (SEQ ID NO: 199762); TG*K*SG (SEQ ID NO: 199763); I*AR*KE (SEQ ID NO: 199764); N*K*NNG (SEQ ID NO: 199765); R*S*STP (SEQ ID NO: 199506); KGNN**G (SEQ ID NO: 199567);
TK*N*QG (SEQ ID NO: 199707); G**QKGG (SEQ ID NO: 199766); K*AT*KD (SEQ ID NO: 199767); K*NQS*G (SEQ ID NO: 199768); KPS*N*A (SEQ ID NO: 199627); R*S*NVA (SEQ ID NO: 199769); RP*GT*A (SEQ ID NO: 199770); SRTT*NG (SEQ ID NO: 199576); TKQ**SA (SEQ ID NO: 199673); and TS**RTP (SEQ ID NO: 199771); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position, wherein a viral particle having a capsid comprising the motif has increased biodistribution to liver relative to a control viral particle;
wherein the library comprises an amino acid sequence motif selected from the group consisting of:
RQSA**T (SEQ ID NO: 199772); RAHS**A (SEQ ID NO: 199773); DG*K*KL (SEQ ID NO: 199774); NR*A*DK (SEQ ID NO: 199775); G*N*ANR (SEQ ID NO: 199776); RQ**NST (SEQ ID NO: 199777); [RKT] [STV] [RQE] **R[ADE];
T*GG*RN (SEQ ID NO: 199778); Q*G*VRG (SEQ ID NO: 199779); MDK**QR (SEQ ID NO: 199780); NGG**GR (SEQ ID NO: 199781); V*RQ*AG (SEQ ID NO: 199782); L*REG*R (SEQ ID NO: 199783); QAG**RG (SEQ ID NO: 199784); QR*VV*A (SEQ ID NO: 199785); RE**ARG (SEQ ID NO: 199786);
RQMA**A (SEQ ID NO: 199787); S**REIR (SEQ ID NO: 199788); TK*R*DT (SEQ ID NO: 199789); V*R*SAG (SEQ ID NO: 199790); V*RS*GG (SEQ ID NO: 199791); and VQR* S*G (SEQ ID NO: 199792); or polynucleotides encoding the same, where “*” represents any amino acid and square brackets denote a list of amino acids that may occur at a position, wherein a viral particle having a capsid comprising the motif has increased transduction efficiency for a liver cell relative to a control viral particle;
wherein the library comprises an amino acid sequence motif selected from the group consisting of:
[RNGPS] * [GKSTY] [RNGKST] * [ANGMSTV]A;
[RNGKST] [NQGKP] * [NQGKPS] [NQKMSTV] *A; K** [AS] [AS] [AK] [EPS];
[RNQGKPSTV] [ARNGKPSTV] [ANDQGPSTY] [NQGKPST] ** [AMPSV];
[RQGKPST] [RQLKPT] [NDQST] * [ANQGSV] * [AGSV];
[RGKMST] [NKPST] ** [AQGKSTV] [ANDQG] [RDGPS];
[RKMPST] [ARGKPST] [ANQPSTYV] ** [ARNGKMST] [ADMPS];
[NDGPT]K* [NQGKPT] * [NQG]A; [KP] [RNGPT] * [QGST] [ANQT] * [GPS];
[QGPS] [QK] ** [ANST] [GMST]A; [NGPT] [NQGK] [NKV] ** [ANGST]A;
[NPST] * [NK] [AQKT] * [QS] [PS]; [RNGK] * [NQST] [ANQGKST] [ARNGPT] *A;
[RNQPST] [QGK] *S* [ANQGKS] [GP]; K[NPT] * [ANGS] * [QTV]A;
[RQPT] [NDGKT] [KS] [AGST] **A; [RNQK] [PST] [NQKT] ** [NQT] [APS];
[KP] [RQP] *T [GT] *A; [RKP] [QS] [NGKPS] * [GT] *A; K* [NS] [AQGT] * [QGS]A;
[RN] [ANSV] [NGK] ** [QMV]G; [NK] [KST] ** [NGP] [AQGT]A;
[NGK] * [QKT] * [NQGP] [QGMTV] [AGS]; [QPST]K* [NGPS] [AMST] * [AG];
[KT] [RT] *SG* [PV]; KQ[GT] [QT] **A; [NKMT] * [RNQPS] [NGK] [ARGT] * [GMPV];
K*N* [NG] [NGV]A; R[PS] * [PS]G*A; [GST] [QGS]K*N*A;
[KS] * [NT] * [GS] [IS] [AP]; NKPA**S (SEQ ID NO: 199793);
[GKPS] [RKST] * [AQS] * [NS] [AGS]; [NK] ** [NPST] [GPT] [ANM]A;
[NQP] *K[PST] [ST] *A; [PT] *KG[AG] *A; SK[MT] **Q[AP]; NTR**SA (SEQ ID NO: 199503); PKS**SG (SEQ ID NO: 199794); TK*PS*S (SEQ ID NO: 199795); [NG] *K[ST] [QT] *[GP]; FGKQ**S (SEQ ID NO: 199796);
[NQ] [RKPSTV] [ARNKT] * [NGT] * [AGM]; [RK] * [NT] [NS] * [NS]P; K*N[QT]S*A (SEQ ID NO: 199797); [KMS] ** [GS] [AN] [NQT] [AG]; [NQ]K* [AT] [AG] *A;
KQ**AKD (SEQ ID NO: 199674); K*[PT]S*[AQG]A; [NMS] *K[PS] *[NG]G;
NAKS**G (SEQ ID NO: 199798); KQ**TQA (SEQ ID NO: 199799);
[NK] [ST] [NK] *[AT] *P; K*TQ*GA (SEQ ID NO: 199800); T**QSGF (SEQ ID NO: 199801); KTQ*T*G (SEQ ID NO: 199802); QK**GAP (SEQ ID NO: 199803); SK**NAA (SEQ ID NO: 199804); K*AT*KD (SEQ ID NO: 199767);
R*N*ANP (SEQ ID NO: 199805); GKQ*S*P (SEQ ID NO: 199806); PQKS**A (SEQ ID NO: 199807); N[GK] *[KT] *SA; SQ**TNP (SEQ ID NO: 199808);
PR*Q*SP (SEQ ID NO: 199809); NG*R*TP (SEQ ID NO: 199810); TKM**QA (SEQ ID NO: 199811); NK*TN*S (SEQ ID NO: 199812); N*K*NNG (SEQ ID NO: 199765); PK*GN*A (SEQ ID NO: 199813); FQKA**G (SEQ ID NO: 199814); G*KT*QA (SEQ ID NO: 199815); K**TSQS (SEQ ID NO: 199816);
K*ASS*A (SEQ ID NO: 199571); MNQ*R*P (SEQ ID NO: 199817); RQ*A*TS (SEQ ID NO: 199818); SK**NQP (SEQ ID NO: 199819); and TKN**QS (SEQ ID NO: 199820); or polynucleotides encoding the same, where “*” represents any amino acid and square brackets denote a list of amino acids that may occur at a position, wherein a viral particle having a capsid comprising the motif has increased biodistribution to heart relative to a control viral particle;
wherein the library comprises an amino acid sequence motif selected from the group consisting of:
[GFTW] [AKM] [NQKPT] [QGKP] ** [AG]; [MT] [RGP] [RQP] [KS] **G;
[QMT] [RGP] [RQGP] * [QKMT] *G; [QG] * [AGP] [GT]K*G; [RF] [KP] ** [AQ] [NQ]G;
KT [AS] ** [GK] [AEP]; G**TKMG (SEQ ID NO: 199821);
[GK] * [PT] [QG] * [QS]G; [GT] [NG] ** [RK]SG; [QT] * [RG]S [KT] *G;
[QK] * [GP] * [KST] [GSV]G; [RM]SS [KT] ** [AS]; [RM] [NQG]S* [GKV] *G;
[MT] *STK*G (SEQ ID NO: 200023); K[GT]S[QT] **G; K*P[NS] *GA (SEQ ID NO: 199822); K[MST] [PS] [GT] **A; K[DT] [AR]S** [AG]; [IY]KP** [KS] [ND];
R[NQ]S [AS] **G; M*SKS*A (SEQ ID NO: 199596); K*S [AN] * [AGS] [AS];
[QGF] [NKS] * [GPV] [NGK] *G; [RK] [DST] [RS] * [GS] * [AGP];
[KT] [GY] *[KT] *TG; R*SN*TG (SEQ ID NO: 199552); AKY*K*E (SEQ ID NO: 199823); [KT] [QG] [AT] **[ST]G; MR*NQ*G (SEQ ID NO: 199604); K*PT*TG (SEQ ID NO: 199824); NRPS**P (SEQ ID NO: 199699); KRPD**G (SEQ ID NO: 199825); R*SSV*G (SEQ ID NO: 199826); KSSS**G (SEQ ID NO: 199827); TK*S*YA (SEQ ID NO: 199828); FK*S*QG (SEQ ID NO: 199829);
K**NTTG (SEQ ID NO: 199830); FK*P*QG (SEQ ID NO: 199831); FKM**QG (SEQ ID NO: 199832); GK*VS*N (SEQ ID NO: 199833); K*SSV*G (SEQ ID NO: 199834); K*ST*NG (SEQ ID NO: 199684); KF**TSG (SEQ ID NO: 199835); KGSS**P (SEQ ID NO: 199836); N*K*GMG (SEQ ID NO: 199837);
and YKP*T*P (SEQ ID NO: 199838); or polynucleotides encoding the same, where “*” represents any amino acid and square brackets denote a list of amino acids that may occur at a position, wherein a viral particle having a capsid comprising the motif has increased biodistribution to spleen relative to a control viral particle;
wherein the library comprises an amino acid sequence motif selected from the group consisting of:
[NEGKMFTWY] [ARDKPST] [ARNQGST] [ANQKPST] ** [AGFS];
[ARQLKM] [ANGHIT] * [RQGHP] * [AGKST] [ARDKS];
[GKFTY] [GKT] **[ARGV] [AQFS]G;
[NGKT] **[NGKMPT] [LKPST] [NGHKMFT] [RDGSY];
[RNQKMT] [RNGIL]P [NDHKST] ** [ANGKPV];
[DEGKMFT] [GKMFP] [ARHMF] ** [NQGKST] [DGKP];
[RQGFYV] * [RGLKPS] [RNGS] * [ANDQPT] [RGKPY];
[RQKM] [RGT] * [NGP] [AQGPV] * [GMY];
[RQGIKMTYV] [RNDQGHPST] [\hat{ }DCEILMFWYV] * [\hat{ }DCEHLKFWY] * [ANGKFPS];
[RGKMSTV] *[ARNHMST] [NQKST] [ARGKTV] *[ARG]; K*PG*QG (SEQ ID NO: 199839); [QGFT] [ANK] * [APTV] [ARNGKP] * [GV]; [TY]KP*T* [PY];
[QGTY] [RK]P** [AGMS] [ANG]; [GLMTY]K* [HPS] * [NFSY] [AQG];
[NKT] [RGY] * [KT] * [TV]G; [RM] [HP] [KP] ** [HP] [AM];
[RKMTY] * [GPT] * [ARNST] [QGKS]G; Q* [RP] [QG] [KT] * [GP]; S*NAH*R (SEQ ID NO: 199840); R**VRDV (SEQ ID NO: 199841); KM**PKD (SEQ ID NO: 199590); RP**[QG] [NG]G; SP**RGG (SEQ ID NO: 199640);
[MST] [HKY] [GP] [AQ] ** [KY]; [QFV] [QP] [RNG]H** [KV];
[RY]P*[KS]S*[AGS]; FK*[PS] *QG (SEQ ID NO: 199842); MGS*K*G (SEQ ID NO: 199843); [NK] * [RS] * [TV] [TY] [GM]; G*TQ*SG (SEQ ID NO: 199844);
TR*VS*G (SEQ ID NO: 199736); YKPP**G (SEQ ID NO: 199845); QR*S*TG (SEQ ID NO: 199525); SK**YGA (SEQ ID NO: 199846); TK*VS*N (SEQ ID NO: 199847); QK*PS*A (SEQ ID NO: 199848); GK*ST*A (SEQ ID NO: 199711); QKS*S*G (SEQ ID NO: 199849); K*TI*KD (SEQ ID NO: 199850);
GK*PS*A (SEQ ID NO: 199851); AKY*K*E (SEQ ID NO: 199823); FK*T*MS (SEQ ID NO: 199852); GK*VS*N (SEQ ID NO: 199833); K*MQ*QG (SEQ ID NO: 199853); Q*KPH*N (SEQ ID NO: 199854); QKT**GG (SEQ ID NO: 199700); QPRG**G (SEQ ID NO: 199855); R**SVAG (SEQ ID NO: 199856);
R*Q*APF (SEQ ID NO: 199857); and SK**NQP (SEQ ID NO: 199819); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position, wherein a viral particle having a capsid comprising the motif has increased biodistribution to kidney relative to a control viral particle;
wherein with the library comprises an amino acid sequence motif selected from the group consisting of: YMNN**K (SEQ ID NO: 199858); I*RS*TG (SEQ ID NO: 199859); NGG**GR (SEQ ID NO: 199781); and RQMA**A (SEQ ID NO: 199787): or polynucleotides encoding the same, where “*” represents any amino acid and square brackets denote a list of amino acids that may occur at a position, wherein a viral particle having a capsid comprising the motif is capable of transducing kidney cells in vivo.
wherein the library comprises an amino acid sequence motif selected from the group consisting of:
GK** [ST] [NMT] [AP]; [NK] * [NS] [KT] [NS] *A; [NGP] [QKS] [KT] * [NGS] * [AG];
K*N[AN] * [NQG] [AP]; [NQKPS] [NQKT] [AQGKST] [AQGST] ** [AGS];
[KP] [KPST] ** [ANK] [ANDG] [ADPS]; [NKV] [NG] * [GK] * [KST] [AG];
[GP] [RNK] [NKPT] ** [GS] [AM]; K*TS* [AS] [AV];
[NGPST] [GK] *[NQGKS] [NKT] *A; PR*Q*SP (SEQ ID NO: 199809);
[NP] [RKS] [NK] * [GT] * [APV]; [NG] [QK] [KT]N**A;
[RPST] [KT] [NMS] ** [AQT] [PS]; [NT] *KS* [ST] [AP];
[KM] [NPT] **G[QG] [ARS]; K*N* [GS] [NI]A;
[RNPS] [ARNK] [ANG] [QGKS] ** [AGP]; K[LKPT] [ND] * [AGS] *A;
KT [NQ] ** [AQGMS] [AS]; K[NQT] * [GPS] [AQGV] * [APV]; [PS] *K[AQ] *QS;
[NP] *KS[ST] *A; KQ[GT] *[TV] *A; K*TS*QA (SEQ ID NO: 199860);
[KT] * [NKP] [NG] [AG] * [AMP]; P*KG*GV (SEQ ID NO: 199861);
[NP] [RG] * [QS] [QK] *P; KP [QS] [NT] ** [AM]; K**PGMA (SEQ ID NO: 199862); N*K*NNG (SEQ ID NO: 199765); RPNN**P (SEQ ID NO: 199863);
K*T*QQA (SEQ ID NO: 199864); [PS]K[PT] **[QG] [AS]; PK*PS*A (SEQ ID NO: 199865); KTV**KD (SEQ ID NO: 199866); KA*TN*A (SEQ ID NO: 199867); KN*A*QA (SEQ ID NO: 199868); K*QTG*A (SEQ ID NO: 199869);
KTNG**P (SEQ ID NO: 199870); K*T*NTS (SEQ ID NO: 199871); KTN*A*P (SEQ ID NO: 199872); KST**TA (SEQ ID NO: 199873); DK*K*GA (SEQ ID NO: 199874); KPNN**P (SEQ ID NO: 199759); KTQ*S*S (SEQ ID NO: 199655); N*QKT*G (SEQ ID NO: 199875); K**TGNA (SEQ ID NO: 199876);
K*N*NVA (SEQ ID NO: 199877); KP*TN*S (SEQ ID NO: 199878); KPA**GA (SEQ ID NO: 199879); P*KG*SA (SEQ ID NO: 199880); QK**GAP (SEQ ID NO: 199803); SK*S*QP (SEQ ID NO: 199881); or polynucleotides encoding the same, where “*” represents any amino acid and square brackets denote a list of amino acids that may occur at a position, wherein a viral particle having a capsid comprising the motif has increased biodistribution to serum relative to a control viral particle;
wherein the library comprises an amino acid sequence motif selected from the group consisting of:
[RQKP] [RQPT] ** [ANGT] [ANGS] [AS];
[RNQKP] [RGST] [RNQKPV] ** [ARNGKMST] [RDGMPS]; K* [NQT] * [QGS] [NQIM]A;
[EGKMPST] [RGKPST] [ANQPSTYV] **L[ANGST] [AKPS];
[RNQGKPW] [ARNQGPST] [ANDQGKS] [NQGKT] ** [AKMPS];
[NGT] * [KS] [NGKS] [ANGMT] *A;
[RNGHKMP] * [NQGPSTYV] [ARNQGK] * [ANQGKS] [ARDG];
[NKT] *[NQPS] [NGK] [ARGT] *L[GMP]; [NGPS] *[NQK] [AQKT] *[NQST] [GPS];
[NQGMPSV] [KS] [ANKSTY] [ANQGT] ** [AP];
[RGKMPSV] [QKPST] [NDQKST] * [ANQGSV] *A; [KMPT] [RPT] [NK] * [GS] * [GPSV];
[QGPSTV]K* [NQGPSV] [ANMST] * [AGPS]; [QK] * [NG] [NS] * [NGV] [GP];
[RGTV] ** [NQS] [QGTY] [NMT] [AKP]; [NPS] *K[GS] * [ST]A;
K** [QG]A [QS] [AS]; [NQPT] [KV] [NQKT] * [AGS] * [GMS];
[RNGKT] [QGPST] * [NQGKS] [ANQGKTV] * [AGV]; [QGT] [GT]K** [AT]A;
[NDKS] * [RKT] * [NGMS] [NPST] [KPS]; [NK] [AGT] * [GKS] [AKS] *P;
[GK] * [QHK] * [NP] [LTV] [AQ]; [QKP] * [ANK] [QST]S*A;
[NV] [RS] [AKP] * [NT] *A; [NST]K[MPT] ** [QG] [AP];
[NQKST] [RNK] [ANDQST]S**A; T**SSNR (SEQ ID NO: 199882);
[KP] [ARQ] *T [NGT] *A; [NQG] [QK] * [KST] [ANGS] * [AM]; [SV] [RK]TS** [GP];
K**GTNA (SEQ ID NO: 199883); NK[NQ] *[AG] *A; K*[PT]S*[QGS] [AV];
[KTW] [NKT] [QGS] **Q[ANKS]; [RNKP] [RNGT] * [QGKPS] * [GST] [APS]; PKS**SG (SEQ ID NO: 199794); RDKS**A (SEQ ID NO: 199612);
[RY] * [ST] [KS] * [ST] [AP]; [GF] *K[QT]T* [PS]; [RN] * [AP] [GK]S* [AG];
RNGS**G (SEQ ID NO: 199884); KT [NQG] * [AS] * [GPS]; K*NSS* [PT](SEQ ID NO: 199885); Q*GSK*G (SEQ ID NO: 199886); KVS*T*A (SEQ ID NO: 199887); NSK*T*P (SEQ ID NO: 199888); [NGP]K* [NGPST] * [ANQG]A;
K[NT] * [AG] *QA; [GKP] [QKT] ** [ANST] [NGMT] [AP]; QK**GAP (SEQ ID NO: 199803); FQK*A*G (SEQ ID NO: 199889); K*QTG*A (SEQ ID NO: 199869);
[KS] [QK] **[QT]Q [AP]; KQTQ**A (SEQ ID NO: 199890);
[GP] *K[GT] *[QG] [AV]; [SV] *[RK] *SAG; MN**GQR (SEQ ID NO: 199891);
[NS]K*A*S[AG]; K**TGNA (SEQ ID NO: 199876); N*K*GQG (SEQ ID NO: 199892); TK*V*QG (SEQ ID NO: 199893); QK*TS*P (SEQ ID NO: 199894);
QK*S*[QS] [PS]; NKV**SA (SEQ ID NO: 199895); K*SGT*A (SEQ ID NO: 199896); PK*S*AG (SEQ ID NO: 199897); G*Q*TQG (SEQ ID NO: 199898);
K**TSQS (SEQ ID NO: 199816); K*TST*A (SEQ ID NO: 199899); KNGS**G (SEQ ID NO: 199900); KQG*T*A (SEQ ID NO: 199901); NVK**QG (SEQ ID NO: 199902); PR*QQ*P (SEQ ID NO: 199903); SN**KSA (SEQ ID NO: 199904); and T*KS* T P (SEQ ID NO: 199905); or polynucleotides encoding the same, where “*” represents any amino acid and square brackets denote a list of amino acids that may occur at a position, wherein a viral particle having a capsid comprising the motif has increased biodistribution to brain relative to a control viral particle.
wherein the library comprises an amino acid sequence motif selected from the group consisting of:
[NPT]K[PY] [GP] **A; KT**PQA (SEQ ID NO: 199906); KT**AKE (SEQ ID NO: 199907); [KPTY] [RQKT] [APSY] ** [AGKS] [ANE];
[RQK] [QPST] [KS] [MST] **G; K* [AGS] [NS] [AST] * [AP];
[RK] [GST] [AS] [GST] ** [APS]; Q*GSK*G (SEQ ID NO: 199886);
[RKMT] [RQGKS] [PS] * [QKSTV] *G; [RKY] [QKT] [PST] * [STV] * [AP];
[NK] [RMS]P[GST] **[AP]; [MT]PK*[AT] *G; K*TTG*G (SEQ ID NO: 199908);
[TV]KP[GS] **G; PK**NGA (SEQ ID NO: 199909); PK*PS*A (SEQ ID NO: 199865); TPR*G*G (SEQ ID NO: 199910); KTQ**QA (SEQ ID NO: 199911);
QPK*G*G (SEQ ID NO: 199912); RNS**NG (SEQ ID NO: 199913); K**NTTG (SEQ ID NO: 199830); K*ST*[NS]G (SEQ ID NO: 199914); YKPP**G (SEQ ID NO: 199845); K*SSV*G (SEQ ID NO: 199834);
[RNQ] *[GKS] *[GK] [MTV]G; KQ*SV*A (SEQ ID NO: 199915); PR*Q*SP (SEQ ID NO: 199809); R*SN* [NT] [AG]; K*PS*QA (SEQ ID NO: 199916);
FK*TA*G (SEQ ID NO: 199917); GK*PS*A (SEQ ID NO: 199851); K*GS*MS (SEQ ID NO: 199918); K*SN*GS (SEQ ID NO: 199919); FK**AQG (SEQ ID NO: 199920); GQ*RV*G (SEQ ID NO: 199510); K*ASG*A (SEQ ID NO: 199921); K*SN*AA (SEQ ID NO: 199922); KY*T*TG (SEQ ID NO: 199923);
P*R*NGA (SEQ ID NO: 199924); QPR*M*G (SEQ ID NO: 199925); RPQ**TG (SEQ ID NO: 199926); T**VNRG (SEQ ID NO: 199927); and TPRS**G (SEQ ID NO: 199928); or polynucleotides encoding the same, where “*” represents any amino acid and square brackets denote a list of amino acids that may occur at a position, wherein a viral particle having a capsid comprising the motif has increased biodistribution to lung relative to a control viral particle;
wherein the library comprises an amino acid sequence motif selected from the group consisting of:
[RNGKS] [KPTV] [NQGS] * [ANQST] *A; K** [NST] [QS] [AQK] [DS];
[NGMPSTW] [RNQGK] [QGKMSTV] ** [AQKST] [ARNDGKP];
[RGKP] [QKPT] ** [NQST] [NQGMT]A; [NMPS] * [NQKS] [AQGHK] * [QGS] [RIPSV];
[RNLPS] [RGHKS] * [AQGKPT] * [ANGS] [ARGP];
[RQGMS] [NDQK] **[NGKS] [NQGS] [ARQKPT];
[RNQGKPSW] [ARNGKPT] [ANQGST] [ANQGKST] ** [AGKMPS];
K[NQT] * [AGS] *Q[AS]; [RKT] [KT] [NQV] ** [ARQKT] [DPS];
[RNGKMPS] [RQST] [NGHKT] * [NQGTV] * [AKYV]; [GT] ** [QS] [SY] [NM] [RK];
[KFSY] * [ANHKP] [NQS] [AGST] * [RMST]; [NKP] [ARGK] [GHPS] ** [AG] [RMST];
[GP]K* [NS] * [AQ] [AG]; [RKP] ** [QT] [QGS] [NG]A;
[QGKP] * [NKS] [KPST] [AS] *A; [GK] * [NQHT] * [NGPV] [QLMTV] [AQS];
[GK] * [NKPS] [ANGTV] * [AQT] [AM]; K*TS* [AQ]A (SEQ ID NO: 199929);
N* [ST] [GK] [RN] *A; [GK]Q [QGT] [QGT] ** [AM]; K*NN*NP (SEQ ID NO: 199930); K[PT] [QS] ** [AQS]A; [QKP] [NST] [KT] ** [GT]A;
[KV] [QT] [QK] *S* [AS]; [RNQGKS] [AQGKPT] * [NGKST] [ANGKTV] * [AMSV];
KN*G*SA (SEQ ID NO: 199570); T**GPGR (SEQ ID NO: 199931); K*TG*KD (SEQ ID NO: 199932); K**G[AT] [NQ]A; [QT] [NQK]K[DT] **A;
[NK]K[ND] *G*A; N*QKT*G (SEQ ID NO: 199875); K[GT]N**[GT]A; GKN**SA (SEQ ID NO: 199933); K[ST] ** [AK] [ND] [DP]; PK* [NGP] [ANS] *A; TK*V*QG (SEQ ID NO: 199893); K*SGT*A (SEQ ID NO: 199896); N*K* [NS]N[GP];
[NG]KT*G* [AM]; [MP] [GK]T*S* [RG]; [NP] *KS* [GS]A; KP** [AG] [AG]S;
SK*S*QP (SEQ ID NO: 199881); S*T*GSP (SEQ ID NO: 199934); HQKP**L (SEQ ID NO: 199935); N*N*GTA (SEQ ID NO: 199936); SRTP**A (SEQ ID NO: 199937); PR*QQ*P (SEQ ID NO: 199903); P*KG*SA (SEQ ID NO: 199880); VK*NN*P (SEQ ID NO: 199938); K*N*SIA (SEQ ID NO: 199939);
KPT*P*S (SEQ ID NO: 199940); T*KS*TP (SEQ ID NO: 199905); N*KST*A (SEQ ID NO: 199941); R*TS*SP (SEQ ID NO: 199519); K*N*GNA (SEQ ID NO: 199942); KTN**GS (SEQ ID NO: 199943); N**GRTA (SEQ ID NO: 199944); NKPP**A (SEQ ID NO: 199945); SK**TNS (SEQ ID NO: 199946);
SK*V*TS (SEQ ID NO: 199947); and TK*QN*A (SEQ ID NO: 199948);
TKQ*S*S (SEQ ID NO: 199949); or polynucleotides encoding the same, where “*” represents any amino acid and square brackets denote a list of amino acids that may occur at a position, wherein a viral particle having a capsid comprising the motif has increased biodistribution to spinal cord relative to a control viral particle;
wherein the library comprises an amino acid sequence motif selected from the group consisting of: GAG**MR (SEQ ID NO: 199950); LQSN**R (SEQ ID NO: 199951); NN*TT*R (SEQ ID NO: 199952); NQ*Q*TK (SEQ ID NO: 199953); and RVG**DK (SEQ ID NO: 199954); Y*AG*SR (SEQ ID NO: 199955); or polynucleotides encoding the same, where “*” represents any amino acid and square brackets denote a list of amino acids that may occur at a position, wherein a viral particle having a capsid comprising the motif has increased biodistribution to kidney relative to a control viral particle;
wherein the library comprises an amino acid sequence motif selected from the group consisting of:
WNG**QK (SEQ ID NO: 199956); [DY] [KT] [FS] ** [QK] [GK];
[QSWY] [NIKM] [NGP] [NQH] ** [KY]; IKH*R*E (SEQ ID NO: 199957);
[IT] [LK] * [DH] * [KV] [RN]; [KF] * [KS] * [PT] [NY] [LM]; YKD**PR (SEQ ID NO: 199958); HN*N*GK (SEQ ID NO: 199959); KFK*E*Y (SEQ ID NO: 199960); NGQ**AR (SEQ ID NO: 199961); QI*H*AK (SEQ ID NO: 199962);
R**NGIQ (SEQ ID NO: 199963); REKP**M (SEQ ID NO: 199964); Y*H*MKG (SEQ ID NO: 199965); or polynucleotides encoding the same, where “*” represents any amino acid and square brackets denote a list of amino acids that may occur at a position, wherein a viral particle having a capsid comprising the motif has increased liver transduction efficiencies relative to a control viral particle; or
wherein the library comprises an amino acid sequence motif selected from the group consisting of:
[\hat{ }ADCEHILKW] [ARNQGKST] [ARNQGKPTV] [ARNQKPSTV] **[NGPS];
[RNQKSV] * [ANQGPST] * [ARNQKT] [ANQGKT] [EGPS];
[\hat{ }ANDCEHILFW] * [ANQGKPST] [ARDQGKPST] [ARNQGKSV] * [ADGS];
[NQGFST] * [NGKT] * [ARNQKST] [ANQGKSTV] [GP];
[NQGKMSTY] [RQGKPST] * [ARDQGKSTV] [\hat{ }ADCEHILFWY] *G;
[RNGKMPT] ** [AQGKST] [ARNQGSV] [AQGKMST] [EGPS];
[NQGKFST] [ARQGKPST] [ADQGKPST] *[ARNQIKST] *G;
[RNQGHKPTV] * [ARNQGMPST] [ARNQKST] * [ANQGKMS] [DEGPSV];
[\hat{ }ARDCEHILKW]K* [ANQGST] * [ANQIKMSTV] [DGPS];
[RNGKTV] [ANQGSV] ** [ARGKSTV] [AQGKMS]G;
[\hat{ }ADCQEHFWY] [ARNQGKMPS] [\hat{ }CEHILKFWYV] ** [ANQGKMSV] [NDGPS];
[RNQGMPSTV] [ARDQGKPST] [NQGKST] [ANGKPST] ** [APS];
[RNGKMST] [RGKST] [RNQMT] **[ARNQMST] [ADPS];
[RNQGMPSTV] *[AQGKST] [AQGKPSTV] *[NQGKSTV] [GPSV];
[NQST] * [QKT] * [GKT] [NQMS] [GP]; [RNQKSTY] [RKPT] [ANQGST] ** [ANQGT]G;
[RNQMTYV] [NQKP] [RNQGS]G**G; [RGKMT] ** [NT] [RNGKS] [ANGT]G;
[RNGT] * [ANGKT] [ARNKMST] [RNQGKS] *G;
[RQGKMPST] [NQKPST] ** [ARNKST] [\hat{ }RCQEHILFPW] [DGPS];
[RKS] [NQGPST] [ANQGKT] [ANQGT] ** [APS];
[RNMPSTY] [ARNQGKSV] * [ANQKST] * [ANGKSTV] [GPS];
[NQGKPSTV] [ARQGKST] * [AQGKPST] [ANKSTV] * [APS];
[RNQGKTY] [RNQGLPST] [ARNQKPST] * [ANQSTV] * [APS];
[NQGFST] [ANGSV] [RK] ** [NQSV] [GP]; K[NQGLKST] [NDQGMPT] [NQST] **G;
[NQGKMFPST] [RQGKPS] [RDQKPST] *G* [AM];
[RQGKMST] [QKP] [NDQGS] * [ANKST] * [ADEPS]; K* [QT] * [QG] [QMT]A;
[NKMST] [ANGKP] [NGKP] * [ARNMPST] *G; [RNQPST] [RNK] ** [ARNGS] [ANGSTV]A;
[QGS]K** [GT] [AMT] [APS]; [NQGMST] [NK] ** [AQGSV] [QKMFS] [RDGPS];
[RDKST] [NQKP] *[AGKS] *[AGSTV]A;
[NQGKSTV] [RNQGK] * [NQGKS] [ANQKST] * [GPS]; [QK] [NKSV] [NDGKT] [ST] **A;
[RKMT] ** [GPS] [AT] [ANGS]G; [RKM] * [NPST] [AGT]T* [GV];
[RKV] ** [NGT] [AGST] [NQG]A; [QKST] [QKT] * [NG]G* [DG];
[RQGKT] * [RNQGPST] [RQGST] * [QGMS]A; [KV] *N* [NS] [AISV]A;
[NQKPT] [RKP] * [AGST]G* [AP]; [RNQKS] [ANMPSV] [RNQK] *G*G;
[RNST] * [QKMST] [RGS] [ARST] * [AP]; [NKS] [KST] [NK] * [NG] * [AS];
[RNGT] * [RQGKST] [NKST] [RNMT] * [AG]; [RKT] [RQT] ** [GT] [NQG]A;
[RNIK] [RNQV] *[AGS] *[NQGT] [GS]; [RGKP] [NKT] [NQTV] **G[AS];
[RNQST] [RQK]S [ANQKS] **G; R[NST] * [ANQG] [ANTV] *G; [QK] [QT] **N[GS]A;
[QPT] [RGK] *N[AKS] *A; [QGT] [QGT]K** [AT]A; RP* [NG] [NT] *G;
[FT]K**NQ[NG]; [NGS] *[GKS] [RGKS] *[AGST]A;
[ANGS] [RGK] * [NKPT] * [ANGS]A; [MT]K* [QM] *QA; [KP] [NP] [KS] ** [AG]A;
[NM] * [NK] [NK] * [QT]G; [RK] **NT [GS]G; RN* [AN] [AN] *A;
N** [NQS] [RGK] [QGS]A; [KT] * [NGT] * [GSV] [ANST] [GS];
K* [NQM] [NG] [ANG] * [GP]; [NQ] * [AGK]G [QT] *G; QKN**SA (SEQ ID NO: 199966); Q*[NK]G* [AN]G; K[NT] *[AG] *QA; F*KT*QA (SEQ ID NO: 199967);
[NST]K[GPT] *G*G; RP*[ST]G*A (SEQ ID NO: 200024); PTK**SG (SEQ ID NO: 199968); R**QQNA (SEQ ID NO: 199969); QPK**GG (SEQ ID NO: 199970); KN*ST*A (SEQ ID NO: 199971); RQ*PT*A (SEQ ID NO: 199515);
K[NQG] *N* [AT]G; R[AN] [QS] * [AT] *G; [NQ] [RN] [GKP] [NQS] **A;
KP[GP]G**G (SEQ ID NO: 199972); KPS[NGS] **[AM]; RS*PG*A (SEQ ID NO: 199973); K*[NQT]ST*A (SEQ ID NO: 199974); [RK] *[NS] [AG]T*A;
KPSQ**P (SEQ ID NO: 199975); [NKM] *N* [GS] [NT]A; RSS*G*A (SEQ ID NO: 199976); RN*PG*G (SEQ ID NO: 199977); K[NQ] *[GS] *SG;
[NT] *K* [QPS] [NGS]A; N*RNT*P (SEQ ID NO: 199978); K* [NG]A* [QG]A;
K*GNQ*A (SEQ ID NO: 199979); S**STQS (SEQ ID NO: 199980);
NK**[GT]GG (SEQ ID NO: 199981); RN**NQA (SEQ ID NO: 199982);
NR**GQA (SEQ ID NO: 199983); K* [NT] [NS] *AA; [RK] [NT] *N*QA; NR*N*QG (SEQ ID NO: 199984); N**STMA (SEQ ID NO: 199669); N*KA*AG (SEQ ID NO: 199985); NKQ*A*A (SEQ ID NO: 199986); MKQ*G*G (SEQ ID NO: 199987); RGN*T*G (SEQ ID NO: 199988); K*NSS*P (SEQ ID NO: 199989);
K*NNP*A (SEQ ID NO: 199990); MKN*G*G (SEQ ID NO: 199991); RN*NG*G (SEQ ID NO: 199992); G*K*NVA (SEQ ID NO: 199993); K*GG*AG (SEQ ID NO: 199626); N**GTKG (SEQ ID NO: 199994); NN**NKG (SEQ ID NO: 199995); RDK**GG (SEQ ID NO: 199996); RQ*N*QG (SEQ ID NO: 199997);
SRTT*NG (SEQ ID NO: 199576); and TSKG**G (SEQ ID NO: 199998); or polynucleotides encoding the same, where “*” represents any amino acid, square brackets surrounding a list of amino acids not preceded by the symbol “\hat{ }” denote a list of amino acids that may occur at a position, and square brackets surrounding a list of amino acids preceded by the symbol “\hat{ }” denote a list of amino acids that may not occur at a position, wherein a viral particle having a capsid comprising the motif has reduced spleen biodistribution relative to a control viral particle.
wherein the capsid polypeptides are capable of forming viral particles with two or more of the following traits: 1) binding to liver cell; 2) transducing liver cell; and 3) biodistributing to the liver of an organism, and wherein the library comprises an amino acid sequence motif selected from the group consisting of:
[NQGIKT] [DGKFPS] [NEHKS] *[RDEHY] *[RQEKY];
[RDS] [DQE] *[RPT] [RNK] *[RKV];
[RDKMSY] ** [RNKSV] [ARDEP] [ARNDEIKS] [ARNKMYV]; Q [RE] ** [RK] [DI] [IK];
[NMT] [RDK] * [ARGTV] * [RDSY] [ANKT]; [GLFTY] * [HKY] * [NMPY] [NKS] [AQGL];
[ANGST] * [ADEG] [RNGH] * [RK] [RNESV];
[REKFY] [ARLTYV] [DGHKP] [RDHIKS] **[ARNEIFT];
[ANQKT] [ARKPS] [RGKM] ** [RSV] [DEGY]; [RMS] * [EK] * [RV] [DPT] [DKT];
[QY] *G*V[RK]G; [IPT] [DEGP]R[AQHV] **[ANK]; IRA**EK (SEQ ID NO: 199999); [DK] *R[NE] * [KT] [AQ]; [QI] [RD] *K[MP] * [RE]; D*KPR*Q (SEQ ID NO: 200000); [ST] [QY] [EK] * [RS] * [NK]; [EY] [ET] *K*RN; ILH**KN (SEQ ID NO: 200001); V*RSD*K (SEQ ID NO: 200002);
[ADE] [ADG] * [GK] *K[LMY]; [KV] [QLT]R* [DS] * [GI]; TD*KR*L (SEQ ID NO: 200003); [RELF] [EK] ** [AQ] [RD] [DGKS]; EK**TRQ (SEQ ID NO: 200004);
[MV] *R* [SV] [AD] [GK]; IL*H*KN (SEQ ID NO: 200005); V*KG*YN (SEQ ID NO: 200006); G**HKQL (SEQ ID NO: 200007); Y*SH*KG (SEQ ID NO: 200008); D[NK] [RH] [KV] ** [RV]; I*RS*TG (SEQ ID NO: 199859);
[IK] [GP]R*[DT] *[AG]; QGR*L*A (SEQ ID NO: 200009); YTS**KG (SEQ ID NO: 200010); G**ETRK (SEQ ID NO: 200011); V*RQ*AG (SEQ ID NO: 199782); DL*K*RA (SEQ ID NO: 200012); D*RPK*V (SEQ ID NO: 200013);
DR**VKQ (SEQ ID NO: 200014); G**TEKK (SEQ ID NO: 200015); I*R*MRE (SEQ ID NO: 200016); NDMR**K (SEQ ID NO: 200017); NE*KR*V (SEQ ID NO: 200018); QAR*E*R (SEQ ID NO: 200019); V*RS*GG (SEQ ID NO: 199791); YTE**KK (SEQ ID NO: 200020); or polynucleotides encoding the same, where “*” represents any amino acid and square brackets denote a list of amino acids that may occur at a position.

32-80. (canceled)

81. A composition comprising an adeno-associated virus (AAV) capsid of claim 1.

82. (canceled)

83. A method for screening a library of adeno-associated virus (AAV) capsid polypeptides for a trait of interest, the method comprising:
A) administering to an organism or contacting a population of cells with AAV particles comprising the library of claim 14;
B) identifying in the library those particles demonstrating the trait of interest in the organism and/or in/on the cells.

84-89. (canceled)

90. A viral particle identified by the method of claim 19.

91. A kit suitable for use in the method of claim 20, wherein the kit comprises adeno-associated virus (AAV) particles comprising the capsid polypeptides of claim 8, or polynucleotides encoding the same.
